(19) United States
(12) Patent Application Publication
(10) Pub. No.: US 2025/0170177 A1
(43) Pub. Date: May 29, 2025

(54) METHODS AND COMPOSITIONS FOR TREATING CANCER

(71) Applicant: THE REGENTS OF THE UNIVERSITY OF CALIFORNIA, Oakland, CA (US)

(72) Inventors: Yvonne Yu-Hsuan CHEN, Los Angeles, CA (US); Andrew J. HOU, Los Angeles, CA (US)

(73) Assignee: THE REGENTS OF THE UNIVERSITY OF CALIFORNIA, Oakland, CA (US)

(21) Appl. No.: 18/841,167

(22) PCT Filed: Feb. 24, 2023
(86) PCT No.: PCT/US2023/063262

§ 371 Date: Aug. 23, 2024

Related U.S. Application Data
(60) Provisional application No. 63/313,899, filed on Feb. 25, 2022.
(60) Provisional application No. 63/339,836, filed on May 9, 2022.
(60) Provisional application No. 63/348,592, filed on Jun. 3, 2022.
(60) Provisional application No. 63/411,433, filed on Sep. 29, 2022.

(51) Int. Cl. A61K 35/17; A61K 40/11; A61K 40/31; A61K 40/42; A61P 35/00; C07K 14/54; C07K 14/705; C07K 14/725; C07K 16/22; C07K 16/28; C07K 16/30

(52) U.S. Cl. CPC A61K 35/17; A61K 40/11; A61K 40/31; A61K 40/4217; A61K 40/4226; A61K 40/4229; A61K 40/4258; A61P 35/00; C07K 14/5437; C07K 14/7051; C07K 14/70521; C07K 14/70578; C07K 16/22; C07K 16/2863; C07K 16/3084; A61K 2239/13; A61K 2239/29; A61K 2239/47; C07K 2317/53; C07K 2317/565; C07K 2317/622; C07K 2319/02; C07K 2319/03

(57) ABSTRACT

Adoptive T-cell therapy has shown tantalizing promise as a cancer treatment strategy, with several clinical trials reporting that T cells expressing chimeric antigen receptors (CARs) can eradicate tumors in patients with relapsed disease. However, CAR-T cells rely on receptor-mediated recognition of surface-bound antigens that are seldom tumor-exclusive, resulting in severe on-target, off-tumor toxicities that have led to patient deaths in clinical trials. There is a growing consensus that the lack of suitable antigens poses a major obstacle to the broad application of engineered tumor-targeting T cells. The ability to overcome T cells' reliance on surface antigen presentation and interrogate intracellular disease signatures would significantly expand the pool of detectable tumor markers and improve tumor-targeting specificity. Here, we present a novel strategy to reprogram T-cell-mediated cytotoxicity to interrogate intracellular disease signatures. We have engineered a switchable form of the cytotoxic protein Granzyme B (GrB) that is produced and delivered by T cells into target cells, but becomes active if and only if a tumor-associated protease is present inside the target cell. As a proof of concept, we have developed a GrB switch that responds to Sentrin-specific protease 1 (SENP1), an oncoprotein known to be overexpressed in prostate, pancreatic, and thyroid oncocytic tumor cells. We demonstrate that this GrB switch, termed cytoplasmic oncoprotein verification evaluator and response trigger (COVERT), is efficiently expressed and packaged by human T cells and properly trafficked to the immunological synapse between T cells and target cells. Furthermore, we show that COVERT is produced as an enzymatically inert protein that is activated by SENP1 in a dose-dependent manner. Finally, we describe designs to adapt COVERT into a modular platform technology that will expand the repertoire of candidate target antigens. We envision that COVERT can be utilized in combination with existing CAR technology to improve the tumor-targeting precision of cell-based immunotherapy.

Copyright ® American Institute of Chemical Engineers. All rights reserved.

This application claims priority of U.S. Provisional Patent Application No. 63/313,899, filed Feb. 25, 2022; U.S. Provisional Patent Application No. 63/339,836, filed May 9, 2022; U.S. Provisional Patent Application No. 63/348,592, filed Jun. 3, 2022; and U.S. Provisional Patent Application No. 63/411,433, filed Sep. 29, 2022, each of which is hereby incorporated by reference in its entirety.

The application contains a Sequence Listing which has been filed electronically in compliance with ST.26 format and is hereby incorporated by reference in its entirety. Said Sequence Listing, created on Feb. 24, 2023 is named UCLAP0163WO.xml and is 226,700 bytes in size.

### BACKGROUND

### 1. Field of the Invention

This invention relates generally to the fields of molecular biology and immunotherapy.

### II. Background

CAR T cell therapy is a type of immunotherapy used to fight cancer with altered immune cells. These specially altered white blood cells, called T cells, are modified to find and attack cancer cells in the body. CAR T cell therapy is now FDA-approved for patients with acute lymphoblastic leukemia, non-Hodgkin lymphoma, and multiple myeloma. There is a need in the art to develop CAR-T cell therapies for other cancer.

### SUMMARY OF THE DISCLOSURE

The current disclosure provides a need in the art by providing for novel multi-specific CAR molecules for the treatment of certain cancers. Accordingly, the disclosure related to methods for stimulating an immune response in a subject and/or for treating a subject with cancer comprising administering to the subject an effective amount of the composition comprising cells comprising a heterologous nucleic acid encoding for a polypeptide, wherein the polypeptide comprises: i.) a multi-specific chimeric antigen receptor (CAR) comprising an IL13 polypeptide, a TGF-β binding region, a peptide spacer, a transmembrane domain, and a cytoplasmic region comprising a co-stimulatory region and a primary intracellular signaling domain; ii.) a multi-specific chimeric antigen receptor comprising an IL13Rα binding region, a tumor antigen binding region, a peptide spacer, a transmembrane domain, and a cytoplasmic region comprising a co-stimulatory region and a primary intracellular signaling domain; wherein the tumor antigen binding region comprises a GD2 or EGFRvIII binding region; iii.) a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises SEQ ID NO:48 (HCDR1), SEQ ID NO: 49 (HCDR2); and SEQ ID NO:50 (HCDR3) and the VL region comprises SEQ ID NO: 51 (LCDR1), SEQ ID NO:52 (LCDR2); and SEQ ID NO:53 (LCDR3); iv.) a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47; or v.) a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an EGFRvIII binding region; wherein the cancer is selected from malignant glioma, diffuse midline glioma, neuroblastoma, sarcoma, osteosarcoma, diffuse intrinsic pontine glioma, and melanoma. The cancer may exclude malignant glioma, diffuse midline glioma, neuroblastoma, sarcoma, osteosarcoma, diffuse intrinsic pontine glioma, and melanoma.

The polypeptide may comprise a multi-specific chimeric antigen receptor comprising an IL13Rα binding region, a tumor antigen binding region, a peptide spacer, a transmembrane domain, and a cytoplasmic region comprising a co-stimulatory region and a primary intracellular signaling domain; wherein the tumor antigen binding region comprises a GD2 or EGFRvIII binding region.

The polypeptide may comprise a multi-specific chimeric antigen receptor comprising a IL13 polypeptide of SEQ ID NO:4 or 20, a tumor antigen binding region, a peptide spacer, a transmembrane domain, and a cytoplasmic region comprising a co-stimulatory region and a primary intracellular signaling domain; wherein the tumor antigen binding region comprises a GD2 or EGFRvIII binding region.

The polypeptide may comprise a multi-specific chimeric antigen receptor (CAR) comprising an IL13 polypeptide of SEQ ID NO:4 or 20, a TGF-β binding region, a peptide spacer, a transmembrane domain, and a cytoplasmic region comprising a co-stimulatory region and a primary intracellular signaling domain.

The polypeptide may comprise a multi-specific chimeric antigen receptor (CAR) comprising an IL13 polypeptide, a TGF-β binding region, a peptide spacer, a transmembrane domain, and a cytoplasmic region comprising a co-stimulatory region and a primary intracellular signaling domain.

The polypeptide may comprise a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3). The polypeptide may comprise a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47.

The polypeptide may comprise a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an EGFRvIII binding region.

The cancer may be defined as glioma. The glioma may be midline glioma or diffuse midline glioma. The glioma may be classified as or further classified as an astrocytoma, oligodendroglioma, mixed glioma, or ependymoma. The cancer may comprise neuroblastoma, sarcoma, osteosarcoma, diffuse intrinsic pontine glioma, or melanoma. The cancer may comprise neuroblastoma. The cancer may comprise sarcoma. The cancer may comprise osteosarcoma. The cancer may comprise diffuse intrinsic pontine glioma. The cancer may comprise melanoma. Midline glioma or diffuse midline glioma may be also be excluded as a patient population in the methods. The methods may also exclude patients having the following classes of cancer: astrocytoma, oligodendroglioma, mixed glioma, or ependymoma. The methods may exclude treatment of a subject or patient with neuroblastoma, sarcoma, osteosarcoma, diffuse intrinsic pontine glioma, or melanoma. The methods may exclude treatment of patients and subjects with diffuse intrinsic pontine glioma or melanoma.

The polypeptide may comprise one or more tumor antigen binding regions. The antigen binding region may comprise a GD2 binding region. The structure of GD2 is known in the art. GD2 is a disialoganglioside belonging to b-series ganglioside. It comprises five monosaccharides linked to ceramide, with the carbohydrate sequence of GalNAcβ1-4 (NeuAcα2-8NeuAcα2-3)Galβ1-4Glcβ1-1. GD2 binding regions, such as anti-GD2 antibody binding regions are known in the art. The GD2 binding region may comprise an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3).

The tumor antigen binding region may comprise a EGFRVIII antigen binding region. EGFRvIII is a variant of EGFR that lacks amino acids 6-273, and deletion of those 268 amino acids creates a junction site with a new glycine residue between amino acids 5 and 274. The EGFRvIII binding region may comprise an anti-EGFRvIII scFv having a variable heavy (VH) and variable light (VL) region, wherein the VH region comprises SEQ ID NO:40 (HCDR1), SEQ ID NO:41 (HCDR2); and SEQ ID NO:42 (HCDR3) and the VL region comprises SEQ ID NO: 43 (LCDR1), SEQ ID NO:44 (LCDR2); and SEQ ID NO:45 (LCDR3).

The polypeptides may comprise a TGF-β binding region. The TGF-β binding region may comprise a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

LCDR1 of a GD2, EGRvIII, or TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 51, 43, or 34, respectively. LCDR2 of a GD2, EGRVIII, or TGF-β binding region may comprise an amino acid sequence with, with at least, with at most, or about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:52, 44, or 35, respectively. LCDR3 of a GD2, EGRVIII, or TGF-β binding region may comprise an amino acid sequence with, with at least, with at most, or about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:53, 45, or 36, respectively. HCDR1 of a GD2, EGRVIII, or TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:48, 40, or 31, respectively. HCDR2 of a GD2, EGRvIII, or TGF-β binding region may comprise an amino acid sequence with, with at least, with at most, or about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:49, 41, or 32, respectively. HCDR3 of a GD2, EGRvIII, or TGF-β binding region may comprise an amino acid sequence with, with at least, with at most, or about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:50, 42, or 33, respectively.

The GD2 binding region may comprise a VH with an amino acid sequence having at least 80% sequence identity to SEQ ID NO:46 and/or a VL with an amino acid sequence having at least 80% sequence identity to SEQ ID NO:47. The GD2 binding region may comprise a VH with the amino acid sequence of SEQ ID NO:46 and/or a VL with the amino acid sequence of SEQ ID NO:47. The GD2 binding region may comprise a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:46 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 47. The GD2 binding region may also be one that comprises an anti-GD2 scFv having an amino acid sequence with at least 80% sequence identity to SEQ ID NO:26. The GD2 binding region may comprise an anti-GD2 scFv having the amino acid sequence of SEQ ID NO:26. Also described are GD2 binding regions that comprise an anti-GD2 scFv having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:26. The GD2 binding region may comprise a binding region that binds to membrane-bound GD2 antigen. The GD2 binding region may comprise a binding region that binds to soluble GD2 antigen. The GD2 binding region may comprise a binding region that binds to membrane-bound and soluble GD2 antigen.

The polypeptides may comprise a TGF-β binding region. The TGF-β binding region may comprise a scFv having a variable heavy (VH) and variable light (VL) region, wherein the VH region comprises SEQ ID NO:56 (HCDR1), SEQ ID NO:57 (HCDR2); and SEQ ID NO:58 (HCDR3) and the VL region comprises SEQ ID NO:59 (LCDR1), SEQ ID NO:60 (LCDR2); and SEQ ID NO:61 (LCDR3). The TGF-β binding region may comprise a scFv having a variable heavy (VH) and variable light (VL) region, wherein the VH region comprises SEQ ID NO: 64 (HCDR1), SEQ ID NO:65 (HCDR2); and SEQ ID NO:66 (HCDR3) and the VL region comprises SEQ ID NO:67 (LCDR1), SEQ ID NO:68 (LCDR2); and SEQ ID NO:69 (LCDR3).

LCDR1 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:59 or 67. LCDR2 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:60 or 68. LCDR3 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:61 or 69. HCDR1 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:56 or 64. HCDR2 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO: 57 or 65. HCDR3 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:58 or 66. The TGF-β binding region may comprise a VH with an amino acid sequence having at least 80% sequence identity to SEQ ID NO:54 and/or a VL with an amino acid sequence having at least 80% sequence identity to SEQ ID NO:55. The TGF-β binding region may comprise a VH with the amino acid sequence of SEQ ID NO:54 and/or a VL with the amino acid sequence of SEQ ID NO: 55. The TGF-β binding region may comprise a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:54 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:55. The TGF-β binding region may comprise a VH with an amino acid sequence having at least 80% sequence identity to SEQ ID NO:62 and/or a VL with an amino acid sequence having at least 80% sequence identity to SEQ ID NO:63. The TGF-β binding region may comprise a VH with the amino acid sequence of SEQ ID NO:62 and/or a VL with the amino acid sequence of SEQ ID NO:63. The TGF-β binding region may comprise a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:62 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:63. LCDR1 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:34. LCDR2 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:35. LCDR3 of a TGF-binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:36. HCDR1 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:31. HCDR2 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:32. HCDR3 of a TGF-β binding region may comprise an amino acid sequence with, with at least, with at most, or with about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:33. The TGF-β binding region may comprise a VH with an amino acid sequence having at least 80% sequence identity to SEQ ID NO:29 and/or a VL with an amino acid sequence having at least 80% sequence identity to SEQ ID NO:30. The TGF-binding region may comprise a VH with the amino acid sequence of SEQ ID NO:29 and/or a VL with the amino acid sequence of SEQ ID NO:30. The TGF-β binding region may comprise a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:29 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:30. The TGF-β binding region may comprise a VH with the amino acid sequence of SEQ ID NO:29 and/or a VL with the amino acid sequence of SEQ ID NO:30.

The EGFRvIII binding region may comprise a VH with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:38 and/or a VL with an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:39. The EGFRvIII binding region may comprise a VH with an amino acid sequence having at least 80% sequence identity to SEQ ID NO:38 and/or a VL with an amino acid sequence having at least 80% sequence identity to SEQ ID NO:39. The EGFRvIII binding region may comprise a VH with the amino acid sequence of SEQ ID NO:38 and/or a VL with the amino acid sequence of SEQ ID NO:39. The EGFRvIII binding region may comprise a VH with the amino acid sequence of SEQ ID NO:38 and/or a VL with the amino acid sequence of SEQ ID NO:39. The EGFRvIII binding region may comprise an anti-EGFRvIII scFv having an amino acid sequence with at least 80% sequence identity to SEQ ID NO:27. The EGFRvIII binding region may comprise an anti-EGFRvIII scFv having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:27. The EGFRvIII binding region may comprise an anti-EGFRvIII scFv having the amino acid sequence of SEQ ID NO:27. The EGFRvIII binding region comprises a binding region that binds to membrane-bound EGFRVIII antigen. The EGFRvIII binding region may comprise a binding region that binds to soluble EGFRvIII antigen. The EGFRvIII binding region may comprise a binding region that binds to membrane-bound and soluble EGFRvIII antigen.

The TGF-β binding region comprises an anti-TGF-β scFv having an amino acid sequence with at least 80% sequence identity to SEQ ID NO:11. The TGF-β binding region may comprise an anti-TGF-scFv having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:11. The TGF-β binding region may comprise an anti-TGF-β scFv having the amino acid sequence of SEQ ID NO:11.

The GD2 binding region may comprise an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47. The EGFRvIII binding region may comprise an anti-EGFRvIII scFv having a variable heavy (VH) and variable light (VL) region, wherein the VH region comprises the HCDR1, HCDR2; and HCDR3 from the VH of SEQ ID NO:38 and the VL region comprises LCDR1, LCDR2; and LCDR3 from the VL of SEQ ID NO:39. The TGF-β binding region may comprise a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30. The TGF-β binding region may comprise a scFv having a variable heavy (VH) and variable light (VL) region, wherein the VH region comprises the HCDR1, HCDR2; and HCDR3 from the VH of SEQ ID NO:54 and the VL region comprises LCDR1. LCDR2; and LCDR3 from the VL of SEQ ID NO:55. The TGF-β binding region may comprise a scFv having a variable heavy (VH) and variable light (VL) region, wherein the VH region comprises the HCDR1. HCDR2; and HCDR3 from the VH of SEQ ID NO:62 and the VL region comprises LCDR1, LCDR2; and LCDR3 from the VL of SEQ ID NO:63. The HCDR1, HCDR2. HCDR3, LCDR1, LCDR2, and LCDR3 may be determined by the Kabat method. The HCDR1, HCDR2, HCDR3, LCDR1, LCDR2, and LCDR3 may be determined by the IMGT method. The HCDR1, HCDR2, HCDR3, LCDR1, LCDR2, and LCDR3 may be determined by the Chothia method. The HCDR1, HCDR2, HCDR3, LCDR1, LCDR2, and LCDR3 may be determined by the paratome method.

“Single-chain Fv” or “scFv” antibody fragments comprise at least a portion of the VH and VL domains of an antibody, such as the CDRs of each, wherein these domains are present in a single polypeptide chain. It is contemplated that an scFv includes a CDR1, CDR2, and/or CDR3 of a heavy chain variable region and a CDR1, CDR2, and/or CDR3 of a light chain variable region. It is further contemplated that a CDR1, CDR2, or CDR3 may comprise or consist of a sequence set forth in a SEQ ID NO provided herein as CDR1, CDR2, or CDR3, respectively. A CDR may also comprise 1, 2, 3, 4, 5, 6, 7, 8, 9, 10, 11, 12, 13, 14, 15, 16, 17, 18, 19, 20, 21, 22, 23, or more contiguous amino acid residues (or any range derivable therein) flanking one or both sides of a particular CDR sequence; therefore, there may be one or more additional amino acids at the N-terminal or C-terminal end of a particular CDR sequence, such as those shown in SEQ ID NOS: 31-36, 40-45, 48-53, 56-61, or 64-69.

The multi-specific polypeptides may comprise an IL13Rα binding region and a tumor antigen binding region. The polypeptide may comprise a chimeric antigen receptor (CAR), wherein the CAR comprises in order from amino-proximal end to carboxy-proximal end: an IL13Rα binding region, a tumor antigen binding region, a peptide spacer, a transmembrane domain, and a cytoplasmic region comprising a co-stimulatory region and a primary intracellular signaling domain.

The multi-specific polypeptides may comprise an IL13 polypeptide and a tumor antigen binding region. The polypeptide may comprise a chimeric antigen receptor (CAR), wherein the CAR comprises in order from amino-proximal end to carboxy-proximal end: an IL13 polypeptide, a tumor antigen binding region, a peptide spacer, a transmembrane domain, and a cytoplasmic region comprising a co-stimulatory region and a primary intracellular signaling domain.

It is contemplated that the IL13Rα binding region or IL13 polypeptide may be amino proximal to the tumor antigen binding region. The IL13Rα binding region or IL13 polypeptide may be carboxy proximal to the tumor antigen binding region. The TGF-binding region may be amino proximal to the tumor antigen binding region or the TGF-β binding region may be carboxy proximal to the tumor antigen binding region. The IL13Rα binding region or IL13 polypeptide may be amino proximal to the TGF-binding region or the IL13Rα binding region or IL13 polypeptide may be carboxy proximal to the TGF-β binding region. It is contemplated that the TGF-β binding region may be adjacent to the IL13Rα binding region or IL13 polypeptide, meaning that there are no intervening binding regions, although any two binding regions that are adjacent may be separated by a linker region. The IL13Rα binding region or IL13 polypeptide may be adjacent to the tumor antigen binding region, or the tumor antigen binding region may be adjacent to the TGF-β binding region.

The polypeptide may comprise or further comprise one or more linkers separating regions. For example, the polypeptide may comprise a linker between two binding regions, such as a linker between the IL13Rα binding region or IL13 polypeptide and the tumor antigen binding region. The polypeptide may comprise a linker between the TGF-β binding region and the tumor antigen binding region, and/or between the IL13Rα binding region or IL13 polypeptide and the TGF-β binding region. The polypeptide may comprise a tri-specific CAR comprising TGF-β binding region. The tri-specific CAR may comprise a TGF-β binding region, an IL13Rα binding region or IL13 polypeptide, and a tumor antigen binding region.

The IL13Rα binding region may be a IL13Rα2 binding region. The IL13 polypeptide may exclude an IL13 polypeptide consisting of amino acids 3-114 of SEQ ID NO: 4. The IL13 polypeptide may exclude an IL13 polypeptide consisting of amino acids 11-122 of SEQ ID NO:4. The IL13 polypeptide may comprise the C-terminal 112 amino acids of SEQ ID NO:4 or 20 and at least one additional amino acid at the N terminus. The IL13 polypeptide may comprise the C-terminal 112 amino acids of SEQ ID NO:4 or 20 and at least, at most, or exactly 1, 2, 3, 4, 5, 6, 7, 8, 9, or 10 additional amino acids at the N-terminus. Additionally or alternatively, the IL13 polypeptide comprises the C-terminal 112 amino acids of SEQ ID NO:4 or 20 and at least, at most, or exactly 1, 2, 3, 4, 5, 6, 7, 8, 9, or 10 additional amino acids at the C-terminus.

A CAR molecule may comprises a tag that can be used to sort and/or identify the CAR molecule in a host cell. The tag may be further defined as a therapeutic control. The tag or therapeutic control may be less than a full-length polypeptide and is truncated. For instance, to remove one or more functional domains from the tag. The truncated protein is EGFR (EGFRt), which can be used to detect expression of the CAR. The truncated protein may also be truncated low-affinity nerve growth factor receptor or (dNGFR). The tag may be colorimetric or fluorescent. The tag may be separated from the CAR by a cleavage site.

The VH may be amino proximal to the VL. The VH may be carboxy proximal to the VL. A first region is carboxy proximal to a second region when the first region is attached to the carboxy terminus of the second region. There may be further intervening amino acid residues between the first and second regions. Thus, the regions need not be immediately adjacent, unless specifically specified as not having intervening amino acid residues. The term “amino-proximal” is similarly defined in that a first region is amino-proximal to a second region when the first region is attached to the amino terminus of the second region. Similarly, there may be further intervening amino acid residues between the first and second regions unless stated otherwise.

In a particular embodiment, the CAR comprises in order from amino-proximal end to carboxy-proximal end: an IL13Rα binding region or IL13 polypeptide, a tumor antigen binding region, a TGF-β binding region, a peptide spacer, a transmembrane domain, and a cytoplasmic region comprising a co-stimulatory region and a primary intracellular signaling domain.

The linker between two regions of the polypeptide, such as between two binding regions or between a VH and VL of the same binding region, may be a linker that comprises glycine and serine amino acids. The linker may comprise or consist of a polypeptide with the amino acid sequence of SEQ ID NO: 10 or 28. The linker may be 4-40 amino acids in length. The linker may be, may be at least, may be at most, or may be about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) amino acid residues in length. The linker may comprise at least 4 glycine and/or serine residues. The linker may comprise at least 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) glycine and/or serine residues. The linker may comprise (GGGGS—SEQ ID NO:161)ₙ, wherein n is 1, 2, 3, 4, 5, 6, 7, 8, 9, or 10 (or any derivable range therein). The linker may comprise or consist of the amino acid sequence: (EAAAK-SEQ ID NO:130)ₙ, wherein n is 1, 2, 3, 4, 5, 6, 7, 8, 9, or 10 (or any derivable range therein).

The IL13Rα binding region may comprise an IL13Rα2-specific binding region. The IL13Rα binding region may comprise an IL13 polypeptide. The IL13 polypeptide may be a fragment of the IL13 protein that is capable of binding to IL13Rα. The IL13 polypeptide may be a polypeptide from the IL13 protein that activates the intracellular signaling domain upon binding with IL13Rα. The IL13Rα may comprise membrane-bound IL13Rα. The IL13 polypeptide may comprise an IL13 mutein. The IL13 polypeptide may comprise an E13Y substitution of the IL13 protein. The E13Y substitution is a substitution of a tyrosine for glutamic acid at a position in IL13 that corresponds to position 11 of the IL13 polypeptide of SEQ ID NO: 147, position 13 of the IL13 polypeptide of SEQ ID NO:4, or position 21 of SEQ ID NO: 20. The IL13 polypeptide may comprise or consist of SEQ ID NO:4. The IL13 polypeptide may comprise or consist of SEQ ID NO:20. The IL13 polypeptide may comprise or consist of SEQ ID NO:147. The IL13Rα binding region can comprise or consist of a polypeptide of SEQ ID NO: 147. It is contemplated that the IL13Rα binding region of SEQ ID NO: 147 may be used in any of the CARs described herein.

The polypeptide may further comprise a second chimeric antigen receptor (CAR) comprising at least one antigen binding region, a second peptide spacer, a second transmembrane domain, and a second cytoplasmic region comprising a second co-stimulatory region and a second primary intracellular signaling domain. The second CAR may be a mono-specific or multi-specific CAR, such as a bi-specific or tri-specific CAR. The second CAR may comprise an antigen binding region to TGF-β. The first CAR and the second CAR may be separated by one or more peptide cleavage site(s). The peptide cleavage site may be a peptide cleavage site known in the art, such as a Furin cleavage site or a 2A cleavage site. The 2A cleavage site may comprise one or more of a P2A, F2A, E2A, or T2A cleavage site. The peptide cleavage site comprises a T2A cleavage site. The T2A cleavage site may comprise an amino acid sequence of SEQ ID NO:24. The cleavage site may have at least 80, 81, 82, 83, 84, 85, 86, 87, 88, 89, 90, 91, 92, 93, 94, 95, 96, 97, 98, 99, or 100% (or any derivable range therein) sequence identity to SEQ ID NO:24.

The CAR of the disclosure may comprise or further comprise a torsional linker between the transmembrane domain and the cytoplasmic region. The torsional linker may comprise or consists of 1, 2, 3, 4, 5, 6, 7, 8, 9, or 10 amino acid residues (or any derivable range therein). The amino acid residues may comprise or consist of alanine residues. The torsional linker may comprise at least, at most, or exactly 1, 2, 3, 4, 5, 6, 7, 8, 9, or 10 (or any derivable range therein) alanine residues. The torsional linker may comprise at least, at most, or exactly 1, 2, 3, 4, 5, 6, 7, 8, 9, or 10 (or any derivable range therein) contiguous alanine residues. The torsional linker may consist of 2 or 4 alanine residues. The torsional linker may comprise at least, at most, or exactly 1, 2, 3, 4, 5, 6, 7, 8, 9, or 10 (or any derivable range therein) contiguous alanine residues. The torsional linker may consist of 2 alanine residues.

The CAR of the disclosure may include a peptide spacer between the antigen binding domains and the transmembrane domain. Similarly, the second CAR of the disclosure may include a peptide spacer is between the antigen binding domains and the second transmembrane domain of the second CAR. The peptide spacer or second peptide spacer may comprise an IgG4 hinge region. The IgG4 hinge region may comprise a polypeptide having an amino acid sequence with at least 80% sequence identity to SEQ ID NO:12. The IgG4 hinge region may comprise a polypeptide having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:12. The IgG4 hinge region may comprise a polypeptide having the amino acid sequence of SEQ ID NO: 12 The IgG4 hinge region may comprise a polypeptide having an amino acid sequence with at least 80% sequence identity to SEQ ID NO:5. The IgG4 hinge region may comprise a polypeptide having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:5. The IgG4 hinge region may comprise a polypeptide having the amino acid sequence of SEQ ID NO:5. The peptide spacer or second peptide spacer may comprise or further comprise an IgG4 CH2 and CH3 region. The peptide spacer or second peptide spacer may comprise or further comprise an IgG4 CH2 and CH3 region. The IgG4 CH2 and CH3 region may comprise a polypeptide having an amino acid sequence with at least 80% sequence identity to SEQ ID NO:37. The IgG4 CH2 and CH3 region may comprise a polypeptide having an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:37. The IgG4 CH2 and CH3 region may comprise a polypeptide having the amino acid sequence of SEQ ID NO:37. The CH2 region may comprise L235E and/or N297Q substitutions. The peptide spacer may be between 8 and 1000 amino acids in length. The peptide spacer may be between 8 and 500 amino acids in length. The peptide spacer may be between 100-300 amino acids in length. The peptide spacer may have fewer than 100 amino acids. The peptide spacer may be at least, at most, or exactly, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 amino acids (or any derivable range therein).

The transmembrane domain or second transmembrane domain may comprise the transmembrane domain from the CD28 protein. The transmembrane domain or second transmembrane domain may comprise a transmembrane domain having an amino acid sequence with at least 80% sequence identity to SEQ ID NO:6. The transmembrane domain or second transmembrane domain may comprise a transmembrane domain having an amino acid sequence with at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:6. The transmembrane domain or second transmembrane domain may comprise a transmembrane domain having the amino acid sequence of SEQ ID NO:6. The transmembrane domain may be an alpha or beta chain of the T cell receptor, CD28, CD38 (epsilon), CD45, CD4, CD5, CD8, CD9, CD16, CD22, CD33, CD37, CD64, CD80, CD86, CD123, CD134, CD137 or CD154 transmembrane domain.

The co-stimulatory region or second co-stimulatory region in the peptides and CARs described herein may comprise the co-stimulatory region from the 4-1BB protein or from the CD28 protein. The co-stimulatory region or second co-stimulatory region may comprise a co-stimulatory region having an amino acid sequence with at least 80% sequence identity to SEQ ID NO: 7, 14 or 18. The co-stimulatory region or second co-stimulatory region may comprise a co-stimulatory region having an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:7, 14, or 18. The co-stimulatory region or second co-stimulatory region may comprise a co-stimulatory region having the amino acid sequence of SEQ ID NO:7, 14, or 18. The cytoplasmic region may comprise two costimulatory domains. The one or more costimulatory domain(s) may comprise a costimulatory domain from one or more of 4-1BB (CD137), CD28, IL-15Rα, OX40, CD2, CD27, CDS, ICAM-1, LFA-1 (CD11a/CD18), and/or ICOS (CD278). The one or more costimulatory domains may comprise a costimulatory domain from CD28 or a costimulatory domain derived from CD28.

The primary intracellular signaling domain or second primary intracellular signaling domain of the polypeptides and CARs described herein may comprise an intracellular signaling domain from the CD33 protein. The primary intracellular signaling domain or second primary intracellular signaling domain may comprise an intracellular signaling domain having an amino acid sequence with at least 80% sequence identity to SEQ ID NO:8 or 15. The primary intracellular signaling domain or second primary intracellular signaling domain may comprise an intracellular signaling domain having an amino acid sequence having or at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:8 or 15. The primary intracellular signaling domain or second primary intracellular signaling domain may comprise an intracellular signaling domain having the amino acid sequence of SEQ ID NO:8 or 15.

The polypeptides may comprise an amino acid sequence of one of SEQ ID NOS: 136-145, 159, or 160 or an amino acid sequence having at least 80% sequence identity to one of SEQ ID NOS: 136-145, 159, or 160. The polypeptides may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS: 136-145, 159, or 160. The polypeptides may comprise an amino acid sequence of one of SEQ ID NOS: 1, 9, 13, 16, 17, 19, 21-23, and 25 or an amino acid sequence having at least 80% sequence identity to one of SEQ ID NOS: 1, 9, 13, 16, 17, 19, 21-23, and 25. The polypeptides may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS: 1, 9, 13, 16, 17, 19, 21-23, and 25. The polypeptides may comprise an amino acid sequence of one of SEQ ID NOS: 146, 148-158, and 161-172 or an amino acid sequence having at least 80% sequence identity to one of SEQ ID NOS: 146, 148-158, and 161-172. The polypeptides may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS: 146, 148-158, and 161-172.

The polypeptides of the disclosure may comprise or further comprise one or more molecular tag(s). The one or more molecular tags may comprise FLAG and/or HA tag. The polypeptides of the disclosure may comprise or further comprise one or more signal sequence(s). The signal sequence(s) may comprise an amino acid sequence with at least 80% sequence identity to SEQ ID NO:2. The signal sequence(s) may comprise an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to SEQ ID NO:2. The signal sequence(s) may comprise the amino acid sequence of SEQ ID NO:2. The polypeptides may also exclude a FLAG tag in the CARs of the disclosure.

The polypeptides may be encoded on nucleic acids and/or expression constructs comprising the nucleic acids. The heterologous nucleic acid may comprise RNA or DNA. The expression construct may be a viral vector, such as a retroviral vector or a vector derived from a retrovirus. The viral vector may be a lentiviral vector or a vector derived from a lentivirus. The vector may be a lentivirus vector comprising a sequence encoding a polypeptide of the disclosure. The expression vector, such as the viral vector, may be one that has integrated into the host cell's genome. The cell may be ex vivo. It is also contemplated that the cell is in vivo. The cells may be ones that are expressing or capable of expressing the polypeptide encoded on the heterologous nucleic acid.

The cells may comprise a nucleic acid that encodes for or comprise an expressed first polypeptide with a bi-specific CAR comprising an IL13Rα binding region and a TGF-beta binding region and a second polypeptide having a CAR comprising a GD2 binding region. The cells may comprise a nucleic acid that encodes for or comprise an expressed first polypeptide with a bi-specific CAR comprising an IL13Rα binding region and a TGF-beta binding region and a second polypeptide having a CAR comprising a EGFRvIII binding region. The cells may comprise a nucleic acid that encodes for or comprise an expressed first polypeptide with a bi-specific CAR comprising an IL13Rα binding region and a GD2 binding region and a second polypeptide having a CAR comprising a TGF-beta binding region. The cells may comprise a nucleic acid that encodes for or comprise an expressed first polypeptide with a bi-specific CAR comprising an IL13Rα binding region and a EGFRvIII binding region and a second polypeptide having a CAR comprising a TGF-beta binding region. The cells may comprise a nucleic acid that encodes for or comprise an expressed first polypeptide with a bi-specific CAR comprising a TGF-beta binding region and a EGFRvIII binding region and a second polypeptide having a CAR comprising an IL13Rα binding region. The cells may comprise a nucleic acid that encodes for or comprise an expressed first polypeptide with a bi-specific CAR comprising a TGF-beta binding region and a GD2 binding region and a second polypeptide having a CAR comprising an IL13Rα binding region. The cells may comprise a nucleic acid that encodes for or comprise an expressed first polypeptide with a bi-specific CAR comprising a TGF-beta binding region and a GD2 binding region and a second polypeptide having a bi-specific CAR comprising an IL13Rα binding region and a EGFRvIII binding region. The cells may comprise a nucleic acid that encodes for or comprise an expressed first polypeptide with a bi-specific CAR comprising a TGF-beta binding region and a EGFRvIII binding region and a second polypeptide having a bi-specific CAR comprising an IL13Rα binding region and a GD2 binding region. The cells may comprise a nucleic acid that encodes for or comprise an expressed first polypeptide with a bi-specific CAR comprising an IL13Rα binding region and a TGFbeta binding region and a second polypeptide having a bi-specific CAR comprising a EGFRvIII binding region and a GD2 binding region.

A nucleic acid may be a molecule involved in gene editing such that a nucleic acid (such as a guide RNA) encoding a CAR is used to incorporate a CAR-coding sequence into a particular locus of the genome, such as the TRAC gene. This may involve a gene editing system such as CRISPR/Cas9. A nucleic acid, polynucleotide, or polynucleotide region (or a polypeptide or polypeptide region) has a certain percentage (for example, 60%, 65%, 70%, 75%, 80%, 85%, 90%, 95%, 98% or 99%—or any range derivable therein) of “sequence identity” or “homology” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. It is contemplated that a nucleic acid may have such sequence identity or homology to any nucleic acid SEQ ID NO provided herein.

the cell may be a population of cells comprising a nucleic acid that encodes all or part of any polypeptide discussed herein. The cell or population of cells may contain within its genome a sequence encoding any of the polypeptides described herein. This includes, but is not limited to, a lentivirus or retrovirus that has integrated into the cell's genome. The cell or population of cells may express all or part of any CAR discussed herein, including, but not limited to those with the amino acid sequence of any of and/or comprising the amino acid sequence of any of SEQ ID NOS: 1-159. Progeny (F1, F2, and beyond) of cells in which a nucleic acid encoding a polypeptide was introduced are included in the cells or populations of cells disclosed herein. The cell or population of cells may be a T cell, a natural killer (NK) cell, a natural killer T cell (NKT), an invariant natural killer T cell (INKT), stem cell, lymphoid progenitor cell, peripheral blood mononuclear cell (PBMC), hematopoietic stem and progenitor cell (HSPC), hematopoietic stem cell (HSC), CD34+ cell, peripheral blood stem cell (PBSC), bone marrow cell, fetal liver cell, embryonic stem cell, cord blood cell, induced pluripotent stem cell (iPS cell). The cell may be a T cell or an NK cell. A T cell may comprise a naïve memory T cell. The naïve memory T cell may comprise a CD4+ or CD8+ T cell. The cells may be a population of cells comprising both CD4+ and CD8+ T cells. The cells may be a population of cells comprising naïve memory T cells comprising CD4+ and CD8+ T cells. The T cell may comprise a T cell from a population of CD14 depleted, CD25 depleted, and/or CD62L enriched PBMCs. The cell may be an immune cell. The cell may be a progenitor cell or stem cell. The progenitor or stem cell may be in vitro differentiated into an immune cell. The cell may be a T cell. The cell may be a CD4+ or CD8+ T cell. The cell may be a natural killer cell. The cell may be ex vivo. The term immune cells includes cells of the immune system that are involved in defending the body against both infectious disease and foreign materials. Immune cells may include, for example, neutrophils, eosinophils, basophils, natural killer cells, lymphocytes such as B cells and T cells, and monocytes. T cells may include, for example, CD4+, CD8+, T helper cells, cytotoxic T cells, γδ T cells, regulatory T cells, suppressor T cells, and natural killer T cells. The T cell may be a regulatory T cell.

The population of cells may comprise 10³-10⁸ cells. The population may be about, may be at least about, or may be at most about 10², 10³, 10⁴, 10⁵, 10⁶, 10⁷, 10⁸, 10⁹, 10¹⁰, 10¹¹, 10¹² cells (or any range derivable therein). Cells may be autologous with respect to a patient who will receive them. Cells may also be defined as non-autologous and/or allogeneic.

The cell may be one that is not yet a T cell or NK cell, and the method may further comprise culturing the cell under conditions that promote the differentiation of the cell into a T cell or an NK cell. The methods may further comprise culturing the cell under conditions to expand the cell before and or after introducing the nucleic acid into the cell. The cell may be cultured with serum-free medium.

The patient may be one that has relapsed or recurrent cancer. The methods may include a step of administering an additional therapy to the patient. The patient may be one that has been diagnosed with the cancer and/or a cancer that has GD+ or EGFRvIII+ cells, as described herein. The patient may be one that has been determined to have the cancer and/or a cancer that has GD+ or EGFRvIII+ cells, as described herein. The subject may be one that is at risk of having cancer and/or GD2+ or EGFRvIII+ cancer. The patient may be one that has been previously treated to the cancer. The patient may be one that has been determined to be resistant to the previous treatment. The previous treatment may be a cancer therapeutic described herein, such as those described as additional therapies. The methods may include a step of administering chemotherapy and/or radiation to the patient. The additional therapy may comprise an immunotherapy. The additional therapy may comprise an additional therapy described herein. The immunotherapy may comprise immune checkpoint inhibitor therapy. The immunotherapy may comprise an immunotherapy described herein. The immune checkpoint inhibitor therapy may comprise a PD-1 inhibitor and/or CTLA-4 inhibitor. The immune checkpoint inhibitor therapy may comprise one or more inhibitors of one or more immune checkpoint proteins described herein.

The cancer may comprise a GD2+ cancer, wherein a GD2+ cancer is one that comprises GD2+ cells or comprises at least 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, or 90% GD2+ cancer cells in a population of tumor cells.

The cancer may comprise a EGFRvIII+ cancer, wherein a EGFRvIII+ cancer is one that comprises EGFRvIII+ cells or comprises at least 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, or 90% EGFRvIII+ cancer cells in a population of tumor cells.

The CAR polypeptides may have a region, domain, linker, spacer, or other portion thereof that comprises or consists of an amino acid sequence that is at least, at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) to all or a portion of the amino acid sequences described herein. A CAR polypeptide may comprise or consist of an amino acid sequence that is, is at least, is at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical (or any range derivable therein) to any one of SEQ ID NOS: 1-172.

The method may comprise stimulating an immune response, wherein stimulating an immune response comprises increasing expression and/or secretion of immune stimulating cytokines and/or molecules. The immune stimulating cytokines and/or molecules may be one or more of TNF-α, IFN-β, IFN-γ, IL-1, IL-2, IL-4, IL-6, IL-8, IL-10, IL-12, IL-18 and granulocyte-macrophage colony stimulating factor. In some methods, 1, 2, 3, 4, 5, 6, 7, 8, 9, 10, or more of these may excluded. Stimulating an immune response may comprise increasing proliferation of immune cells. The immune cells may be T cells. The cells may be ex vivo. The cell may also be in vivo in a subject in need of immune stimulation. The subject may be one that produces endogenous TGF-β and/or an excess of endogenous TGF-β. An increase in expression or proliferation as described herein may be at least, at most, or exactly 1, 2, 3, 4, 5, 6, 7, 8, 9, 10, 50, 100, 200, 300, 500, or 1000 fold increase (or any range derivable therein) over a base-line expression level such as a control (non-disease, non-TGF-β or non-antigen binding polypeptide control).

The subject may be a mammal, such as a human, rat, mouse, or non-human primate The subject may be a human. The subject may also be a goat, pig, horse, cat, or dog. The route of administration of the compositions, polypeptides, cells, and nucleic acids of the disclosure may be a route of administration described herein. The compositions may be administered intraventricularly, intracerebroventricularly, intratumorally, intravenously, or into a tumor resection cavity. The compositions may be formulated for intraventricular, intracerebroventricular, intratumoral, or intravenous administration or for administration into a tumor resection cavity.

The methods may further comprise administering TGF-β to the subject. In compositions of the disclosure, the composition may comprise 1-50 ng/mL of TGF-β. The composition may comprise at least, at most, or about 0.5, 1, 1.5, 2, 2.5, 3, 3.5, 4, 4.5, 5, 5.5, 6, 6.5, 7, 7.5, 8, 8.5, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, or 100 ng/ml of TGF-β (or any range derivable therein).

The composition may further comprise IL-2. The composition may comprise 20-400 U/mL of IL-2. The composition may comprise at least, at most, or about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600 U/mL of IL-2 (or any range derivable therein). the composition may further comprise IL-15. The composition may comprise 0.1-10 ng/ml of IL-15. The composition may comprise at least, at most, or about 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 490, 495, 5.00, 5.05, 5.10, 5.15, 5.20, 5.25, 5.30, 5.35, 5.40, 5.45, 5.50, 5.55, 5.60, 5.65, 5.70, 5.75, 580, 5.85, 5.90, 5.95, 6.00, 6.5, 7, 7.5, 8, 8.5, 9, 9.5, 10, 11, 12, 13, 14, 15, 16, 17, 18, 19, 20, 30, 40, or 50 ng/ml of IL-15 (or any range derivable therein). The composition may comprise or further comprise IL-7, IL-12, and/or IL-21. The composition may comprise at least, at most, or about 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 490, 495, 5.00, 5.05, 5.10, 5.15, 5.20, 5.25, 5.30, 5.35, 5.40, 5.45, 5.50, 5.55, 5.60, 5.65, 5.70, 5.75, 580, 5.85, 5.90, 5.95, 6.00, 6.5, 7, 7.5, 8, 8.5, 9, 9.5, 10, 11, 12, 13, 14, 15, 16, 17, 18, 19, 20, 30, 40, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600 μg/mL, ng/ml, μg/mL, or mg/ml of IL-7, IL-12, and/or IL-21 (or any range derivable therein).

The methods may further comprise contacting the cells with feeder cells. The feeder cells may be irradiated. Feeder cells or support cells can include, for example, fibroblasts, mouse embryonic fibroblasts, JK1 cells, SNL 76/7 cells, human fetal skin cells, human fibroblasts, and human foreskin fibroblasts.

The methods may exclude contacting T cells with feeder cells. In some cases, the excluded feeder cells are from a different animal species as the T cells.

Polypeptides described throughout this disclosure may be isolated, meaning they are not found in the cellular milieu. In some cases, they are purified, which means it is mostly if not completely separated from polypeptides having a different amino acid sequence and/or chemical formula.

The present disclosure provides a method for treating a subject with cancer comprising administering to the subject an effective amount of a population of cells or pharmaceutical composition comprising a chimeric polypeptide or nucleic acid encoding a chimeric polypeptide.

“Treatment” or “Treating” may refer to any treatment of a disease in a mammal, including: (i) suppressing the disease, that is, causing the clinical symptoms of the disease not to develop by administration of a protective composition after the inductive event but prior to the clinical appearance or reappearance of the disease; (ii) inhibiting the disease, that is, arresting the development of clinical symptoms by administration of a protective composition after their initial appearance; and/or (iii) relieving the disease, that is, causing the regression of clinical symptoms by administration of a protective composition after their initial appearance. The treatment may exclude prevention of the disease.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Example section are understood to be embodiments that are applicable to all aspects of the technology described herein.

It is specifically contemplated that any method, composition, cell, polypeptide, or nucleic acid embodiment or aspect described herein may be used interchangeably and in combination with each other. Furthermore, it is contemplated that aspects and embodiments of the disclosure may specifically exclude an aspect or embodiment described herein.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more.” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z.” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that aspects or embodiments described in the context of the term “comprising” may also be implemented in the context of the term “consisting of” or “consisting essentially of.”

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments or aspects discussed elsewhere in a different Example or elsewhere in the application, such as in the Summary of Invention, Detailed Description, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B: (1A) Panel of CAR constructs used in the aspects of the disclosure. (1B) Expression of the single-input and bispecific CARs on the surface of T cells.

FIG. 2: Stimulation of CAR-T cells with 5 ng/ml or 10 ng/ml of exogenous TGF-β. Each set of three bars represents, from left to right, data for: 0, 5, and 10 ng/ml TGF-β.

FIGS. 3A-C: CAR-T cells were labeled with CellTrace Violet (CTV) dye and then co-incubated with patient-derived PBT106 GBM neurosphere cells at a 1:8 effector-to-target ratio for 94 hours, in the presence or absence of metalloprotease 9 (MMP-9). The number of surviving tumor cells (3A), number of FLAG+ CAR-T cells (3B), as well as CTV dye intensity among FLAG+ CAR-T cells (3C) were quantified by flow cytometry. Each set of four bars represents, from left to right, data for: IL13Rα2BBz (CD4tm); IL13Rα2BBz (CD28tm); IL13Rα2-(G4S) 3-TGFβ.BBz (CD28tm); and IL13Rα2-(G4S) 4-TGFβ.BBz (CD28tm).

FIG. 4: Additional contemplated CAR aspects.

FIG. 5: TGF-β Activates Bispecific IL-13Rα2/TGF-β CAR-T Cells. Primary human T cells were transduced with the indicated constructs and seeded at 7.5×10⁴ CAR+ T cells in 100 μL per well of a 96-well plate, with the indicated concentration of TGF-β. Cells were harvested after 21 hours for surface staining with anti-CD69 and anti-CD25 antibodies followed by flow cytometry analysis. The mean value of technical triplicates are shown with error bars indicating ±1 standard deviation (s.d.). Each set of three bars represents, from left to right, data for: 0, 5, and 10 ng/ml TGF-β.

FIG. 6: IL-13Rα2/TGF-β CAR-T Cells Exhibit Increased Proliferation Upon Tumor Challenge. Patient-derived PBT-106 glioblastoma neurosphere cells that stably express EGFP-firefly luciferase fusion protein were sorted for IL-13Rα2 expression and seeded at 4×10⁴ per well in 96-well plate. Primary human T cells were transduced with the indicated constructs and stained with CellTrace Violet (CTV) dye. CTV-stained T cells were co-incubated with seeded PBT-106 cells at 1:8 effector-to-target ratio for 94 hours. Flow cytometry was performed to quantify the number of viable EGFP+ tumor cells, viable CTV+ T cells, and CTV dye intensity in T cells. The mean value of technical triplicates are shown with error bars indicating ±1 s.d.

FIGS. 7A-B: IL-13Rα2/TGF-β CAR-T Cells Exhibit Superior In Vivo Tumor Control. Patient-derived PBT-106 glioblastoma neurosphere cells that stably express EGFP-firefly luciferase fusion protein were sorted for IL-13Rα2 expression. NSG mice were engrafted with 2×10⁵ sorted PBT-106 cells via intracranial injection (1.5 mm lateral, 0.5 mm posterior of bregma. 2.5 mm into dura). Seven days later, tumor-bearing mice were treated with cither 5×10⁵ T cells expressing the indicated construct or PBS alone (7A, left side). Tumor progression was quantified by bioluminescence imaging; each line in the radiance plots indicate an individual mouse (7A, right). Survival data are displayed as Kaplan-Meier curve (7B); two deaths in the bispecific CAR group were censored based on determination that the deaths unrelated to tumor burden (e.g., exhibiting clear signs of graft-versus-host disease while showing no tumor signal by luciferase imaging and no sign of tumor upon brain dissection).

FIG. 8: IL-13Rα2/TGF-β CAR-T Cells Exhibit Superior In Vivo Tumor Control. Patient-derived PBT-106 glioblastoma neurosphere cells that stably express EGFP-firefly luciferase fusion protein were sorted for IL-13Rα2 expression. NSG mice were engrafted with 2×10⁵ sorted PBT-106 cells via intracranial injection (1.5 mm lateral, 0.5 mm posterior of bregma, 2.5 mm into dura). Seven days later, tumor-bearing mice were treated with either 5×10⁵ T cells expressing the indicated construct or PBS alone. Tumor progression was quantified by bioluminescence imaging; each line in the radiance plots indicate an individual mouse. Survival data are displayed as Kaplan-Meier curve.

FIG. 9: TGF-β CAR Shows No In Vivo Toxicity Despite Cross-reactivity with Murine TGF-β. Primary human T cells expressing a TGF-β CAR with CD28 costimulatory domain was incubated with 0.5, 1.5, 5, 15, 50, 150, and 500 ng/mL of human or mouse TGF-β1, in triplicate, at 1×10⁵ cells/100 μL media per well in a 96-well plate. All wells were treated with 1× Brefeldin A (diluted from 1000× stock from BioLegend). The following day, intracellular staining was performed on the cells for IFN-γ, TNF-α, and IL-2.

FIG. 10: No Systemic Toxicity with Murine TGF-β CAR-T Cells in C57BL/6 Mice. C57BL/6 mice were administered 4×10⁶ T cells expressing the indicated construct via tail-vein injection (n=3 per treatment group). Animal weight was measured at the indicated time point. On Day 31 post T-cell injection, all animals were sacrificed, and their liver, spleen, and kidneys were collected for histopathology analysis. No significant difference was observed between animals treated with mock-transduced vs. TGF-β CAR-T cells in either weight or histopathology results.

FIG. 11: FLAG (CAR) Surface Expression (No Antigen Stimulation). Averages of triplicates are shown, with error bars representing ±1 standard deviation. Each set of two bars represents data, from left to right, of the SP and Full IL13R construct.

FIGS. 12A-D: (12A) CD69 Activation Marker Expression after 21-hr Stimulation. (12B) CD25 Activation Marker Expression after 21-hr Stimulation. (12C) FLAG (CAR) Surface Expression after 21-hr Stimulation. (12D) FLAG (CAR) Surface Expression after 21-hr Stimulation. PBT106 NS is a tumor line that expresses IL-13Rα2. Averages of triplicates are shown, with error bars representing ±1 standard deviation. Each set of three bars represents the data, from left to right, of 1) media only; 2) 5 ng/ml TGF-β; and 3) 100% IL13Rα2+PBT 106 NS.

FIGS. 13A-F: (13A-13B) Viable Tumor Count after 92-hr Coincubation. (13C-13D) Viable T-cell Count after 92-hr Coincubation (13E-13F) CTV Dilution among T Cells after 92-hr Coincubation. T cells were stained with CellTrace Violet (CTV) dye, which dilutes with each T-cell division. Therefore, the lower the CTV MFI, the more times the T cells have divided. Averages of triplicates are shown, with error bars representing ±1 standard deviation. Each set of 16 bars represents the data, from left to right, of 1) SP-IL13Rα2.BBz; 2) Full-IL13Rα2.BBz; 3) SP-IL13Rα2/TGF-β.BBz; 4) Full-IL13Rα2/TGF-β.BBz; 5) SP-IL13Rα2.BBz KR; 6) Full-IL13Rα2.BBz KR; 7) SP-IL13Rα2/TGF-β.BBz KR; 8) SP-IL13Rα2.28z; 9) Full-IL13Rα2.28z; 10) SP-IL13Rα2/TGF-β.28z; 11) Full-IL13Rα2/TGF-β.28z; 12) SP-IL13Rα2/TGF-β.BBz+GD2.AA.28z; 13) Full-IL13Rα2/TGF-β.BBz+GD2.AA.28z; 14) SP-IL13Rα2.BBz+TGF-β DNR; 15) Full-IL13Rα2.BBz+TGF-β DNR; and 16) scFv-less CAR.

FIG. 14. NOD/scid/γ−/− (NSG) mice were intracranially engrafted with 2.5×10\hat{ }5 PBT106 glioblastoma multiforme (GBM) neurosphere cells that stably express firefly luciferase. Tumor-bearing mice were treated with 0.5×10\hat{ }6 CAR+ cells 7 days after tumor injection. Tumor progression was monitored by bioluminescence imaging. Each trace represents one mouse, with “x” marking time of sacrifice for mice that reached the humane end point. Survival is shown in Kaplan-Meier curve.

FIGS. 15A-E. Bispecific IL-13Rα2/TGF-β tanCARs exhibit robust cytotoxicity in vitro against a panel of patient-derived neurosphere lines. (15A) Schematic of CAR constructs. All CARs encode an N-terminal FLAG tag to enable flow cytometric analysis of cell-surface CAR expression in transduced T cells. (15B) IL-13Rα2 expression by patient-derived GBM neurospheres. Intraoperative samples from newly diagnosed and recurrent GBM patients were used to establish a panel of neurosphere lines with varying IL-13Rα2 expression levels as quantified by surface antibody staining. (15C) Total TGF-β production for the panel of GBM neurosphere lines. Values shown are the means of triplicates with error bars indicating ±1 standard deviation (SD). (15D) Killing of patient-derived neurosphere lines by CAR-T cells. Neurospheres were seeded overnight and subsequently co-incubated with CAR-T cells at a 3:1 E:T ratio. To account for differences in transduction efficiency between CAR constructs, untransduced cells were added as necessary to achieve equal numbers of CAR⁺ as well as total T cells. Cytolysis was measured by xCelligence assay. Pairwise comparisons of % cytolysis were performed on the last measurement value taken, and statistics were computed using the two-tailed, unpaired, two-sample Student's t test, with the Sidak correction for multiple comparisons (**** p<0.0001). (15E) Time to half-maximal killing (T50; units in hours following addition of T cells to co-cultures) of neurospheres by CAR-T cells. Data shown in (D) were fitted to a sigmoidal curve, and T50 values with 95% confidence intervals (CI) are shown. The width of the bar corresponds to the 95% CI.

FIGS. 16A-I. Bispecific IL-13Rα2/TGF-β tanCARs re-wire TGF-β signaling and exhibit superior control of orthotopically implanted xenografts. (16A) Schematic of bicistronic construct encoding single-input IL-13Rα2 CAR and dominant-negative TGF-β receptor (sCAR+DNR). (16B) Inhibition of endogenous TGF-β signaling in T cells expressing sCAR+DNR and tanCAR. CAR-T cells were cultured in serum-free media, and incubated with or without TGF-β for 1 hour prior to cell lysis. Cell lysates were subsequently analyzed for phosphorylated SMAD by Western blot. (16C) Sequestration of TGF-β by T cells expressing sCAR+DNR and tanCAR. CAR-T cells were incubated in either media alone or with 5 ng/ml recombinant active TGF-β. After 24 hours, cell-culture supernatant was collected and concentrations of active TGF-β were quantified by ELISA. For each group of two bars, the left bar represents media only, and the right bar represents 5 ng/ml TGF-β. (16D) TanCAR-T cell activation in response to TGF-β. CAR-T cells were incubated with or without TGF-β overnight. CD69 (left) and CD25 (right) expression were measured by flow cytometry. For each group of two bars, the left bar represents media only, and the right bar represents 5 ng/ml TGF-β. (16E) Cytokine production by tanCAR-T cells in response to TGF-β. Two-hundred-thousand human CAR-T cells were incubated for 24 hours with or without exogenous TGF-β before supernatant was analyzed for IFNγ and TNFα content by ELISA. For each group of two bars, the left bar represents media only, and the right bar represents 5 ng/ml TGF-β. (16F) Schematic of human GBM xenograft model in NSG mice. (16G) Survival curve of mice from a pilot study (n=5 for scFv-less CAR and tanCAR treatment groups, n=9 for sCAR+DNR treatment group). (16H) Survival curve of mice in full-scale study (n=5 for scFv-less CAR treatment group, n=7-8 for on-target treatment groups). (16I) Tumor burden of mice in full-scale study as measured by bioluminescence imaging. Each trace represents an individual mouse and ends at the last imaging data point prior to euthanasia. Data shown are representative of results from two independent experiments performed with T cells derived from different donors.

Values in (16C-16E) show means of triplicates with error bars indicating ±1 SD. Statistics in (16C) were computed using two-way ANOVA with Bonferroni's correction for multiple comparisons. Statistics in (16D) and (16E) were computed using the two-tailed, unpaired, two-sample Student's t test, with the Holm-Sidak correction for multiple comparisons. Statistics in (16G) and (16H) were computed using the log-rank test with P values adjusted by the Bonferroni correction for multiple comparisons.

FIGS. 17A-J. Bispecific IL-13Rα2/TGF-β tanCAR-T cells re-shape the TME of murine gliomas. (17A) IL-13Rα2 staining in CT-2A tumor cells engineered to overexpress human IL-13Rα2. Parental cells were included as negative controls. (17B) Total TGF-β levels as measured by ELISA in cell culture supernatant of IL-13Rα2+CT-2A glioma cells, collected 24 and 48 hours after cell seeding. Error bars represent+1 standard deviation from the mean. (17C-17D) Reactivity of murine CAR-T cells to CT-2A tumor cells. Murine CAR-T cells were labeled with CellTrace Yellow (CTY) dye and co-incubated with IL-13Rα2+CT-2A glioma cells at specified E:T ratios. To account for differences in transduction efficiency between CAR constructs, untransduced cells were added as necessary to achieve equal numbers of total T cells. (17C) Surviving tumor cell counts were measured after four days. Each set of three bars represents, from left to right, untreated, sCAR, and tanCAR. (17D) CTY dilution in T cells following co-culture with tumor cells at a 1:1 ET ratio were measured after four days. Dye dilution was quantified by fold-change in MFI (left), with representative histograms (right). The bars represent, from left to right, untreated, sCAR, and tanCAR. (17E) Study schematic for CyTOF analysis. Brains from 3 mice per treatment group were pooled for analysis. Each set of three bars represents, from left to right, untreated, sCAR, and tanCAR. (17F-G) Proportions of (17F) suppressive myeloid cells (M-MDSCs) and (17G) T cells (CD8+ and CD4+) among total brain-infiltrating leukocytes in CT-2A tumor-bearing mice. (17H) Ratio of T cells to myeloid cells present in the brains of CT-2A tumor-bearing mice. For (17F)-(17H), the bars represent, from left to right, scFv-less, sCAR, sCAR+DNR, and tanCAR. (17I) Violin plots depicting PD-L1 expression among myeloid cells. (17J) Violin plots depicting PD-1 expression among T cells. Statistics in (17C) and (17D) were calculated using the two-tailed, unpaired, two-sample Student's t test with the Holm-Sidak correction for multiple comparisons (* P<0.05, ** P<0.01, *** P<0.001, **** P<0.0001). Hatched lines in violin plots shown in (17I)-(17J) represent, from top to bottom, the 75^{th} percentile, median, and 25^{th} percentile of expression. Statistics for panels (17I) and (17J) were computed by one-way ANOVA using Tukey's method to correct for multiple comparisons (**** P<0.0001). For (17I)-(17J), the plots represent, from left to right, scFv-less, sCAR, sCAR+DNR, and tanCAR.

FIGS. 18A-H. Single-cell transcriptomics of CT-2A tumor-bearing brains reveal enhanced immune activation and reduced immunosuppression with tanCAR-T cell treatment. (18A) UMAP representation of single-cell RNA sequencing data pooled from all treatment groups. MG, microglia; CD8, CD8⁺ T cells; CD4, CD4⁺ T cells; NK, natural killer cells; DC, dendritic cells. For cell types spanning multiple clusters (e.g., 5 MG clusters), an additional number was appended to maintain unique cluster identities. (18B) Cells in the tumor cluster as a proportion of total cells from each treatment group. Brains from 3 mice per treatment group were pooled for analysis. (18C) Cells in the peripheral macrophage cluster as a proportion of total cells from each treatment group. (18D) Ratio of all T cells to peripheral macrophages for each treatment group. (18E) Transcriptional expression in peripheral macrophages. Expressions of the activation marker CD83, TGF-β-induced protein (TGF-βi), and immunosuppressive LGALS-family markers are shown for treatment group. Expression level is obtained by normalizing feature transcriptional content to total cellular transcriptional content followed by log-transformation and scalar multiplication as defined in Seurat tutorials. (18F) Identification of re-clustered T cell populations. T cells (clusters 1, 6, 12, 13, 19, and 25) from the original dataset were re-clustered after removal of cells exhibiting nonsensical dual lineage marker expression (e.g., CD8⁺/P2ryl2⁺). (18G) Proportional composition of the CD4⁺ T cells (clusters 0 and 6) split by treatment group. (18H) Proportional composition of the CD8⁺ T cells (clusters 1, 2, 3, 4, 5, and 7) split by treatment group.

FIGS. 19A-C. Bispecific IL-13Rα2/TGF-β tanCAR-T cells exhibit superior control of syngeneic GBM compared to single-input IL-13Rα2 CAR-T cells. (19A) Schematic of CT-2A syngeneic GBM model in C57BL/6 mice (n=13 for scFv-less CAR treatment group, n=24 for tanCAR and sCAR+DNR treatment groups, n=27 for sCAR treatment group; data were pooled from two independent experiments). (19B) Survival curves of treated mice. Statistics were computed using the log-rank test with P values adjusted by the Bonferroni correction for multiple comparisons. (19C) Tumor radiance as measured by bioluminescence imaging. Each trace represents an individual mouse and ends at the last imaging data point prior to euthanasia.

FIGS. 20A-M. Bispecific IL-13Rα2/TGF-β tanCAR-T cells do not induce systemic toxicity in mouse models. (20A) Study schematic for toxicity evaluation of tanCAR-T cells delivered through intracranial injections. (20B) Cells expressing human CD3 and human CD45 as a fraction of viable singlets in peripheral blood recovered from NSG mice treated with human T cells. (20C) Cells expressing FLAG, murine CD3, and murine CD45 as a fraction of viable singlets in peripheral blood recovered from C57BL/6 mice treated with murine T cells. (20D) Fold-change in NSG mouse weight. Mouse weight over time was normalized to day-0 values and plotted as means±1 SD for each treatment group (n=5 for scFv-less CAR and tanCAR treatment groups; n=9 for sCAR+DNR treatment group). (20E) Fold-change in C57BL/6 mouse weight. Mouse weight over time was normalized to day-0 values and plotted as means±1 SD for each treatment group (n=7 for scFv-less CAR treatment group, n=15-16 for IL-13Rα2-targeting CAR treatment groups). (20F) Study schematic for toxicity evaluation of TGF-β CAR-T cells delivered through intravenous injection. (20G) Cells expressing murine CD3 as a fraction of viable singlets in peripheral blood recovered from C57BL/6 mice treated with murine T cells. (20H) Fold-change in C57BL/6 mouse weight. Mouse weight over time was normalized to day −0 values and plotted as means±1 SD for each treatment group (n=3 mice per group). (20I) Study schematic for toxicity evaluation of tanCAR-T cells delivered through intravenous injection. (20J) Cells expressing murine CD3 as a fraction of viable singlets in peripheral blood recovered from tumor-bearing C57BL/6 mice treated with murine CAR-T cells. (20K) Cells expressing murine CD3 as a fraction of viable singlets in peripheral blood recovered from healthy C57BL/6 mice treated with murine CAR-T cells. (20L) Fold-change in C57BL/6 mouse weight. Mouse weight over time was normalized to values from day-16 post T-cell injection. Values are plotted as means±1 SD for each treatment group (n=4 mice per group, though one mouse in each group reached human endpoint before RO blood collection). (20M) Fold-change in C57BL/6 mouse weight. Mouse weight over time was normalized to values from day-16 post T-cell injection. Values are plotted as means±1 SD for each treatment group (n=4 mice per group). Statistics in (20B), (20C), (20G), (20J), and (20K) were calculated using the two-tailed, unpaired, two-sample Student's t test. Statistics in (20D), (20E), (20H), and (20M) were calculated by two-way ANOVA (n.s., not significant). Statistics in (20L) were calculated by a mixed effects model (n.s., not significant). There were no statistically significant differences detected among all pairwise comparisons.

FIGS. 21A-D. TGF-β-mediated activation of tanCAR-T cells leads to superior therapeutic outcomes compared to sCAR+DNR-T cells. (21A) Representative histogram of tanCAR expression in human T cells quantified via surface FLAG tag staining. (21B) Patient characteristics for the neurosphere lines investigated in FIGS. 15B-E. (21C) Tumor radiance traces corresponding to survival curve data shown in FIG. 2G. Each trace represents a single mouse and ends at the last imaging data point prior to euthanasia (n=5 for scFv-less CAR and tanCAR-T cell treatment groups, n=9 for sCAR+DNR-T cells). (21D) H&E staining and IL-13Rα2 IHC staining on paraffin-embedded brain slides prepared from mouse treated with sCAR+DNR-T cells. (Top) Representative brain section from mice that died without detectable tumor radiance. Staining shows outgrowth of IL-13Rα2⁺ tumors, confirming cause of death to be tumor-related. (Bottom) Representative slide of healthy NSG mouse brain section included as negative controls. H&E images were taken at 10× magnification; immunofluorescent images were taken at 20× magnification. Scalebars represent 1 mm (left) and 100 μm (right). Boxed regions are approximate.

FIGS. 22A-D. Bispecific IL-13Rα2/TGF-β tanCAR cross-reacts with murine TGF-β and are functional in murine T cells. (22A) Schematic of single-input TGF-β CAR, as previously described⁴³. (22B) Cytokine production by human T cells expressing the single-input TGF-β CAR and stimulated by human or murine TGF-β. The CAR contains human CD28 and CD3ζ signaling domains. TNF-α (left) and IFN-γ (right) expression was measured by intracellular flow cytometry following overnight culture. (22C) Activation of murine T cells expressing single-input TGF-β CAR containing either human or murine CD28 or CD3ζ signaling domains. CAR-T cells were incubated in the presence of TGF-β overnight, then CD69 (left) and CD25 (right) expression were measured by flow cytometry. (22D) Murine T cells transduced with single-input IL-13Rα2 CAR (sCAR) or IL-13Rα2/TGF-β bispecific tanCAR encoding human 4-1BB and CD33 signaling domains were cultured in the presence of either human or murine TGF-β. CD69 (left) and CD25 (right) expression was measured by flow cytometry following overnight culture. Statistics in (22B) and (22C) were computed by two-way ANOVA, with Tukey's method to correct for multiple comparisons. Statistics in (22D) were calculated using the two-tailed, unpaired, two-sample Student's t test with the Holm-Sidak correction for multiple comparisons (** P<0.01. *** P<0.001, **** P<0.0001).

FIGS. 23A-D. Immune-cell types in murine gliomas, with comparable numbers of brain-infiltrating leukocytes across treatment groups. (23A) UMAP projections and clusters identified from CyTOF analysis of CT-2A tumor-bearing brains. Cellular phenotypes corresponding to each cluster label are shown on the right. (23B) Total CD45+ cells recovered from individual mouse brains as quantified by flow cytometry. Similar total numbers of brain-infiltrating leukocytes were observed in each treatment group. Statistics were calculated using the two-tailed, unpaired, two-sample Student's t test (n.s., not significant). (23C) FOXP3 expression levels overlaid on UMAP projections of CyTOF analyses on CT-2A tumor-bearing brains. The CD4⁺ T cell cluster (circled) has little to no detectable FOXP3⁺ expression. (23D) NK. T. B, and myeloid cell lineage marker expression levels overlaid on UMAP projections of CyTOF analyses on CT-2A tumor-bearing brains. Cluster 14 (which accounts for <1% of all analyzed CD45⁺ cells) exhibits high expression of multiple lineage markers and was therefore classified as “Unknown.”

FIG. 24. Tumor radiance in mice sacrificed for CyTOF and scRNA-seq sample collection. Data correspond to mice whose brain-tissue analyses are shown in FIGS. 17-18.

FIG. 25. Bispecific IL-13Rα2/TGF-β tanCAR-T cells do not induce systemic toxicity in immunocompetent mice. Tumor radiance (top) and fold-change in mouse weights relative to baseline (bottom) are plotted for mice corresponding to each treatment group. Each individual trace represents a single mouse and ends at the last imaging data point prior to euthanasia. Data shown are from the same experiment as depicted in FIG. 20E. Weight loss corresponded to tumor outgrowth, not CAR-T cell treatment.

FIGS. 26A-C. Exemplary flow-cytometry and CyTOF gating paths. (26A) For all flow cytometric analyses, dead cells and debris were excluded by gating on SSC-A vs. FSC-A plots, followed by exclusion of doublets by gating on FSC-H vs. FSC-A plots. (26B) For flow cytometric analysis of activation marker expression (FIG. 16D, FIGS. 22C, 22D), unactivated and untransduced T cells were used as negative gating controls. (26C) For CyTOF analyses performed in FIG. 17 and FIG. 23, data were manually gated for CD45⁺ viable singlets as depicted prior to unsupervised clustering analysis.

FIG. 27. CAR-T cells were evaluated in aggressive GL261 murine glioma cells. As shown in the figure (bottom), only bispecific IL-13Rα2/TGF-β CAR-T cells are able to mount anti-tumor response.

FIGS. 28A-G. Immune-cell profiling of the GL261 TME via CyTOF. (28A) Schematic describing experimental design. (28B) % of CD45+ cells. Each group of bars shows data for scFv-less CAR, IL13Rα2.BBz, IL13Rα2.BBz+TGF-β DNR, and IL-13Rα2/TGF-β.BBZ, respectively. As shown in FIG. 28B, fewer suppressive myeloid cells are detected in mice treated with bispecific IL-13Rα2/TGF-β CAR-T cells. (28C) Total CD45+ Counts. As shown in FIG. 28C, similar numbers of CD45+ cells were recovered from brains for each treatment group. (28D-28E) Violin plots of PD-L1 expression following IL-13Rα2 CAR-T cell treatment (28D) and IL-13Rα2/TGF-β CAR-T cell treatment (28E). As shown in FIG. 28D-28E, myeloid cells upregulate PD-L1 expression following IL-13Rα2 CAR-T cell treatment and to al lesser extent following bispecific IL-13Rα2/TGF-β CAR-T cell treatment. (28F-28G) Violin plots of PD-1 and PD-L1 expression following bispecific IL-13Rα2/TGF-β CAR-T cell treatment. As shown in FIG. 28F, intratumoral CD8+ T cells exhibit less exhausted and more effector-like phenotypes following bispecific IL-13Rα2/TGF-β CAR-T cell treatment. As shown in FIG. 28G, bispecific IL-13Rα2/TGF-β CAR-T cells counter PD-1/PD-L1-mediated suppression in GL261 TME.

FIGS. 29A-E. CAR-T cells were evaluated in CT-2A murine glioma cells. (29A) Schematic describing experimental design. (29B) Tumor outgrowth. Single-input IL13Rα2 CAR-T cells exhibit the poorest control over tumor outgrowth. (29C) Schematic for evaluating bispecific IL-13Rα2/TGF-β CAR-T cells against CT-2A. (29D) Tumor outgrowth of IL13Rα2-tumors observed in (most) naïve mice. (29E) Some control over antigen-negative tumor outgrowth is observed with bispecific IL-13Rα2/TGF-β CAR-T cell treatment but not will single-input IL-13Rα2 CAR-T cell treatment.

FIGS. 30A-F. Profiling changes to the CT-2A TME via CyTOF and scRNAseq. (30A) Schematic describing experimental design. (30B) Total CD45+ cell counts. Similar numbers of CD45+ cells recovered from brains for each treatment group. (30C) T cell distribution in CT-2A and GL261 tumors. CT-2A tumors are more heavily infiltrated with T cells compared to GL261 tumors. (30D) Violin plots of PD-L1 expression following IL-13Rα2 CAR-T cell treatment. In contrast to GL261, myeloid cells appear to be less suppressive following IL-13Rα2 CAR-T cell treatment. (30E) Violin plots of PD-1 expression following IL-13Rα2 CAR-T cell treatment. Reduced PD-L1 among myeloid cells correlates with reduced PD-1 among T cells following IL-13Rα2 CAR-T cell treatment. (30F) Violin plots of PD-L1 expression following IL-13Rα2/TGF-β CAR-T cell treatment. Intratumoral DCs in IL-13Rα2/TGF-β CAR-T cell treated mice appear to be the most functional.

FIG. 31. % of CD45+ cells. IL-13Rα2/TGF-β CAR-T cell treatment favorably re-shapes the immune-cell compositions of the TME. Each group of four bars shows data for scFv-less CAR, IL13Rα2.BBz, IL13Rα2.BBz+TGF-β DNR, and IL-13Rα2/TGF-β.BBz, respectively.

### DETAILED DESCRIPTION

### I. Definitions

The peptides of the disclosure relate to peptides comprising chimeric antigen receptors, or CARs. CARs are engineered receptors, which are capable of grafting an arbitrary specificity onto an immune effector cell. In some cases, these receptors are used to graft the specificity of a monoclonal antibody onto a T cell. The receptors are called chimeric because they are composed of parts from different sources.

The terms “protein,” “polypeptide,” and “peptide” are used interchangeably herein when referring to a gene product.

“Homology.” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino proximal.” “N-terminus,” “amino terminus,” and the like as used herein are used to refer to order of the regions of the polypeptide. Furthermore, when something is N-terminal or amino proximal to a region it is not necessarily at the terminus (or end) of the entire polypeptide, but just at the N-terminus of the region or domain. Similarly, the terms “carboxy proximal.” “C-terminus,” “carboxy terminus,” and the like as used herein is used to refer to order of the regions of the polypeptide, and when something is C-terminal or carboxy proximal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment or aspect of this invention that is a polynucleotide encompasses both the double-stranded form and each of two complementary single-stranded forms known or predicted to make up the double-stranded form.

A “gene,” “polynucleotide.” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof” means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)2, Fab, Fv, rIgG, and scFv fragments. The term scFv refers to a single chain Fv antibody in which the variable domains of the heavy chain and of the light chain of a traditional two chain antibody have been joined to form one chain.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

“Individual, “subject,” and “patient” are used interchangeably and can refer to a human or non-human.

The terms “lower,” “reduced,” “reduction,” “decrease,” or “inhibit” are all used herein generally to mean a decrease by a statistically significant amount. However, for avoidance of doubt, “lower,” “reduced,” “reduction, “decrease,” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase,” “enhance,” or “activate” are all used herein to generally mean an increase by a statically significant amount; for the avoidance of any doubt, the terms “increased,” “increase,” “enhance,” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

### II. Polypeptides

### A. Signal Peptide

Polypeptides of the present disclosure may comprise a signal peptide. A “signal peptide” refers to a peptide sequence that directs the transport and localization of the protein within a cell, e.g., to a certain cell organelle (such as the endoplasmic reticulum) and/or the cell surface. In some aspects, a signal peptide directs the nascent protein into the endoplasmic reticulum. This is essential if a receptor is to be glycosylated and anchored in the cell membrane. Generally, the signal peptide natively attached to the amino-terminal most component is used (e.g. in an scFv with orientation light chain-linker-heavy chain, the native signal of the light-chain is used).

In some aspects, the signal peptide is cleaved after passage of the endoplasmic reticulum (ER), i.e., is a cleavable signal peptide. In some aspects, a restriction site is at the carboxy end of the signal peptide to facilitate cleavage.

### B. Antigen Binding Domain

Polypeptides of the present disclosure may comprise one or more antigen binding domains. An “antigen binding domain” describes a region of a polypeptide capable of binding to an antigen under appropriate conditions. In some aspects, an antigen binding domain is a single-chain variable fragment (scFv) based on one or more antibodies (e.g., CD20 antibodies). In some aspects, an antigen binding domain comprise a variable heavy (VH) region and a variable light (VL) region, with the VH and VL regions being on the same polypeptide. In some aspects, the antigen binding domain comprises a linker between the VH and VL regions. A linker may enable the antigen binding domain to form a desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M. In some aspects, the KD of the antigen binding region, such as the variable regions (heavy chain and/or light chain variable region), or of the CDRs may be at least 10-5M, 10-6M, 10-7M, 10-8M, 10-9M, 10-10M, 10-11M, 10-12M, or 10-13M (or any derivable range therein).

Binding affinity, KA, or KD can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some aspects, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity and/or expression level in host cells, compared to a polypeptide comprising a non-humanized binding region, such as a binding region from a mouse.

In some aspects, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some aspects, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

### C. Peptide Spacer

A peptide spacer, such as an extracellular spacer may link an antigen-binding domain to a transmembrane domain. In some aspects, a peptide spacer is flexible enough to allow the antigen-binding domain to orient in different directions to facilitate antigen binding. In one aspect, the spacer comprises the hinge region from IgG. In some aspects, the spacer comprises or further comprises the CH2CH3 region of immunoglobulin and portions of CD3. In some aspects, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some aspects, the spacer is from IgG4. An extracellular spacer may comprise a hinge region.

As used herein, the term “hinge” refers to a flexible polypeptide connector region (also referred to herein as “hinge region”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22:161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425, incorporated by reference herein. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some aspects, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87:162; and Huck et al. (1986) Nucl. Acids Res.

The length of an extracellular spacer may have effects on the CAR's signaling activity and/or the CAR-T cells' expansion properties in response to antigen-stimulated CAR signaling. In some aspects, a shorter spacer such as less than 50, 45, 40, 30, 35, 30, 25, 20, 15, 14, 13, 12, 11, or 10 amino acids is used. In some aspects, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences:

Table: Exemplary Hinge Regions

| SEQUENCE | SEQ ID NO: |
|---|---|
| DKTHT | 70 |
| CPPC | 71 |
| CPEPKSCDTPPPCPR | 72 |
| ELKTPLGDTTHT | 73 |
| KSCDKTHTCP | 74 |
| KCCVDCP | 75 |
| KYGPPCP | 76 |
| EPKSCDKTHTCPPCP | 77 |
| ELKTPLGDTTHTCPRCP | 78 |
| SPNMVPHAHHAQ | 79 |
| ESKYGPPCPPCP | 80 |
| EPKSCDKTYTCPPCP | 81 |

The extracellular spacer can comprise an amino acid sequence of a human IgG1, IgG2, IgG3, or IgG4, hinge region. The extracellular spacer may also include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:81).

The extracellular spacer can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:82), or a variant thereof.

The extracellular spacer may comprise or further comprise a CH2 region. An exemplary CH2 region is APEFEGGPSVFLFPPKPKDTLMISRTPEVTCVVVDVSQEDPEVQFNWYVDGVEVHNA KTKPREEQFQSTYRVVSVLTVLHQDWLNGKEYKCKVSNKGLPSSIEKTISKAK (SEQ ID NO: 83). The extracellular spacer may comprise or further comprise a CH3 region. An exemplary CH3 region is GQPREPQVYTLPPSQEEMTKNQVSLTCLVKGFYPSDIAVEWESNGQPENNYKTTPPV LDSDGSFFLYSRLTVDKSRWQEGNVFSCSVMHEALHNHYTQKSLSLSLGK (SEQ ID NO: 84).

When the extracellular spacer comprises multiple parts, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some aspects, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

### D. Transmembrane Domain

Polypeptides of the present disclosure may comprise a transmembrane domain. In some aspects, a transmembrane domain is a hydrophobic alpha helix that spans the membrane. Different transmembrane domains may result in different receptor stability.

In some aspects, the transmembrane domain is interposed between the extracellular spacer and the cytoplasmic region. In some aspects, the transmembrane domain is interposed between the extracellular spacer and one or more costimulatory regions. In some aspects, a linker is between the transmembrane domain and the one or more costimulatory regions.

Any transmembrane domain that provides for insertion of a polypeptide into the cell membrane of a eukaryotic (e.g., mammalian) cell may be suitable for use. In some aspects, the transmembrane domain is derived from CD28, CD8, CD4, CD3-zeta, CD134, or CD7.

Exemplary transmembrane domains useful in any of the aspects of the disclosure include those in the table below:

Table: Exemplary transmembrane domain sequences

| | | SEQ ID |
|---|---|---|
| Description | Sequence | NO: |
| CD28-derived | FWVLVVVGGVLACYSLLVTVAFIIFWV | 85 |
| CD8 beta derived | LGLLVAGVLVLLVSLGVAIHLCC | 86 |
| CD4 derived | ALIVLGGVAGLLLFIGLGIFFCVRC | 87 |
| CD3 zeta derived | LCYLLDGILFIYGVILTALFLRV | 88 |
| CD28 derived | WVLVVVGGVLACYSLLVTVAFIIFWV | 89 |
| CD134 (OX40) derived | VAAILGLGLVLGLLGPLAILLALYLL | 90 |
| CD7 derived | ALPAALAVISFLLGLGLGVACVLA | 91 |

### E. Cytoplasmic Region

After antigen recognition, receptors of the present disclosure may cluster and a signal transmitted to the cell through the cytoplasmic region. In some aspects, the costimulatory domains described herein are part of the cytoplasmic region. In some aspects, the cytoplasmic region comprises an intracellular signaling domain. An intracellular signaling domain may comprise a primary signaling domain and one or more costimulatory domains.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some aspects, the cytoplasmic region includes at least one (e.g., one, two, three, four, five, six, etc.) ITAM motif as described herein. In some aspects, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX1X2(L/I), where X1 and X2 are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX1X2(L/I))(X3)n(YX1X2(L/I)), where n is an integer from 6 to 8, and each of the 6-8 X3 can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

Exemplary cytoplasmic regions are known in the art. The cytoplasmic regions shown below also provide examples of regions that may be incorporated in a CAR of the disclosure:

In some aspects, a suitable cytoplasmic region can comprise an ITAM motif-containing portion of the full length DAP12 amino acid sequence. In some aspects, the cytoplasmic region is derived from FCER1G (also known as FCRG; Fc epsilon receptor I gamma chain; Fc receptor gamma-chain; fc-epsilon RI-gamma; fcRgamma; fceRI gamma; high affinity immunoglobulin epsilon receptor subunit gamma; immunoglobulin E receptor, high affinity, gamma chain; etc.). In some aspects, a suitable cytoplasmic region can comprise an ITAM motif-containing portion of the full length FCER1G amino acid sequence.

In some aspects, the cytoplasmic region is derived from T cell surface glycoprotein CD3 delta chain (also known as CD3D; CD3-DELTA; T3D; CD3 antigen, delta subunit; CD3 delta; CD38; CD3d antigen, delta polypeptide (TiT3 complex); OKT3, delta chain; T cell receptor T3 delta chain; T cell surface glycoprotein CD3 delta chain; etc.). In some aspects, a suitable cytoplasmic region can comprise an ITAM motif-containing portion of the full length CD3 delta amino acid sequence. In some aspects, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, AI504783, CD3, CD3-epsilon, T3c, etc.). In some aspects, a suitable cytoplasmic region can comprise an ITAM motif-containing portion of the full length CD3 epsilon amino acid sequence. In some aspects, the cytoplasmic region is derived from T cell surface glycoprotein CD3 gamma chain (also known as CD3G, CD3γ. T cell receptor T3 gamma chain, CD3-GAMMA, T3G, gamma polypeptide (TiT3 complex), etc.). In some aspects, a suitable cytoplasmic region can comprise an ITAM motif-containing portion of the full length CD3 gamma amino acid sequence. In some aspects, the cytoplasmic region is derived from T cell surface glycoprotein CD3 zeta chain (also known as CD3Z, CD3ζ. T cell receptor T3 zeta chain, CD247, CD3-ZETA, CD3H, CD3Q, T3Z, TCRZ, etc.). In some aspects, a suitable cytoplasmic region can comprise an ITAM motif-containing portion of the full length CD3 zeta amino acid sequence.

In some aspects, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In some aspects, a suitable cytoplasmic region can comprise an ITAM motif-containing portion of the full length CD79A amino acid sequence.

Specific exemplary cytoplasmic regions are known in the art and further shown in the table below.

Table: Cytoplasmic Regions

| SEQUENCE | SEQ ID NO: |
|---|---|
| MGGLEPCSRLLLLPLLLAVSGLRPVQAQAQSDCSCSTVSPGVLAGIVMGD | 92 |
| LVLTVLIALAVYFLGRLVPRGRGAAEAATRKQRITETESPYQELQGQRSD | |
| VYSDLNTQRPYYK | |
| MGGLEPCSRLLLLPLLLAVSGLRPVQAQAQSDCSCSTVSPGVLAGIVMGD | 93 |
| LVLTVLIALAVYFLGRLVPRGRGAAEATRKQRITETESPYQELQGQRSDV | |
| YSDLNTQRPYYK | |
| MGGLEPCSRLLLLPLLLAVSDCSCSTVSPGVLAGIVMGDLVLTVLIALAV | 94 |
| YFLGRLVPRGRGAAEAATRKQRITETESPYQELQGQRSDVYSDLNTQRPY | |
| YK | |
| MGGLEPCSRLLLLPLLLAVSDCSCSTVSPGVLAGIVMGDLVLTVLIALAV | 95 |
| YFLGRLVPRGRGAAEATRKQRITETESPYQELQGQRSDVYSDLNTQRPYY | |
| K | |
| MIPAVVLLLLLLVEQAAALGEPQLCYILDAILFLYGIVLTLLYCRLKIQVR | 96 |
| KAAITSYEKSDGVYTGLSTRNQETYETLKHEKPPQ | |
| DGVYTGLSTRNQETYETLKHE | 97 |
| MEHSTFLSGLVLATLLSQVSPFKIPIEELEDRVFVNCNTSITWVEGTVGTLL | 98 |
| SDITRLDLGKRILDPRGIYRCNGTDIYKDKESTVQVHYRMCQSCVELDPA | |
| TVAGIIVTDVIATLLLALGVFCFAGHETGRLSGAADTQALLRNDQVYQPL | |
| RDRDDAQYSHLGGNWARNK | |
| MEHSTFLSGLVLATLLSQVSPFKIPIEELEDRVFVNCNTSITWVEGTVGTLL | 99 |
| SDITRLDLGKRILDPRGIYRCNGTDIYKDKESTVQVHYRTADTQALLRND | |
| QVYQPLRDRDDAQYSHLGGNWARNK | |
| DQVYQPLRDRDDAQYSHLGGN | 100 |
| MQSGTHWRVLGLCLLSVGVWGQDGNEEMGGITQTPYKVSISGTTVILTC | 101 |
| PQYPGSEILWQHNDKNIGGDEDDKNIGSDEDHLSLKEFSELEQSGYYVCY | |
| PRGSKPEDANFYLYLRARVCENCMEMDVMSVATIVIVDICITGGLLLLVY | |
| YWSKNRKAKAKPVTRGAGAGGRQRGQNKERPPPVPNPDYEPIRKGQRD | |
| LYSGLNQRRI | |
| NPDYEPIRKGQRDLYSGLNQR | 102 |
| MEQGKGLAVLILAIILLQGTLAQSIKGNHLVKVYDYQEDGSVLLTCDAEA | 103 |
| KNITWFKDGKMIGFLTEDKKKWNLGSNAKDPRGMYQCKGSQNKSKPLQ | |
| VYYRMCQNCIELNAATISGFLFAEIVSIFVLAVGVYFIAGQDGVRQSRASD | |
| KQTLLPNDQLYQPLKDREDDQYSHLQGNQLRRN | |
| DQLYQPLKDREDDQYSHLQGN | 104 |
| MKWKALFTAAILQAQLPITEAQSFGLLDPKLCYLLDGILFIYGVILTALFLR | 105 |
| VKFSRSADAPAYQQGQNQLYNELNLGRREEYDVLDKRRGRDPEMGGKP | |
| RRKNPQEGLYNELQKDKMAEAYSEIGMKGERRRGKGHDGLYQGLSTAT | |
| KDTYDALHMQALPPR | |
| MKWKALFTAAILQAQLPITEAQSFGLLDPKLCYLLDGILFIYGVILTALFLR | 106 |
| VKFSRSADAPAYQQGQNQLYNELNLGRREEYDVLDKRRGRDPEMGGKP | |
| QRRKNPQEGLYNELQKDKMAEAYSEIGMKGERRRGKGHDGLYQGLSTA | |
| TKDTYDALHMQALPPR | |
| RVKFSRSADAPAYQQGQNQLYNELNLGRREEYDVLDKRRGRDPEMGGK | 8 |
| PRRKNPQEGLYNELQKDKMAEAYSEIGMKGERRRGKGHDGLYQGLSTA | |
| TKDTYDALHMQALPPR | |
| NQLYNELNLGRREEYDVLDKR | 107 |
| EGLYNELQKDKMAEAYSEIGMK | 108 |
| DGLYQGLSTATKDTYDALHMQ | 109 |
| MPGGPGVLQALPATIFLLFLLSAVYLGPGCQALWMHKVPASLMVSLGED | 110 |
| AHFQCPHNSSNNANVTWWRVLHGNYTWPPEFLGPGEDPNGTLIIQNVNK | |
| SHGGIYVCRVQEGNESYQQSCGTYLRVRQPPPRPFLDMGEGTKNRIITAE | |
| GIILLFCAVVPGTLLLFRKRWQNEKLGLDAGDEYEDENLYEGLNLDDCS | |
| MYEDISRGLQGTYQDVGSLNIGDVQLEKP | |
| MPGGPGVLQALPATIFLLFLLSAVYLGPGCQALWMHKVPASLMVSLGED | 111 |
| AHFQCPHNSSNNANVTWWRVLHGNYTWPPEFLGPGEDPNEPPPRPFLDM | |
| GEGTKNRIITAEGIILLFCAVVPGTLLLFRKRWQNEKLGLDAGDEYEDENL | |
| YEGLNLDDCSMYEDISRGLQGTYQDVGSLNIGDVQLEKP | |
| ENLYEGLNLDDCSMYEDISRG | 112 |
| FWVLVVVGGVLACYSLLVTVAFIIFWVRSKRSRLLHSDYMNMTPRRPGP | 113 |
| TRKHYQPYAPPRDFAAYRS | |

### F. Costimulatory Region

Non-limiting examples of suitable costimulatory regions, such as those included in the cytoplasmic region, include, but are not limited to, polypeptides from 4-IBB (CD137), CD28, ICOS, OX-40, BTLA, CD27, CD30, GITR, and HVEM.

A costimulatory region may have a length of at least, at most, or exactly 20, 25, 30, 35, 40, 50, 60, 70, 80, 90, 100, 150, 200, or 300 amino acids or any range derivable therein. In some aspects, the costimulatory region is derived from an intracellular portion of the transmembrane protein 4-1BB (also known as TNFRSF9; CD137; CDw137; ILA; etc.). In some aspects, the costimulatory region is derived from an intracellular portion of the transmembrane protein CD28 (also known as Tp44). In some aspects, the costimulatory region is derived from an intracellular portion of the transmembrane protein ICOS (also known as AILIM, CD278, and CVID1). In some aspects, the costimulatory region is derived from an intracellular portion of the transmembrane protein OX-40 (also known as TNFRSF4, RP5-902P8.3, ACT35, CD134, OX40, TXGP1L). In some aspects, the costimulatory region is derived from an intracellular portion of the transmembrane protein BTLA (also known as BTLA1 and CD272). In some aspects, the costimulatory region is derived from an intracellular portion of the transmembrane protein CD27 (also known as S 152, T14, TNFRSF7, and Tp55). In some aspects, the costimulatory region is derived from an intracellular portion of the transmembrane protein CD30 (also known as TNFRSF8, D1S166E, and Ki-1). In some aspects, the costimulatory region is derived from an intracellular portion of the transmembrane protein GITR (also known as TNFRSF18, RP5-902P8.2, AITR, CD357, and GITR-D). In some aspects, the costimulatory region derived from an intracellular portion of the transmembrane protein HVEM (also known as TNFRSF14, RP3-395M20.6, ATAR, CD270, HVEA, HVEM, LIGHTR, and TR2).

Specific exemplary co-stimulatory domains are represented by the amino acid sequences below:

Table: Co-stimulatory domains

| SEQUENCE | SEQ ID NO: |
|---|---|
| KRGRKKLLYIFKQPFMRPVQTTQEEDGCSCRFPEEEEGGCEL | 7 |
| FWVRSKRSRLLHSDYMNMTPRRPGPTRKHYQPYAPPRDFAAYRS | 114 |
| TKKKYSSSVHDPNGEYMFMRAVNTAKKSRLTDVTL | 115 |
| RRDQRLPPDAHKPPGGGSFRTPIQEEQADAHSTLAKI | 116 |
| CCLRRHQGKQNELSDTAGREINLVDAHLKSEQTEASTRQNSQVLLSETGI | 117 |
| YDNDPDLCFRMQEGSEVYSNPCLEENKPGIVYASLNHSVIGPNSRLARN | |
| VKEAPTEYASICVRS | |
| HQRRKYRSNKGESPVEPAEPCRYSCPREEEGSTIPIQEDYRKPEPACSP | 118 |
| RRACRKRIRQKLHLCYPVQTSQPKLELVDSRPRRSSTQLRSGASVTEPVA | 119 |
| EERGLMSQPLMETCHSVGAAYLESLPLQDASPAGGPSSPRDLPEPRVSTE | |
| HTNNKIEKIYIMKADTVIVGTVKAELPEGRGLAGPAEPELEEELEADHTP | |
| HYPEQETEPPLGSCSDVMLSVEEEGKEDPLPTAASGK | |
| HIWQLRSQCMWPRETQLLLEVPPSTEDARSCQFPEEERGERSAEEKGRL | 120 |
| GDLWV | |
| CVKRRKPRGDVVKVIVSVQRKRQEAEGEATVIEALQAPPDVTTVAVEET | 121 |
| IPSFTGRSPNH | |
| RSKRSRGGHSDYMNMTPRRPGPTRKHYQPYAPPRDFAAYRS | 18 |

### G. Detection Peptides

In some aspects, the polypeptides described herein may further comprise a detection peptide. Suitable detection peptides include hemagglutinin (HA; e.g., YPYDVPDYA (SEQ ID NO: 122); FLAG (e.g., DYKDDDDK (SEQ ID NO:3); c-myc (e.g., EQKLISEEDL; SEQ ID NO: 123), and the like. Other suitable detection peptides are known in the art.

### H. Peptide Linkers

In some aspects, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Example flexible linkers include glycine polymers (G)n, glycine-serine polymers (including, for example, (GS)n, (GSGGS-SEQ ID NO:159)n, (G4S)n and (GGGS-SEQ ID NO: 160)n, where n is an integer of at least one. In some aspects, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:125), GGSGG (SEQ ID NO:126), GSGSG (SEQ ID NO: 127), GSGGG (SEQ ID NO:128), GGGSG (SEQ ID NO:129), GSSSG (SEQ ID NO:124), SEQ ID NO:10, SEQ ID NO:28, and the like. In some aspects, the linker comprises a repeat, such as a contiguous repeat of one or more of SEQ ID NOS: 124-129, 10, and 28, such as a linker comprising an amino acid sequence that corresponds to one of SEQ ID NOS: 124-129, 10, and 28 repeated at least, at most, or exactly 2, 3, 4, 5, 6, 7, 8, 9, or 10 times, or any range derivable therein.

In further aspects, the linker comprises (EAAAK)n(SEQ ID NO:130), wherein n is an integer of at least one. In some aspects, n is at least, at most, or exactly 1, 2, 3, 4, 5, 6, 7, 8, 9, or 10 (or any derivable range therein).

### I. Therapeutic Controls

In some aspects of the methods and compositions described herein, the CAR molecule is co-expressed with a therapeutic control.

Therapeutic controls regulate cell proliferation, facilitate cell selection (for example selecting cells which express the chimeric antigen receptors of the invention) or a combination thereof. In one aspect, regulating cell proliferation comprises up-regulating cell proliferation to promote cell propagation. In another aspect, regulating cell proliferation comprises down-regulating cell proliferation so as to reduce or inhibit cell propagation. In some aspects, the agents that serve as therapeutic controls may promote enrichment of cells which express the chimeric antigen receptors which may result in a therapeutic advantage. In some aspects, agents which serve as therapeutic controls may biochemically interact with additional compositions so as to regulate the functioning of the therapeutic controls. For example, EGFRt (a therapeutic control) may biochemically interact with cetuximab so as to regulate the function of EGFRt in selection, tracking, cell ablation or a combination thereof.

Exemplary therapeutic controls include truncated epidermal growth factor receptor (EGFRt), chimeric cytokine receptors (CCR) and/or dihydroxyfolate receptor (DHFR) (e.g., mutant DHFR). The polynucleotides encoding the CAR and the therapeutic control(s) may be linked via IRES sequences or via polynucleotide sequences encoding cleavable linkers. The CARs of the invention are constructed so that they may be expressed in cells, which in turn proliferate in response to the presence of at least one molecule that interacts with at least one antigen-specific targeting region, for instance, an antigen. In further aspects, the therapeutic control comprises a cell-surface protein wherein the protein lacks intracellular signaling domains. It is contemplated that any cell surface protein lacking intracellular signaling or modified (e.g. by truncation) to lack intracellular signaling may be used. Further examples of a therapeutic control include truncated LNGFR, truncated CD19, etc., wherein the truncated proteins lack intracellular signaling domains.

“Co-express” as used herein refers to simultaneous expression of two or more genes. Genes may be nucleic acids encoding, for example, a single protein or a chimeric protein as a single polypeptide chain. For example, the CARs of the disclosure may be co-expressed with a therapeutic control (for example truncated epidermal growth factor (EGFRt)), wherein the CAR is encoded by a first polynucleotide chain and the therapeutic control is encoded by a second polynucleotide chain. In one aspect, the first and second polynucleotide chains are linked by a nucleic acid sequence that encodes a cleavable linker The polynucleotides encoding the CAR and the therapeutic control system may be linked by IRES sequences. Alternately, the CAR and the therapeutic control are encoded by two different polynucleotides that are not linked via a linker but are instead encoded by, for example, two different vectors. Further, the CARs of the disclosure may be co-expressed with a therapeutic control and CCR, a therapeutic control and DHFR (for example mutant DHFR) or a therapeutic control and CCR and DHFR (for example mutant DHFR). The CAR, therapeutic control and CCR may be co-expressed and encoded by first, second and third polynucleotide sequences, respectively, wherein the first, second and third polynucleotide sequences are linked via IRES sequences or sequences encoding cleavable linkers (e.g., T2A). Alternately, these sequences are not linked via linkers but instead are encoded via, for example, separate vectors. The CAR, therapeutic control and DHFR (for example mutant DHFR) may be co-expressed and encoded by first, second and fourth polynucleotide sequences, respectively, wherein the first, second and fourth polynucleotide sequences are linked via IRES sequences or via sequences encoding cleavable linkers. Alternately, these sequences are not linked via linkers but instead encoded via, for example, separate vectors. The CAR, therapeutic control, CCR and DHFR (for example mutant DHFR) may be co-expressed and encoded by first, second, third and fourth polynucleotide sequences, respectively, wherein the first, second, third and fourth polynucleotide sequences are linked via IRES sequences or sequences encoding cleavable linkers. Alternately, these sequences are not linked via linkers but instead are encoded via, for example, separate vectors. If the aforementioned sequences are encoded by separate vectors, these vectors may be simultaneously or sequentially transfected.

Further aspects of the therapeutic controls, CAR molecules, and methods of use for the compositions of the disclosure can be found in U.S. Pat. No. 9,447,194, which is herein incorporated by reference for all purposes.

### J. Additional Modifications and Polypeptide Enhancements

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

It is contemplated that a region or fragment of a polypeptide of the disclosure may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOS: 1-172. Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOS: 1-172. Moreover, in some aspects, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOS: 1-172 (where position 1 is at the N-terminus of the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 600, or more contiguous amino acids, or any range derivable therein, of any of SEQ ID NOS: 1-172.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 650 of any of SEQ ID NOS: 1-172 (or any derivable range therein) and may be a substitution with any amino acid or may be a substitution with a alanine, arginine, asparagine, aspartic acid, cysteine, glutamine, glutamic acid, glycine, histidine, isoleucine, leusine, lysine, methionine, phenylalanine, proline, serine, threonine, tryptophan, tyrosine, or valine.

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other aspects, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific aspects, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One aspect includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. Cells

Certain aspects relate to cells comprising polypeptides or nucleic acids of the disclosure. In some aspects the cell is an immune cell or a T cell. “T cell” includes all types of immune cells expressing CD3 including T-helper cells, invariant natural killer T (INKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### IV. Methods for Modifying Genomic DNA

In certain aspects, the genomic DNA is modified either to include additional mutations, insertions, or deletions, or to integrate certain molecular constructs of the disclosure so that the constructs are expressed from the genomic DNA. In some aspects, a nucleic acid encoding a polypeptide of the disclosure is integrated into the genomic DNA of a cell. In some aspects, a nucleic acid is integrated into a cell via viral transduction, such as gene transfer by lentiviral or retroviral transduction. In some aspects, genomic DNA is modified by integration of nucleic acid encoding a polypeptide of the present disclosure (e.g., a CAR) into the genome of a host cell via a retroviral vector, a lentiviral vector, or an adeno-associated viral vector.

In some aspects, the integration is targeted integration. In some aspects, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T cell receptor alpha constant) locus. For instance, cells would first be electroporated with a ribonucleoprotein (RNP) complex consisting of Cas9 protein complexed with a single-guide RNA (sgRNA) targeting the TRAC (T cell receptor alpha constant) locus. Fifteen minutes post electroporation, the cells would be treated with AAV6 carrying the HDR template that encodes for the CAR. In another example, double stranded or single stranded DNA comprises the HDR template and is introduced into the cell via electroporation together with the RNP complex.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, ΦC31 integrase, Bxb1-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRISPR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31 (11): 2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7 (1): 171-192; Bogdanove A J, Voytas D F., 2011, Science, 333 (6051): 1843-6; Bradley P, Bogdanove A J, Stoddard B L., 2013, Curr Opin Struct Biol., 23 (1): 93-9.

### V. Methods

Aspects of the current disclosure relate to methods for treating cancer, such as malignant glioma, diffuse midline glioma, neuroblastoma, sarcoma, osteosarcoma, diffuse intrinsic pontine glioma, and melanoma. In further aspects, the therapeutic receptors (e.g., CARs) described herein may be used for stimulating an immune response. The immune response stimulation may be done in vitro, in vivo, or ex vivo. In some aspects, the therapeutic receptors described herein are for preventing relapse. The method generally involves genetically modifying a mammalian cell with an expression vector, or a DNA, an RNA (e.g., in vitro transcribed RNA), or an adeno-associated virus (AAV) comprising nucleotide sequences encoding a polypeptide of the disclosure or directly transferring the polypeptide to the cell. The cell can be an immune cell (e.g., a T lymphocyte or NK cell), a stem cell, a progenitor cell, etc. In some aspects, the cell is a cell described herein.

In some aspects, the genetic modification is carried out ex vivo. For example, a T lymphocyte, a stem cell, or an NK cell (or cell described herein) is obtained from an individual; and the cell obtained from the individual is genetically modified to express a polypeptide of the disclosure. In some cases, the genetically modified cell is activated ex vivo. In other cases, the genetically modified cell is introduced into an individual (e.g., the individual from whom the cell was obtained); and the genetically modified cell is activated in vivo.

In some aspects, the methods relate to administration of the cells or peptides described herein for the treatment of a cancer or administration to a person with a cancer. In some aspects, the cancer is diffuse midline glioma. In some aspects, the cancer is selected from malignant glioma, diffuse midline glioma, neuroblastoma, sarcoma, osteosarcoma, diffuse intrinsic pontine glioma, and melanoma.

### VI. Additional Therapies

### A. Immunotherapy

In some aspects, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

### 1. Checkpoint Inhibitors and Combination Treatment

Aspects of the disclosure may include administration of immune checkpoint inhibitors (also referred to as checkpoint inhibitor therapy), which are further described below. The checkpoint inhibitor therapy may be a monotherapy, targeting only one cellular checkpoint proteins or may be combination therapy that targets at least two cellular checkpoint proteins. For example, the checkpoint inhibitor monotherapy may comprise one of: a PD-1, PD-L1, or PD-L2 inhibitor or may comprise one of a CTLA-4, B7-1, or B7-2 inhibitor. The checkpoint inhibitor combination therapy may comprise one of: a PD-1, PD-L1, or PD-L2 inhibitor and, in combination, may further comprise one of a CTLA-4, B7-1, or B7-2 inhibitor. The combination of inhibitors in combination therapy need not be in the same composition, but can be administered either at the same time, at substantially the same time, or in a dosing regimen that includes periodic administration of both of the inhibitors, wherein the period may be a time period described herein.

a. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some aspects, PD-1, PD-L1, and PD-L2 are human PD-1, PD-L1 and PD-L2.

In some aspects, the PD-1 inhibitor is a molecule that inhibits the binding of PD-1 to its ligand binding partners. In a specific aspect, the PD-1 ligand binding partners are PD-L1 and/or PD-L2. In another aspect, a PD-L1 inhibitor is a molecule that inhibits the binding of PD-L1 to its binding partners. In a specific aspect, PD-L1 binding partners are PD-1 and/or B7-1. In another aspect, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some aspects, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHlgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PD-L1, or PD-L2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 2. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNΔ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Tumor targeted T cells can be generated through gene therapy. Tumor targeted T cells can be expanded by exposing the T cells to tumor antigens.

In some aspects, therapeutic cells used in adoptive cell therapies express chimeric antigen receptors (CARs). CARs are fusion proteins that are commonly composed of an extracellular antigen-binding domain (which may be an scFv), an extracellular spacer, a transmembrane domain, costimulatory signaling regions (the number of which varies depending on the specific CAR design), and a CD3-zeta signaling domain/endodomain.

In some aspects, therapeutic cells used in adoptive cell therapies express engineered T-cell receptors (TCRs), which are heterologous TCR molecules that target tumor antigens. Immune cells, including T cells and natural killer (NK) cells, can be engineered to express CARs or TCRs by a variety of methods known in the art, including viral transduction, DNA nucleofection, and RNA nucleofection. Binding of the CAR or TCR to the antigen target can activate human T cells expressing the CAR or TCR, which may result in killing of the cell bearing the antigen or some other immunological response.

In some aspects, the cells comprise a cancer-specific CAR or TCR. The term “cancer-specific” in the context of CAR or TCR polypeptides refers to a polypeptide that has an antigen binding specificity for a cancer-specific molecule, such as a cancer-specific antigen. In some aspects, the cancer-specific CAR and another CAR are on separate polypeptides.

### B. Oncolytic Virus

In some aspects, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### C. Polysaccharides

In some aspects, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### D. Neoantigens

In some aspects, the additional therapy comprises targeting of neoantigen mutations. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### E. Chemotherapies

In some aspects, the additional therapy comprises a chemotherapy. Suitable classes of chemotherapeutic agents include (a) Alkylating Agents, such as nitrogen mustards (e.g., mechlorethamine, cyclophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophyllotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydrazine derivatives (e.g., procarbazine), and adrenocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40.41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### VII. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intratumorally, intramuscular, intraperitoneal, intraperitoneally, intraorbitally, by implantation, by inhalation, intraventricularly, intracerebroventricularly, intranasally, intravenous injection, or into a tumor resection cavity.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In aspects where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some aspects, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some aspects, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one aspect, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50μ; or about 1μ M to 40μ; or about 1μ M to 30μ; or about 1μ M to 20μ; or about 1 μM to 10μ; or about 10μ M to 150μ; or about 10μ M to 100μ; or about 10μ M to 50 μM; or about 25 μM to 150 μM; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 UM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. Therapeutic Methods

The compositions of the disclosure may be used for in vivo, in vitro, or ex vivo administration. The route of administration of the composition may be, for example, intracutaneous, subcutaneous, intravenous, local, topical, and intraperitoneal administrations.

In some aspects, the disclosed methods are directed to methods for treating cancer. The cancer may be a solid tumor, metastatic cancer, or non-metastatic cancer. In certain aspects, the cancer may be recurrent, metastatic, relapsed, or of a Stage I, II, III, or IV.

### IX. Sequences

The amino acid sequence of example chimeric polypeptides and CAR molecules useful in the methods and compositions of the present disclosure are provided in Table 1 below.

| | | SEQ |
|---|---|---|
| DESCRIPTION | SEQUENCE | ID NO: |
| IL13Rα2.BBz | METDTLLLWVLLLWVPGSTGAGGSDYKDDDDKGGSG | 146 |
| (MKleader-FLAG- | PVPPSTALRYLIEELVNITQNQKAPLCNGSMVWSINLTA | |
| IL13op-IgG4 | GMYCAALESLINVSGCSAIEKTQRMLSGFCPHKVSAGQ | |
| hinge_IgG4 CH2 CH3 | FSSLHVRDTKIEVAQFVKDLLLHLKKLFREGRFNESKY | |
| L235E N297Q peptide | GPPCPPCPAPEFEGGPSVFLFPPKPKDTLMISRTPEVTCV | |
| spacer-CD28 | VVDVSQEDPEVQFNWYVDGVEVHNAKTKPREEQFQST | |
| transmembrane | YRVVSVLTVLHQDWLNGKEYKCKVSNKGLPSSIEKTIS | |
| domain-4-1BB Co- | KAKGQPREPQVYTLPPSQEEMTKNQVSLTCLVKGFYPS | |
| stimulatory-CD3 Zeta) | DIAVEWESNGQPENNYKTTPPVLDSDGSFFLYSRLTVD | |
| | KSRWQEGNVFSCSVMHEALHNHYTQKSLSLSLGKMF | |
| | WVLVVVGGVLACYSLLVTVAFIIFWVKRGRKKLLYIFK | |
| | QPFMRPVQTTQEEDGCSCRFPEEEEGGCELRVKFSRSA | |
| | DAPAYQQGQNQLYNELNLGRREEYDVLDKRRGRDPE | |
| | MGGKPRRKNPQEGLYNELQKDKMAEAYSEIGMKGER | |
| | RRGKGHDGLYQGLSTATKDTYDALHMQALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| IgG4 hinge: IgG4 CH2 | ESKYGPPCPPCPAPEFEGGPSVFLFPPKPKDTLMISRTPE | 5 |
| CH3 L235E N297Q | VTCVVVDVSQEDPEVQFNWYVDGVEVHNAKTKPREE | |
| peptide spacer | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-(G4S)x3- | METDTLLLWVLLLWVPGSTGAGGSDYKDDDDKGGSG | 148 |
| TGF-β.BBz | PVPPSTALRYLIEELVNITQNQKAPLCNGSMVWSINLTA | |
| (MKleader-FLAG- | GMYCAALESLINVSGCSAIEKTQRMLSGFCPHKVSAGQ | |
| IL13op-(G4S)x3-TGF- | FSSLHVRDTKIEVAQFVKDLLLHLKKLFREGRENGGGG | |
| β scFv-IgG4 hinge | SGGGGSGGGGSQVQLVQSGAEVKKPGSSVKVSCKASG | |
| peptide spacer-CD28 | YTFSSNVISWVRQAPGQGLEWMGGVIPIVDIANYAQRF | |
| transmembrane | KGRVTITADESTSTTYMELSSLRSEDTAVYYCALPRAF | |
| domain-4-1BB Co- | VLDAMDYWGQGTLVTVSSGGGGSGGGGSGGGGSETV | |
| stimulatory-CD3 Zeta) | LTQSPGTLSLSPGERATLSCRASQSLGSSYLAWYQQKP | |
| | GQAPRLLIYGASSRAPGIPDRFSGSGSGTDFTLTISRLEP | |
| | EDFAVYYCQQYADSPITFGQGTRLEIKESKYGPPCPPCP | |
| | MFWVLVVVGGVLACYSLLVTVAFIIFWVKRGRKKLLY | |
| | IFKQPFMRPVQTTQEEDGCSCRFPEEEEGGCELRVKFSR | |
| | SADAPAYQQGQNQLYNELNLGRREEYDVLDKRRGRDP | |
| | EMGGKPRRKNPQEGLYNELQKDKMAEAYSEIGMKGE | |
| | RRRGKGHDGLYQGLSTATKDTYDALHMQALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x3 | GGGGSGGGGSGGGGS | 28 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-(G4S)x4- | METDTLLLWVLLLWVPGSTGAGGSDYKDDDDKGGSG | 149 |
| TGF-β.BBz | PVPPSTALRYLIEELVNITQNQKAPLCNGSMVWSINLTA | |
| (MKleader-FLAG- | GMYCAALESLINVSGCSAIEKTQRMLSGFCPHKVSAGQ | |
| IL13op-(G4S)x4-TGF- | FSSLHVRDTKIEVAQFVKDLLLHLKKLFREGRENGGGG | |
| β scFv-IgG4 hinge | SGGGGSGGGGSGGGGSQVQLVQSGAEVKKPGSSVKVS | |
| peptide spacer-CD28 | CKASGYTFSSNVISWVRQAPGQGLEWMGGVIPIVDIAN | |
| transmembrane | YAQRFKGRVTITADESTSTTYMELSSLRSEDTAVYYCA | |
| domain-4-1BB Co- | LPRAFVLDAMDYWGQGTLVTVSSGGGGSGGGGSGGG | |
| stimulatory-CD3 Zeta) | GSETVLTQSPGTLSLSPGERATLSCRASQSLGSSYLAWY | |
| | QQKPGQAPRLLIYGASSRAPGIPDRFSGSGSGTDFTLTIS | |
| | RLEPEDFAVYYCQQYADSPITFGQGTRLEIKESKYGPPC | |
| | PPCPMFWVLVVVGGVLACYSLLVTVAFIIFWVKRGRK | |
| | KLLYIFKQPFMRPVQTTQEEDGCSCRFPEEEEGGCELRV | |
| | KFSRSADAPAYQQGQNQLYNELNLGRREEYDVLDKRR | |
| | GRDPEMGGKPRRKNPQEGLYNELQKDKMAEAYSEIG | |
| | MKGERRRGKGHDGLYQGLSTATKDTYDALHMQALPP | |
| | R | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-(G4S)x4- | METDTLLLWVLLLWVPGSTGAGGSDYKDDDDKGGSG | 150 |
| EGFRvIII.BBz | PVPPSTALRYLIEELVNITQNQKAPLCNGSMVWSINLTA | |
| (MKleader-FLAG- | GMYCAALESLINVSGCSAIEKTQRMLSGFCPHKVSAGQ | |
| IL13op-(G4S)x4- | FSSLHVRDTKIEVAQFVKDLLLHLKKLFREGRENGGGG | |
| EGFRvIII scFv-IgG4 | SGGGGSGGGGSGGGGSEIQLVQSGAEVKKPGESLRISC | |
| hinge peptide spacer | KGSGFNIEDYYIHWVRQMPGKGLEWMGRIDPENDETK | |
| transmembrane | YGPIFQGHVTISADTSINTVYLQWSSLKASDTAMYYCA | |
| domain-4-1BB Co- | FRGGVYWGQGTTVTVSSGSTSGSGKPGSGEGSTKGDV | |
| stimulatory-CD3 Zeta) | VMTQSPDSLAVSLGERATINCKSSQSLLDSDGKTYLNW | |
| | LQQKPGQPPKRLISLVSKLDSGVPDRFSGSGSGTDFTLTI | |
| | SSLQAEDVAVYYCWQGTHFPGTFGGGTKVEIKESKYG | |
| | PPCPPCPMFWVLVVVGGVLACYSLLVTVAFIIFWVKRG | |
| | RKKLLYIFKQPFMRPVQTTQEEDGCSCRFPEEEEGGCEL | |
| | RVKFSRSADAPAYQQGQNQLYNELNLGRREEYDVLDK | |
| | RRGRDPEMGGKPRRKNPQEGLYNELQKDKMAEAYSEI | |
| | GMKGERRRGKGHDGLYQGLSTATKDTYDALHMQALP | |
| | PR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| EGFRvIII scFv | EIQLVQSGAEVKKPGESLRISCKGSGFNIEDYYIHWVRQ | 27 |
| | MPGKGLEWMGRIDPENDETKYGPIFQGHVTISADTSIN | |
| | TVYLQWSSLKASDTAMYYCAFRGGVYWGQGTTVTVS | |
| | SGSTSGSGKPGSGEGSTKGDVVMTQSPDSLAVSLGERA | |
| | TINCKSSQSLLDSDGKTYLNWLQQKPGQPPKRLISLVSK | |
| | LDSGVPDRFSGSGSGTDFTLTISSLQAEDVAVYYCWQG | |
| | THFPGTFGGGTKVEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-(G4S)x4- | METDTLLLWVLLLWVPGSTGAGGSDYKDDDDKGGSG | 151 |
| EGFRvIII.BBz | PVPPSTALRYLIEELVNITQNQKAPLCNGSMVWSINLTA | |
| (MKleader-FLAG- | GMYCAALESLINVSGCSAIEKTQRMLSGFCPHKVSAGQ | |
| IL13op-(G4S)x4- | FSSLHVRDTKIEVAQFVKDLLLHLKKLFREGRENGGGG | |
| EGFRvIII scFv-IgG4 | SGGGGSGGGGSGGGGSEIQLVQSGAEVKKPGESLRISC | |
| hinge: IgG4 CH2 CH3 | KGSGFNIEDYYIHWVRQMPGKGLEWMGRIDPENDETK | |
| L235E N297Q peptide | YGPIFQGHVTISADTSINTVYLQWSSLKASDTAMYYCA | |
| spacer-CD28 | FRGGVYWGQGTTVTVSSGSTSGSGKPGSGEGSTKGDV | |
| transmembrane | VMTQSPDSLAVSLGERATINCKSSQSLLDSDGKTYLNW | |
| domain-4-1BB Co- | LQQKPGQPPKRLISLVSKLDSGVPDRFSGSGSGTDFTLTI | |
| stimulatory-CD3 Zeta) | SSLQAEDVAVYYCWQGTHFPGTFGGGTKVEIKESKYG | |
| | PPCPPCPAPEFEGGPSVFLFPPKPKDTLMISRTPEVTCVV | |
| | VDVSQEDPEVQFNWYVDGVEVHNAKTKPREEQFQSTY | |
| | RVVSVLTVLHQDWLNGKEYKCKVSNKGLPSSIEKTISK | |
| | AKGQPREPQVYTLPPSQEEMTKNQVSLTCLVKGFYPSD | |
| | IAVEWESNGQPENNYKTTPPVLDSDGSFFLYSRLTVDK | |
| | SRWQEGNVFSCSVMHEALHNHYTQKSLSLSLGKMFW | |
| | VLVVVGGVLACYSLLVTVAFIIFWVKRGRKKLLYIFKQ | |
| | PFMRPVQTTQEEDGCSCRFPEEEEGGCELRVKFSRSAD | |
| | APAYQQGQNQLYNELNLGRREEYDVLDKRRGRDPEM | |
| | GGKPRRKNPQEGLYNELQKDKMAEAYSEIGMKGERRR | |
| | GKGHDGLYQGLSTATKDTYDALHMQALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| EGFRvIII scFv | EIQLVQSGAEVKKPGESLRISCKGSGFNIEDYYIHWVRQ | 27 |
| | MPGKGLEWMGRIDPENDETKYGPIFQGHVTISADTSIN | |
| | TVYLQWSSLKASDTAMYYCAFRGGVYWGQGTTVTVS | |
| | SGSTSGSGKPGSGEGSTKGDVVMTQSPDSLAVSLGERA | |
| | TINCKSSQSLLDSDGKTYLNWLQQKPGQPPKRLISLVSK | |
| | LDSGVPDRFSGSGSGTDFTLTISSLQAEDVAVYYCWQG | |
| | THFPGTFGGGTKVEIK | |
| IgG4 hinge: IgG4 CH2 | ESKYGPPCPPCPAPEFEGGPSVFLFPPKPKDTLMISRTPE | 5 |
| CH3 L235E N297Q | VTCVVVDVSQEDPEVQFNWYVDGVEVHNAKTKPREE | |
| peptide spacer | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-(G4S)x4- | METDTLLLWVLLLWVPGSTGAGGSDYKDDDDKGGSG | 152 |
| GD2.BBz (MKleader- | PVPPSTALRYLIEELVNITQNQKAPLCNGSMVWSINLTA | |
| FLAG-IL13op- | GMYCAALESLINVSGCSAIEKTQRMLSGFCPHKVSAGQ | |
| (G4S)x4-GD2 scFv- | FSSLHVRDTKIEVAQFVKDLLLHLKKLFREGRENGGGG | |
| IgG4 hinge peptide | SGGGGSGGGGSGGGGSEVQLLQSGPELEKPGASVMISC | |
| spacer-CD28 | KASGSSFTGYNMNWVRQNIGKSLEWIGAIDPYYGGTS | |
| transmembrane | YNQKFKGRATLTVDKSSSTAYMHLKSLTSEDSAVYYC | |
| domain-4-1BB co- | VSGMEYWGQGTSVTVSSGSTSGSGKPGSGEGSTKGDV | |
| stimulatory-CD3 Zeta) | VMTQTPLSLPVSLGDQASISCRSSQSLVHRNGNTYLHW | |
| | YLQKPGQSPKLLIHKVSNRFSGVPDRFSGSGSGTDFTLK | |
| | ISRVEAEDLGVYFCSQSTHVPPLTFGAGTKLELKRAESK | |
| | YGPPCPPCPMFWVLVVVGGVLACYSLLVTVAFIIFWVK | |
| | RGRKKLLYIFKQPFMRPVQTTQEEDGCSCRFPEEEEGG | |
| | CELRVKFSRSADAPAYQQGQNQLYNELNLGRREEYDV | |
| | LDKRRGRDPEMGGKPRRKNPQEGLYNELQKDKMAEA | |
| | YSEIGMKGERRRGKGHDGLYQGLSTATKDTYDALHM | |
| | QALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| GD2 scFv | EVQLLQSGPELEKPGASVMISCKASGSSFTGYNMNWV | 26 |
| | RQNIGKSLEWIGAIDPYYGGTSYNQKFKGRATLTVDKS | |
| | SSTAYMHLKSLTSEDSAVYYCVSGMEYWGQGTSVTVS | |
| | SGSTSGSGKPGSGEGSTKGDVVMTQTPLSLPVSLGDQA | |
| | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRA | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-(G4S)x4- | METDTLLLWVLLLWVPGSTGAGGSDYKDDDDKGGSG | 153 |
| GD2.BBz (MKleader- | PVPPSTALRYLIEELVNITQNQKAPLCNGSMVWSINLTA | |
| FLAG-IL13op- | GMYCAALESLINVSGCSAIEKTQRMLSGFCPHKVSAGQ | |
| (G4S)x4-GD2 scFv- | FSSLHVRDTKIEVAQFVKDLLLHLKKLFREGRENGGGG | |
| IgG4 hinge:IgG4 CH2 | SGGGGSGGGGSGGGGSEVQLLQSGPELEKPGASVMISC | |
| CH3 L235E N297Q | KASGSSFTGYNMNWVRQNIGKSLEWIGAIDPYYGGTS | |
| peptide spacer-CD28 | YNQKFKGRATLTVDKSSSTAYMHLKSLTSEDSAVYYC | |
| transmembrane | VSGMEYWGQGTSVTVSSGSTSGSGKPGSGEGSTKGDV | |
| domain-4-1BB co- | VMTQTPLSLPVSLGDQASISCRSSQSLVHRNGNTYLHW | |
| stimulatory-CD3 Zeta) | YLQKPGQSPKLLIHKVSNRFSGVPDRFSGSGSGTDFTLK | |
| | ISRVEAEDLGVYFCSQSTHVPPLTFGAGTKLELKRAESK | |
| | YGPPCPPCPAPEFEGGPSVFLFPPKPKDTLMISRTPEVTC | |
| | VVVDVSQEDPEVQFNWYVDGVEVHNAKTKPREEQFQ | |
| | STYRVVSVLTVLHQDWLNGKEYKCKVSNKGLPSSIEKT | |
| | ISKAKGQPREPQVYTLPPSQEEMTKNQVSLTCLVKGFY | |
| | PSDIAVEWESNGQPENNYKTTPPVLDSDGSFFLYSRLTV | |
| | DKSRWQEGNVFSCSVMHEALHNHYTQKSLSLSLGKMF | |
| | WVLVVVGGVLACYSLLVTVAFIIFWVKRGRKKLLYIFK | |
| | QPFMRPVQTTQEEDGCSCRFPEEEEGGCELRVKFSRSA | |
| | DAPAYQQGQNQLYNELNLGRREEYDVLDKRRGRDPE | |
| | MGGKPRRKNPQEGLYNELQKDKMAEAYSEIGMKGER | |
| | RRGKGHDGLYQGLSTATKDTYDALHMQALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| GD2 scFv | EVQLLQSGPELEKPGASVMISCKASGSSFTGYNMNWV | 26 |
| | RQNIGKSLEWIGAIDPYYGGTSYNQKFKGRATLTVDKS | |
| | SSTAYMHLKSLTSEDSAVYYCVSGMEYWGQGTSVTVS | |
| | SGSTSGSGKPGSGEGSTKGDVVMTQTPLSLPVSLGDQA | |
| | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRA | |
| IgG4 hinge: IgG4 CH2 | ESKYGPPCPPCPAPEFEGGPSVFLFPPKPKDTLMISRTPE | 5 |
| CH3 L235E N297Q | VTCVVVDVSQEDPEVQFNWYVDGVEVHNAKTKPREE | |
| peptide spacer | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2.BBz + TGF- | METDTLLLWVLLLWVPGSTGAGGSDYKDDDDKGGSG | 154 |
| β.28z (MKleader- | PVPPSTALRYLIEELVNITQNQKAPLCNGSMVWSINLTA | |
| FLAG-IL13op-IgG4 | GMYCAALESLINVSGCSAIEKTQRMLSGFCPHKVSAGQ | |
| hinge: IgG4 CH2 CH3 | FSSLHVRDTKIEVAQFVKDLLLHLKKLFREGRFNESKY | |
| L235E N297Q peptide | GPPCPPCPAPEFEGGPSVFLFPPKPKDTLMISRTPEVTCV | |
| spacer-CD28 | VVDVSQEDPEVQFNWYVDGVEVHNAKTKPREEQFQST | |
| transmembrane | YRVVSVLTVLHQDWLNGKEYKCKVSNKGLPSSIEKTIS | |
| domain-4-1BB co- | KAKGQPREPQVYTLPPSQEEMTKNQVSLTCLVKGFYPS | |
| stimulatory-CD3 Zeta- | DIAVEWESNGQPENNYKTTPPVLDSDGSFFLYSRLTVD | |
| T2A-MKleader-HA- | KSRWQEGNVFSCSVMHEALHNHYTQKSLSLSLGKMF | |
| TGF-β scFv-IgG4 | WVLVVVGGVLACYSLLVTVAFIIFWVKRGRKKLLYIFK | |
| hinge peptide spacer- | QPFMRPVQTTQEEDGCSCRFPEEEEGGCELRVKFSRSA | |
| CD28 transmembrane | DAPAYQQGQNQLYNELNLGRREEYDVLDKRRGRDPE | |
| domain-CD28cyto | MGGKPRRKNPQEGLYNELQKDKMAEAYSEIGMKGER | |
| (with gg mutations)- | RRGKGHDGLYQGLSTATKDTYDALHMQALPPRLEGG | |
| CD3 Zeta) | GEGRGSLLTCGDVEENPGPRMETDTLLLWVLLLWVPG | |
| | STGTSYPYDVPDYAGGSQVQLVQSGAEVKKPGSSVKV | |
| | SCKASGYTFSSNVISWVRQAPGQGLEWMGGVIPIVDIA | |
| | NYAQRFKGRVTITADESTSTTYMELSSLRSEDTAVYYC | |
| | ALPRAFVLDAMDYWGQGTLVTVSSGGGGSGGGGSGG | |
| | GGSETVLTQSPGTLSLSPGERATLSCRASQSLGSSYLAW | |
| | YQQKPGQAPRLLIYGASSRAPGIPDRESGSGSGTDFTLTI | |
| | SRLEPEDFAVYYCQQYADSPITFGQGTRLEIKESKYGPP | |
| | CPPCPMFWVLVVVGGVLACYSLLVTVAFIIFWVRSKRS | |
| | RGGHSDYMNMTPRRPGPTRKHYQPYAPPRDFAAYRSG | |
| | GGRVKFSRSADAPAYQQGQNQLYNELNLGRREEYDVL | |
| | DKRRGRDPEMGGKPRRKNPQEGLYNELQKDKMAEAY | |
| | SEIGMKGERRRGKGHDGLYQGLSTATKDTYDALHMQ | |
| | ALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| IgG4 hinge: IgG4 CH2 | ESKYGPPCPPCPAPEFEGGPSVFLFPPKPKDTLMISRTPE | 5 |
| CH3 L235E N297Q | VTCVVVDVSQEDPEVQFNWYVDGVEVHNAKTKPREE | |
| peptide spacer | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| T2A | EGRGSLLTCGDVEENPGPR | 24 |
| MKleader | METDTLLLWVLLLWVPGSTG | 2 |
| HA | YPYDVPDYA | 122 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRESGSGSGTDFTLTISRLEPEDFAVYYCQQYA | |
| | DSPITFGQGTRLEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| CD28cyto (with gg | RSKRSRGGHSDYMNMTPRRPGPTRKHYQPYAPPRDFA | 18 |
| mutations) | AYRS | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2- | METDTLLLWVLLLWVPGSTGAGGSDYKDDDDKGGSG | 155 |
| EGFRvIII.BBz + TGF- | PVPPSTALRYLIEELVNITQNQKAPLCNGSMVWSINLTA | |
| β.28z (MKleader- | GMYCAALESLINVSGCSAIEKTQRMLSGFCPHKVSAGQ | |
| FLAG-IL13op- | FSSLHVRDTKIEVAQFVKDLLLHLKKLFREGRENGGGG | |
| (G4S)x4-EGFRvIII | SGGGGSGGGGSGGGGSEIQLVQSGAEVKKPGESLRISC | |
| scFv-IgG4 hinge-CD28 | KGSGFNIEDYYIHWVRQMPGKGLEWMGRIDPENDETK | |
| transmembrane | YGPIFQGHVTISADTSINTVYLQWSSLKASDTAMYYCA | |
| domain-4-1BB co- | FRGGVYWGQGTTVTVSSGSTSGSGKPGSGEGSTKGDV | |
| stimulatory-CD3 Zeta- | VMTQSPDSLAVSLGERATINCKSSQSLLDSDGKTYLNW | |
| T2A-MKleader-HA- | LQQKPGQPPKRLISLVSKLDSGVPDRFSGSGSGTDFTLTI | |
| TGF-β scFv-IgG4 | SSLQAEDVAVYYCWQGTHFPGTFGGGTKVEIKESKYG | |
| hinge peptide spacer- | PPCPPCPMFWVLVVVGGVLACYSLLVTVAFIIFWVKRG | |
| CD28 transmembrane | RKKLLYIFKQPFMRPVQTTQEEDGCSCRFPEEEEGGCEL | |
| domain-CD28cyto | RVKFSRSADAPAYQQGQNQLYNELNLGRREEYDVLDK | |
| (with gg mutations)- | RRGRDPEMGGKPRRKNPQEGLYNELQKDKMAEAYSEI | |
| CD3 Zeta) | GMKGERRRGKGHDGLYQGLSTATKDTYDALHMQALP | |
| | PRLEGGGEGRGSLLTCGDVEENPGPRMETDTLLLWVLL | |
| | LWVPGSTGTSYPYDVPDYAGGSQVQLVQSGAEVKKPG | |
| | SSVKVSCKASGYTFSSNVISWVRQAPGQGLEWMGGVIP | |
| | IVDIANYAQRFKGRVTITADESTSTTYMELSSLRSEDTA | |
| | VYYCALPRAFVLDAMDYWGQGTLVTVSSGGGGSGGG | |
| | GSGGGGSETVLTQSPGTLSLSPGERATLSCRASQSLGSS | |
| | YLAWYQQKPGQAPRLLIYGASSRAPGIPDRFSGSGSGT | |
| | DFTLTISRLEPEDFAVYYCQQYADSPITFGQGTRLEIKES | |
| | KYGPPCPPCPMFWVLVVVGGVLACYSLLVTVAFIIFWV | |
| | RSKRSRGGHSDYMNMTPRRPGPTRKHYQPYAPPRDFA | |
| | AYRSGGGRVKFSRSADAPAYQQGQNQLYNELNLGRRE | |
| | EYDVLDKRRGRDPEMGGKPRRKNPQEGLYNELQKDK | |
| | MAEAYSEIGMKGERRRGKGHDGLYQGLSTATKDTYD | |
| | ALHMQALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| EGFRvIII scFv | EIQLVQSGAEVKKPGESLRISCKGSGFNIEDYYIHWVRQ | 27 |
| | MPGKGLEWMGRIDPENDETKYGPIFQGHVTISADTSIN | |
| | TVYLQWSSLKASDTAMYYCAFRGGVYWGQGTTVTVS | |
| | SGSTSGSGKPGSGEGSTKGDVVMTQSPDSLAVSLGERA | |
| | TINCKSSQSLLDSDGKTYLNWLQQKPGQPPKRLISLVSK | |
| | LDSGVPDRFSGSGSGTDFTLTISSLQAEDVAVYYCWQG | |
| | THFPGTFGGGTKVEIK | |
| IgG4 hinge | ESKYGPPCPPCP | 12 |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| T2A | EGRGSLLTCGDVEENPGPR | 24 |
| MKleader | METDTLLLWVLLLWVPGSTG | 2 |
| HA | YPYDVPDYA | 122 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| CD28cyto (with gg | RSKRSRGGHSDYMNMTPRRPGPTRKHYQPYAPPRDFA | 18 |
| mutations) | AYRS | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-GD2.BBz + | METDTLLLWVLLLWVPGSTGAGGSDYKDDDDKGGSG | 156 |
| TGF-β.28z (MKleader- | PVPPSTALRYLIEELVNITQNQKAPLCNGSMVWSINLTA | |
| FLAG-IL13op- | GMYCAALESLINVSGCSAIEKTQRMLSGFCPHKVSAGQ | |
| (G4S)x4-GD2 scFv- | FSSLHVRDTKIEVAQFVKDLLLHLKKLFREGRENGGGG | |
| IgG4 hinge: IgG4 CH2 | SGGGGSGGGGSGGGGSEVQLLQSGPELEKPGASVMISC | |
| CH3 L235E N297Q | KASGSSFTGYNMNWVRQNIGKSLEWIGAIDPYYGGTS | |
| peptide spacer-CD28 | YNQKFKGRATLTVDKSSSTAYMHLKSLTSEDSAVYYC | |
| transmembrane | VSGMEYWGQGTSVTVSSGSTSGSGKPGSGEGSTKGDV | |
| domain-4-1BB co- | VMTQTPLSLPVSLGDQASISCRSSQSLVHRNGNTYLHW | |
| stimulatory-CD3 Zeta- | YLQKPGQSPKLLIHKVSNRFSGVPDRFSGSGSGTDFTLK | |
| T2A-MKleader-HA- | ISRVEAEDLGVYFCSQSTHVPPLTFGAGTKLELKRAESK | |
| TGF-β scFv-IgG4 | YGPPCPPCPAPEFEGGPSVFLFPPKPKDTLMISRTPEVTC | |
| hinge peptide spacer- | VVVDVSQEDPEVQFNWYVDGVEVHNAKTKPREEQFQ | |
| CD28 transmembrane | STYRVVSVLTVLHQDWLNGKEYKCKVSNKGLPSSIEKT | |
| domain-CD28cyto | ISKAKGQPREPQVYTLPPSQEEMTKNQVSLTCLVKGFY | |
| (with gg mutations)- | PSDIAVEWESNGQPENNYKTTPPVLDSDGSFFLYSRLTV | |
| CD3 Zeta) | DKSRWQEGNVFSCSVMHEALHNHYTQKSLSLSLGKMF | |
| | WVLVVVGGVLACYSLLVTVAFIIFWVKRGRKKLLYIFK | |
| | QPFMRPVQTTQEEDGCSCRFPEEEEGGCELRVKFSRSA | |
| | DAPAYQQGQNQLYNELNLGRREEYDVLDKRRGRDPE | |
| | MGGKPRRKNPQEGLYNELQKDKMAEAYSEIGMKGER | |
| | RRGKGHDGLYQGLSTATKDTYDALHMQALPPRLEGG | |
| | GEGRGSLLTCGDVEENPGPRMETDTLLLWVLLLWVPG | |
| | STGTSYPYDVPDYAGGSQVQLVQSGAEVKKPGSSVKV | |
| | SCKASGYTFSSNVISWVRQAPGQGLEWMGGVIPIVDIA | |
| | NYAQRFKGRVTITADESTSTTYMELSSLRSEDTAVYYC | |
| | ALPRAFVLDAMDYWGQGTLVTVSSGGGGSGGGGSGG | |
| | GGSETVLTQSPGTLSLSPGERATLSCRASQSLGSSYLAW | |
| | YQQKPGQAPRLLIYGASSRAPGIPDRESGSGSGTDFTLTI | |
| | SRLEPEDFAVYYCQQYADSPITFGQGTRLEIKESKYGPP | |
| | CPPCPMFWVLVVVGGVLACYSLLVTVAFIIFWVRSKRS | |
| | RGGHSDYMNMTPRRPGPTRKHYQPYAPPRDFAAYRSG | |
| | GGRVKFSRSADAPAYQQGQNQLYNELNLGRREEYDVL | |
| | DKRRGRDPEMGGKPRRKNPQEGLYNELQKDKMAEAY | |
| | SEIGMKGERRRGKGHDGLYQGLSTATKDTYDALHMQ | |
| | ALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| GD2 scFv | EVQLLQSGPELEKPGASVMISCKASGSSFTGYNMNWV | 26 |
| | RQNIGKSLEWIGAIDPYYGGTSYNQKFKGRATLTVDKS | |
| | SSTAYMHLKSLTSEDSAVYYCVSGMEYWGQGTSVTVS | |
| | SGSTSGSGKPGSGEGSTKGDVVMTQTPLSLPVSLGDQA | |
| | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRA | |
| IgG4 hinge: IgG4 CH2 | ESKYGPPCPPCPAPEFEGGPSVFLFPPKPKDTLMISRTPE | 5 |
| CH3 L235E N297Q | VTCVVVDVSQEDPEVQFNWYVDGVEVHNAKTKPREE | |
| peptide spacer | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| T2A | EGRGSLLTCGDVEENPGPR | 24 |
| MKleader | METDTLLLWVLLLWVPGSTG | 2 |
| HA | YPYDVPDYA | 122 |
| TGF- scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| CD28cyto (with gg | RSKRSRGGHSDYMNMTPRRPGPTRKHYQPYAPPRDFA | 18 |
| mutations) | AYRS | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-GD2-TGF- | METDTLLLWVLLLWVPGSTGAGGSDYKDDDDKGGSG | 157 |
| β.BBz (MKleader- | PVPPSTALRYLIEELVNITQNQKAPLCNGSMVWSINLTA | |
| FLAG-IL13op- | GMYCAALESLINVSGCSAIEKTQRMLSGFCPHKVSAGQ | |
| (G4S)x4-GD2 scFv- | FSSLHVRDTKIEVAQFVKDLLLHLKKLFREGRENGGGG | |
| (G4S)x4-TGF-β scFv- | SGGGGSGGGGSGGGGSEVQLLQSGPELEKPGASVMISC | |
| IgG4 hinge peptide | KASGSSFTGYNMNWVRQNIGKSLEWIGAIDPYYGGTS | |
| spacer-CD28 | YNQKFKGRATLTVDKSSSTAYMHLKSLTSEDSAVYYC | |
| transmembrane | VSGMEYWGQGTSVTVSSGSTSGSGKPGSGEGSTKGDV | |
| domain-4-1BB co- | VMTQTPLSLPVSLGDQASISCRSSQSLVHRNGNTYLHW | |
| stimulatory-CD3 Zeta) | YLQKPGQSPKLLIHKVSNRFSGVPDRFSGSGSGTDFTLK | |
| | ISRVEAEDLGVYFCSQSTHVPPLTFGAGTKLELKRAGG | |
| | GGSGGGGSGGGGSGGGGSQVQLVQSGAEVKKPGSSVK | |
| | VSCKASGYTFSSNVISWVRQAPGQGLEWMGGVIPIVDI | |
| | ANYAQRFKGRVTITADESTSTTYMELSSLRSEDTAVYY | |
| | CALPRAFVLDAMDYWGQGTLVTVSSGGGGSGGGGSG | |
| | GGGSETVLTQSPGTLSLSPGERATLSCRASQSLGSSYLA | |
| | WYQQKPGQAPRLLIYGASSRAPGIPDRFSGSGSGTDFTL | |
| | TISRLEPEDFAVYYCQQYADSPITFGQGTRLEIKESKYG | |
| | PPCPPCPMFWVLVVVGGVLACYSLLVTVAFIIFWVKRG | |
| | RKKLLYIFKQPFMRPVQTTQEEDGCSCRFPEEEEGGCEL | |
| | RVKFSRSADAPAYQQGQNQLYNELNLGRREEYDVLDK | |
| | RRGRDPEMGGKPRRKNPQEGLYNELQKDKMAEAYSEI | |
| | GMKGERRRGKGHDGLYQGLSTATKDTYDALHMQALP | |
| | PR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| GD2 scFv | EVQLLQSGPELEKPGASVMISCKASGSSFTGYNMNWV | 26 |
| | RQNIGKSLEWIGAIDPYYGGTSYNQKFKGRATLTVDKS | |
| | SSTAYMHLKSLTSEDSAVYYCVSGMEYWGQGTSVTVS | |
| | SGSTSGSGKPGSGEGSTKGDVVMTQTPLSLPVSLGDQA | |
| | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRA | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRESGSGSGTDFTLTISRLEPEDFAVYYCQQYA | |
| | DSPITFGQGTRLEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| IL13Rα2-EGFRvIII- | METDTLLLWVLLLWVPGSTGAGGSDYKDDDDKGGSG | 158 |
| TGF-β.BBz | PVPPSTALRYLIEELVNITQNQKAPLCNGSMVWSINLTA | |
| (MKleader-FLAG- | GMYCAALESLINVSGCSAIEKTQRMLSGFCPHKVSAGQ | |
| IL13op-(G4S)x4- | FSSLHVRDTKIEVAQFVKDLLLHLKKLFREGRENGGGG | |
| EGFRvIII scFv- | SGGGGSGGGGSGGGGSEIQLVQSGAEVKKPGESLRISC | |
| (G4S)x4-TGF-β scFv- | KGSGFNIEDYYIHWVRQMPGKGLEWMGRIDPENDETK | |
| IgG4 hinge peptide | YGPIFQGHVTISADTSINTVYLQWSSLKASDTAMYYCA | |
| spacer-CD28 | FRGGVYWGQGTTVTVSSGSTSGSGKPGSGEGSTKGDV | |
| transmembrane | VMTQSPDSLAVSLGERATINCKSSQSLLDSDGKTYLNW | |
| domain-4-1BB co- | LQQKPGQPPKRLISLVSKLDSGVPDRFSGSGSGTDFTLTI | |
| stimulatory-CD3 Zeta) | SSLQAEDVAVYYCWQGTHFPGTFGGGTKVEIKGGGGS | |
| | GGGGSGGGGSGGGGSQVQLVQSGAEVKKPGSSVKVSC | |
| | KASGYTFSSNVISWVRQAPGQGLEWMGGVIPIVDIANY | |
| | AQRFKGRVTITADESTSTTYMELSSLRSEDTAVYYCAL | |
| | PRAFVLDAMDYWGQGTLVTVSSGGGGSGGGGSGGGG | |
| | SETVLTQSPGTLSLSPGERATLSCRASQSLGSSYLAWYQ | |
| | QKPGQAPRLLIYGASSRAPGIPDRFSGSGSGTDFTLTISR | |
| | LEPEDFAVYYCQQYADSPITFGQGTRLEIKESKYGPPCP | |
| | PCPMFWVLVVVGGVLACYSLLVTVAFIIFWVKRGRKK | |
| | LLYIFKQPFMRPVQTTQEEDGCSCRFPEEEEGGCELRVK | |
| | FSRSADAPAYQQGQNQLYNELNLGRREEYDVLDKRRG | |
| | RDPEMGGKPRRKNPQEGLYNELQKDKMAEAYSEIGM | |
| | KGERRRGKGHDGLYQGLSTATKDTYDALHMQALPPR | |
| MKleader | METDTLLLWVLLLWVPGSTG | 2 |
| FLAG | DYKDDDDK | 3 |
| IL13op | GPVPPSTALRYLIEELVNITQNQKAPLCNGSMVWSINLT | 147 |
| | AGMYCAALESLINVSGCSAIEKTQRMLSGFCPHKVSAG | |
| | QFSSLHVRDTKIEVAQFVKDLLLHLKKLFREGREN | |
| (G4S)x4 | GGGGSGGGGSGGGGSGGGGS | 10 |
| EGFRvIII scFv | EIQLVQSGAEVKKPGESLRISCKGSGFNIEDYYIHWVRQ | 27 |
| | MPGKGLEWMGRIDPENDETKYGPIFQGHVTISADTSIN | |
| | TVYLQWSSLKASDTAMYYCAFRGGVYWGQGTTVTVS | |
| | SGSTSGSGKPGSGEGSTKGDVVMTQSPDSLAVSLGERA | |
| | TINCKSSQSLLDSDGKTYLNWLQQKPGQPPKRLISLVSK | |
| | LDSGVPDRFSGSGSGTDFTLTISSLQAEDVAVYYCWQG | |
| | THFPGTFGGGTKVEIK | |
| (G4S)x4 | GGGGSGGGGSGGGGSGGGGS | 10 |
| TGF-B scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge peptide | ESKYGPPCPPCP | 12 |
| spacer | | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2.BBz | METDTLLLWVLLLWVPGSTGAGGSDYKDDDDKGGSS | 1 |
| CAR; (Murine kappa | PGPVPPSTALRYLIEELVNITQNQKAPLCNGSMVWSINL | |
| signal | TAGMYCAALESLINVSGCSAIEKTQRMLSGFCPHKVSA | |
| sequence_FLAG_SP | GQFSSLHVRDTKIEVAQFVKDLLLHLKKLFREGRFNES | |
| IL13 mutein_IgG4 | KYGPPCPPCPAPEFEGGPSVFLFPPKPKDTLMISRTPEVT | |
| (L235E, | CVVVDVSQEDPEVQFNWYVDGVEVHNAKTKPREEQF | |
| N297Q)_CD28tm_4- | QSTYRVVSVLTVLHQDWLNGKEYKCKVSNKGLPSSIE | |
| 1BB_CD3zeta) | KTISKAKGQPREPQVYTLPPSQEEMTKNQVSLTCLVKG | |
| | FYPSDIAVEWESNGQPENNYKTTPPVLDSDGSFFLYSRL | |
| | TVDKSRWQEGNVFSCSVMHEALHNHYTQKSLSLSLGK | |
| | MFWVLVVVGGVLACYSLLVTVAFIIFWVKRGRKKLLY | |
| | IFKQPFMRPVQTTQEEDGCSCRFPEEEEGGCELRVKFSR | |
| | SADAPAYQQGQNQLYNELNLGRREEYDVLDKRRGRDP | |
| | EMGGKPRRKNPQEGLYNELQKDKMAEAYSEIGMKGE | |
| | RRRGKGHDGLYQGLSTATKDTYDALHMQALPPR | |
| SP-IL-13Rα2.BBz | METDTLLLWVLLLWVSPGSTGSPGPVPPSTALRYLIEEL | 136 |
| CAR; (Murine kappa | VNITQNQKAPLCNGSMVWSINLTAGMYCAALESLINVS | |
| signal sequence_SP | GCSAIEKTQRMLSGFCPHKVSAGQFSSLHVRDTKIEVA | |
| IL13 mutein_IgG4 | QFVKDLLLHLKKLFREGRFNESKYGPPCPPCPAPEFEGG | |
| (L235E, | PSVFLFPPKPKDTLMISRTPEVTCVVVDVSQEDPEVQFN | |
| N297Q)_CD28tm_4- | WYVDGVEVHNAKTKPREEQFQSTYRVVSVLTVLHQD | |
| 1BB_CD3zeta) | WLNGKEYKCKVSNKGLPSSIEKTISKAKGQPREPQVYT | |
| | LPPSQEEMTKNQVSLTCLVKGFYPSDIAVEWESNGQPE | |
| | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVKRGRKKLLYIFKQPFMRPVQTTQEED | |
| | GCSCRFPEEEEGGCELRVKFSRSADAPAYQQGQNQLYN | |
| | ELNLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGREN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2/TGF- | METDTLLLWVLLLWVPGSTGAGGSDYKDDDDKGGSS | 9 |
| β.BBz CAR; (Murine | PGPVPPSTALRYLIEELVNITQNQKAPLCNGSMVWSINL | |
| kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRENGG | |
| IL13 | GGSGGGGSGGGGSGGGGSQVQLVQSGAEVKKPGSSVK | |
| mutein_(G4S)x4_TGF- | VSCKASGYTFSSNVISWVRQAPGQGLEWMGGVIPIVDI | |
| β scFv_IgG4 | ANYAQRFKGRVTITADESTSTTYMELSSLRSEDTAVYY | |
| hinge_CD28tm_4- | CALPRAFVLDAMDYWGQGTLVTVSSGGGGSGGGGSG | |
| 1BB_CD3zeta) | GGGSETVLTQSPGTLSLSPGERATLSCRASQSLGSSYLA | |
| | WYQQKPGQAPRLLIYGASSRAPGIPDRFSGSGSGTDFTL | |
| | TISRLEPEDFAVYYCQQYADSPITFGQGTRLEIKESKYG | |
| | PPCPPCPMFWVLVVVGGVLACYSLLVTVAFIIFWVKRG | |
| | RKKLLYIFKQPFMRPVQTTQEEDGCSCRFPEEEEGGCEL | |
| | RVKFSRSADAPAYQQGQNQLYNELNLGRREEYDVLDK | |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2/TGF- | METDTLLLWVLLLWVPGSTGSPGPVPPSTALRYLIEEL | 137 |
| β.BBz CAR; (Murine | VNITQNQKAPLCNGSMVWSINLTAGMYCAALESLINVS | |
| kappa signal sequence | GCSAIEKTQRMLSGFCPHKVSAGQFSSLHVRDTKIEVA | |
| SP IL13 | QFVKDLLLHLKKLFREGRENGGGGSGGGGSGGGGSGG | |
| mutein_(G4S)x4_TGF- | GGSQVQLVQSGAEVKKPGSSVKVSCKASGYTFSSNVIS | |
| β scFv_IgG4 | WVRQAPGQGLEWMGGVIPIVDIANYAQRFKGRVTITA | |
| hinge_CD28tm_4- | DESTSTTYMELSSLRSEDTAVYYCALPRAFVLDAMDY | |
| 1BB_CD3zeta) | WGQGTLVTVSSGGGGSGGGGSGGGGSETVLTQSPGTL | |
| | SLSPGERATLSCRASQSLGSSYLAWYQQKPGQAPRLLI | |
| | YGASSRAPGIPDRFSGSGSGTDFTLTISRLEPEDFAVYYC | |
| | QQYADSPITFGQGTRLEIKESKYGPPCPPCPMFWVLVV | |
| | VGGVLACYSLLVTVAFIIFWVKRGRKKLLYIFKQPFMR | |
| | PVQTTQEEDGCSCRFPEEEEGGCELRVKFSRSADAPAY | |
| | QQGQNQLYNELNLGRREEYDVLDKRRGRDPEMGGKP | |
| | RRKNPQEGLYNELQKDKMAEAYSEIGMKGERRRGKG | |
| | HDGLYQGLSTATKDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIK | |
| IgG4 hinge | ESKYGPPCPPCP | 12 |
| CD28 transmembrane | MFWVLVVVGGVLACYSLLVTVAFIIFWV | 6 |
| domain | | |
| 4-1BB co-stimulatory | KRGRKKLLYIFKQPFMRPVQTTQEEDGCSCRFPEEEEG | 7 |
| | GCEL | |
| CD3 Zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2.BBz | METDTLLLWVLLLWVPGSTGAGGSDYKDDDDKGGSS | 13 |
| (KR) CAR; (Murine | PGPVPPSTALRYLIEELVNITQNQKAPLCNGSMVWSINL | |
| kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRFNES | |
| IL13 mutein_IgG4 | KYGPPCPPCPAPEFEGGPSVFLFPPKPKDTLMISRTPEVT | |
| (L235E, | CVVVDVSQEDPEVQFNWYVDGVEVHNAKTKPREEQF | |
| N297Q)_CD28tm_4- | QSTYRVVSVLTVLHQDWLNGKEYKCKVSNKGLPSSIE | |
| 1BB(KR)_CD3zeta(KR)) | KTISKAKGQPREPQVYTLPPSQEEMTKNQVSLTCLVKG | |
| | FYPSDIAVEWESNGQPENNYKTTPPVLDSDGSFFLYSRL | |
| | TVDKSRWQEGNVFSCSVMHEALHNHYTQKSLSLSLGK | |
| | MFWVLVVVGGVLACYSLLVTVAFIIFWVRRGRRRLLYI | |
| | FRQPFMRPVQTTQEEDGCSCRFPEEEEGGCELRVRFSRS | |
| | ADAPAYQQGQNQLYNELNLGRREEYDVLDRRRGRDPE | |
| | MGGRPRRRNPQEGLYNELQRDRMAEAYSEIGMRGERR | |
| | RGRGHDGLYQGLSTATRDTYDALHMQALPPR | |
| SP-IL-13Rα2.BBz | METDTLLLWVLLLWVPGSTGSPGPVPPSTALRYLIEEL | 138 |
| (KR) CAR; (Murine | VNITQNQKAPLCNGSMVWSINLTAGMYCAALESLINVS | |
| kappa signal sequence | GCSAIEKTQRMLSGFCPHKVSAGQFSSLHVRDTKIEVA | |
| SP IL13 mutein_IgG4 | QFVKDLLLHLKKLFREGRFNESKYGPPCPPCPAPEFEGG | |
| (L235E, | PSVFLFPPKPKDTLMISRTPEVTCVVVDVSQEDPEVQFN | |
| N297Q)_CD28tm_4- | WYVDGVEVHNAKTKPREEQFQSTYRVVSVLTVLHQD | |
| 1BB(KR)_CD3zeta(KR)) | WLNGKEYKCKVSNKGLPSSIEKTISKAKGQPREPQVYT | |
| | LPPSQEEMTKNQVSLTCLVKGFYPSDIAVEWESNGQPE | |
| | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVRRGRRRLLYIFRQPFMRPVQTTQEED | |
| | GCSCRFPEEEEGGCELRVRFSRSADAPAYQQGQNQLYN | |
| | ELNLGRREEYDVLDRRRGRDPEMGGRPRRRNPQEGLY | |
| | NELQRDRMAEAYSEIGMRGERRRGRGHDGLYQGLSTA | |
| | TRDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGREN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28 transmembrane | MFWVLVVVGGVLACYSLLVTVAFIIFWV | 6 |
| domain | | |
| 4-1BB(KR) co- | RRGRRRLLYIFRQPFMRPVQTTQEEDGCSCRFPEEEEGG | 14 |
| stimulatory | CEL | |
| CD3 Zeta(KR) | RVRFSRSADAPAYQQGQNQLYNELNLGRREEYDVLDR | 15 |
| | RRGRDPEMGGRPRRRNPQEGLYNELQRDRMAEAYSEI | |
| | GMRGERRRGRGHDGLYQGLSTATRDTYDALHMQALP | |
| | PR | |
| SP-IL-13Rα2/TGF- | METDTLLLWVLLLWVPGSTGAGGSDYKDDDDKGGSS | 16 |
| β.BBz (KR) CAR; | PGPVPPSTALRYLIEELVNITQNQKAPLCNGSMVWSINL | |
| (Murine kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRENGG | |
| IL13 | GGSGGGGSGGGGSGGGGSQVQLVQSGAEVKKPGSSVK | |
| mutein_(G4S)x4_TGF- | VSCKASGYTFSSNVISWVRQAPGQGLEWMGGVIPIVDI | |
| β scFv_IgG4 | ANYAQRFKGRVTITADESTSTTYMELSSLRSEDTAVYY | |
| hinge_CD28tm_4- | CALPRAFVLDAMDYWGQGTLVTVSSGGGGSGGGGSG | |
| 1BB(KR)_CD3zeta(KR)) | GGGSETVLTQSPGTLSLSPGERATLSCRASQSLGSSYLA | |
| | WYQQKPGQAPRLLIYGASSRAPGIPDRFSGSGSGTDFTL | |
| | TISRLEPEDFAVYYCQQYADSPITFGQGTRLEIKESKYG | |
| | PPCPPCPMFWVLVVVGGVLACYSLLVTVAFIIFWVRRG | |
| | RRRLLYIFRQPFMRPVQTTQEEDGCSCRFPEEEEGGCEL | |
| | RVRFSRSADAPAYQQGQNQLYNELNLGRREEYDVLDR | |
| | RRGRDPEMGGRPRRRNPQEGLYNELQRDRMAEAYSEI | |
| | GMRGERRRGRGHDGLYQGLSTATRDTYDALHMQALP | |
| | PR | |
| SP-IL-13Rα2/TGF- | METDTLLLWVLLLWVPGSTGSPGPVPPSTALRYLIEEL | 139 |
| β.BBz (KR) CAR; | VNITQNQKAPLCNGSMVWSINLTAGMYCAALESLINVS | |
| (Murine kappa signal | GCSAIEKTQRMLSGFCPHKVSAGQFSSLHVRDTKIEVA | |
| sequence_SP IL13 | QFVKDLLLHLKKLFREGRFNGGGGSGGGGSGGGGSGG | |
| mutein_(G4S)x4_TGF- | GGSQVQLVQSGAEVKKPGSSVKVSCKASGYTFSSNVIS | |
| β scFv_IgG4 | WVRQAPGQGLEWMGGVIPIVDIANYAQRFKGRVTITA | |
| hinge_CD28tm_4- | DESTSTTYMELSSLRSEDTAVYYCALPRAFVLDAMDY | |
| 1BB(KR)_CD3zeta(KR)) | WGQGTLVTVSSGGGGSGGGGSGGGGSETVLTQSPGTL | |
| | SLSPGERATLSCRASQSLGSSYLAWYQQKPGQAPRLLI | |
| | YGASSRAPGIPDRFSGSGSGTDFTLTISRLEPEDFAVYYC | |
| | QQYADSPITFGQGTRLEIKESKYGPPCPPCPMFWVLVV | |
| | VGGVLACYSLLVTVAFIIFWVRRGRRRLLYIFRQPFMRP | |
| | VQTTQEEDGCSCRFPEEEEGGCELRVRFSRSADAPAYQ | |
| | QGQNQLYNELNLGRREEYDVLDRRRGRDPEMGGRPRR | |
| | RNPQEGLYNELQRDRMAEAYSEIGMRGERRRGRGHDG | |
| | LYQGLSTATRDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRESGSGSGTDFTLTISRLEPEDFAVYYCQQYA | |
| | DSPITFGQGTRLEIK | |
| IgG4 hinge | ESKYGPPCPPCP | 12 |
| CD28 transmembrane | MFWVLVVVGGVLACYSLLVTVAFIIFWV | 6 |
| domain | | |
| 4-1BB(KR) co- | RRGRRRLLYIFRQPFMRPVQTTQEEDGCSCRFPEEEEGG | 14 |
| stimulatory | CEL | |
| CD3zeta(KR) | RVRFSRSADAPAYQQGQNQLYNELNLGRREEYDVLDR | 15 |
| | RRGRDPEMGGRPRRRNPQEGLYNELQRDRMAEAYSEI | |
| | GMRGERRRGRGHDGLYQGLSTATRDTYDALHMQALP | |
| | PR | |
| SP-IL-13Rα2.28z | METDTLLLWVLLLWVPGSTGAGGSDYKDDDDKGGSS | 17 |
| CAR; (Murine kappa | PGPVPPSTALRYLIEELVNITQNQKAPLCNGSMVWSINL | |
| signal | TAGMYCAALESLINVSGCSAIEKTQRMLSGFCPHKVSA | |
| sequence_FLAG_SP | GQFSSLHVRDTKIEVAQFVKDLLLHLKKLFREGRFNES | |
| IL13 mutein_IgG4 | KYGPPCPPCPAPEFEGGPSVFLFPPKPKDTLMISRTPEVT | |
| (L235E, | CVVVDVSQEDPEVQFNWYVDGVEVHNAKTKPREEQF | |
| N297Q)_CD28tm_ | QSTYRVVSVLTVLHQDWLNGKEYKCKVSNKGLPSSIE | |
| CD28cyto_CD3zeta) | KTISKAKGQPREPQVYTLPPSQEEMTKNQVSLTCLVKG | |
| | FYPSDIAVEWESNGQPENNYKTTPPVLDSDGSFFLYSRL | |
| | TVDKSRWQEGNVFSCSVMHEALHNHYTQKSLSLSLGK | |
| | MFWVLVVVGGVLACYSLLVTVAFIIFWVRSKRSRGGH | |
| | SDYMNMTPRRPGPTRKHYQPYAPPRDFAAYRSGGGRV | |
| | KFSRSADAPAYQQGQNQLYNELNLGRREEYDVLDKRR | |
| | GRDPEMGGKPRRKNPQEGLYNELQKDKMAEAYSEIG | |
| | MKGERRRGKGHDGLYQGLSTATKDTYDALHMQALPP | |
| | R | |
| SP-IL-13Rα2.28z | METDTLLLWVLLLWVPGSTGSPGPVPPSTALRYLIEEL | 140 |
| CAR; (Murine kappa | VNITQNQKAPLCNGSMVWSINLTAGMYCAALESLINVS | |
| signal sequence_SP | GCSAIEKTQRMLSGFCPHKVSAGQFSSLHVRDTKIEVA | |
| IL13 mutein_IgG4 | QFVKDLLLHLKKLFREGRFNESKYGPPCPPCPAPEFEGG | |
| (L235E, | PSVFLFPPKPKDTLMISRTPEVTCVVVDVSQEDPEVQFN | |
| N297Q)_CD28tm_ | WYVDGVEVHNAKTKPREEQFQSTYRVVSVLTVLHQD | |
| CD28cyto_CD3zeta) | WLNGKEYKCKVSNKGLPSSIEKTISKAKGQPREPQVYT | |
| | LPPSQEEMTKNQVSLTCLVKGFYPSDIAVEWESNGQPE | |
| | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVRSKRSRGGHSDYMNMTPRRPGPTRK | |
| | HYQPYAPPRDFAAYRSGGGRVKFSRSADAPAYQQGQN | |
| | QLYNELNLGRREEYDVLDKRRGRDPEMGGKPRRKNPQ | |
| | EGLYNELQKDKMAEAYSEIGMKGERRRGKGHDGLYQ | |
| | GLSTATKDTYDALHMQALPPR | |
| MK signal sequence | METDTLLLWVLLLWVPGSTG | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGREN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| SP-IL-13Rα2/TGF- | METDTLLLWVLLLWVPGSTGAGGSDYKDDDDKGGSS | 25 |
| β.28z CAR; (Murine | PGPVPPSTALRYLIEELVNITQNQKAPLCNGSMVWSINL | |
| kappa signal | TAGMYCAALESLINVSGCSAIEKTQRMLSGFCPHKVSA | |
| sequence_FLAG_SP | GQFSSLHVRDTKIEVAQFVKDLLLHLKKLFREGRENGG | |
| IL13 | GGSGGGGSGGGGSGGGGSQVQLVQSGAEVKKPGSSVK | |
| mutein_(G4S)x4_TGF- | VSCKASGYTFSSNVISWVRQAPGQGLEWMGGVIPIVDI | |
| β scFv_IgG4 | ANYAQRFKGRVTITADESTSTTYMELSSLRSEDTAVYY | |
| hinge_CD28tm_ | CALPRAFVLDAMDYWGQGTLVTVSSGGGGSGGGGSG | |
| CD28cyto_CD3zeta) | GGGSETVLTQSPGTLSLSPGERATLSCRASQSLGSSYLA | |
| | WYQQKPGQAPRLLIYGASSRAPGIPDRFSGSGSGTDFTL | |
| | TISRLEPEDFAVYYCQQYADSPITFGQGTRLEIKESKYG | |
| | PPCPPCPMFWVLVVVGGVLACYSLLVTVAFIIFWVRSK | |
| | RSRGGHSDYMNMTPRRPGPTRKHYQPYAPPRDFAAYR | |
| | SGGGRVKFSRSADAPAYQQGQNQLYNELNLGRREEYD | |
| | VLDKRRGRDPEMGGKPRRKNPQEGLYNELQKDKMAE | |
| | AYSEIGMKGERRRGKGHDGLYQGLSTATKDTYDALH | |
| | MQALPPR | |
| SP-IL-13Rα2/TGF- | METDTLLLWVLLLWVPGSTGSPGPVPPSTALRYLIEEL | 141 |
| β.28z CAR; (Murine | VNITQNQKAPLCNGSMVWSINLTAGMYCAALESLINVS | |
| kappa signal sequence | GCSAIEKTQRMLSGFCPHKVSAGQFSSLHVRDTKIEVA | |
| SP IL13 | QFVKDLLLHLKKLFREGRFNGGGGSGGGGSGGGGSGG | |
| mutein_(G4S)x4_TGF- | GGSQVQLVQSGAEVKKPGSSVKVSCKASGYTFSSNVIS | |
| β scFv_IgG4 | WVRQAPGQGLEWMGGVIPIVDIANYAQRFKGRVTITA | |
| hinge_CD28tm_ | DESTSTTYMELSSLRSEDTAVYYCALPRAFVLDAMDY | |
| CD28cyto_CD3zeta | WGQGTLVTVSSGGGGSGGGGSGGGGSETVLTQSPGTL | |
| | SLSPGERATLSCRASQSLGSSYLAWYQQKPGQAPRLLI | |
| | YGASSRAPGIPDRFSGSGSGTDFTLTISRLEPEDFAVYYC | |
| | QQYADSPITFGQGTRLEIKESKYGPPCPPCPMFWVLVV | |
| | VGGVLACYSLLVTVAFIIFWVRSKRSRGGHSDYMNMT | |
| | PRRPGPTRKHYQPYAPPRDFAAYRSGGGRVKFSRSADA | |
| | PAYQQGQNQLYNELNLGRREEYDVLDKRRGRDPEMG | |
| | GKPRRKNPQEGLYNELQKDKMAEAYSEIGMKGERRRG | |
| | KGHDGLYQGLSTATKDTYDALHMQALPPR | |
| MK signal sequence | METDTLLLWVLLLWVPGST | 2 |
| FLAG | DYKDDDDK | 3 |
| SP IL13 mutein | SPGPVPPSTALRYLIEELVNITQNQKAPLCNGSMVWSIN | 4 |
| | LTAGMYCAALESLINVSGCSAIEKTQRMLSGFCPHKVS | |
| | AGQFSSLHVRDTKIEVAQFVKDLLLHLKKLFREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRESGSGSGTDFTLTISRLEPEDFAVYYCQQYA | |
| | DSPITFGQGTRLEIK | |
| IgG4 hinge | ESKYGPPCPPCP | 12 |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full IL-13Rα2.BBz | METDTLLLWVLLLWVPGSTGAGGSDYKDDDDKGGSL | 19 |
| CAR; (Murine kappa | TCLGGFASPGPVPPSTALRYLIEELVNITQNQKAPLCNG | |
| signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 mutein_IgG4 | REGRFNESKYGPPCPPCPAPEFEGGPSVFLFPPKPKDTL | |
| (L235E, | MISRTPEVTCVVVDVSQEDPEVQFNWYVDGVEVHNAK | |
| N297Q)_CD28tm_4- | TKPREEQFQSTYRVVSVLTVLHQDWLNGKEYKCKVSN | |
| 1BB_CD3zeta) | KGLPSSIEKTISKAKGQPREPQVYTLPPSQEEMTKNQVS | |
| | LTCLVKGFYPSDIAVEWESNGQPENNYKTTPPVLDSDG | |
| | SFFLYSRLTVDKSRWQEGNVFSCSVMHEALHNHYTQK | |
| | SLSLSLGKMFWVLVVVGGVLACYSLLVTVAFIIFWVKR | |
| | GRKKLLYIFKQPFMRPVQTTQEEDGCSCRFPEEEEGGC | |
| | ELRVKFSRSADAPAYQQGQNQLYNELNLGRREEYDVL | |
| | DKRRGRDPEMGGKPRRKNPQEGLYNELQKDKMAEAY | |
| | SEIGMKGERRRGKGHDGLYQGLSTATKDTYDALHMQ | |
| | ALPPR | |
| Full IL-13Rα2.BBz | METDTLLLWVLLLWVPGSTGLTCLGGFASPGPVPPSTA | 142 |
| CAR; (Murine kappa | LRYLIEELVNITQNQKAPLCNGSMVWSINLTAGMYCAA | |
| signal sequence_Full | LESLINVSGCSAIEKTQRMLSGFCPHKVSAGQFSSLHVR | |
| IL13 mutein_IgG4 | DTKIEVAQFVKDLLLHLKKLFREGRFNESKYGPPCPPCP | |
| (L235E, | APEFEGGPSVFLFPPKPKDTLMISRTPEVTCVVVDVSQE | |
| N297Q)_CD28tm_4- | DPEVQFNWYVDGVEVHNAKTKPREEQFQSTYRVVSVL | |
| 1BB_CD3zeta) | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGKMFWVLVVVG | |
| | GVLACYSLLVTVAFIIFWVKRGRKKLLYIFKQPFMRPV | |
| | QTTQEEDGCSCRFPEEEEGGCELRVKFSRSADAPAYQQ | |
| | GQNQLYNELNLGRREEYDVLDKRRGRDPEMGGKPRR | |
| | KNPQEGLYNELQKDKMAEAYSEIGMKGERRRGKGHD | |
| | GLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28tm | MFWVLVVVGGVLACYSLLVTVAFIIFWV | 6 |
| 4-1BB | KRGRKKLLYIFKQPFMRPVQTTQEEDGCSCRFPEEEEG | 7 |
| | GCEL | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full-IL-13Rα2/TGF- | METDTLLLWVLLLWVPGSTGAGGSDYKDDDDKGGSL | 21 |
| β.BBz CAR; (Murine | TCLGGFASPGPVPPSTALRYLIEELVNITQNQKAPLCNG | |
| kappa signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 | REGRFNGGGGSGGGGSGGGGSGGGGSQVQLVQSGAE | |
| mutein_(G4S)x4_TGF- | VKKPGSSVKVSCKASGYTFSSNVISWVRQAPGQGLEW | |
| β scFv_IgG4 | MGGVIPIVDIANYAQRFKGRVTITADESTSTTYMELSSL | |
| hinge_CD28tm_4- | RSEDTAVYYCALPRAFVLDAMDYWGQGTLVTVSSGG | |
| 1BB_CD3zeta) | GGSGGGGSGGGGSETVLTQSPGTLSLSPGERATLSCRA | |
| | SQSLGSSYLAWYQQKPGQAPRLLIYGASSRAPGIPDRFS | |
| | GSGSGTDFTLTISRLEPEDFAVYYCQQYADSPITFGQGT | |
| | RLEIKESKYGPPCPPCPMFWVLVVVGGVLACYSLLVTV | |
| | AFIIFWVKRGRKKLLYIFKQPFMRPVQTTQEEDGCSCRF | |
| | PEEEEGGCELRVKFSRSADAPAYQQGQNQLYNELNLG | |
| | RREEYDVLDKRRGRDPEMGGKPRRKNPQEGLYNELQK | |
| | DKMAEAYSEIGMKGERRRGKGHDGLYQGLSTATKDT | |
| | YDALHMQALPPR | |
| Full IL-13Rα2/TGF- | METDTLLLWVLLLWVPGSTGLTCLGGFASPGPVPPSTA | 143 |
| β.BBz CAR; (Murine | LRYLIEELVNITQNQKAPLCNGSMVWSINLTAGMYCAA | |
| kappa signal sequence | LESLINVSGCSAIEKTQRMLSGFCPHKVSAGQFSSLHVR | |
| Full IL13 | DTKIEVAQFVKDLLLHLKKLFREGRENGGGGSGGGGS | |
| mutein_(G4S)x4_TGF- | GGGGSGGGGSQVQLVQSGAEVKKPGSSVKVSCKASGY | |
| β scFv_IgG4 | TFSSNVISWVRQAPGQGLEWMGGVIPIVDIANYAQRFK | |
| hinge_CD28tm_4- | GRVTITADESTSTTYMELSSLRSEDTAVYYCALPRAFVL | |
| 1BB_CD3zeta) | DAMDYWGQGTLVTVSSGGGGSGGGGSGGGGSETVLT | |
| | QSPGTLSLSPGERATLSCRASQSLGSSYLAWYQQKPGQ | |
| | APRLLIYGASSRAPGIPDRFSGSGSGTDFTLTISRLEPEDF | |
| | AVYYCQQYADSPITFGQGTRLEIKESKYGPPCPPCPMF | |
| | WVLVVVGGVLACYSLLVTVAFIIFWVKRGRKKLLYIFK | |
| | QPFMRPVQTTQEEDGCSCRFPEEEEGGCELRVKFSRSA | |
| | DAPAYQQGQNQLYNELNLGRREEYDVLDKRRGRDPE | |
| | MGGKPRRKNPQEGLYNELQKDKMAEAYSEIGMKGER | |
| | RRGKGHDGLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRESGSGSGTDFTLTISRLEPEDFAVYYCQQYA | |
| | DSPITFGQGTRLEIK | |
| IgG4 hinge | ESKYGPPCPPCP | 12 |
| CD28tm | MFWVLVVVGGVLACYSLLVTVAFIIFWV | 6 |
| 4-1BB | KRGRKKLLYIFKQPFMRPVQTTQEEDGCSCRFPEEEEG | 7 |
| | GCEL | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full-IL-13Rα2.28z | METDTLLLWVLLLWVPGSTGAGGSDYKDDDDKGGSL | 22 |
| CAR; (Murine kappa | TCLGGFASPGPVPPSTALRYLIEELVNITQNQKAPLCNG | |
| signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 mutein_IgG4 | REGRFNESKYGPPCPPCPAPEFEGGPSVFLFPPKPKDTL | |
| (L235E, | MISRTPEVTCVVVDVSQEDPEVQFNWYVDGVEVHNAK | |
| N297Q)_CD28tm_ | TKPREEQFQSTYRVVSVLTVLHQDWLNGKEYKCKVSN | |
| CD28cyto_CD3zeta) | KGLPSSIEKTISKAKGQPREPQVYTLPPSQEEMTKNQVS | |
| | LTCLVKGFYPSDIAVEWESNGQPENNYKTTPPVLDSDG | |
| | SFFLYSRLTVDKSRWQEGNVFSCSVMHEALHNHYTQK | |
| | SLSLSLGKMFWVLVVVGGVLACYSLLVTVAFIIFWVRS | |
| | KRSRGGHSDYMNMTPRRPGPTRKHYQPYAPPRDFAAY | |
| | RSGGGRVKFSRSADAPAYQQGQNQLYNELNLGRREEY | |
| | DVLDKRRGRDPEMGGKPRRKNPQEGLYNELQKDKMA | |
| | EAYSEIGMKGERRRGKGHDGLYQGLSTATKDTYDALH | |
| | MQALPPR | |
| Full-IL-13Rα2.28z | METDTLLLWVLLLWVPGSTGLTCLGGFASPGPVPPSTA | 144 |
| CAR; (Murine kappa | LRYLIEELVNITQNQKAPLCNGSMVWSINLTAGMYCAA | |
| signal sequence_ Full | LESLINVSGCSAIEKTQRMLSGFCPHKVSAGQFSSLHVR | |
| IL13 mutein_IgG4 | DTKIEVAQFVKDLLLHLKKLFREGRFNESKYGPPCPPCP | |
| (L235E, | APEFEGGPSVFLFPPKPKDTLMISRTPEVTCVVVDVSQE | |
| N297Q)_CD28tm_ | DPEVQFNWYVDGVEVHNAKTKPREEQFQSTYRVVSVL | |
| CD28cyto_CD3zeta) | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGKMFWVLVVVG | |
| | GVLACYSLLVTVAFIIFWVRSKRSRGGHSDYMNMTPRR | |
| | PGPTRKHYQPYAPPRDFAAYRSGGGRVKFSRSADAPA | |
| | YQQGQNQLYNELNLGRREEYDVLDKRRGRDPEMGGK | |
| | PRRKNPQEGLYNELQKDKMAEAYSEIGMKGERRRGKG | |
| | HDGLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| IgG4 (L235E, N297Q) | ESKYGPPCPPCPAPEFEGGPSVFLFPPKPKDTLMISRTPE | 5 |
| | VTCVVVDVSQEDPEVQFNWYVDGVEVHNAKTKPREE | |
| | QFQSTYRVVSVLTVLHQDWLNGKEYKCKVSNKGLPSS | |
| | IEKTISKAKGQPREPQVYTLPPSQEEMTKNQVSLTCLVK | |
| | GFYPSDIAVEWESNGQPENNYKTTPPVLDSDGSFFLYSR | |
| | LTVDKSRWQEGNVFSCSVMHEALHNHYTQKSLSLSLG | |
| | K | |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| Full-IL-13Rα2/TGF- | METDTLLLWVLLLWVPGSTGAGGSDYKDDDDKGGSL | 23 |
| β.28z CAR; (Murine | TCLGGFASPGPVPPSTALRYLIEELVNITQNQKAPLCNG | |
| kappa signal | SMVWSINLTAGMYCAALESLINVSGCSAIEKTQRMLSG | |
| sequence_FLAG_Full | FCPHKVSAGQFSSLHVRDTKIEVAQFVKDLLLHLKKLF | |
| IL13 | REGRFNGGGGSGGGGSGGGGSGGGGSQVQLVQSGAE | |
| mutein_(G4S)x4_TGF- | VKKPGSSVKVSCKASGYTFSSNVISWVRQAPGQGLEW | |
| β scFv_IgG4 | MGGVIPIVDIANYAQRFKGRVTITADESTSTTYMELSSL | |
| hinge_CD28tm | RSEDTAVYYCALPRAFVLDAMDYWGQGTLVTVSSGG | |
| CD28cyto_CD3zeta) | GGSGGGGSGGGGSETVLTQSPGTLSLSPGERATLSCRA | |
| | SQSLGSSYLAWYQQKPGQAPRLLIYGASSRAPGIPDRFS | |
| | GSGSGTDFTLTISRLEPEDFAVYYCQQYADSPITFGQGT | |
| | RLEIKESKYGPPCPPCPMFWVLVVVGGVLACYSLLVTV | |
| | AFIIFWVRSKRSRGGHSDYMNMTPRRPGPTRKHYQPYA | |
| | PPRDFAAYRSGGGRVKFSRSADAPAYQQGQNQLYNEL | |
| | NLGRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| Full-IL-13Rα2/TGF- | METDTLLLWVLLLWVPGSTGLTCLGGFASPGPVPPSTA | 145 |
| β.28z CAR; (Murine | LRYLIEELVNITQNQKAPLCNGSMVWSINLTAGMYCAA | |
| kappa signal sequence_ | LESLINVSGCSAIEKTQRMLSGFCPHKVSAGQFSSLHVR | |
| Full IL13 | DTKIEVAQFVKDLLLHLKKLFREGRENGGGGSGGGGS | |
| mutein_(G4S)x4_TGF- | GGGGSGGGGSQVQLVQSGAEVKKPGSSVKVSCKASGY | |
| β scFv_IgG4 | TFSSNVISWVRQAPGQGLEWMGGVIPIVDIANYAQRFK | |
| hinge_CD28tm_ | GRVTITADESTSTTYMELSSLRSEDTAVYYCALPRAFVL | |
| CD28cyto_CD3zeta) | DAMDYWGQGTLVTVSSGGGGSGGGGSGGGGSETVLT | |
| | QSPGTLSLSPGERATLSCRASQSLGSSYLAWYQQKPGQ | |
| | APRLLIYGASSRAPGIPDRFSGSGSGTDFTLTISRLEPEDF | |
| | AVYYCQQYADSPITFGQGTRLEIKESKYGPPCPPCPMF | |
| | WVLVVVGGVLACYSLLVTVAFIIFWVRSKRSRGGHSD | |
| | YMNMTPRRPGPTRKHYQPYAPPRDFAAYRSGGGRVKF | |
| | SRSADAPAYQQGQNQLYNELNLGRREEYDVLDKRRGR | |
| | DPEMGGKPRRKNPQEGLYNELQKDKMAEAYSEIGMK | |
| | GERRRGKGHDGLYQGLSTATKDTYDALHMQALPPR | |
| MK signal sequence | METDTLLLWVLLLWVPGSTG | 2 |
| FLAG | DYKDDDDK | 3 |
| Full IL13 mutein | LTCLGGFASPGPVPPSTALRYLIEELVNITQNQKAPLCN | 20 |
| | GSMVWSINLTAGMYCAALESLINVSGCSAIEKTQRMLS | |
| | GFCPHKVSAGQFSSLHVRDTKIEVAQFVKDLLLHLKKL | |
| | FREGREN | |
| (G4S)x4 | GGGGSGGGGSGGGGSGGGGS | 10 |
| TGF-β scFv | QVQLVQSGAEVKKPGSSVKVSCKASGYTFSSNVISWVR | 11 |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRESGSGSGTDFTLTISRLEPEDFAVYYCQQYA | |
| | DSPITFGQGTRLEIK | |
| IgG4 hinge | ESKYGPPCPPCP | 12 |
| CD28tm | MFWVLVVVGGVLACYSLLVTVAFIIFWV | 6 |
| CD28cyto | RSKRSRGGHSDYMNMTPRRPGPTRKHYQPYAPPRDFA | 18 |
| | AYRS | |
| CD3zeta | RVKFSRSADAPAYQQGQNQLYNELNLGRREEYDVLDK | 8 |
| | RRGRDPEMGGKPRRKNPQEGLYNELQKDKMAEAYSEI | |
| | GMKGERRRGKGHDGLYQGLSTATKDTYDALHMQALP | |
| | PR | |
| TGF-β scFv VH | QVQLVQSGAEVKKPGSSVKVSCKASGYTFSSNVISWVR | 29 |
| | QAPGQGLEWMGGVIPIVDIANYAQRFKGRVTITADEST | |
| | STTYMELSSLRSEDTAVYYCALPRAFVLDAMDYWGQG | |
| | TLVTVSS | |
| TGF-β scFv VL | ETVLTQSPGTLSLSPGERATLSCRASQSLGSSYLAWYQ | 30 |
| | QKPGQAPRLLIYGASSRAPGIPDRFSGSGSGTDFTLTISR | |
| | LEPEDFAVYYCQQYADSPITFGQGTRLEIK | |
| TGF-β scFv HCDR1 | SNVIS | 31 |
| TGF-β scFv HCDR2 | GVIPIVDIANYAQRFKG | 32 |
| TGF-β scFv HCDR3 | PRAFVLDAMDY | 33 |
| TGF-β scFv LCDR1 | RASQSLGSSYLA | 34 |
| TGF-β scFv LCDR2 | GASSRAP | 35 |
| TGF-β scFv LCDR3 | QQYADSPIT | 36 |
| IgG4 CH2 CH3 L235E | APEFEGGPSVFLFPPKPKDTLMISRTPEVTCVVVDVSQE | 37 |
| N297Q peptide spacer | DPEVQFNWYVDGVEVHNAKTKPREEQFQSTYRVVSVL | |
| | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGK | |
| EGFRvIII scFv VH | EIQLVQSGAEVKKPGESLRISCKGSGFNIEDYYIHWVRQ | 38 |
| | MPGKGLEWMGRIDPENDETKYGPIFQGHVTISADTSIN | |
| | TVYLQWSSLKASDTAMYYCAFRGGVYWGQGTTVTVS | |
| | S | |
| EGFRvIII scFv VL | DVVMTQSPDSLAVSLGERATINCKSSQSLLDSDGKTYL | 39 |
| | NWLQQKPGQPPKRLISLVSKLDSGVPDRFSGSGSGTDF | |
| | TLTISSLQAEDVAVYYCWQGTHFPGTFGGGTKVEIK | |
| EGFRvIII scFv | DYYIH | 40 |
| HCDR1 | | |
| EGFRvIII scFv | RIDPENDETKYGPIFQG | 41 |
| HCDR2 | | |
| EGFRvIII scFv | RGGVY | 42 |
| HCDR3 | | |
| EGFRvIII scFv LCDR1 | KSSQSLLDSDGKTYLN | 43 |
| EGFRvIII scFv LCDR2 | LVSKLDS | 44 |
| EGFRvIII scFv LCDR3 | WQGTHFPGT | 45 |
| GD2 (14g2a scFv) VH | EVQLLQSGPELEKPGASVMISCKASGSSFTGYNMNWV | 46 |
| | RQNIGKSLEWIGAIDPYYGGTSYNQKFKGRATLTVDKS | |
| | SSTAYMHLKSLTSEDSAVYYCVSGMEYWGQGTSVTVS | |
| | S | |
| GD2 (14g2a scFv) VL | DVVMTQTPLSLPVSLGDQASISCRSSQSLVHRNGNTYL | 47 |
| | HWYLQKPGQSPKLLIHKVSNRFSGVPDRFSGSGSGTDF | |
| | TLKISRVEAEDLGVYFCSQSTHVPPLTFGAGTKLELKRA | |
| GD2scFv HCDR1 | GYNMN | 48 |
| GD2scFv HCDR2 | AIDPYYGGTSYNQKFKG | 49 |
| GD2scFv HCDR3 | GMEY | 50 |
| GD2scFv LCDR1 | RSSQSLVHRNGNTYLH | 51 |
| GD2scFv LCDR2 | KVSNRFS | 52 |
| GD2scFv LCDR3 | SQSTHVPPLT | 53 |
| TGF-β scFv VH #2 | EVQLVESGGGLVQPGGSLRLSCAASGYAFTNYLIEWVR | 54 |
| | QAPGKGLEWVGVINPGSGGSNYNEKFKGRATISADNS | |
| | KNTLYLQMNSLRAEDTAVYYCARSGGFYFDYWGQGT | |
| | LVTVSSASTKGPS | |
| TGF-β scFv VL #2 | DIQMTQSPSSLSASVGDRVTITCRASQSVLYSSNQKNYL | 55 |
| | AWYQQKPGKAPKLLIYWASTRESGVPSRFSGSGSGTDF | |
| | TLTISSLQPEDFATYYCHQYLSSDTFGQGTKVEIKRTVA | |
| TGF-β scFv #2 | GYAFTNYLIE | 56 |
| HCDR1 | | |
| TGF-β scFv #2 | VINPGSGGSNYNEKFKG | 57 |
| HCDR2 | | |
| TGF-β scFv #2 | SGGFYFDY | 58 |
| HCDR3 | | |
| TGF-β scFv #2 LCDR1 | RASQSVLYSSNQKNYLA | 59 |
| TGF-β scFv #2 LCDR2 | WASTRES | 60 |
| TGF-β scFv #2 LCDR3 | HQYLSSDT | 61 |
| TGF-β scFv VH #3 | EVQLVESGGGVVQPGRSLRLSCAASGFTFSSYGMHWV | 62 |
| | RQAPGKELEWVAVISYDGSIKYYADSVKGRFTISRDNS | |
| | KNTLYLQMNSLRAEDTAVYYCARTGEYSGYDTDPQYS | |
| | WGQGTTVTVSS | |
| TGF-β scFv VL #3 | EIVLTQSPSSLSASVGDRVTITCRSSQGIGDDLGWYQQK | 63 |
| | PGKAPILLIYGTSTLQSGVPSRFSGSGSGTDFTLTINSLQP | |
| | EDFATYYCLQDSNYPLTFGGGTRLEIK | |
| TGF-β scFv #3 | SYGMH | 64 |
| HCDR1 | | |
| TGF-β scFv #3 | VISYDGSIKYYADSVKG | 65 |
| HCDR2 | | |
| TGF-β scFv #3 | TGEYSGYDTDPQYS | 66 |
| HCDR3 | | |
| TGF-β scFv #3 LCDR1 | RSSQGIGDDLG | 67 |
| TGF-β scFv #3 LCDR2 | GTSTLQS | 68 |
| TGF-β scFv #3 LCDR3 | LQDSNYPLT | 69 |
| Murine kappa signal | METDTLLLWVLLLWVPGSTGLTCLGGFASPGPVPPSTA | 159 |
| sequence_Full IL13 | LRYLIEELVNITQNQKAPLCNGSMVWSINLTAGMYCAA | |
| mutein_IgG4 (L235E, | LESLINVSGCSAIEKTQRMLSGFCPHKVSAGQFSSLHVR | |
| N297Q)_CD28tm_4- | DTKIEVAQFVKDLLLHLKKLFREGRFNESKYGPPCPPCP | |
| 1BB (KR)_CD3zeta | APEFEGGPSVFLFPPKPKDTLMISRTPEVTCVVVDVSQE | |
| (KR) | DPEVQFNWYVDGVEVHNAKTKPREEQFQSTYRVVSVL | |
| | TVLHQDWLNGKEYKCKVSNKGLPSSIEKTISKAKGQPR | |
| | EPQVYTLPPSQEEMTKNQVSLTCLVKGFYPSDIAVEWE | |
| | SNGQPENNYKTTPPVLDSDGSFFLYSRLTVDKSRWQEG | |
| | NVFSCSVMHEALHNHYTQKSLSLSLGKMFWVLVVVG | |
| | GVLACYSLLVTVAFIIFWVRRGRRRLLYIFRQPFMRPVQ | |
| | TTQEEDGCSCRFPEEEEGGCELRVRFSRSADAPAYQQG | |
| | QNQLYNELNLGRREEYDVLDRRRGRDPEMGGRPRRRN | |
| | PQEGLYNELQRDRMAEAYSEIGMRGERRRGRGHDGLY | |
| | QGLSTATRDTYDALHMQALPPR | |
| Murine kappa signal | METDTLLLWVLLLWVPGSTGLTCLGGFASPGPVPPSTA | 160 |
| sequence_Full IL13 | LRYLIEELVNITQNQKAPLCNGSMVWSINLTAGMYCAA | |
| mutein_(G4S)x4_TGF- | LESLINVSGCSAIEKTQRMLSGFCPHKVSAGQFSSLHVR | |
| β scFv_IgG4 | DTKIEVAQFVKDLLLHLKKLFREGRENGGGGSGGGGS | |
| hinge_CD28tm_4-1BB | GGGGSGGGGSQVQLVQSGAEVKKPGSSVKVSCKASGY | |
| (KR)_CD3zeta (KR) | TFSSNVISWVRQAPGQGLEWMGGVIPIVDIANYAQRFK | |
| | GRVTITADESTSTTYMELSSLRSEDTAVYYCALPRAFVL | |
| | DAMDYWGQGTLVTVSSGGGGSGGGGSGGGGSETVLT | |
| | QSPGTLSLSPGERATLSCRASQSLGSSYLAWYQQKPGQ | |
| | APRLLIYGASSRAPGIPDRFSGSGSGTDFTLTISRLEPEDF | |
| | AVYYCQQYADSPITFGQGTRLEIKESKYGPPCPPCPMF | |
| | WVLVVVGGVLACYSLLVTVAFIIFWVRRGRRRLLYIFR | |
| | QPFMRPVQTTQEEDGCSCRFPEEEEGGCELRVRFSRSA | |
| | DAPAYQQGQNQLYNELNLGRREEYDVLDRRRGRDPE | |
| | MGGRPRRRNPQEGLYNELQRDRMAEAYSEIGMRGERR | |
| | RGRGHDGLYQGLSTATRDTYDALHMQALPPR | |
| IL13Rα2.BBz | METDTLLLWVLLLWVPGSTGGPVPPSTALRYLIEELVNI | 161 |
| (MKleader-IL13op- | TQNQKAPLCNGSMVWSINLTAGMYCAALESLINVSGC | |
| IgG4 hinge_IgG4 CH2 | SAIEKTQRMLSGFCPHKVSAGQFSSLHVRDTKIEVAQF | |
| CH3 L235E N297Q | VKDLLLHLKKLFREGRFNESKYGPPCPPCPAPEFEGGPS | |
| peptide spacer-CD28 | VFLFPPKPKDTLMISRTPEVTCVVVDVSQEDPEVQFNW | |
| transmembrane | YVDGVEVHNAKTKPREEQFQSTYRVVSVLTVLHQDWL | |
| domain-4-1BB Co- | NGKEYKCKVSNKGLPSSIEKTISKAKGQPREPQVYTLPP | |
| stimulatory-CD3 Zeta) | SQEEMTKNQVSLTCLVKGFYPSDIAVEWESNGQPENN | |
| | YKTTPPVLDSDGSFFLYSRLTVDKSRWQEGNVFSCSVM | |
| | HEALHNHYTQKSLSLSLGKMFWVLVVVGGVLACYSLL | |
| | VTVAFIIFWVKRGRKKLLYIFKQPFMRPVQTTQEEDGC | |
| | SCRFPEEEEGGCELRVKFSRSADAPAYQQGQNQLYNEL | |
| | NLGRREEYDVLDKRRGRDPEMGGKPRRKNPQEGLYNE | |
| | LQKDKMAEAYSEIGMKGERRRGKGHDGLYQGLSTAT | |
| | KDTYDALHMQALPPR | |
| IL13Rα2-(G4S)x3- | METDTLLLWVLLLWVPGSTGGPVPPSTALRYLIEELVNI | 162 |
| TGF-β.BBz | TQNQKAPLCNGSMVWSINLTAGMYCAALESLINVSGC | |
| (MKleader-IL13op- | SAIEKTQRMLSGFCPHKVSAGQFSSLHVRDTKIEVAQF | |
| (G4S)x3-TGF-β scFv- | VKDLLLHLKKLFREGRENGGGGSGGGGSGGGGSQVQL | |
| IgG4 hinge peptide | VQSGAEVKKPGSSVKVSCKASGYTFSSNVISWVRQAPG | |
| spacer-CD28 | QGLEWMGGVIPIVDIANYAQRFKGRVTITADESTSTTY | |
| transmembrane | MELSSLRSEDTAVYYCALPRAFVLDAMDYWGQGTLVT | |
| domain-4-1BB Co- | VSSGGGGSGGGGSGGGGSETVLTQSPGTLSLSPGERAT | |
| stimulatory-CD3 Zeta) | LSCRASQSLGSSYLAWYQQKPGQAPRLLIYGASSRAPGI | |
| | PDRFSGSGSGTDFTLTISRLEPEDFAVYYCQQYADSPITF | |
| | GQGTRLEIKESKYGPPCPPCPMFWVLVVVGGVLACYSL | |
| | LVTVAFIIFWVKRGRKKLLYIFKQPFMRPVQTTQEEDG | |
| | CSCRFPEEEEGGCELRVKFSRSADAPAYQQGQNQLYNE | |
| | LNLGRREEYDVLDKRRGRDPEMGGKPRRKNPQEGLYN | |
| | ELQKDKMAEAYSEIGMKGERRRGKGHDGLYQGLSTAT | |
| | KDTYDALHMQALPPR | |
| IL13Rα2-(G4S)x4- | METDTLLLWVLLLWVPGSTGGPVPPSTALRYLIEELVNI | 163 |
| TGF-β.BBz | TQNQKAPLCNGSMVWSINLTAGMYCAALESLINVSGC | |
| (MKleader-IL13op- | SAIEKTQRMLSGFCPHKVSAGQFSSLHVRDTKIEVAQF | |
| (G4S)x4-TGF-β scFv- | VKDLLLHLKKLFREGRFNGGGGSGGGGSGGGGSGGGG | |
| IgG4 hinge peptide | SQVQLVQSGAEVKKPGSSVKVSCKASGYTFSSNVISWV | |
| spacer-CD28 | RQAPGQGLEWMGGVIPIVDIANYAQRFKGRVTITADES | |
| transmembrane | TSTTYMELSSLRSEDTAVYYCALPRAFVLDAMDYWGQ | |
| domain-4-1BB Co- | GTLVTVSSGGGGSGGGGSGGGGSETVLTQSPGTLSLSP | |
| stimulatory-CD3 Zeta) | GERATLSCRASQSLGSSYLAWYQQKPGQAPRLLIYGAS | |
| | SRAPGIPDRFSGSGSGTDFTLTISRLEPEDFAVYYCQQY | |
| | ADSPITFGQGTRLEIKESKYGPPCPPCPMFWVLVVVGG | |
| | VLACYSLLVTVAFIIFWVKRGRKKLLYIFKQPFMRPVQ | |
| | TTQEEDGCSCRFPEEEEGGCELRVKFSRSADAPAYQQG | |
| | QNQLYNELNLGRREEYDVLDKRRGRDPEMGGKPRRK | |
| | NPQEGLYNELQKDKMAEAYSEIGMKGERRRGKGHDG | |
| | LYQGLSTATKDTYDALHMQALPPR | |
| IL13Rα2-(G4S)x4- | METDTLLLWVLLLWVPGSTGGPVPPSTALRYLIEELVNI | 164 |
| EGFRvIII.BBz | TQNQKAPLCNGSMVWSINLTAGMYCAALESLINVSGC | |
| (MKleader-IL13op- | SAIEKTQRMLSGFCPHKVSAGQFSSLHVRDTKIEVAQF | |
| (G4S)x4-EGFRvIII | VKDLLLHLKKLFREGRFNGGGGSGGGGSGGGGSGGGG | |
| scFv-IgG4 hinge | SEIQLVQSGAEVKKPGESLRISCKGSGFNIEDYYIHWVR | |
| peptide spacer | QMPGKGLEWMGRIDPENDETKYGPIFQGHVTISADTSI | |
| transmembrane | NTVYLQWSSLKASDTAMYYCAFRGGVYWGQGTTVTV | |
| domain-4-1BB Co- | SSGSTSGSGKPGSGEGSTKGDVVMTQSPDSLAVSLGER | |
| stimulatory-CD3 Zeta) | ATINCKSSQSLLDSDGKTYLNWLQQKPGQPPKRLISLVS | |
| | KLDSGVPDRFSGSGSGTDFTLTISSLQAEDVAVYYCWQ | |
| | GTHFPGTFGGGTKVEIKESKYGPPCPPCPMFWVLVVVG | |
| | GVLACYSLLVTVAFIIFWVKRGRKKLLYIFKQPFMRPV | |
| | QTTQEEDGCSCRFPEEEEGGCELRVKFSRSADAPAYQQ | |
| | GQNQLYNELNLGRREEYDVLDKRRGRDPEMGGKPRR | |
| | KNPQEGLYNELQKDKMAEAYSEIGMKGERRRGKGHD | |
| | GLYQGLSTATKDTYDALHMQALPPR | |
| IL13Rα2-(G4S)x4- | METDTLLLWVLLLWVPGSTGGPVPPSTALRYLIEELVNI | 165 |
| EGFRvIII.BBz | TQNQKAPLCNGSMVWSINLTAGMYCAALESLINVSGC | |
| (MKleader-IL13op- | SAIEKTQRMLSGFCPHKVSAGQFSSLHVRDTKIEVAQF | |
| (G4S)x4-EGFRvIII | VKDLLLHLKKLFREGRENGGGGSGGGGSGGGGSGGGG | |
| scFv-IgG4 hinge: IgG4 | SEIQLVQSGAEVKKPGESLRISCKGSGFNIEDYYIHWVR | |
| CH2 CH3 L235E | QMPGKGLEWMGRIDPENDETKYGPIFQGHVTISADTSI | |
| N297Q peptide spacer- | NTVYLQWSSLKASDTAMYYCAFRGGVYWGQGTTVTV | |
| CD28 transmembrane | SSGSTSGSGKPGSGEGSTKGDVVMTQSPDSLAVSLGER | |
| domain-4-1BB Co- | ATINCKSSQSLLDSDGKTYLNWLQQKPGQPPKRLISLVS | |
| stimulatory-CD3 Zeta) | KLDSGVPDRFSGSGSGTDFTLTISSLQAEDVAVYYCWQ | |
| | GTHFPGTFGGGTKVEIKESKYGPPCPPCPAPEFEGGPSV | |
| | FLFPPKPKDTLMISRTPEVTCVVVDVSQEDPEVQFNWY | |
| | VDGVEVHNAKTKPREEQFQSTYRVVSVLTVLHQDWLN | |
| | GKEYKCKVSNKGLPSSIEKTISKAKGQPREPQVYTLPPS | |
| | QEEMTKNQVSLTCLVKGFYPSDIAVEWESNGQPENNY | |
| | KTTPPVLDSDGSFFLYSRLTVDKSRWQEGNVFSCSVMH | |
| | EALHNHYTQKSLSLSLGKMFWVLVVVGGVLACYSLLV | |
| | TVAFIIFWVKRGRKKLLYIFKQPFMRPVQTTQEEDGCSC | |
| | RFPEEEEGGCELRVKFSRSADAPAYQQGQNQLYNELNL | |
| | GRREEYDVLDKRRGRDPEMGGKPRRKNPQEGLYNELQ | |
| | KDKMAEAYSEIGMKGERRRGKGHDGLYQGLSTATKD | |
| | TYDALHMQALPPR | |
| IL13Rα2-(G4S)x4- | METDTLLLWVLLLWVPGSTGGPVPPSTALRYLIEELVNI | 166 |
| GD2.BBz (MKleader- | TQNQKAPLCNGSMVWSINLTAGMYCAALESLINVSGC | |
| IL13op-(G4S)x4-GD2 | SAIEKTQRMLSGFCPHKVSAGQFSSLHVRDTKIEVAQF | |
| scFv-IgG4 hinge | VKDLLLHLKKLFREGRFNGGGGSGGGGSGGGGSGGGG | |
| peptide spacer-CD28 | SEVQLLQSGPELEKPGASVMISCKASGSSFTGYNMNWV | |
| transmembrane | RQNIGKSLEWIGAIDPYYGGTSYNQKFKGRATLTVDKS | |
| domain-4-1BB co- | SSTAYMHLKSLTSEDSAVYYCVSGMEYWGQGTSVTVS | |
| stimulatory-CD3 Zeta) | SGSTSGSGKPGSGEGSTKGDVVMTQTPLSLPVSLGDQA | |
| | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRAESKYGPPCPPCPMFWVLVV | |
| | VGGVLACYSLLVTVAFIIFWVKRGRKKLLYIFKQPFMR | |
| | PVQTTQEEDGCSCRFPEEEEGGCELRVKFSRSADAPAY | |
| | QQGQNQLYNELNLGRREEYDVLDKRRGRDPEMGGKP | |
| | RRKNPQEGLYNELQKDKMAEAYSEIGMKGERRRGKG | |
| | HDGLYQGLSTATKDTYDALHMQALPPR | |
| IL13Rα2-(G4S)x4- | METDTLLLWVLLLWVPGSTGGPVPPSTALRYLIEELVNI | 167 |
| GD2.BBz (MKleader- | TQNQKAPLCNGSMVWSINLTAGMYCAALESLINVSGC | |
| IL13op-(G4S)x4-GD2 | SAIEKTQRMLSGFCPHKVSAGQFSSLHVRDTKIEVAQF | |
| scFv-IgG4 hinge:IgG4 | VKDLLLHLKKLFREGRENGGGGSGGGGSGGGGSGGGG | |
| CH2 CH3 L235E | SEVQLLQSGPELEKPGASVMISCKASGSSFTGYNMNWV | |
| N297Q peptide spacer- | RQNIGKSLEWIGAIDPYYGGTSYNQKFKGRATLTVDKS | |
| CD28 transmembrane | SSTAYMHLKSLTSEDSAVYYCVSGMEYWGQGTSVTVS | |
| domain-4-1BB co- | SGSTSGSGKPGSGEGSTKGDVVMTQTPLSLPVSLGDQA | |
| stimulatory-CD3 Zeta) | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRAESKYGPPCPPCPAPEFEGGP | |
| | SVFLFPPKPKDTLMISRTPEVTCVVVDVSQEDPEVQFN | |
| | WYVDGVEVHNAKTKPREEQFQSTYRVVSVLTVLHQD | |
| | WLNGKEYKCKVSNKGLPSSIEKTISKAKGQPREPQVYT | |
| | LPPSQEEMTKNQVSLTCLVKGFYPSDIAVEWESNGQPE | |
| | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVKRGRKKLLYIFKQPFMRPVQTTQEED | |
| | GCSCRFPEEEEGGCELRVKFSRSADAPAYQQGQNQLYN | |
| | ELNLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPR | |
| IL13Rα2.BBz + TGF- | METDTLLLWVLLLWVPGSTGGPVPPSTALRYLIEELVNI | 168 |
| β.28z (MKleader- | TQNQKAPLCNGSMVWSINLTAGMYCAALESLINVSGC | |
| IL13op-IgG4 | SAIEKTQRMLSGFCPHKVSAGQFSSLHVRDTKIEVAQF | |
| hinge: IgG4 CH2 CH3 | VKDLLLHLKKLFREGRFNESKYGPPCPPCPAPEFEGGPS | |
| L235E N297Q peptide | VFLFPPKPKDTLMISRTPEVTCVVVDVSQEDPEVQFNW | |
| spacer-CD28 | YVDGVEVHNAKTKPREEQFQSTYRVVSVLTVLHQDWL | |
| transmembrane | NGKEYKCKVSNKGLPSSIEKTISKAKGQPREPQVYTLPP | |
| domain-4-1BB co- | SQEEMTKNQVSLTCLVKGFYPSDIAVEWESNGQPENN | |
| stimulatory-CD3 Zeta- | YKTTPPVLDSDGSFFLYSRLTVDKSRWQEGNVFSCSVM | |
| T2A-MKleader-HA- | HEALHNHYTQKSLSLSLGKMFWVLVVVGGVLACYSLL | |
| TGF-β scFv-IgG4 | VTVAFIIFWVKRGRKKLLYIFKQPFMRPVQTTQEEDGC | |
| hinge peptide spacer- | SCRFPEEEEGGCELRVKFSRSADAPAYQQGQNQLYNEL | |
| CD28 transmembrane | NLGRREEYDVLDKRRGRDPEMGGKPRRKNPQEGLYNE | |
| domain-CD28cyto | LQKDKMAEAYSEIGMKGERRRGKGHDGLYQGLSTAT | |
| (with gg mutations)- | KDTYDALHMQALPPRLEGGGEGRGSLLTCGDVEENPG | |
| CD3 Zeta) | PRMETDTLLLWVLLLWVPGSTGTSYPYDVPDYAGGSQ | |
| | VQLVQSGAEVKKPGSSVKVSCKASGYTFSSNVISWVRQ | |
| | APGQGLEWMGGVIPIVDIANYAQRFKGRVTITADESTS | |
| | TTYMELSSLRSEDTAVYYCALPRAFVLDAMDYWGQGT | |
| | LVTVSSGGGGSGGGGSGGGGSETVLTQSPGTLSLSPGE | |
| | RATLSCRASQSLGSSYLAWYQQKPGQAPRLLIYGASSR | |
| | APGIPDRFSGSGSGTDFTLTISRLEPEDFAVYYCQQYAD | |
| | SPITFGQGTRLEIKESKYGPPCPPCPMFWVLVVVGGVLA | |
| | CYSLLVTVAFIIFWVRSKRSRGGHSDYMNMTPRRPGPT | |
| | RKHYQPYAPPRDFAAYRSGGGRVKFSRSADAPAYQQG | |
| | QNQLYNELNLGRREEYDVLDKRRGRDPEMGGKPRRK | |
| | NPQEGLYNELQKDKMAEAYSEIGMKGERRRGKGHDG | |
| | LYQGLSTATKDTYDALHMQALPPR | |
| IL13Rα2- | METDTLLLWVLLLWVPGSTGGPVPPSTALRYLIEELVNI | 169 |
| EGFRvIII.BBz + TGF- | TQNQKAPLCNGSMVWSINLTAGMYCAALESLINVSGC | |
| β.28z (MKleader- | SAIEKTQRMLSGFCPHKVSAGQFSSLHVRDTKIEVAQF | |
| IL13op-(G4S)x4- | VKDLLLHLKKLFREGRENGGGGSGGGGSGGGGSGGGG | |
| EGFRvIII scFv-IgG4 | SEIQLVQSGAEVKKPGESLRISCKGSGFNIEDYYIHWVR | |
| hinge-CD28 | QMPGKGLEWMGRIDPENDETKYGPIFQGHVTISADTSI | |
| transmembrane | NTVYLQWSSLKASDTAMYYCAFRGGVYWGQGTTVTV | |
| domain-4-1BB co- | SSGSTSGSGKPGSGEGSTKGDVVMTQSPDSLAVSLGER | |
| stimulatory-CD3 Zeta- | ATINCKSSQSLLDSDGKTYLNWLQQKPGQPPKRLISLVS | |
| T2A-MKleader-HA- | KLDSGVPDRFSGSGSGTDFTLTISSLQAEDVAVYYCWQ | |
| TGF-β scFv-IgG4 | GTHFPGTFGGGTKVEIKESKYGPPCPPCPMFWVLVVVG | |
| hinge peptide spacer- | GVLACYSLLVTVAFIIFWVKRGRKKLLYIFKQPFMRPV | |
| CD28 transmembrane | QTTQEEDGCSCRFPEEEEGGCELRVKFSRSADAPAYQQ | |
| domain-CD28cyto | GQNQLYNELNLGRREEYDVLDKRRGRDPEMGGKPRR | |
| (with gg mutations)- | KNPQEGLYNELQKDKMAEAYSEIGMKGERRRGKGHD | |
| CD3 Zeta) | GLYQGLSTATKDTYDALHMQALPPRLEGGGEGRGSLL | |
| | TCGDVEENPGPRMETDTLLLWVLLLWVPGSTGTSYPY | |
| | DVPDYAGGSQVQLVQSGAEVKKPGSSVKVSCKASGYT | |
| | FSSNVISWVRQAPGQGLEWMGGVIPIVDIANYAQRFKG | |
| | RVTITADESTSTTYMELSSLRSEDTAVYYCALPRAFVLD | |
| | AMDYWGQGTLVTVSSGGGGSGGGGSGGGGSETVLTQ | |
| | SPGTLSLSPGERATLSCRASQSLGSSYLAWYQQKPGQA | |
| | PRLLIYGASSRAPGIPDRFSGSGSGTDFTLTISRLEPEDFA | |
| | VYYCQQYADSPITFGQGTRLEIKESKYGPPCPPCPMFW | |
| | VLVVVGGVLACYSLLVTVAFIIFWVRSKRSRGGHSDY | |
| | MNMTPRRPGPTRKHYQPYAPPRDFAAYRSGGGRVKFS | |
| | RSADAPAYQQGQNQLYNELNLGRREEYDVLDKRRGR | |
| | DPEMGGKPRRKNPQEGLYNELQKDKMAEAYSEIGMK | |
| | GERRRGKGHDGLYQGLSTATKDTYDALHMQALPPR | |
| IL13Rα2-GD2.BBz + | METDTLLLWVLLLWVPGSTGGPVPPSTALRYLIEELVNI | 170 |
| TGF-β.28z (MKleader- | TQNQKAPLCNGSMVWSINLTAGMYCAALESLINVSGC | |
| IL13op-(G4S)x4-GD2 | SAIEKTQRMLSGFCPHKVSAGQFSSLHVRDTKIEVAQF | |
| scFv-IgG4 hinge: IgG4 | VKDLLLHLKKLFREGRENGGGGSGGGGSGGGGSGGGG | |
| CH2 CH3 L235E | SEVQLLQSGPELEKPGASVMISCKASGSSFTGYNMNWV | |
| N297Q peptide spacer- | RQNIGKSLEWIGAIDPYYGGTSYNQKFKGRATLTVDKS | |
| CD28 transmembrane | SSTAYMHLKSLTSEDSAVYYCVSGMEYWGQGTSVTVS | |
| domain-4-1BB co- | SGSTSGSGKPGSGEGSTKGDVVMTQTPLSLPVSLGDQA | |
| stimulatory-CD3 Zeta- | SISCRSSQSLVHRNGNTYLHWYLQKPGQSPKLLIHKVS | |
| T2A-MKleader-HA- | NRFSGVPDRFSGSGSGTDFTLKISRVEAEDLGVYFCSQS | |
| TGF-β scFv-IgG4 | THVPPLTFGAGTKLELKRAESKYGPPCPPCPAPEFEGGP | |
| hinge peptide spacer- | SVFLFPPKPKDTLMISRTPEVTCVVVDVSQEDPEVQFN | |
| CD28 transmembrane | WYVDGVEVHNAKTKPREEQFQSTYRVVSVLTVLHQD | |
| domain-CD28cyto | WLNGKEYKCKVSNKGLPSSIEKTISKAKGQPREPQVYT | |
| (with gg mutations)- | LPPSQEEMTKNQVSLTCLVKGFYPSDIAVEWESNGQPE | |
| CD3 Zeta) | NNYKTTPPVLDSDGSFFLYSRLTVDKSRWQEGNVFSCS | |
| | VMHEALHNHYTQKSLSLSLGKMFWVLVVVGGVLACY | |
| | SLLVTVAFIIFWVKRGRKKLLYIFKQPFMRPVQTTQEED | |
| | GCSCRFPEEEEGGCELRVKFSRSADAPAYQQGQNQLYN | |
| | ELNLGRREEYDVLDKRRGRDPEMGGKPRRKNPQEGLY | |
| | NELQKDKMAEAYSEIGMKGERRRGKGHDGLYQGLST | |
| | ATKDTYDALHMQALPPRLEGGGEGRGSLLTCGDVEEN | |
| | PGPRMETDTLLLWVLLLWVPGSTGTSYPYDVPDYAGG | |
| | SQVQLVQSGAEVKKPGSSVKVSCKASGYTFSSNVISWV | |
| | RQAPGQGLEWMGGVIPIVDIANYAQRFKGRVTITADES | |
| | TSTTYMELSSLRSEDTAVYYCALPRAFVLDAMDYWGQ | |
| | GTLVTVSSGGGGSGGGGSGGGGSETVLTQSPGTLSLSP | |
| | GERATLSCRASQSLGSSYLAWYQQKPGQAPRLLIYGAS | |
| | SRAPGIPDRFSGSGSGTDFTLTISRLEPEDFAVYYCQQY | |
| | ADSPITFGQGTRLEIKESKYGPPCPPCPMFWVLVVVGG | |
| | VLACYSLLVTVAFIIFWVRSKRSRGGHSDYMNMTPRRP | |
| | GPTRKHYQPYAPPRDFAAYRSGGGRVKFSRSADAPAY | |
| | QQGQNQLYNELNLGRREEYDVLDKRRGRDPEMGGKP | |
| | RRKNPQEGLYNELQKDKMAEAYSEIGMKGERRRGKG | |
| | HDGLYQGLSTATKDTYDALHMQALPPR | |
| IL13Rα2-GD2-TGF- | METDTLLLWVLLLWVPGSTGGPVPPSTALRYLIEELVNI | 171 |
| β.BBz (MKleader- | TQNQKAPLCNGSMVWSINLTAGMYCAALESLINVSGC | |
| IL13op-(G4S)x4-GD2 | SAIEKTQRMLSGFCPHKVSAGQFSSLHVRDTKIEVAQF | |
| scFv-(G4S)x4-TGF-β | VKDLLLHLKKLFREGRENGGGGSGGGGSGGGGSGGGG | |
| scFv-IgG4 hinge | SEVQLLQSGPELEKPGASVMISCKASGSSFTGYNMNWV | |
| peptide spacer-CD28 | RQNIGKSLEWIGAIDPYYGGTSYNQKFKGRATLTVDKS | |
| transmembrane | SSTAYMHLKSLTSEDSAVYYCVSGMEYWGQGTSVTVS | |
| domain-4-1BB co- | SGSTSGSGKPGSGEGSTKGDVVMTQTPLSLPVSLGDQA | |
| stimulatory-CD3 Zeta) | SISCRSSQSLVHRNGNTYLHWYLQKPGQSPKLLIHKVS | |
| | NRFSGVPDRFSGSGSGTDFTLKISRVEAEDLGVYFCSQS | |
| | THVPPLTFGAGTKLELKRAGGGGSGGGGSGGGGSGGG | |
| | GSQVQLVQSGAEVKKPGSSVKVSCKASGYTFSSNVISW | |
| | VRQAPGQGLEWMGGVIPIVDIANYAQRFKGRVTITADE | |
| | STSTTYMELSSLRSEDTAVYYCALPRAFVLDAMDYWG | |
| | QGTLVTVSSGGGGSGGGGSGGGGSETVLTQSPGTLSLS | |
| | PGERATLSCRASQSLGSSYLAWYQQKPGQAPRLLIYGA | |
| | SSRAPGIPDRFSGSGSGTDFTLTISRLEPEDFAVYYCQQY | |
| | ADSPITFGQGTRLEIKESKYGPPCPPCPMFWVLVVVGG | |
| | VLACYSLLVTVAFIIFWVKRGRKKLLYIFKQPFMRPVQ | |
| | TTQEEDGCSCRFPEEEEGGCELRVKFSRSADAPAYQQG | |
| | QNQLYNELNLGRREEYDVLDKRRGRDPEMGGKPRRK | |
| | NPQEGLYNELQKDKMAEAYSEIGMKGERRRGKGHDG | |
| | LYQGLSTATKDTYDALHMQALPPR | |
| IL13Rα2-EGFRvIII- | METDTLLLWVLLLWVPGSTGGPVPPSTALRYLIEELVNI | 172 |
| TGF-β.BBz | TQNQKAPLCNGSMVWSINLTAGMYCAALESLINVSGC | |
| (MKleader-IL13op- | SAIEKTQRMLSGFCPHKVSAGQFSSLHVRDTKIEVAQF | |
| (G4S)x4-EGFRvIII | VKDLLLHLKKLFREGRFNGGGGSGGGGSGGGGSGGGG | |
| scFv-(G4S)x4-TGF-β | SEIQLVQSGAEVKKPGESLRISCKGSGFNIEDYYIHWVR | |
| scFv-IgG4 hinge | QMPGKGLEWMGRIDPENDETKYGPIFQGHVTISADTSI | |
| peptide spacer-CD28 | NTVYLQWSSLKASDTAMYYCAFRGGVYWGQGTTVTV | |
| transmembrane | SSGSTSGSGKPGSGEGSTKGDVVMTQSPDSLAVSLGER | |
| domain-4-1BB co- | ATINCKSSQSLLDSDGKTYLNWLQQKPGQPPKRLISLVS | |
| stimulatory-CD3 Zeta) | KLDSGVPDRFSGSGSGTDFTLTISSLQAEDVAVYYCWQ | |
| | GTHFPGTFGGGTKVEIKGGGGSGGGGSGGGGSGGGGS | |
| | QVQLVQSGAEVKKPGSSVKVSCKASGYTFSSNVISWVR | |
| | QAPGQGLEWMGGVIPIVDIANYAQRFKGRVTITADEST | |
| | STTYMELSSLRSEDTAVYYCALPRAFVLDAMDYWGQG | |
| | TLVTVSSGGGGSGGGGSGGGGSETVLTQSPGTLSLSPG | |
| | ERATLSCRASQSLGSSYLAWYQQKPGQAPRLLIYGASS | |
| | RAPGIPDRFSGSGSGTDFTLTISRLEPEDFAVYYCQQYA | |
| | DSPITFGQGTRLEIKESKYGPPCPPCPMFWVLVVVGGVL | |
| | ACYSLLVTVAFIIFWVKRGRKKLLYIFKQPFMRPVQTT | |
| | QEEDGCSCRFPEEEEGGCELRVKFSRSADAPAYQQGQN | |
| | QLYNELNLGRREEYDVLDKRRGRDPEMGGKPRRKNPQ | |
| | EGLYNELQKDKMAEAYSEIGMKGERRRGKGHDGLYQ | |
| | GLSTATKDTYDALHMQALPPR | |

### X. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. The Examples should not be construed as limiting in any way. The contents of all cited references (including literature references, issued patents, published patent applications, and GenBank Accession numbers as cited throughout this application) are hereby expressly incorporated by reference. When definitions of terms in documents that are incorporated by reference herein conflict with those used herein, the definitions used herein govern.

### Example 1: Treatment of Glioblastoma Multiforme with Multispecific Chimeric Antigen Receptors

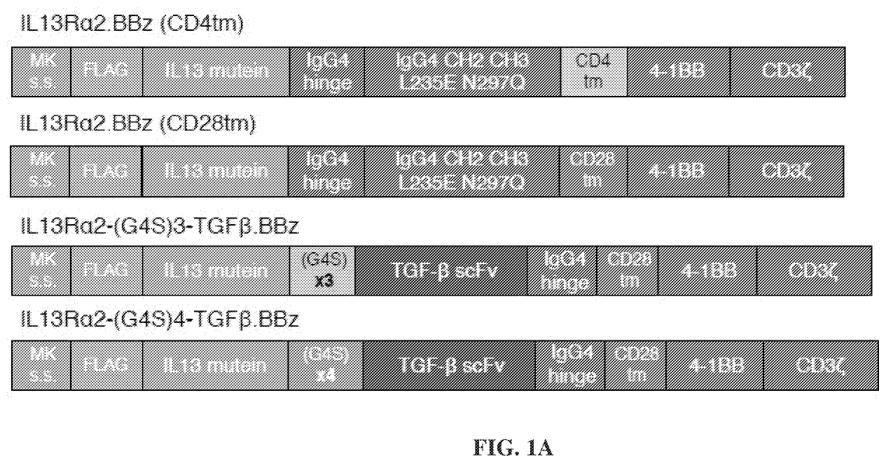

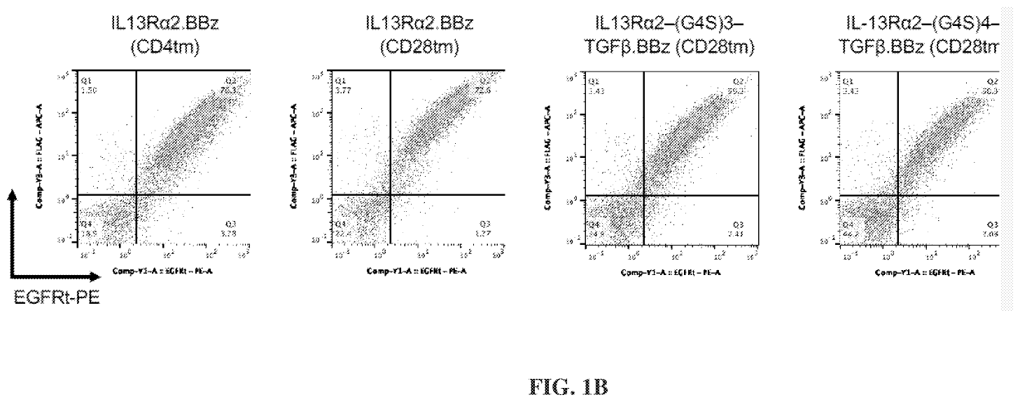

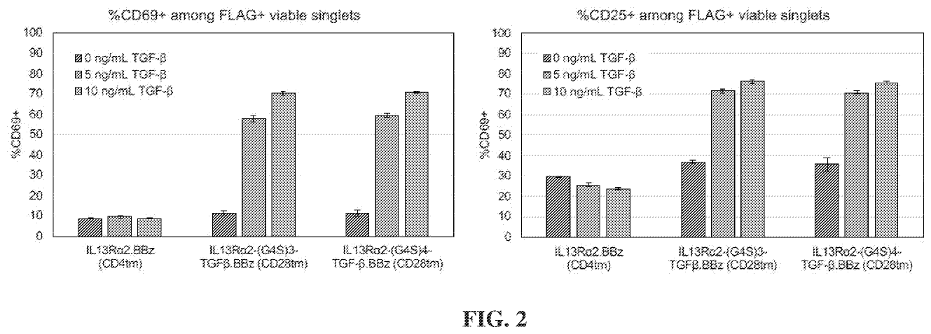

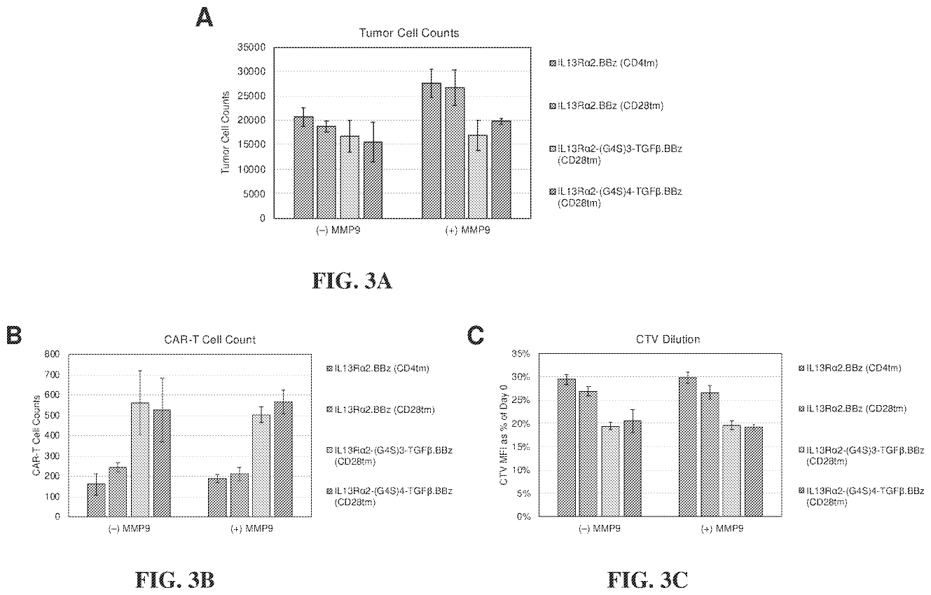

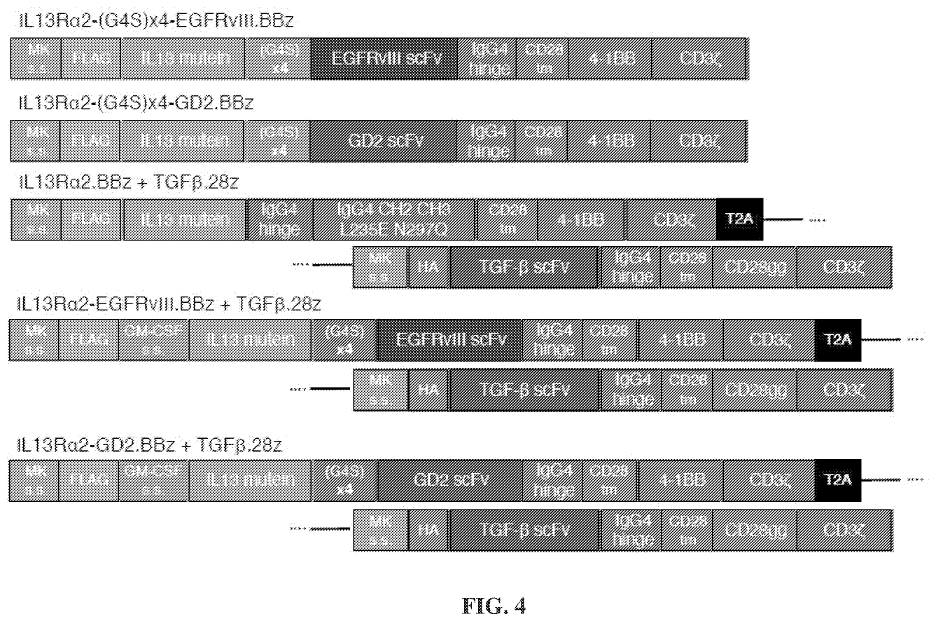

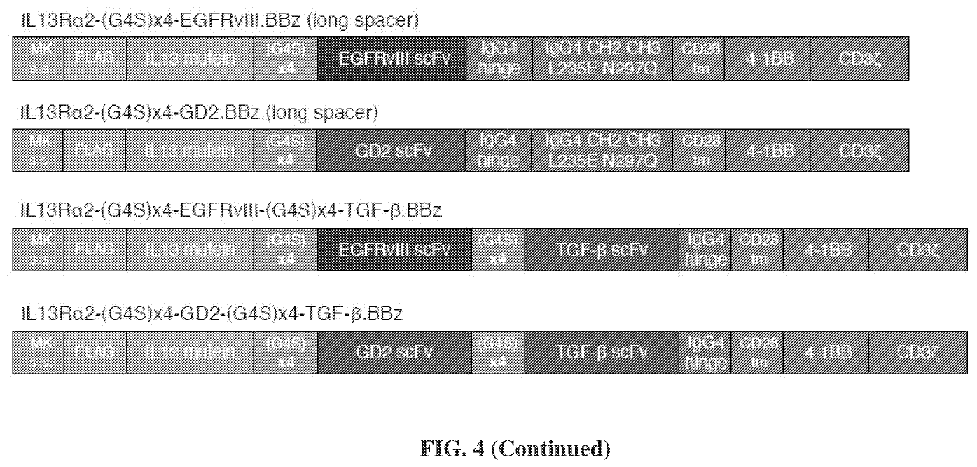

Glioblastoma multiforme (GBM) is a highly aggressive disease with poor prognosis, and targeted immunotherapy for GBM is particularly challenging due to a highly immunosuppressive tumor microenvironment characterized by high transforming growth factor beta (TGF-β) levels. Single-chain bispecific CARs that simultaneously target IL-13Rα2, a clinically relevant GBM antigen, and TGF-β were constructed by connecting the IL-13 mutein with a TGF-β-specific scFv via a peptide linker, and fusing the dual-targeting ligand-binding domain to IgG4 hinge followed by CD28 transmembrane domain, 4-1BB co-stimulatory domain, and CD32 signaling domain. The peptide linkers evaluated include 3 or 4 repeats of Gly-Gly-Gly-Gly-Ser (i.e., (G4S) 3 or (G4S) 4). The bispecific CARs were compared against the single-input IL-13Rα2 CAR. Since the bispecific CAR contains the CD28 transmembrane domain (CD28tm) whereas the original IL-13Rα2 CAR that had been evaluated in the clinic contained the CD4 transmembrane domain (CD28tm; Brown et al., NEJM, 2016, 375 (26): 2561-2569), single-input IL-13Rα2 CARs containing either CD4tm or CD28tm were evaluated (FIG. 1A). Both single-input and bispecific CARs were efficiently expressed on the surface of T cells as reflected by surface antibody staining of a FLAG tag that is fused to the N terminus of each CAR, together with truncated EGFR (EGFRt), which is fused to the C terminus of each CAR via a self-cleaving T2A peptide. CAR-T cells were stimulated with 5 ng/ml or 10 ng/ml of exogenous TGF-β, and antibody staining for the activation markers CD69 and CD25 confirm the bispecific CARs, but not the single-input CARs, respond to TGF-β by triggering T-cell activation (FIG. 2). Furthermore, CAR-T cells were labeled with CellTrace Violet (CTV) dye and then co-incubated with patient-derived PBT106 GBM neurosphere cells at a 1:8 effector-to-target ratio for 94 hours, in the presence or absence of metalloprotease 9 (MMP-9). MMP-9 is known to activate TGF-β by releasing the mature form of TGF-β through proteolytic processing. The number of surviving tumor cells, number of FLAG+ CAR-T cells, as well as CTV dye intensity among FLAG+ CAR-T cells were quantified by flow cytometry. Results indicate that the bispecific CAR-T cells exhibit superior cytotoxicity compared to single-input IL-13Rα2 CAR-T cells in the presence of MMP-9 (FIG. 3A). Furthermore, bispecific CAR-T cells show superior antigen-stimulated T-cell proliferation both in the presence and in the absence of MMP-9 compared to single-input IL-13Rα2 CAR-T cells (FIG. 3B).

### Example 2: Treatment of Glioblastoma Multiforme with Multispecific Chimeric Antigen Receptors

Glioblastoma multiforme (GBM) is the most common type of primary brain tumors in adults, and the median survival period has remained at 12-16 months from the time of diagnosis over the past few decades. Conventional therapies such as surgery, chemotherapy, and radiation almost invariably fail to eradicate tumor, resulting in relapse within weeks or months. Consequently, GBM has been an active area of research for new treatment options such as adoptive T-cell therapy. Two major challenges have limited the efficacy of T-cell therapy for GBM thus far. First, the GBM tumor microenvironment is strongly immunosuppressive, characterized by a high level of transforming growth factor beta (TGF-β), which simultaneously promote tumor growth and potently suppress the function of T cells. Second, GBM tumors are highly heterogeneous in antigen expression, thus T cells engineered to target a single antigen are generally unable to recognize and eradicate all tumor cells present.

The inventors propose to overcome the two main challenges of adoptive T-cell therapy against GBM through the use of bispecific chimeric antigen receptor (CAR)-T cells that can simultaneously target a GBM-associated surface antigen and convert TGF-β from an immunosuppressive cytokine into a potent stimulant for the engineered T cells. Importantly, the TGF-β CAR can both inhibit endogenous TGF-β signaling (by competing against endogenous TGF-β receptors for binding to TGF-β ligands) and trigger T-cell activation in the presence of both soluble and immobilized TGF-β. The concept is that the TGF-β conversion function of the CAR-T cells could modify the tumor microenvironment, thus promoting the anti-tumor function of both the engineered T cells and endogenous immune cells.

The inventors have built a series of bispecific CARs that simultaneously respond to TGF-β plus IL-13Rα2, and antigen found on the surface of brain-tumor cells. Specifically, they have constructed the following bispecific CARs:

| | | SEQ ID |
|---|---|---|
| CAR | SEQUENCE | NO: |
| SP-IL- | METDTLLLWVLLLWVPGSTGAGGSDYKDDDDKGGSSPGPV | 1 |
| 13Rα2.BBz | PPSTALRYLIEELVNITQNQKAPLCNGSMVWSINLTAGMYC | |
| CAR; (Murine | AALESLINVSGCSAIEKTQRMLSGFCPHKVSAGQFSSLHVRD | |
| kappa signal | TKIEVAQFVKDLLLHLKKLFREGRFNESKYGPPCPPCPAPEF | |
| sequence_FLAG_ | EGGPSVFLFPPKPKDTLMISRTPEVTCVVVDVSQEDPEVQFN | |
| SP IL13 | WYVDGVEVHNAKTKPREEQFQSTYRVVSVLTVLHQDWLN | |
| mutein_IgG4 | GKEYKCKVSNKGLPSSIEKTISKAKGQPREPQVYTLPPSQEE | |
| (L235E, | MTKNQVSLTCLVKGFYPSDIAVEWESNGQPENNYKTTPPVL | |
| N297Q)_CD28tm_ | DSDGSFFLYSRLTVDKSRWQEGNVFSCSVMHEALHNHYTQ | |
| 4- | KSLSLSLGKMFWVLVVVGGVLACYSLLVTVAFIIFWVKRGR | |
| 1BB_CD3zeta) | KKLLYIFKQPFMRPVQTTQEEDGCSCRFPEEEEGGCELRVKF | |
| | SRSADAPAYQQGQNQLYNELNLGRREEYDVLDKRRGRDPE | |
| | MGGKPRRKNPQEGLYNELQKDKMAEAYSEIGMKGERRRG | |
| | KGHDGLYQGLSTATKDTYDALHMQALPPR | |
| SP-IL- | METDTLLLWVLLLWVPGSTGAGGSDYKDDDDKGGSSPGPV | 9 |
| 13Rα2/TGF- | PPSTALRYLIEELVNITQNQKAPLCNGSMVWSINLTAGMYC | |
| β.BBz CAR; | AALESLINVSGCSAIEKTQRMLSGFCPHKVSAGQFSSLHVRD | |
| (Murine kappa | TKIEVAQFVKDLLLHLKKLFREGRENGGGGSGGGGSGGGGS | |
| signal | GGGGSQVQLVQSGAEVKKPGSSVKVSCKASGYTFSSNVISW | |
| sequence_FLAG_ | VRQAPGQGLEWMGGVIPIVDIANYAQRFKGRVTITADESTS | |
| SP IL13 | TTYMELSSLRSEDTAVYYCALPRAFVLDAMDYWGQGTLVT | |
| mutein_(G4S)x4 | VSSGGGGSGGGGSGGGGSETVLTQSPGTLSLSPGERATLSCR | |
| TGF-β | ASQSLGSSYLAWYQQKPGQAPRLLIYGASSRAPGIPDRESGS | |
| scFv_IgG4 | GSGTDFTLTISRLEPEDFAVYYCQQYADSPITFGQGTRLEIKE | |
| hinge_CD28tm_4- | SKYGPPCPPCPMFWVLVVVGGVLACYSLLVTVAFIIFWVKR | |
| 1BB_CD3zeta) | GRKKLLYIFKQPFMRPVQTTQEEDGCSCRFPEEEEGGCELRV | |
| | KFSRSADAPAYQQGQNQLYNELNLGRREEYDVLDKRRGRD | |
| | PEMGGKPRRKNPQEGLYNELQKDKMAEAYSEIGMKGERRR | |
| | GKGHDGLYQGLSTATKDTYDALHMQALPPR | |
| SP-IL- | METDTLLLWVLLLWVPGSTGAGGSDYKDDDDKGGSSPGPV | 13 |
| 13Rα2.BBz (KR) | PPSTALRYLIEELVNITQNQKAPLCNGSMVWSINLTAGMYC | |
| CAR; (Murine | AALESLINVSGCSAIEKTQRMLSGFCPHKVSAGQFSSLHVRD | |
| kappa signal | TKIEVAQFVKDLLLHLKKLFREGRFNESKYGPPCPPCPAPEF | |
| sequence_FLAG_ | EGGPSVFLFPPKPKDTLMISRTPEVTCVVVDVSQEDPEVQFN | |
| SP IL13 | WYVDGVEVHNAKTKPREEQFQSTYRVVSVLTVLHQDWLN | |
| mutein_IgG4 | GKEYKCKVSNKGLPSSIEKTISKAKGQPREPQVYTLPPSQEE | |
| (L235E, | MTKNQVSLTCLVKGFYPSDIAVEWESNGQPENNYKTTPPVL | |
| N297Q)_CD28tm_ | DSDGSFFLYSRLTVDKSRWQEGNVFSCSVMHEALHNHYTQ | |
| 4- | KSLSLSLGKMFWVLVVVGGVLACYSLLVTVAFIIFWVRRGR | |
| 1BB(KR)_CD3zeta | RRLLYIFRQPFMRPVQTTQEEDGCSCRFPEEEEGGCELRVRF | |
| (KR)) | SRSADAPAYQQGQNQLYNELNLGRREEYDVLDRRRGRDPE | |
| | MGGRPRRRNPQEGLYNELQRDRMAEAYSEIGMRGERRRGR | |
| | GHDGLYQGLSTATRDTYDALHMQALPPR | |
| SP-IL- | METDTLLLWVLLLWVPGSTGAGGSDYKDDDDKGGSSPGPV | 16 |
| 13Rα2/TGF- | PPSTALRYLIEELVNITQNQKAPLCNGSMVWSINLTAGMYC | |
| β.BBz (KR) | AALESLINVSGCSAIEKTQRMLSGFCPHKVSAGQFSSLHVRD | |
| CAR; (Murine | TKIEVAQFVKDLLLHLKKLFREGRENGGGGSGGGGSGGGGS | |
| kappa signal | GGGGSQVQLVQSGAEVKKPGSSVKVSCKASGYTFSSNVISW | |
| sequence_FLAG_ | VRQAPGQGLEWMGGVIPIVDIANYAQRFKGRVTITADESTS | |
| SP IL13 | TTYMELSSLRSEDTAVYYCALPRAFVLDAMDYWGQGTLVT | |
| mutein_(G4S)x4_ | VSSGGGGSGGGGSGGGGSETVLTQSPGTLSLSPGERATLSCR | |
| TGF-β | ASQSLGSSYLAWYQQKPGQAPRLLIYGASSRAPGIPDRESGS | |
| scFv_IgG4 | GSGTDFTLTISRLEPEDFAVYYCQQYADSPITFGQGTRLEIKE | |
| hinge_CD28tm_4- | SKYGPPCPPCPMFWVLVVVGGVLACYSLLVTVAFIIFWVRR | |
| 1BB(KR)_ | GRRRLLYIFRQPFMRPVQTTQEEDGCSCRFPEEEEGGCELRV | |
| CD3zeta(KR)) | RFSRSADAPAYQQGQNQLYNELNLGRREEYDVLDRRRGRD | |
| | PEMGGRPRRRNPQEGLYNELQRDRMAEAYSEIGMRGERRR | |
| | GRGHDGLYQGLSTATRDTYDALHMQALPPR | |
| SP-IL-13Rα2.28z | METDTLLLWVLLLWVPGSTGAGGSDYKDDDDKGGSSPGPV | 17 |
| CAR; (Murine | PPSTALRYLIEELVNITQNQKAPLCNGSMVWSINLTAGMYC | |
| kappa signal | AALESLINVSGCSAIEKTQRMLSGFCPHKVSAGQFSSLHVRD | |
| sequence_FLAG_ | TKIEVAQFVKDLLLHLKKLFREGRFNESKYGPPCPPCPAPEF | |
| SP IL13 | EGGPSVFLFPPKPKDTLMISRTPEVTCVVVDVSQEDPEVQFN | |
| mutein_IgG4 | WYVDGVEVHNAKTKPREEQFQSTYRVVSVLTVLHQDWLN | |
| (L235E, | GKEYKCKVSNKGLPSSIEKTISKAKGQPREPQVYTLPPSQEE | |
| N297Q)_CD28tm_ | MTKNQVSLTCLVKGFYPSDIAVEWESNGQPENNYKTTPPVL | |
| CD28cyto_ | DSDGSFFLYSRLTVDKSRWQEGNVFSCSVMHEALHNHYTQ | |
| CD3zeta) | KSLSLSLGKMFWVLVVVGGVLACYSLLVTVAFIIFWVRSKR | |
| | SRGGHSDYMNMTPRRPGPTRKHYQPYAPPRDFAAYRSGGG | |
| | RVKFSRSADAPAYQQGQNQLYNELNLGRREEYDVLDKRRG | |
| | RDPEMGGKPRRKNPQEGLYNELQKDKMAEAYSEIGMKGER | |
| | RRGKGHDGLYQGLSTATKDTYDALHMQALPPR | |
| SP-IL- | METDTLLLWVLLLWVPGSTGAGGSDYKDDDDKGGSSPGPV | 25 |
| 13Rα2/TGF- | PPSTALRYLIEELVNITQNQKAPLCNGSMVWSINLTAGMYC | |
| β.28z CAR; | AALESLINVSGCSAIEKTQRMLSGFCPHKVSAGQFSSLHVRD | |
| (Murine kappa | TKIEVAQFVKDLLLHLKKLFREGRENGGGGSGGGGSGGGGS | |
| signal | GGGGSQVQLVQSGAEVKKPGSSVKVSCKASGYTFSSNVISW | |
| sequence_FLAG_ | VRQAPGQGLEWMGGVIPIVDIANYAQRFKGRVTITADESTS | |
| SP IL13 | TTYMELSSLRSEDTAVYYCALPRAFVLDAMDYWGQGTLVT | |
| mutein_(G4S)x4_ | VSSGGGGSGGGGSGGGGSETVLTQSPGTLSLSPGERATLSCR | |
| TGF-β | ASQSLGSSYLAWYQQKPGQAPRLLIYGASSRAPGIPDRESGS | |
| scFv_IgG4 | GSGTDFTLTISRLEPEDFAVYYCQQYADSPITFGQGTRLEIKE | |
| hinge_CD28tm_ | SKYGPPCPPCPMFWVLVVVGGVLACYSLLVTVAFIIFWVRS | |
| CD28cyto_CD3z | KRSRGGHSDYMNMTPRRPGPTRKHYQPYAPPRDFAAYRSG | |
| eta) | GGRVKFSRSADAPAYQQGQNQLYNELNLGRREEYDVLDKR | |
| | RGRDPEMGGKPRRKNPQEGLYNELQKDKMAEAYSEIGMK | |
| | GERRRGKGHDGLYQGLSTATKDTYDALHMQALPPR | |
| Murine kappa | METDTLLLWVLLLWVPGSTGAGGSDYKDDDDKGGSSPGPV | 131 |
| signal | PPSTALRYLIEELVNITQNQKAPLCNGSMVWSINLTAGMYC | |
| sequence_FLAG_ | AALESLINVSGCSAIEKTQRMLSGFCPHKVSAGQFSSLHVRD | |
| SP IL13 | TKIEVAQFVKDLLLHLKKLFREGRFNESKYGPPCPPCPAPEF | |
| mutein_IgG4 | EGGPSVFLFPPKPKDTLMISRTPEVTCVVVDVSQEDPEVQFN | |
| (L235E,N297Q)_ | WYVDGVEVHNAKTKPREEQFQSTYRVVSVLTVLHQDWLN | |
| CD28tm_4- | GKEYKCKVSNKGLPSSIEKTISKAKGQPREPQVYTLPPSQEE | |
| 1BB_CD3zeta_ | MTKNQVSLTCLVKGFYPSDIAVEWESNGQPENNYKTTPPVL | |
| T2A_HA_ | DSDGSFFLYSRLTVDKSRWQEGNVFSCSVMHEALHNHYTQ | |
| TGFBR2tr(DNR) | KSLSLSLGKMFWVLVVVGGVLACYSLLVTVAFIIFWVKRGR | |
| | KKLLYIFKQPFMRPVQTTQEEDGCSCRFPEEEEGGCELRVKF | |
| | SRSADAPAYQQGQNQLYNELNLGRREEYDVLDKRRGRDPE | |
| | MGGKPRRKNPQEGLYNELQKDKMAEAYSEIGMKGERRRG | |
| | KGHDGLYQGLSTATKDTYDALHMQALPPRLEGGGEGRGSL | |
| | LTCGDVEENPGPRMGRGLLRGLWPLHIVLWTRIASTIPPYPY | |
| | DVPDYAHVQKSVNNDMIVTDNNGAVKFPQLCKFCDVRFST | |
| | CDNQKSCMSNCSITSICEKPQEVCVAVWRKNDENITLETVC | |
| | HDPKLPYHDFILEDAASPKCIMKEKKKPGETFFMCSCSSDEC | |
| | NDNIIFSEEYNTSNPDLLLVIFQVTGISLLPPLGVAISVIIIFYC | |
| | YRVNRQQKLSS | |
| Full IL- | METDTLLLWVLLLWVPGSTGAGGSDYKDDDDKGGSLTCL | 19 |
| 13Rα2.BBz | GGFASPGPVPPSTALRYLIEELVNITQNQKAPLCNGSMVWSI | |
| CAR; (Murine | NLTAGMYCAALESLINVSGCSAIEKTQRMLSGFCPHKVSAG | |
| kappa signal | QFSSLHVRDTKIEVAQFVKDLLLHLKKLFREGRFNESKYGPP | |
| sequence_FLAG_ | CPPCPAPEFEGGPSVFLFPPKPKDTLMISRTPEVTCVVVDVSQ | |
| Full IL13 | EDPEVQFNWYVDGVEVHNAKTKPREEQFQSTYRVVSVLTV | |
| mutein_IgG4 | LHQDWLNGKEYKCKVSNKGLPSSIEKTISKAKGQPREPQVY | |
| (L235E, | TLPPSQEEMTKNQVSLTCLVKGFYPSDIAVEWESNGQPENN | |
| N297Q)_CD28tm_ | YKTTPPVLDSDGSFFLYSRLTVDKSRWQEGNVFSCSVMHEA | |
| 4- | LHNHYTQKSLSLSLGKMFWVLVVVGGVLACYSLLVTVAFII | |
| 1BB_CD3zeta) | FWVKRGRKKLLYIFKQPFMRPVQTTQEEDGCSCRFPEEEEG | |
| | GCELRVKFSRSADAPAYQQGQNQLYNELNLGRREEYDVLD | |
| | KRRGRDPEMGGKPRRKNPQEGLYNELQKDKMAEAYSEIG | |
| | MKGERRRGKGHDGLYQGLSTATKDTYDALHMQALPPR | |
| Full-IL- | METDTLLLWVLLLWVPGSTGAGGSDYKDDDDKGGSLTCL | 21 |
| 13Rα2/TGF- | GGFASPGPVPPSTALRYLIEELVNITQNQKAPLCNGSMVWSI | |
| β.BBz CAR; | NLTAGMYCAALESLINVSGCSAIEKTQRMLSGFCPHKVSAG | |
| (Murine kappa | QFSSLHVRDTKIEVAQFVKDLLLHLKKLFREGRENGGGGSG | |
| signal | GGGSGGGGSGGGGSQVQLVQSGAEVKKPGSSVKVSCKASG | |
| sequence_FLAG_ | YTFSSNVISWVRQAPGQGLEWMGGVIPIVDIANYAQRFKGR | |
| Full IL13 | VTITADESTSTTYMELSSLRSEDTAVYYCALPRAFVLDAMD | |
| mutein_(G4S)x4_ | YWGQGTLVTVSSGGGGSGGGGSGGGGSETVLTQSPGTLSLS | |
| TGF-β | PGERATLSCRASQSLGSSYLAWYQQKPGQAPRLLIYGASSR | |
| scFv_IgG4 | APGIPDRFSGSGSGTDFTLTISRLEPEDFAVYYCQQYADSPIT | |
| hinge_CD28tm_ | FGQGTRLEIKESKYGPPCPPCPMFWVLVVVGGVLACYSLLV | |
| 4-1BB_CD3zeta) | TVAFIIFWVKRGRKKLLYIFKQPFMRPVQTTQEEDGCSCRFP | |
| | EEEEGGCELRVKFSRSADAPAYQQGQNQLYNELNLGRREE | |
| | YDVLDKRRGRDPEMGGKPRRKNPQEGLYNELQKDKMAEA | |
| | YSEIGMKGERRRGKGHDGLYQGLSTATKDTYDALHMQALP | |
| | PR | |
| Murine kappa | METDTLLLWVLLLWVPGSTGAGGSDYKDDDDKGGSLTCL | 132 |
| signal | GGFASPGPVPPSTALRYLIEELVNITQNQKAPLCNGSMVWSI | |
| sequence_FLAG_ | NLTAGMYCAALESLINVSGCSAIEKTQRMLSGFCPHKVSAG | |
| Full IL13 | QFSSLHVRDTKIEVAQFVKDLLLHLKKLFREGRFNESKYGPP | |
| mutein_IgG4 | CPPCPAPEFEGGPSVFLFPPKPKDTLMISRTPEVTCVVVDVSQ | |
| (L235E, | EDPEVQFNWYVDGVEVHNAKTKPREEQFQSTYRVVSVLTV | |
| N297Q)_CD28tm_ | LHQDWLNGKEYKCKVSNKGLPSSIEKTISKAKGQPREPQVY | |
| 4-1BB | TLPPSQEEMTKNQVSLTCLVKGFYPSDIAVEWESNGQPENN | |
| (KR)_CD3zeta | YKTTPPVLDSDGSFFLYSRLTVDKSRWQEGNVFSCSVMHEA | |
| (KR) | LHNHYTQKSLSLSLGKMFWVLVVVGGVLACYSLLVTVAFII | |
| | FWVRRGRRRLLYIFRQPFMRPVQTTQEEDGCSCRFPEEEEG | |
| | GCELRVRFSRSADAPAYQQGQNQLYNELNLGRREEYDVLD | |
| | RRRGRDPEMGGRPRRRNPQEGLYNELQRDRMAEAYSEIGM | |
| | RGERRRGRGHDGLYQGLSTATRDTYDALHMQALPPR | |
| Murine kappa | METDTLLLWVLLLWVPGSTGAGGSDYKDDDDKGGSLTCL | 133 |
| signal | GGFASPGPVPPSTALRYLIEELVNITQNQKAPLCNGSMVWSI | |
| sequence_FLAG_ | NLTAGMYCAALESLINVSGCSAIEKTQRMLSGFCPHKVSAG | |
| Full IL13 | QFSSLHVRDTKIEVAQFVKDLLLHLKKLFREGRENGGGGSG | |
| mutein_(G4S)x4_ | GGGSGGGGSGGGGSQVQLVQSGAEVKKPGSSVKVSCKASG | |
| TGF-β | YTFSSNVISWVRQAPGQGLEWMGGVIPIVDIANYAQRFKGR | |
| scFv_IgG4 | VTITADESTSTTYMELSSLRSEDTAVYYCALPRAFVLDAMD | |
| hinge_CD28tm_ | YWGQGTLVTVSSGGGGSGGGGSGGGGSETVLTQSPGTLSLS | |
| 4-1BB | PGERATLSCRASQSLGSSYLAWYQQKPGQAPRLLIYGASSR | |
| (KR)_CD3zeta | APGIPDRFSGSGSGTDFTLTISRLEPEDFAVYYCQQYADSPIT | |
| (KR) | FGQGTRLEIKESKYGPPCPPCPMFWVLVVVGGVLACYSLLV | |
| | TVAFIIFWVRRGRRRLLYIFRQPFMRPVQTTQEEDGCSCRFP | |
| | EEEEGGCELRVRFSRSADAPAYQQGQNQLYNELNLGRREE | |
| | YDVLDRRRGRDPEMGGRPRRRNPQEGLYNELQRDRMAEA | |
| | YSEIGMRGERRRGRGHDGLYQGLSTATRDTYDALHMQALP | |
| | PR | |
| Full-IL- | METDTLLLWVLLLWVPGSTGAGGSDYKDDDDKGGSLTCL | 22 |
| 13Rα2.28z CAR; | GGFASPGPVPPSTALRYLIEELVNITQNQKAPLCNGSMVWSI | |
| (Murine kappa | NLTAGMYCAALESLINVSGCSAIEKTQRMLSGFCPHKVSAG | |
| signal | QFSSLHVRDTKIEVAQFVKDLLLHLKKLFREGRFNESKYGPP | |
| sequence_FLAG_ | CPPCPAPEFEGGPSVFLFPPKPKDTLMISRTPEVTCVVVDVSQ | |
| Full IL13 | EDPEVQFNWYVDGVEVHNAKTKPREEQFQSTYRVVSVLTV | |
| mutein_IgG4 | LHQDWLNGKEYKCKVSNKGLPSSIEKTISKAKGQPREPQVY | |
| (L235E, | TLPPSQEEMTKNQVSLTCLVKGFYPSDIAVEWESNGQPENN | |
| N297Q)_CD28tm_ | YKTTPPVLDSDGSFFLYSRLTVDKSRWQEGNVFSCSVMHEA | |
| CD28cyto_ | LHNHYTQKSLSLSLGKMFWVLVVVGGVLACYSLLVTVAFII | |
| CD3zeta) | FWVRSKRSRGGHSDYMNMTPRRPGPTRKHYQPYAPPRDFA | |
| | AYRSGGGRVKFSRSADAPAYQQGQNQLYNELNLGRREEYD | |
| | VLDKRRGRDPEMGGKPRRKNPQEGLYNELQKDKMAEAYS | |
| | EIGMKGERRRGKGHDGLYQGLSTATKDTYDALHMQALPPR | |
| Full-IL- | METDTLLLWVLLLWVPGSTGAGGSDYKDDDDKGGSLTCL | 23 |
| 13Rα2/TGF- | GGFASPGPVPPSTALRYLIEELVNITQNQKAPLCNGSMVWSI | |
| β.28z CAR; | NLTAGMYCAALESLINVSGCSAIEKTQRMLSGFCPHKVSAG | |
| (Murine kappa | QFSSLHVRDTKIEVAQFVKDLLLHLKKLFREGRENGGGGSG | |
| signal | GGGSGGGGSGGGGSQVQLVQSGAEVKKPGSSVKVSCKASG | |
| sequence_FLAG_ | YTFSSNVISWVRQAPGQGLEWMGGVIPIVDIANYAQRFKGR | |
| Full IL13 | VTITADESTSTTYMELSSLRSEDTAVYYCALPRAFVLDAMD | |
| mutein_(G4S)x4_ | YWGQGTLVTVSSGGGGSGGGGSGGGGSETVLTQSPGTLSLS | |
| TGF-β | PGERATLSCRASQSLGSSYLAWYQQKPGQAPRLLIYGASSR | |
| scFv_IgG4 | APGIPDRFSGSGSGTDFTLTISRLEPEDFAVYYCQQYADSPIT | |
| hinge_CD28tm_ | FGQGTRLEIKESKYGPPCPPCPMFWVLVVVGGVLACYSLLV | |
| CD28cyto_ | TVAFIIFWVRSKRSRGGHSDYMNMTPRRPGPTRKHYQPYAP | |
| CD3zeta) | PRDFAAYRSGGGRVKFSRSADAPAYQQGQNQLYNELNLGR | |
| | REEYDVLDKRRGRDPEMGGKPRRKNPQEGLYNELQKDKM | |
| | AEAYSEIGMKGERRRGKGHDGLYQGLSTATKDTYDALHM | |
| | QALPPR | |
| Murine kappa | METDTLLLWVLLLWVPGSTGAGGSDYKDDDDKGGSLTCL | 134 |
| signal | GGFASPGPVPPSTALRYLIEELVNITQNQKAPLCNGSMVWSI | |
| sequence_FLAG_ | NLTAGMYCAALESLINVSGCSAIEKTQRMLSGFCPHKVSAG | |
| Full IL13 | QFSSLHVRDTKIEVAQFVKDLLLHLKKLFREGRFNESKYGPP | |
| mutein_IgG4 | CPPCPAPEFEGGPSVFLFPPKPKDTLMISRTPEVTCVVVDVSQ | |
| (L235E, N297Q)_ | EDPEVQFNWYVDGVEVHNAKTKPREEQFQSTYRVVSVLTV | |
| CD28tm_4- | LHQDWLNGKEYKCKVSNKGLPSSIEKTISKAKGQPREPQVY | |
| 1BB_CD3zeta_ | TLPPSQEEMTKNQVSLTCLVKGFYPSDIAVEWESNGQPENN | |
| T2A_HA_TGFBR2tr | YKTTPPVLDSDGSFFLYSRLTVDKSRWQEGNVFSCSVMHEA | |
| (DNR) | LHNHYTQKSLSLSLGKMFWVLVVVGGVLACYSLLVTVAFII | |
| | FWVKRGRKKLLYIFKQPFMRPVQTTQEEDGCSCRFPEEEEG | |
| | GCELRVKFSRSADAPAYQQGQNQLYNELNLGRREEYDVLD | |
| | KRRGRDPEMGGKPRRKNPQEGLYNELQKDKMAEAYSEIG | |
| | MKGERRRGKGHDGLYQGLSTATKDTYDALHMQALPPRLE | |
| | GGGEGRGSLLTCGDVEENPGPRMGRGLLRGLWPLHIVLWT | |
| | RIASTIPPYPYDVPDYAHVQKSVNNDMIVTDNNGAVKFPQL | |
| | CKFCDVRFSTCDNQKSCMSNCSITSICEKPQEVCVAVWRKN | |
| | DENITLETVCHDPKLPYHDFILEDAASPKCIMKEKKKPGETF | |
| | FMCSCSSDECNDNIIFSEEYNTSNPDLLLVIFQVTGISLLPPLG | |
| | VAISVIIIFYCYRVNRQQKLSS | |
| Murine kappa | METDTLLLWVLLLWVPGSTGAGGSDYKDDDDKGGSSPGPV | 135 |
| signal | PPSTALRYLIEELVNITQNQKAPLCNGSMVWSINLTAGMYC | |
| sequence_FLAG_ | AALESLINVSGCSAIEKTQRMLSGFCPHKVSAGQFSSLHVRD | |
| SP IL13 | TKIEVAQFVKDLLLHLKKLFREGRFNESKYGPPCPPCPAPEF | |
| mutein_IgG4 | EGGPSVFLFPPKPKDTLMISRTPEVTCVVVDVSQEDPEVQFN | |
| (L235E, N297Q)_ | WYVDGVEVHNAKTKPREEQFQSTYRVVSVLTVLHQDWLN | |
| CD28tm_ | GKEYKCKVSNKGLPSSIEKTISKAKGQPREPQVYTLPPSQEE | |
| CD28cyto_ | MTKNQVSLTCLVKGFYPSDIAVEWESNGQPENNYKTTPPVL | |
| CD3zeta_T2A_ | DSDGSFFLYSRLTVDKSRWQEGNVFSCSVMHEALHNHYTQ | |
| HA_TGFBR2tr | KSLSLSLGKMFWVLVVVGGVLACYSLLVTVAFIIFWVRSKR | |
| (DNR) | SRGGHSDYMNMTPRRPGPTRKHYQPYAPPRDFAAYRSGGG | |
| | RVKFSRSADAPAYQQGQNQLYNELNLGRREEYDVLDKRRG | |
| | RDPEMGGKPRRKNPQEGLYNELQKDKMAEAYSEIGMKGER | |
| | RRGKGHDGLYQGLSTATKDTYDALHMQALPPRLEGGGEGR | |
| | GSLLTCGDVEENPGPRMGRGLLRGLWPLHIVLWTRIASTIPP | |
| | YPYDVPDYAHVQKSVNNDMIVTDNNGAVKFPQLCKFCDV | |
| | RFSTCDNQKSCMSNCSITSICEKPQEVCVAVWRKNDENITLE | |
| | TVCHDPKLPYHDFILEDAASPKCIMKEKKKPGETFFMCSCSS | |
| | DECNDNIIFSEEYNTSNPDLLLVIFQVTGISLLPPLGVAISVIIIF | |
| | YCYRVNRQQKLSS | |

All of the constructs contain, from N terminal to C terminal of the protein, the following components: murine kappa chain signal sequence, binding domains each separated by (Gly4 Ser1)x4, IgG4 hinge, CD28 transmembrane domain, and CD35 cytoplasmic domain. Some CARs contain CD28 cytoplasmic domain between the transmembrane and CD35 domains; others contain 4-1BB cytoplasmic domain instead of CD28 cytoplasmic domain.

Constructs 7, 14, and 15 above co-express the single-input IL-13Rα2 CAR with a dominant-negative TGF-β receptor (DNR), which is TGF-β receptor chain 2 missing its cytoplasmic domain. These constructs are built as controls to compare against the IL-13Rα2/TGF-β bispecific CARs.

T cells were transduced with a panel of single-input IL-13Rα2 or bispecific IL-13Rα2/TGF-β CARs, bearing either an SP dipeptide or LTCLGGFASP (“Full”) polypeptide at the N-terminus of the IL-13 mutein. Each CAR was fused to an N-terminal FLAG tag to enable surface detection by antibody staining. On day 7 of culture, transduced T cells were stained for surface expression of FLAG-tagged CARs. The relative strength of CAR expression for IL-13 muteins with an N-terminal SP versus full N-terminus was construct-dependent. Averages of triplicates are shown, with error bars representing ±1 standard deviation. (FIG. 11).

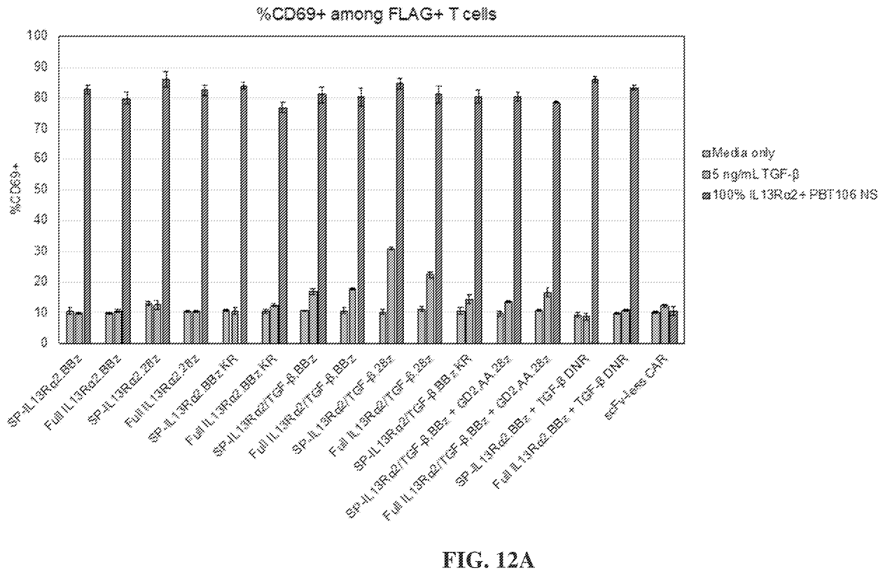

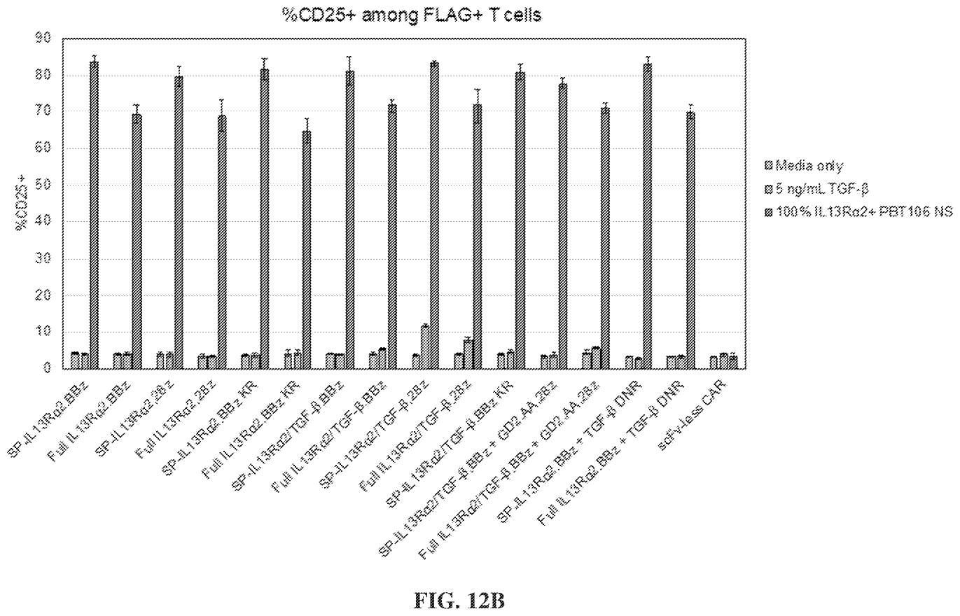

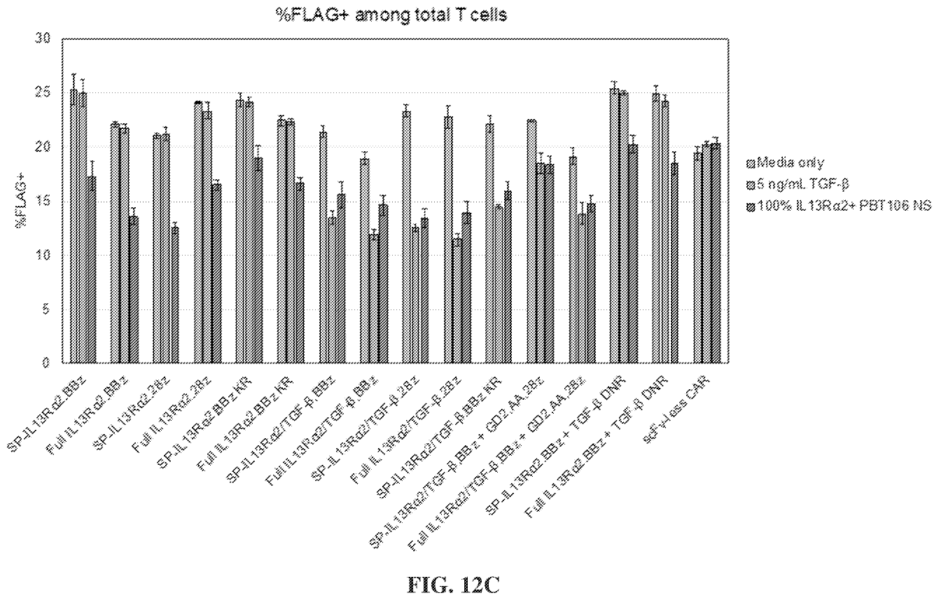

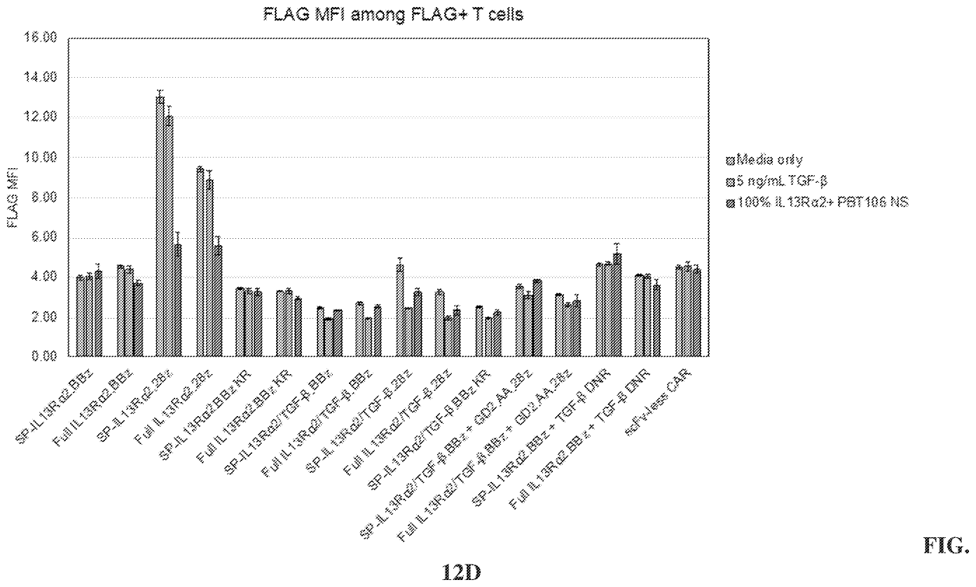

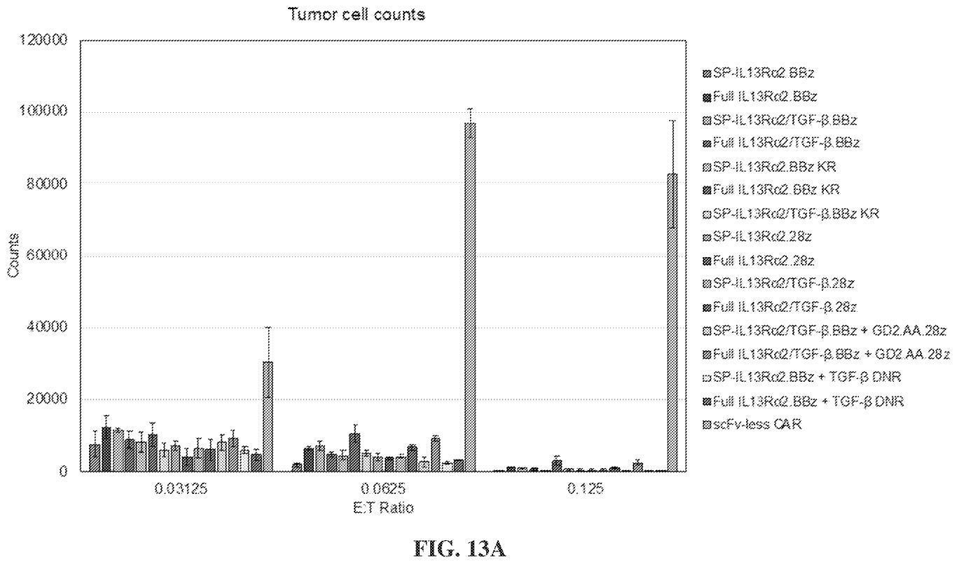

To evaluate whether CARs signal in response to antigen, CAR-T cells were cultured for 21 hours in either media alone, or in the presence of 5 ng/mL recombinant human TGF-β1 or IL-13Rα2+ PBT106 neurospheres, respectively. T cells were subsequently stained for surface expression of CD69 (FIG. 12A), CD25 (FIG. 12B), and FLAG (FIG. 12C,D). Both single-input IL-13Rα2 and bispecific IL-13Rα2/TGF-β CAR-T cells are strongly activated by antigen-expressing PBT106 tumor cells, while only bispecific IL-13Rα2/TGF-β CARs are activated by TGF-β, as evidenced by upregulation of CD69 and CD25, and downregulation of surface FLAG expression (FIG. 12A-D). Moreover, CARs with an N-terminal SP conferred greater CD25 upregulation compared to CARs with the full IL-13 mutein N-terminus, suggesting that the shorter SP N-terminus of the IL-13 mutein confers greater functionality compared to the full N-terminus. Averages of triplicates are shown, with error bars representing ±1 standard deviation.

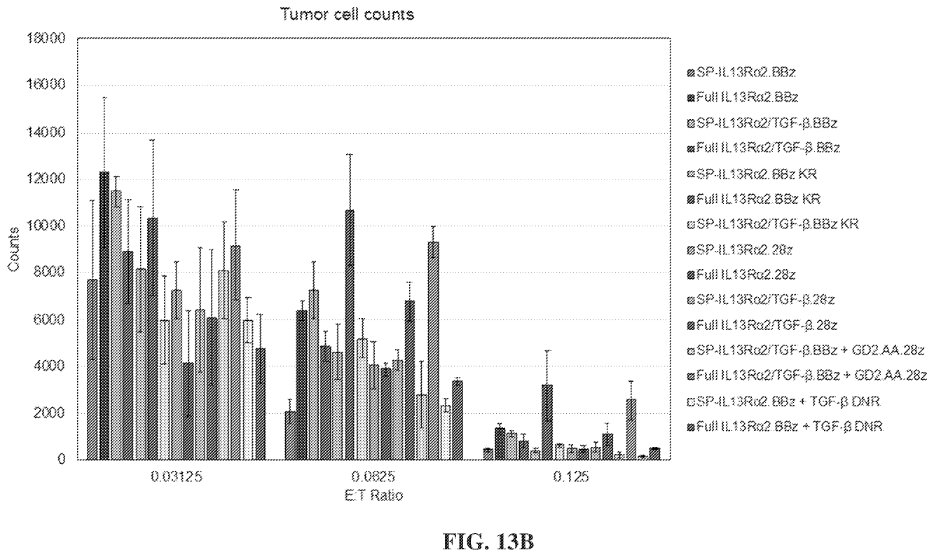

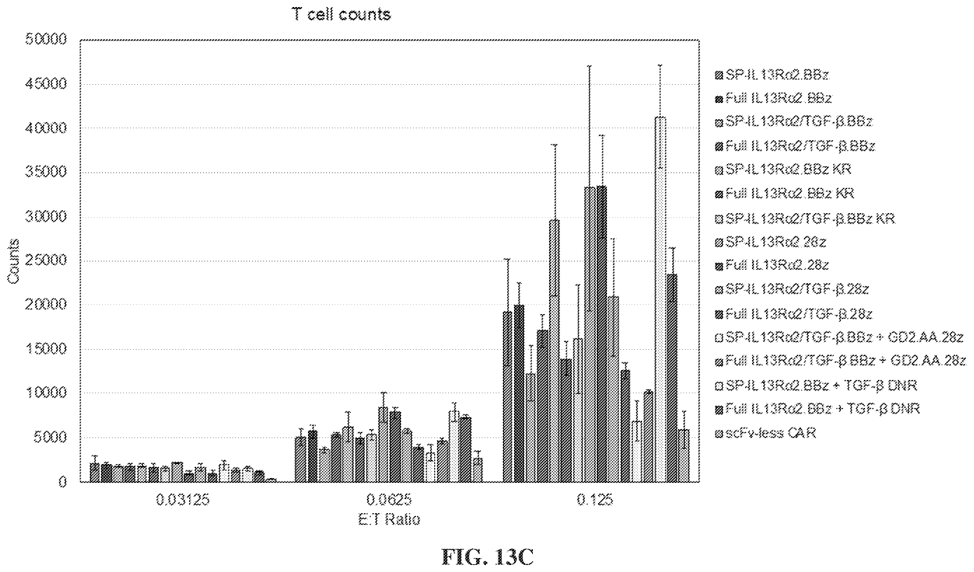

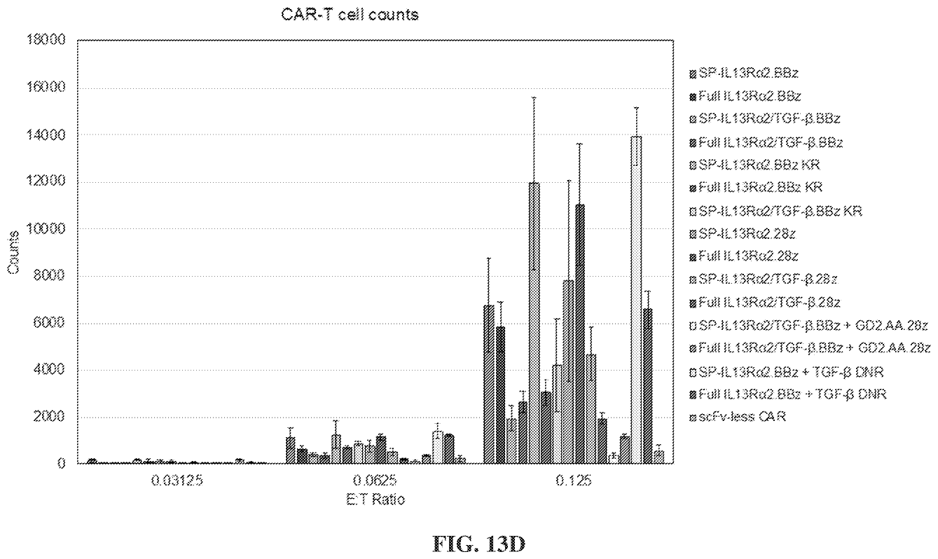

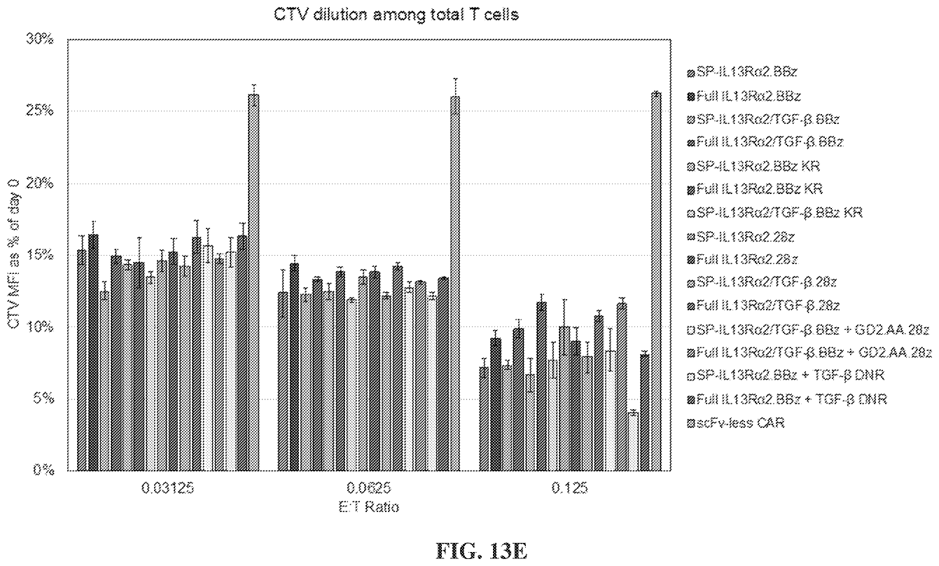

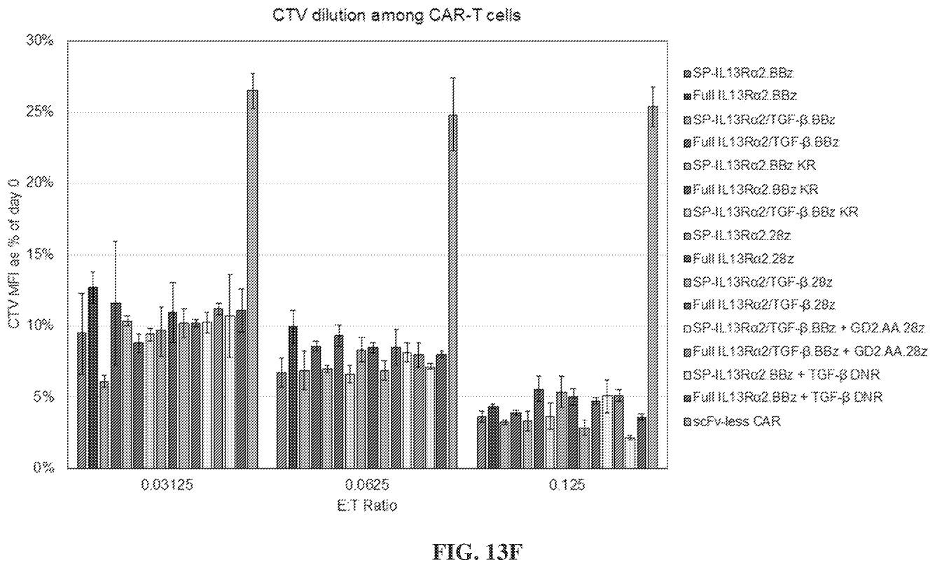

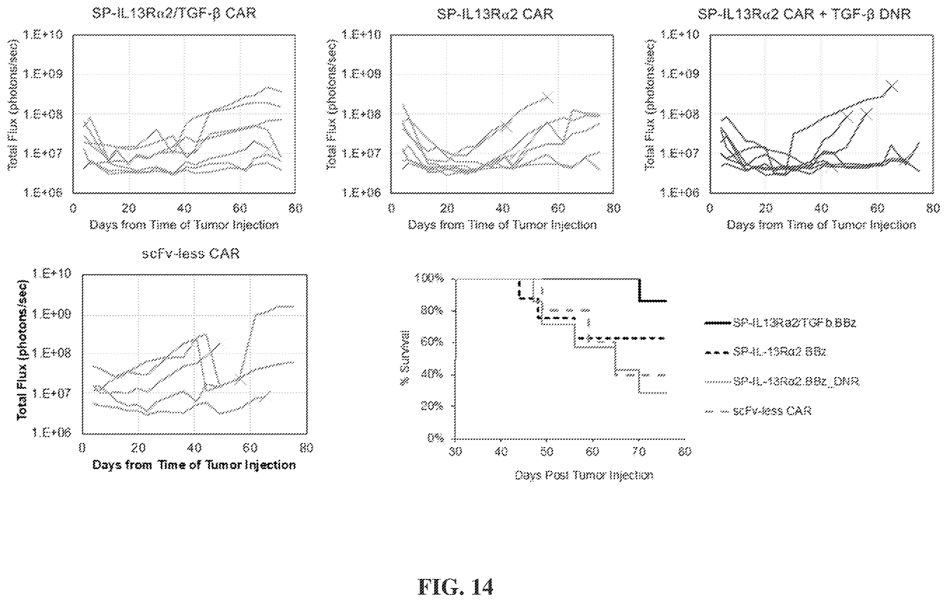

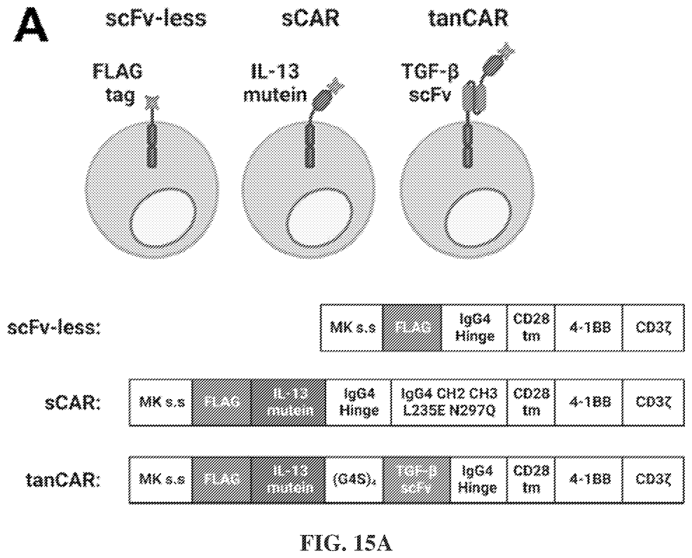

To assess anti-tumor function in vitro, CAR-T cells were labeled with CellTrace Violet (CTV) dye and co-cultured for 4 days with IL13Rα2+ PBT106 neurospheres at the indicated E:T ratios. Flow cytometry was performed to quantify viable tumor-cell count (FIG. 13A,B), viable T-cell count (FIG. 13C), viable CAR+ T-cell count (FIG. 13D), CTV dilution among all T cells (FIG. 13E), and CTV dilution among CAR+ T cells (FIG. 13F). FIG. 13B shows the same data as FIG. 13A, but with the scFv-less CAR condition removed to enable clear visualization. CARs bearing an N-terminal SP for the IL-13 mutein conferred more potent tumor-cell killing and greater T-cell proliferation (as assessed by CTV dilution) compared to CARs bearing the full IL-13 mutein N-terminus. Averages of triplicates are shown, with error bars representing ±1 standard deviation (FIG. 13).

### Example 3: Bispecific CAR-T Cells Counter TGF-β-Mediated Immune Suppression and Potentiate Anti-Tumor Responses in Glioblastoma

Glioblastoma multiforme (GBM) is one of the deadliest forms of cancer, with a median survival time of 12-15 months despite surgical resection, chemotherapy, and radiation, with no known cure to date. Chimeric antigen receptor (CAR)-T cell therapies targeting GBM-associated antigens such as interleukin-13 receptor subunit alpha-2 (IL-13Rα2) have achieved limited clinical efficacy to date, in part due to an immunosuppressive tumor microenvironment (TME) characterized by an abundance of inhibitory molecules such as transforming growth factor-beta (TGF-β). To more effectively combat GBM, the inventors engineered a single-chain, bispecific CAR targeting IL-13Rα2 and TGF-β, which programs tumor-specific T cells to convert TGF-β from an immunosuppressant to an immunostimulant. Bispecific IL-13Rα2/TGF-β CAR-T cells confer greater therapeutic outcomes compared to single-input IL-13Rα2 CAR-T cells against both patient-derived GBM xenografts and syngeneic models of murine glioma. Mechanistically, treatment with bispecific IL-13Rα2/TGF-β CAR-T cells drives greater T-cell infiltration and reduced suppressive myeloid cell presence compared to treatment with single-input IL-13Rα2 CAR-T cells, even when the single-input CAR-T cells are armed with a TGF-β dominant-negative receptor. This work reports the first single-chain bispecific CAR that can simultaneously target a membrane-bound tumor antigen and a soluble factor in the tumor microenvironment, converting the latter into not only a co-stimulatory signal but also a direct T-cell activation trigger. These findings demonstrate that by reprogramming tumor-specific T-cell responses to TGF-β, bispecific IL-13Rα2/TGF-β CAR-T cells resist and remodel the immunosuppressive TME to drive potent anti-tumor responses in GBM.

### A. Introduction

Glioblastoma multiforme (GBM) is the most prevalent primary brain tumor among adults, with poor patient prognosis despite aggressive treatment regimens combining chemotherapy, surgery, and radiation (1-5). Engineering T cells to express tumor-targeting chimeric antigen receptors (CARs), which has proven to be remarkably effective against hematological malignancies (6-10), offers a tantalizing means to program de novo anti-tumor immunity. Several GBM-associated antigens-including interleukin-13 receptor subunit alpha-2 (IL-13Rα2), epidermal growth factor receptor variant III (EGFRvIII), the disialoganglioside GD2, the checkpoint molecule B7-H3, and human epidermal growth factor receptor 2 (HER2)—are under active clinical evaluation as targets for CAR-T cell therapies (11-15). A 2016 report described one patient with GBM who experienced a complete radiographic response after receiving CAR-T cell therapy targeting IL-13Rα2 (11), an antigen that is overexpressed by 58-78% of gliomas (16,17) and correlates with poor prognosis (16). Multiple clinical trials evaluating different IL-13Rα2—targeted therapeutic modalities, ranging from CAR-T cell therapy to dendritic-cell vaccination to IL-13—conjugated toxins, have demonstrated IL-13Rα2 as a safe clinical target for malignant gliomas (11,18-21). However, therapeutic efficacy of these strategies has been very limited thus far, with little to no improvement to overall or progression-free survival.

As with many solid tumors, the highly immunosuppressive tumor microenvironment (TME) is a major barrier hindering the development of effective therapies against GBM (22). Transforming growth factor-beta (TGF-β), commonly overexpressed in a wide variety of solid tumor types, plays a prominent role in the GBM TME (23). TGF-β can be produced not only by malignant glioma cells but also by cells in the tumor stroma, and it plays a pivotal role in disease initiation and progression (24). Besides maintaining tumorigenicity of glioma-initiating stem cells, promoting tumor-cell proliferation, and increasing tumor invasiveness (25-27), TGF-β also modulates immune-cell composition and function in the TME. For instance, TGF-β directly inhibits CD8⁺ T-cell cytotoxicity and drives differentiation of naïve CD4⁺ T cells into the regulatory phenotype (28-30). Furthermore, TGF-β recruits and polarizes suppressive myeloid cells such as M2-like tumor-associated macrophages (TAMs) and myeloid-derived suppressor cells (MDSCs); these cells can themselves produce TGF-β, thus effecting a positive feedback loop for maintenance of the immunosuppressive TME (31-33). In light of its prominent role in shaping the immunosuppressive GBM microenvironment, TGF-β represents a promising therapeutic target. However, conventional strategies for blockade of TGF-β signaling, such as systemic administration of galunisertib, a small-molecule inhibitor of TGF-β receptor chain I, have had limited clinical success in the treatment of gliomas (34-36). A relative lack of infiltrating effector lymphocytes in immunologically cold tumors such as GBM (37.38), together with inefficient trafficking across the blood-brain barrier (39), may underlie the limited efficacy of TGF-β signaling blockade as a monotherapy. Furthermore, given the important functions that TGF-β plays in normal biological processes such as wound healing and angiogenesis (40), systemic administration of TGF-β signaling inhibitors raises concerns of toxicity (41,42).

Considering the programmability of cell-based immunotherapies, the inventors sought to engineer CAR-T cells that can simultaneously target GBM tumor cells and overcome TGF-β-mediated immune suppression. They previously demonstrated that a TGF-β-responsive CAR can effectively convert TGF-β from an immunosuppressant into a stimulant of engineered primary human T cells (43). Like the dominant-negative TGF-β receptor (DNR) (44-47) and switch receptors that convert TGF-β binding into a co-stimulatory signal (48-50) the TGF-β CAR can serve as a sink for TGF-β by outcompeting the endogenous TGF-β receptor for ligand binding, as well as provide co-stimulation in the presence of TGF-β. However, unlike these alternative designs, the TGF-β CAR is uniquely capable of directly activating the T cell, resulting in robust T-cell proliferation and cytokine production upon exposure to soluble TGF-β, without the need for a separate “signal one.” (43) This unique feature allows TGF-β CAR-T cells to be less prone to polarizing towards the regulatory phenotype in the presence of TGF-β and to protect neighboring tumor-specific T cells from TGF-β-mediated suppression of cytotoxicity (51). Although the TGF-β CAR can directly trigger T-cell activation as evidenced by T-cell proliferation and cytokine production, soluble TGF-β does not elicit cytotoxicity by TGF-β CAR-T cells due to the lack of an immunological synapse, actin re-organization, and directional degranulation (43). Consequently, the single-input TGF-β CAR is limited in clinical potential as a single-agent therapy. Here, the inventors combine the IL-13Rα2 CAR's ability to trigger direct tumor-cell killing with the TGF-β CAR's ability to convert TGF-β from an immunosuppressant into a T-cell activating stimulant, and report the first single-chain bispecific CAR that can simultaneously target a membrane-bound tumor-associated antigen and a soluble factor in the tumor microenvironment. The inventors demonstrate that the IL-13Rα2/TGF-β bispecific CAR enhances anti-tumor immunity by both conferring resistance to TGF-β-mediated suppression in engineered T cells and reprogramming neighboring immune cells in the TME from tolerogenic to inflammatory phenotypes.

### B. Results

1. Bispecific IL-13Rα2/TGF-β CAR-T Cells Target IL-13Rα2⁺ GBM and Convert TGF-β into a T-Cell Stimulant

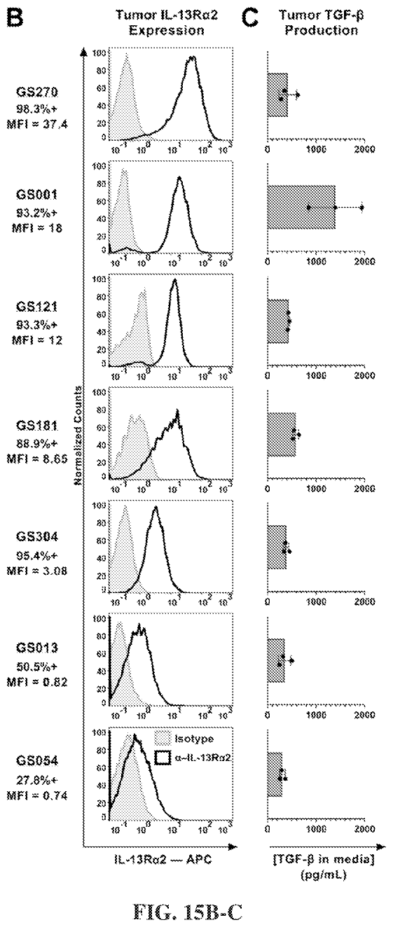

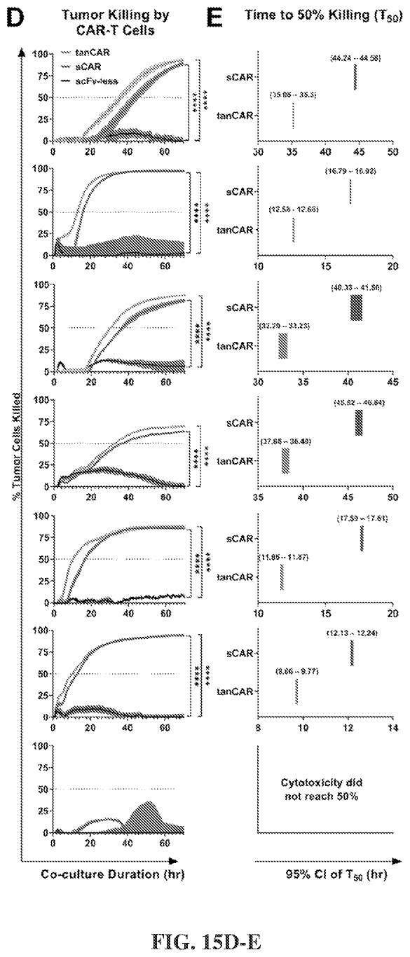

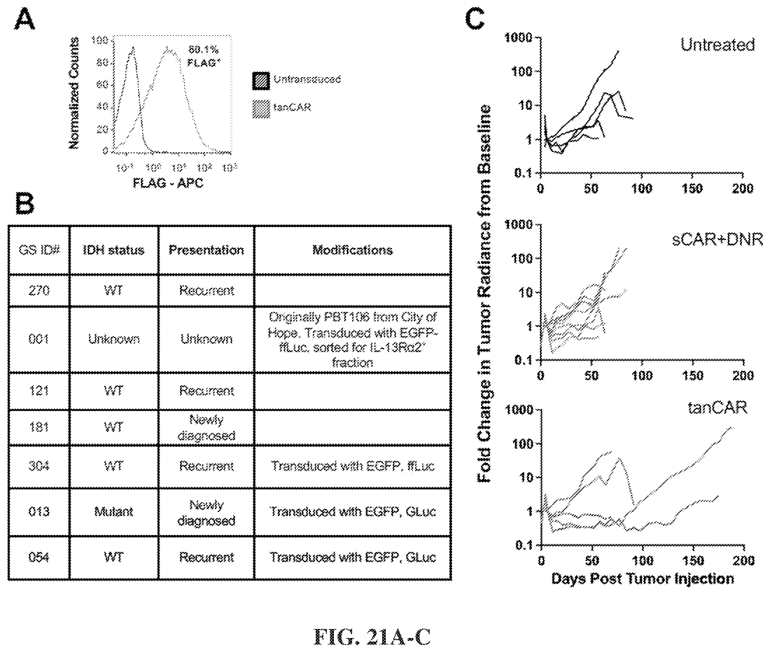

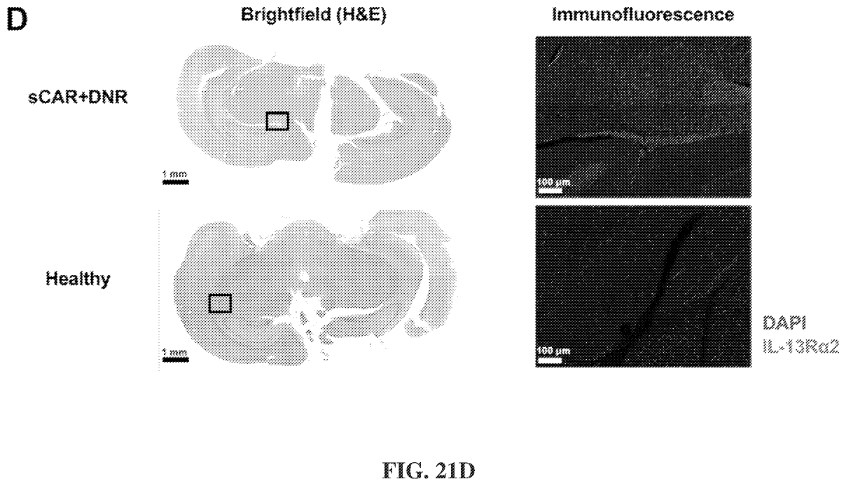

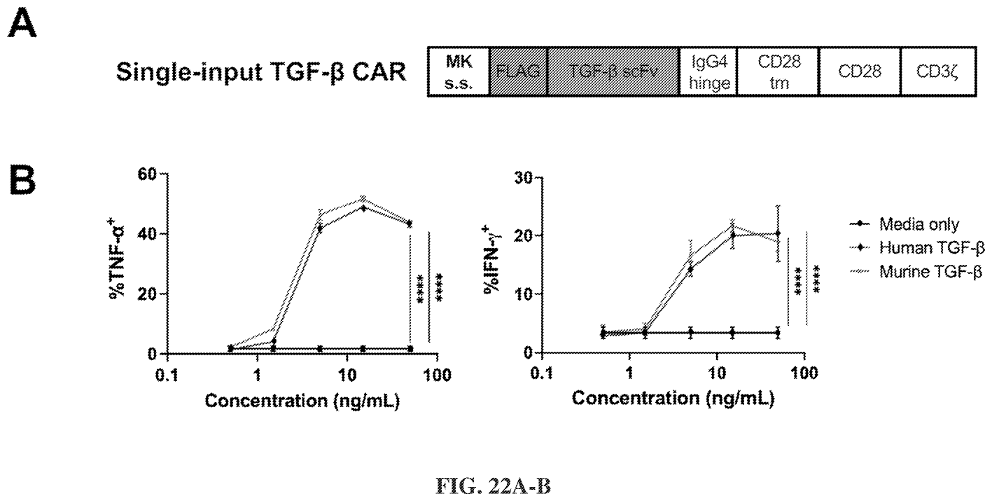

To assess whether anti-tumor function of a GBM-targeting CAR can be augmented by re-programming T-cell responses to TGF-β, the inventors designed a single-chain bispecific IL-13Rα2/TGF-β CAR (also known as a tandem CAR, or tanCAR), whose ligand-binding domain consists of the IL-13 E13Y mutein (52,53) fused to a TGF-β-specific single-chain variable fragment (scFv) (FIG. 15A). The bispecific CAR, which contains 4-1BB and CD35 signaling domains, is expressed efficiently on the surface of transduced primary human T cells (FIG. 21A). The performance of IL-13Rα2/TGF-β tanCAR-T cells was compared against T cells expressing either the single-input IL-13Rα2 CAR (sCAR) (54) or an scFv-less CAR that lacks a ligand-binding domain (FIG. 15A). As target cells, a panel of patient-derived GBM neurospheres covering a range of expression levels of IL-13Rα2 and TGF-β was assembled (FIG. 15B, C; FIG. 21B). As previously reported, soluble TGF-β is not expected to trigger cytotoxicity, (43) and the inventors indeed observed that tanCAR-T cells showed similar overall efficacy compared to sCAR-T cells (FIG. 15D). However, tanCAR-T cells showed faster killing against patient-derived GBM neurospheres relative to sCAR-T cells (FIG. 15E), potentially due to increased activation intensity resulting from TGF-β-mediated stimulation of tanCAR-T cells. Efficient killing was observed against GBM neurospheres with a wide range of IL-13Rα2 expression levels, including samples that were weak and heterogenous in IL-13Rα2 expression (e.g., GS013 neurospheres at 50% IL-13Rα2+). However, both sCAR-T cells and tanCAR-T cells failed to lyse GS054 neurospheres (27.8% IL-13Rα2+) (FIG. 15D, E). These results demonstrate that the CAR-T cells can efficiently target clinically relevant tumor samples but are unable to eliminate tumor cells with very low or no IL-13Rα2 expression in vitro. This observation underscores the need to shape the GBM TME to facilitate recruitment of endogenous anti-tumor immune responses.

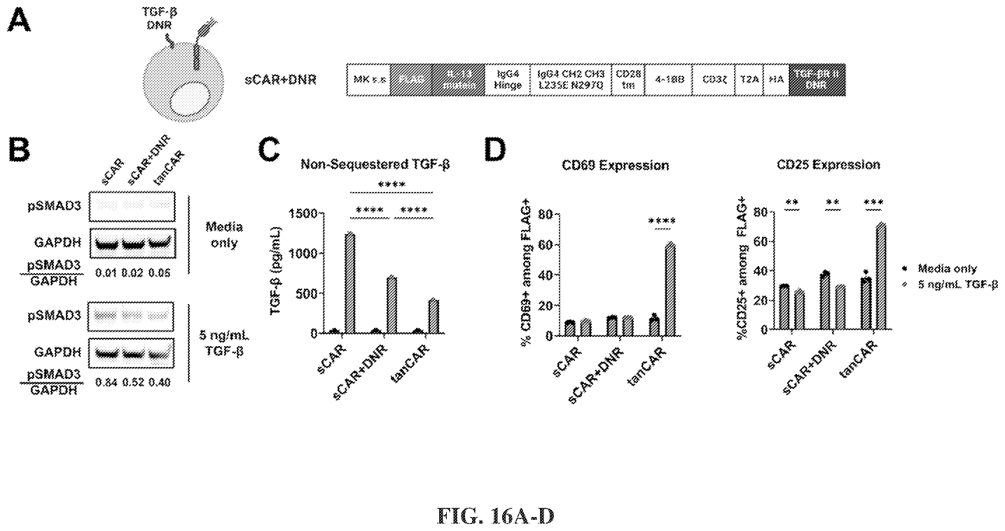

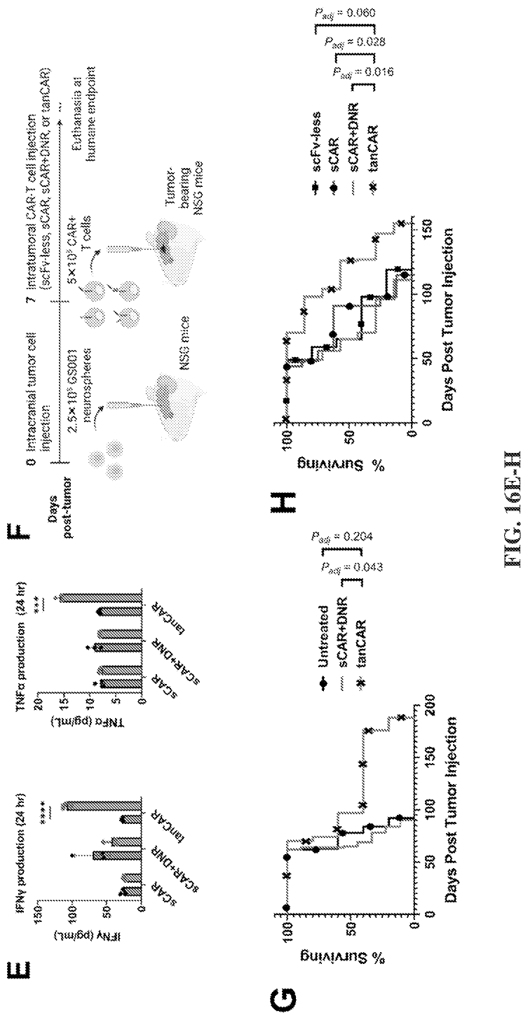

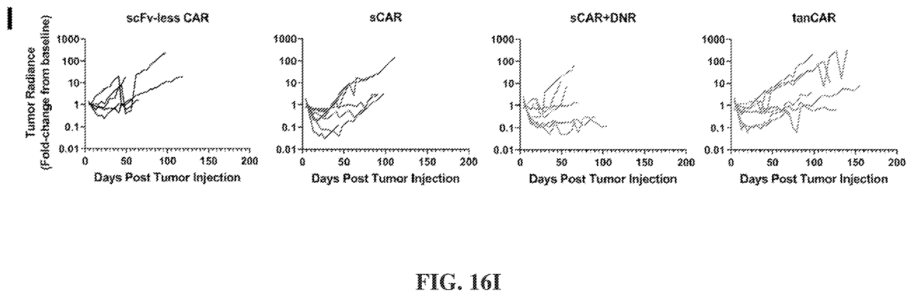

The inventors aim to remodel the TME with the TGF-β-targeting moiety of the IL-13Rα2/TGF-β tanCAR, which is expected to confer three capabilities to engineered T cells: (1) inhibit endogenous TGF-β signaling in engineered T cells through competition against endogenous TGF-β receptors for ligand binding, (2) inhibit TGF-β signaling in nearby, endogenous immune cells by serving as a sink for TGF-β, and (3) activate engineered T cells in the presence of TGF-β, resulting in both T-cell intrinsic and paracrine mechanisms of immune stimulation. The last capability distinguishes the TGF-β-targeted tanCAR from the TGF-β dominant-negative receptor (DNR), which is a truncated version of TGF-β receptor chain 2 that lacks the cytoplasmic signaling domain (44). The DNR has been evaluated in clinical trials and has shown the ability to reduce endogenous TGF-β signaling and enhance T-cell function (45-47), but it lacks the capacity to trigger T-cell activation. The inventors thus compared the functionality of IL-13Rα2/TGF-β tanCAR-T cells with IL-13Rα2 CAR-T cells that co-express the DNR (sCAR+DNR; FIG. 16A).

T cells expressing either the tanCAR or sCAR+DNR both showed reduced SMAD3 phosphorylation upon exposure to TGF-β compared to sCAR-T cells, confirming efficient abatement of endogenous TGF-β signaling by both the tanCAR and the DNR (FIG. 16B). When cultured in media supplemented with recombinant active TGF-β. T cells expressing either the tanCAR or sCAR+DNR removed significantly more TGF-β from the culture media compared to sCAR-T cells, with tanCAR-T cells being the most effective at TGF-β sequestration (FIG. 16C). Furthermore, only tanCAR-T cells were activated by TGF-β, as evidenced by the increased expression of CD69 and CD25 (FIG. 16D) as well as the production of Th1 cytokines interferon gamma (IFNγ) and tumor necrosis factor alpha (TNFα) (FIG. 16E). Therefore, while both the tanCAR and the DNR can decrease endogenous immunosuppressive TGF-β signaling, only the tanCAR converts TGF-β into an immunostimulant.

### 2. Bispecific IL-13Rα2/TGF-β CAR-T Cells Exhibit Superior In Vivo Anti-Tumor Efficacy Against Patient-Derived GBM Neurospheres Compared to Conventional IL-13Rα2 CAR-T Cells

The in vitro assays shown in FIGS. 16B-E were performed using mature TGF-β protein, and the TGF-β CAR does not respond to the latent form of TGF-β (51). Since TGF-β is generally produced in the latent form and only converted into the mature, active form in the presence of metalloproteases characteristic of the TME (55-58), the inventors focused subsequent characterization efforts in vivo in tumor-bearing hosts. The inventors first evaluated tanCAR-T cell function in an orthotopic model of patient-derived GBM. GS001 neurospheres, which naturally secrete TGF-β at high levels (FIG. 15C), were engineered to express firefly luciferase (ffLuc) and intracranially implanted in NOD/scid/γ^{-/-} (NSG) mice. CAR-T cells were administered intratumorally after tumor engraftment was confirmed by bioluminescence imaging (FIG. 16F). A pilot study showed superior tumor control by tanCAR-T cells compared to T cells expressing sCAR+DNR (FIG. 16G). In a larger-scale confirmatory study, tanCAR-T cells again exhibited greater control over tumor outgrowth and conferred more favorable survival outcomes compared to sCAR-T cells, with or without DNR co-expression (FIGS. 16H, I). The inventors noted that several mice in both studies reached humane endpoint without detectable tumor radiance signal (FIG. 16I; FIG. 21C). Post-mortem examination of the brains of these mice showed clear abnormalities in tissue color and morphology, and immunohistochemistry (IHC) staining confirmed the presence of IL-13Rα2+ cells (FIG. 21D), suggesting the possibility of neurosphere differentiation upon engraftment in the brain that resulted in the loss of luciferase transgene expression, as has been previously described (59). Taken together, these findings demonstrate that by not only blocking but also re-wiring suppressive TGF-β signaling, tanCAR-T cells exert more potent anti-tumor function compared to sCAR-T cells, with or without DNR co-expression.

3. Human tanCAR is Functional in Murine T Cells and Responds to Murine TGF-β

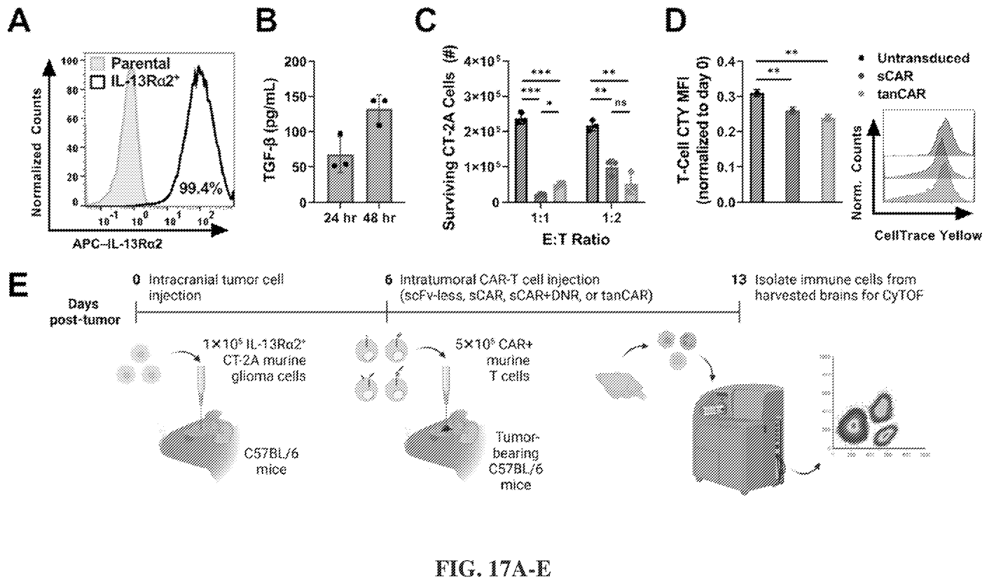

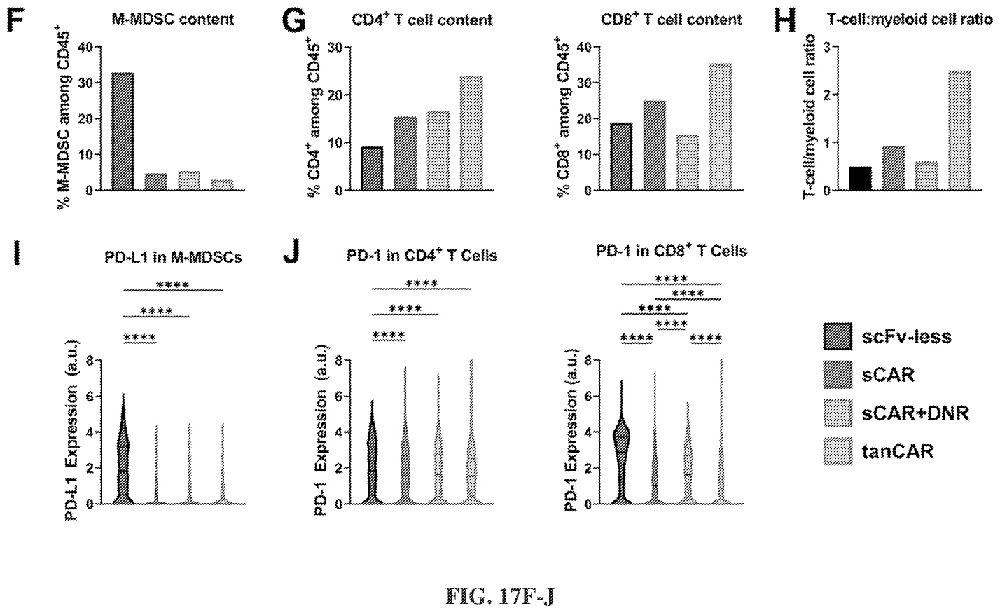

Given the major role of TGF-β in regulating immunosuppressive cell types such as TAMs and MDSCs, the inventors next sought to understand how tanCAR-T cells might influence, or be influenced by, endogenous immune cells in the TME. Such an exploration requires the use of syngeneic tumor models in immunocompetent mice, and the inventors chose to utilize the murine CT-2A glioma line. To properly evaluate tanCAR-T cell efficacy in the CT-2A model, the tumor must express antigens recognizable to the CAR. The inventors thus engineered CT-2A cells to express human IL-13Rα2 (FIG. 17A) as well as ffLuc to track tumor progression via bioluminescence imaging. The inventors also verified that engineered IL-13Rα2+CT-2A cells naturally secrete murine TGF-β both in vitro (FIG. 17B) and upon intracranial implantation into C57BL/6 mice (data not shown). Importantly, murine TGF-β was detected by IHC in not only tumor cells, but also among tumor-infiltrating myeloid cells in the tumor-bearing brain, confirming that endogenous immune cells are an important source of TGF-β in the CT-2A TME (data not shown).

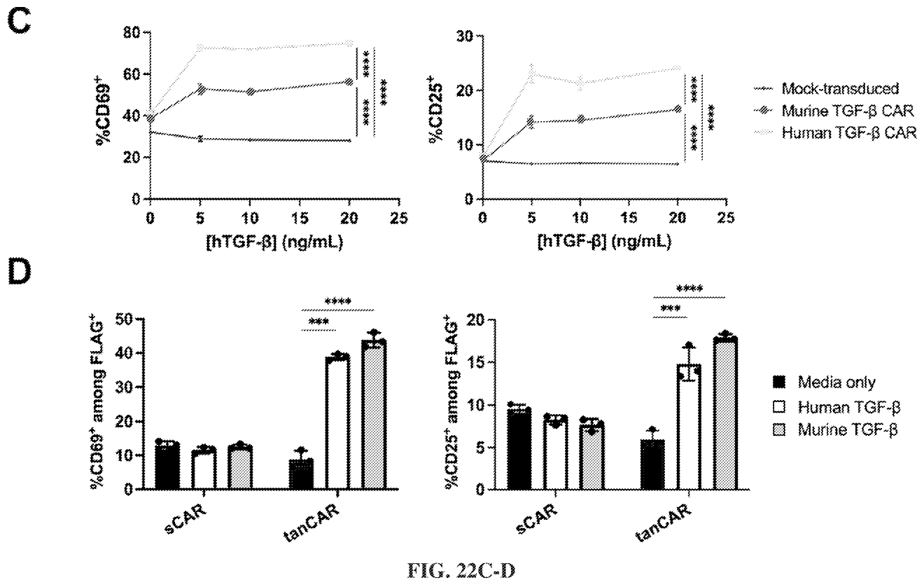

As human and murine TGF-β are highly conserved in amino-acid sequence, the inventors first verified that tanCARs designed to target human TGF-β could cross-recognize murine TGF-β, thus obviating the need to engineer CT-2A cells to also express human TGF-β, and ensuring that tanCARs are also reactive towards TGF-β produced by endogenous mouse cells in the TME. The inventors first evaluated the previously described single-input TGF-β CAR containing a human CD28 co-stimulatory domain (43), and found that human TGF-β CAR-T cells exhibit the same dose-dependent response to both human and murine TGF-β (FIGS. 22A, B). Furthermore, murine T cells expressing the human TGF-β CAR robustly respond to TGF-β (yellow curve; FIG. 22C), indicating the human CAR protein could function in murine T cells without further modification. In fact, the human TGF-β CAR signals more strongly than an equivalent CAR containing murine signaling domains when expressed in murine T cells (FIG. 22C), suggesting underlying differences in murine and human T-cell biology. Finally, the inventors confirmed that murine T cells expressing the IL-13Rα2/TGF-β tanCAR encoding human 4-1BB and CD35 signaling domains are activated in response to both human and murine TGF-β (FIG. 22D). Since the human CAR is the clinically relevant candidate, the inventors proceeded to test CARs with human signaling domains in the in vitro functional assays and in vivo syngeneic studies.

To evaluate tanCAR-T cell killing ability and activation-induced proliferation, the inventors co-incubated IL-13Rα2+CT-2A cells with CellTrace Yellow (CTY)-stained murine CAR-T cells (FIG. 17C, D). After four days of co-incubation, both sCAR-T cells and tanCAR-T cells exhibited efficient killing of target cells (FIG. 17C) and significantly more proliferation compared to untransduced T cells, as measured by CTY dilution (FIG. 17D). Taken together, these results indicate that the human tanCAR can be directly evaluated in immunocompetent mouse models bearing syngeneic GBM tumors.

4. Treatment with IL-13Rα2/TGF-β tanCAR-T Cells Reduces Suppressive Immune-Cell Infiltration and Enriches for Activated T Cells in the GBM TME

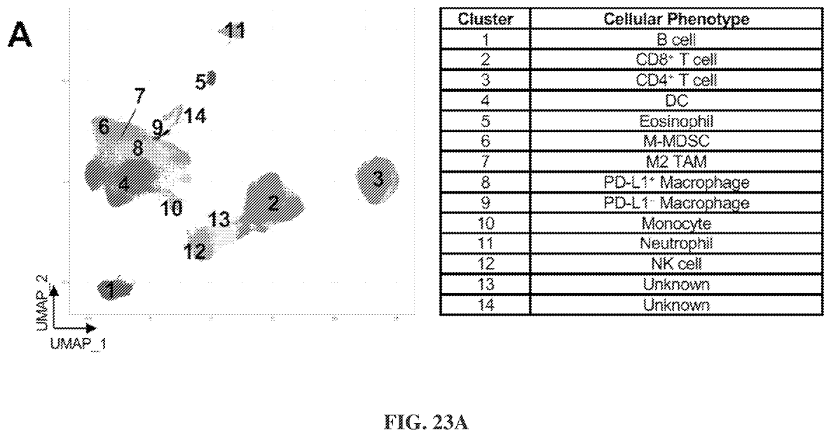

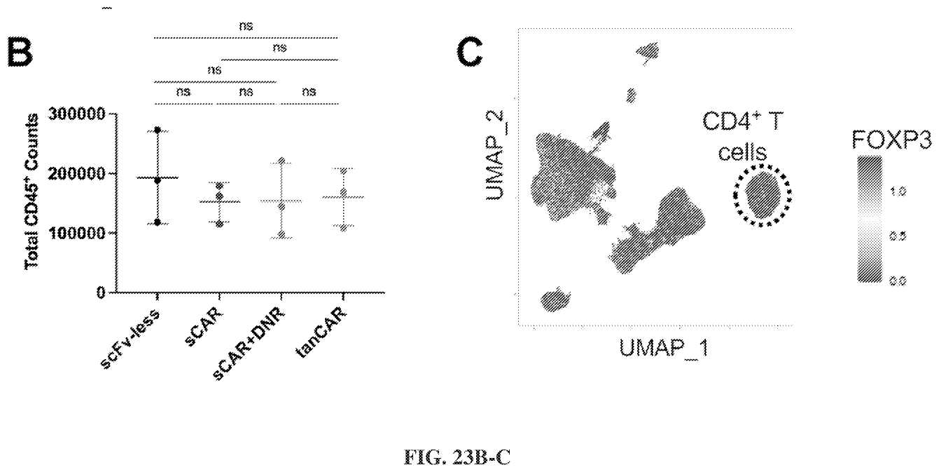

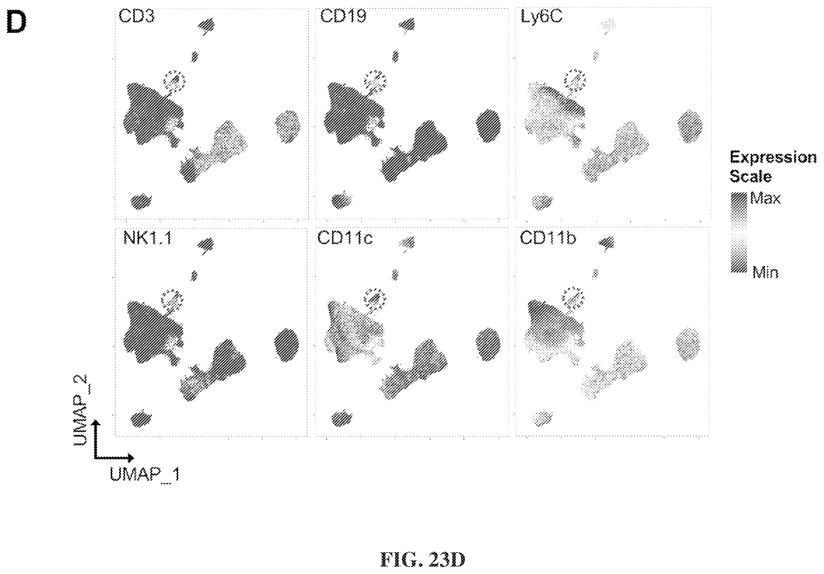

To understand the influence of CAR-T cells on the surrounding TME, C57BL/6 mice were intracranially implanted with IL-13Rα2+CT-2A glioma cells and treated with either scFv-less or various IL-13Rα2-targeting CAR-T cells. Cytometry by time-of-flight (CyTOF) was performed on brain tissue (pooled from n=3 mice per treatment group) collected seven days following adoptive T-cell transfer (FIG. 17E, FIG. 23A). Varying the treatment condition did not significantly alter the total number of CD45+ cells recovered from each tumor-bearing brain (FIG. 23B). Among the treatment groups, animals treated with tanCAR-T cells had the lowest frequency of M-MDSCs and the greatest enrichment of CD4⁺ and CD8⁺ T cells, leading to the highest ratio of T cells to myeloid cells (FIGS. 17F-H). High intratumoral T-cell:myeloid cell ratios have been positively correlated with response to immunotherapy in patients with recurrent glioblastoma and in preclinical models of pancreatic cancer (60,61). Importantly, the CD4⁺ T cells detected by CyTOF (cluster 3) were not FOXP3⁺ regulatory T cells (Tregs; FIG. 23C). Instead, FOXP3 signal was detected among M-MDSCs (cluster 6), a subset of DCs (cluster 4), and most prominently in cluster 14, which showed high signal intensity for nearly all markers in the CyTOF panel, including NK. T. B. and myeloid cell lineage markers (FIG. 23D). This cluster, which accounts for <1% of all analyzed CD45⁺ cells, was likely artifactual and thus classified as unknown.

Previous work has demonstrated that tumor-infiltrating CD8⁺ T cells in mice bearing CT-2A gliomas exhibit a highly exhausted phenotype marked by strong expression of PD-1 (62,63). Furthermore, others have shown that myeloid cells drive T-cell dysfunction in the GBM TME through PD-1/PD-L1 checkpoint interactions (64). Consistent with these reports, the inventors observed high PD-L1 and PD-1 expression among myeloid cells and T cells, respectively, in brains recovered from mice treated with scFv-less CAR-T cells (FIGS. 17I, J). However, treatment with on-target CAR-T cells (sCAR, sCAR+DNR, and tanCAR) resulted in a reduction of PD-L1 expression among myeloid cells and a reduction of PD-1 expression among T cells in the brain compared to treatment with scFv-less CAR-T cells, suggesting tumor-targeting by CAR-T cells favorably altered the immune environment in the brain (FIGS. 17I, J). Of note, brain-infiltrating CD8⁺ T cells from the tanCAR-T cell-treated group exhibited significantly lower PD-1 levels compared to those from sCAR-T cell-treated groups, with or without DNR co-expression (FIG. 17J). Taken together with the finding that tanCAR-T cell treatment resulted in the greatest enrichment of both CD4⁺ and CD8⁺ T cells, these findings suggest that tanCAR-T cells promote anti-tumor adaptive immunity in the TME.

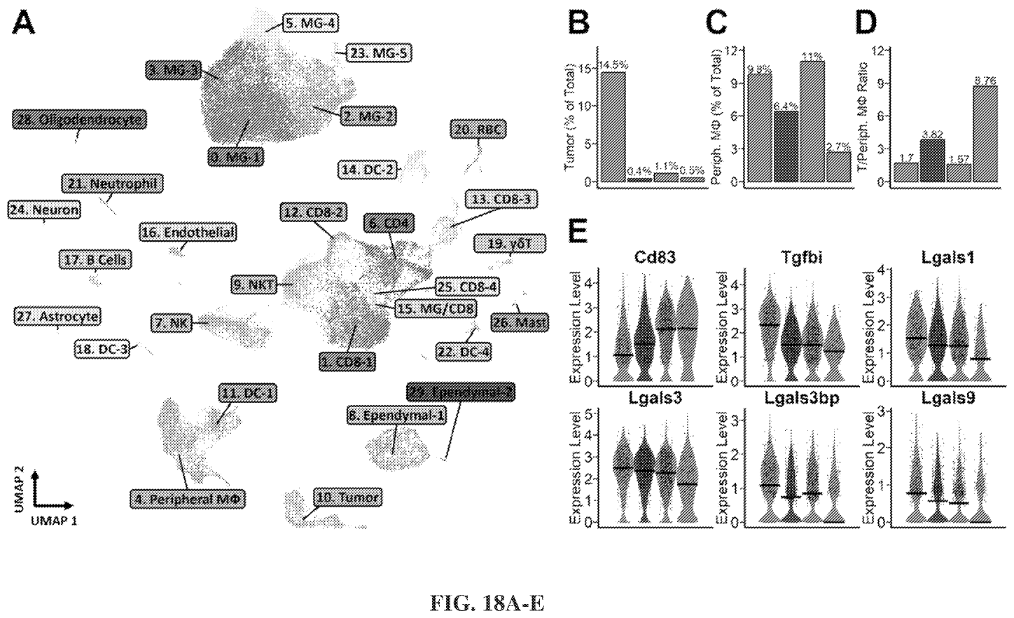

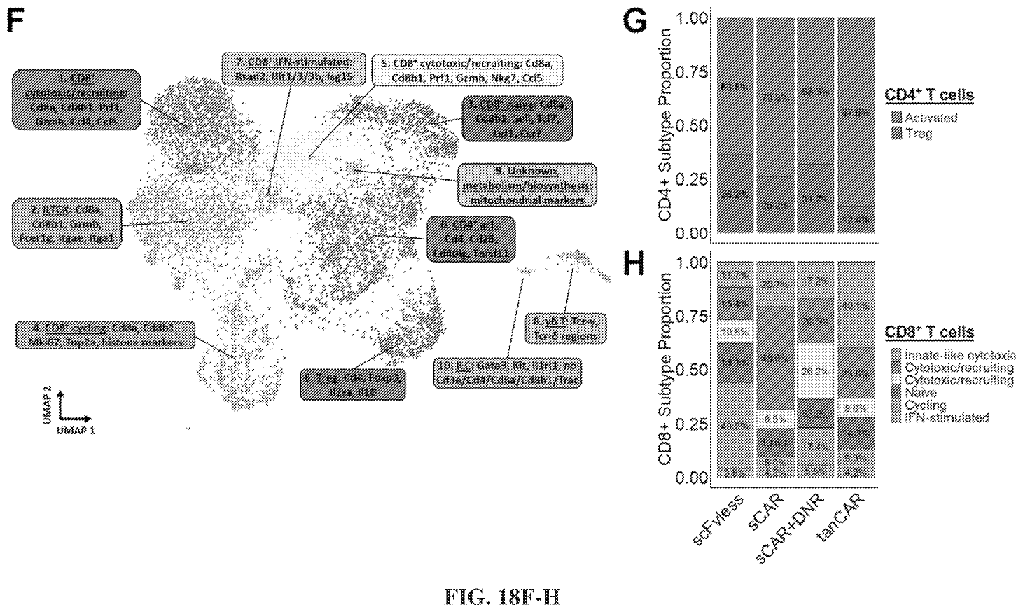

To further interrogate interactions between CAR-T cells and endogenous immune cells in the CT-2A TME, single-cell RNA sequencing (scRNA-seq) was performed on whole brains (pooled from n=3 mice per treatment group) collected seven days following adoptive CAR-T cell transfer (i.e., 13 days post tumor injection). Transcriptomic sequencing data were stringently filtered and integrated across treatment groups for visualization by uniform manifold approximation and projection (UMAP. FIG. 18A), and all thirty clusters were annotated according to published canonical and functional markers (data not shown).

Consistent with expectations, all on-target CAR-T cell treatments demonstrated robust tumor control compared to the scFv-less CAR-T cell treatment at this time point (FIG. 18B). Importantly, the frequency of infiltrating peripheral macrophages, which is correlated with poor survival in both human patients (65) and mouse models (66) of GBM, was markedly lower in mice treated with tanCAR-T cells compared to all other treatment groups (FIG. 18C). Similar to observations from CyTOF, the ratio of tumor-infiltrating T-cells to infiltrating peripheral macrophages was also the highest in mice treated with tanCAR-T cells (FIG. 18D), providing consistent evidence that tanCAR-T cells favorably modify the composition of the TME. Furthermore, peripheral macrophages that trafficked to the brain of tanCAR-T cell-treated mice had the highest per-cell expression of transcripts encoding CD83, which is transiently upregulated in activated macrophages (67), as well as the lowest per-cell expression of transcripts encoding the TGF-β-induced protein (TGF-βi), which is produced downstream of the TGF-β signaling axis (FIG. 18E). Additionally, peripheral macrophages infiltrating the brains of mice treated with tanCAR-T cells exhibited the lowest transcript levels for galectin-1 (LGALS1), galectin-3 (LGALS3), galectin-9 (LGALS9), and galectin-3-binding protein (LGALS3 bp) (FIG. 18E), which have each been correlated with immunosuppression, tumor progression, and worse survival in glioma (68-71). These galectin-family proteins can promote tumor-cell migration and metastasis and negatively regulate both innate and adaptive anti-tumor immunity (72,73). Notably, galectin-1 and galectin-9 expression can be upregulated by TGF-β (74,75), which is consistent with the observation that peripheral macrophages exhibit weaker galectin expression following tanCAR-T cell treatment. These findings indicate that tanCAR-T cells not only reduce the influx of, but also dampen immunosuppressive programs expressed in, tumor-associated macrophages.

The inventors had previously demonstrated the protective effects of TGF-β-responsive CAR-T cells on surrounding, non-engineered T cells (51). Here, the inventors further interrogated the lymphoid compartment in the scRNA-seq data set. Re-clustering CD8⁺, CD4⁺, and γδ T cells yielded 11 new clusters with greater resolution in T-cell subtypes (FIG. 18F; data not shown). Compared to all other treatment groups, treatment with tanCAR-T cells resulted in a marked increase of pro-inflammatory CD40LG⁺ activated CD4⁺ T cells and a corresponding decrease in FOXP3⁺ Tregs (FIG. 18G). Furthermore, mice treated with tanCAR-T cells showed an enrichment of FCERG1⁺ innate-like cytotoxic T cells, which were recently demonstrated to have a uniquely high cytotoxic potential in the context of anti-tumor response (76) (FIG. 18H). Taken together, scRNA-seq analysis indicates that, compared to sCAR-T cells with or without DNR co-expression, treatment with tanCAR-T cells reduces the influx of immunosuppressive peripheral macrophages, reduces the frequency of CD4⁺ Tregs, and increases the frequency of activated effector T cells in the GBM TME.

5. IL-13Rα2/TGF-β tanCAR-T Cells Exhibit Superior In Vivo Anti-Tumor Efficacy Against Syngeneic GBM Tumor Compared to Conventional IL-13Rα2 CAR-T Cells

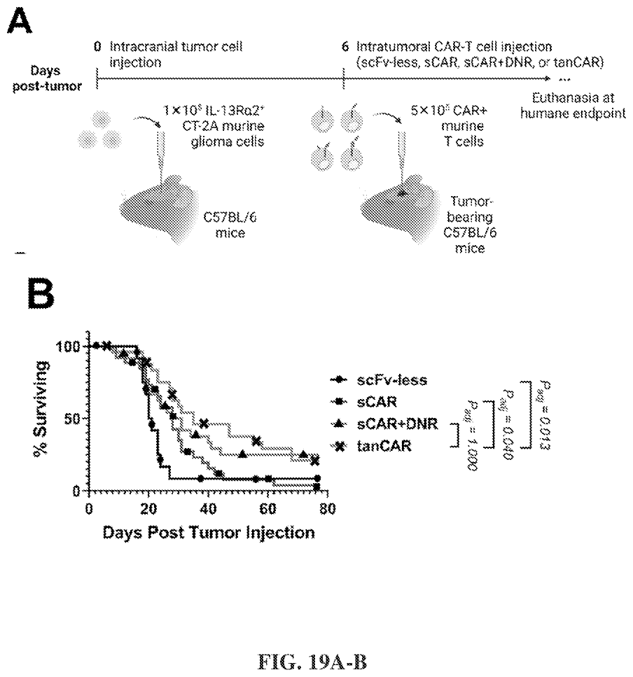

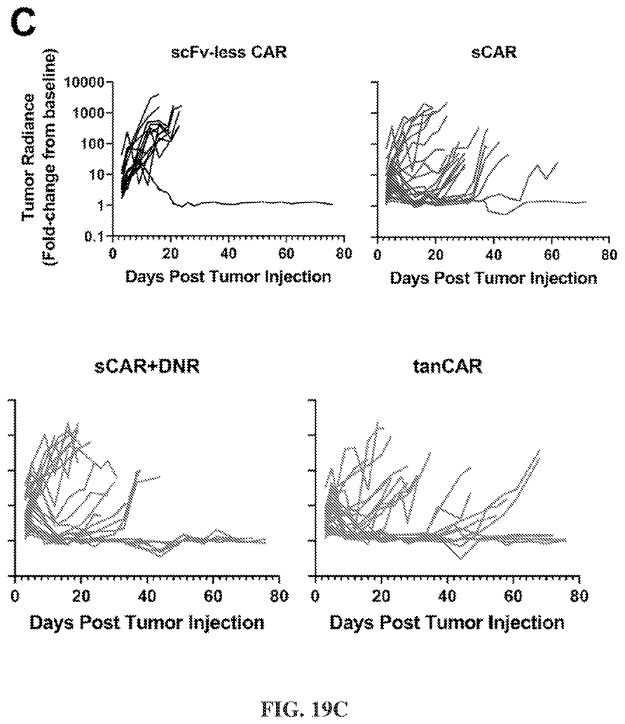

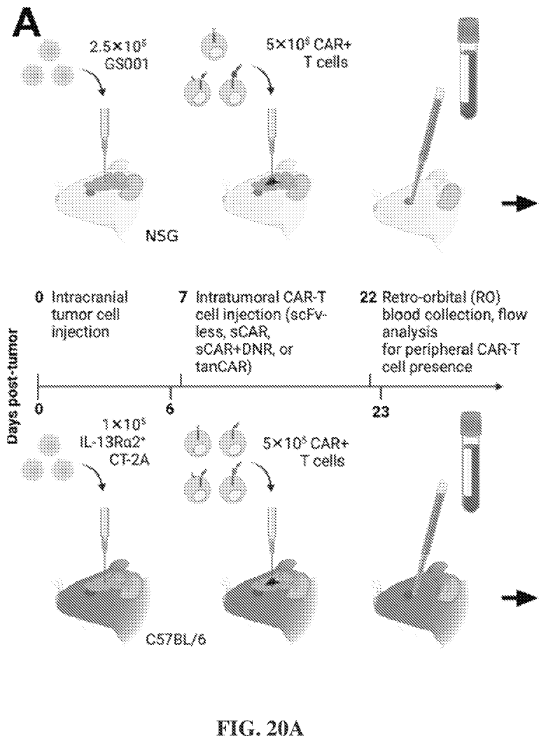

The inventors next evaluated whether the changes in the immune landscape of the GBM TME observed by CyTOF and scRNA-seq analyses would translate into differential tumor control and survival in the CT-2A model. C57BL/6 mice were intracranially implanted with IL-13Rα2+CT-2A glioma cells and treated with either scFv-less or various IL-13Rα2-targeting CAR-T cells (FIG. 19A). Consistent with earlier results observed in the human xenograft model (FIG. 16H), tanCAR-T cells exhibited superior anti-tumor function compared to sCAR-T cells, resulting in a significant increase in survival period and confirming the advantage of utilizing a bispecific CAR design over the conventional single-input CAR (FIG. 19B).

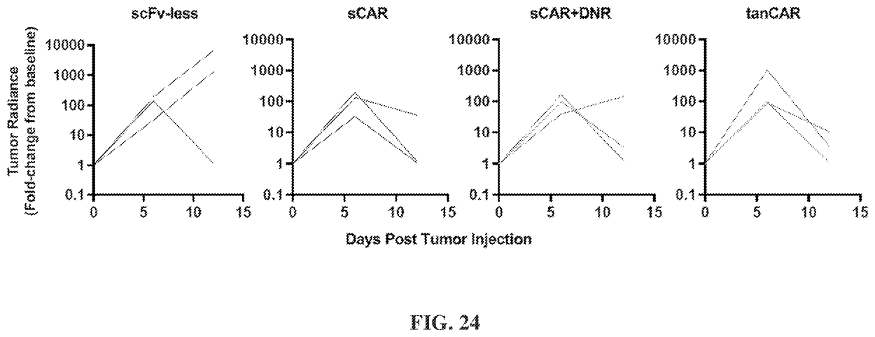

Interestingly, although the sCAR+DNR design performed poorly in the human xenograft model (no different than untreated control or scFv-less CAR; FIGS. 16G, H), it exhibited improved functionality in the syngeneic CT-2A model and outperformed the sCAR alone (FIG. 19B). Compared to tanCAR-T cells, treatment with T cells expressing the sCAR+DNR resulted in more mice with uninhibited tumor outgrowth within the first three weeks (10/24 in sCAR+DNR vs. 4/24 in tanCAR) but also a substantial number of mice that eventually eradicated the tumor (6/24 in sCAR+DNR vs. 5/24 in tanCAR), leading to no significant difference in overall survival between the two treatment groups (FIG. 19B, C). Of note, the sCAR+DNR group was also more heterogeneous than the tanCAR group in its anti-tumor response in the mice that were sacrificed to collect samples for CyTOF and scRNA-seq analyses (FIG. 24).

Taken together, these results indicate that the IL-13Rα2/TGF-β bispecific tanCAR significantly outperforms the conventional, single-input IL-13Rα2 sCAR in the control of syngeneic GBM. Furthermore, the sCAR+DNR can also confer increased anti-tumor efficacy compared to the sCAR in the syngeneic CT-2A model, albeit with a tendency toward bimodal response of either no efficacy (early tumor progression) or strong efficacy (tumor eradication).

6. IL-13Rα2/TGF-β tanCAR-T Cells are Well-Tolerated

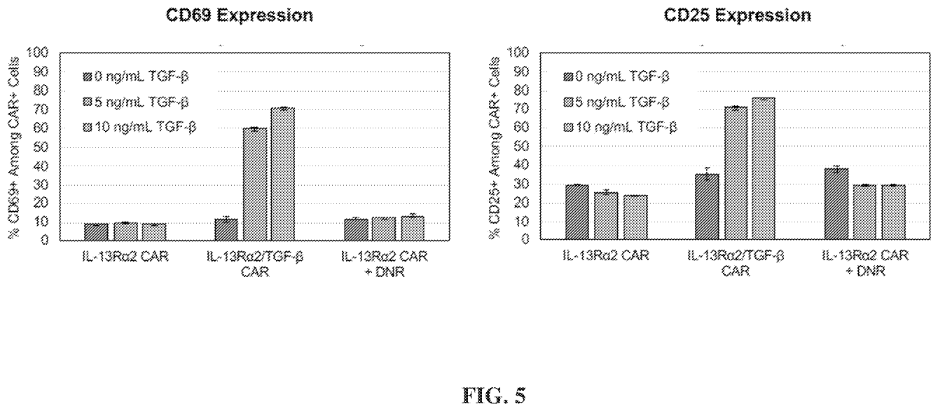

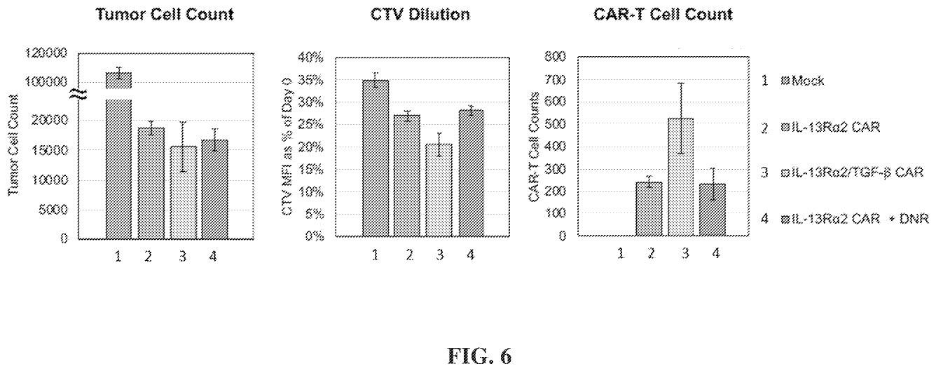

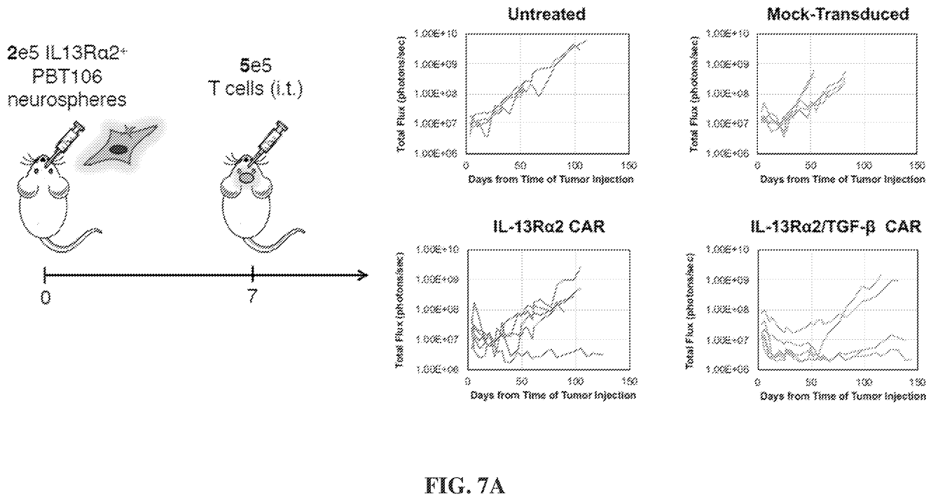

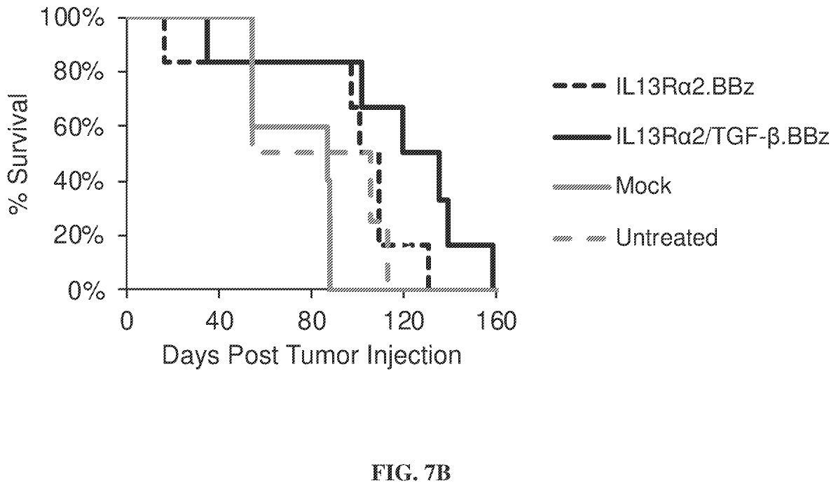

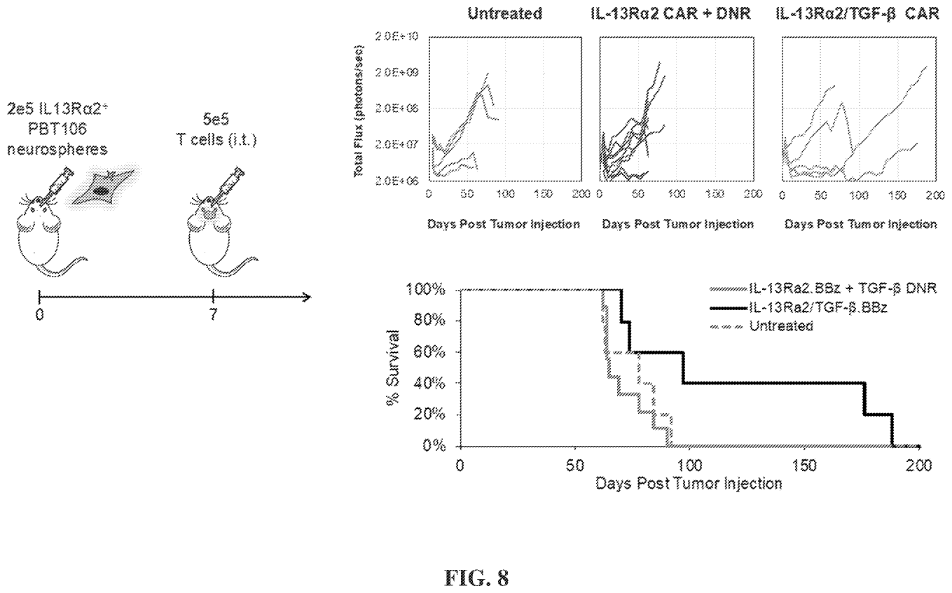

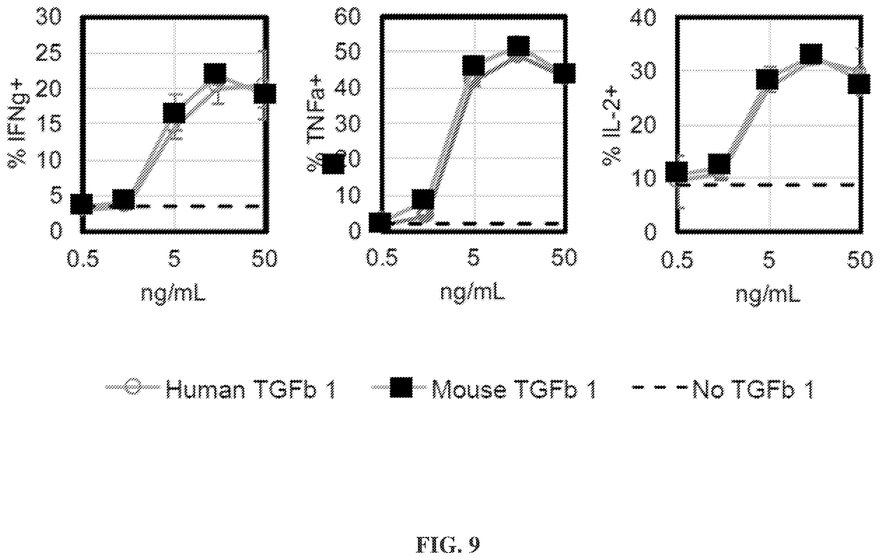

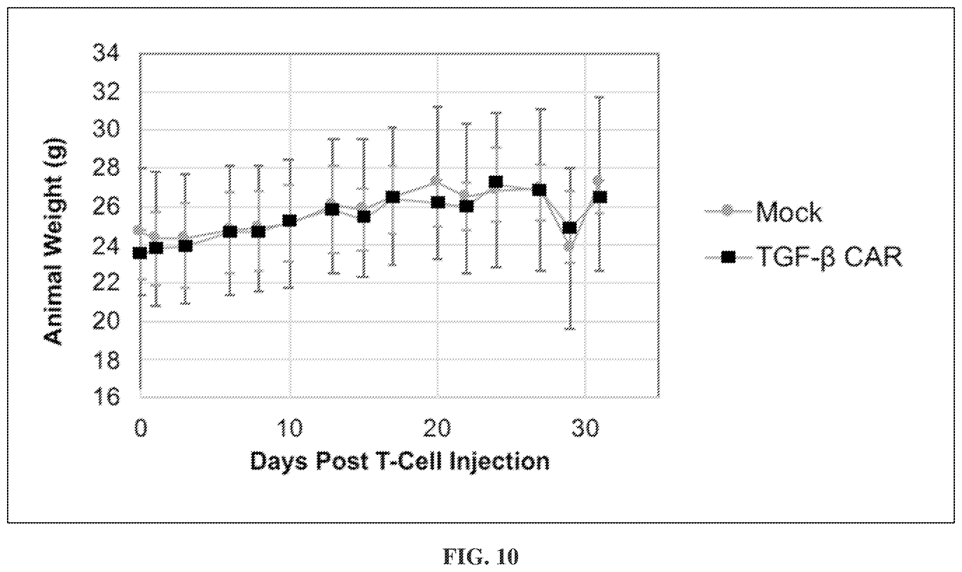

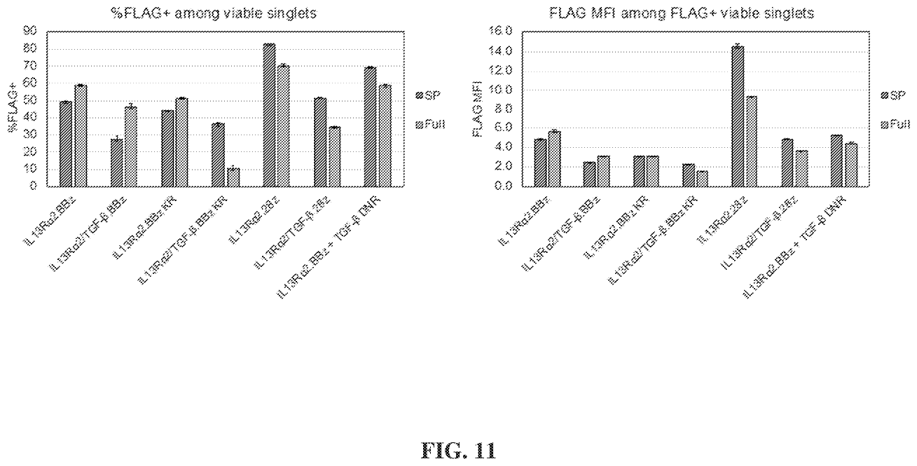

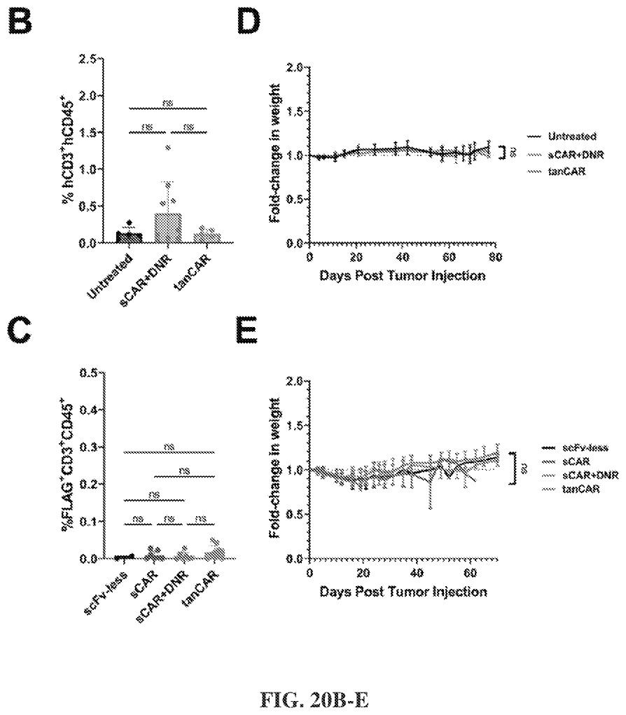

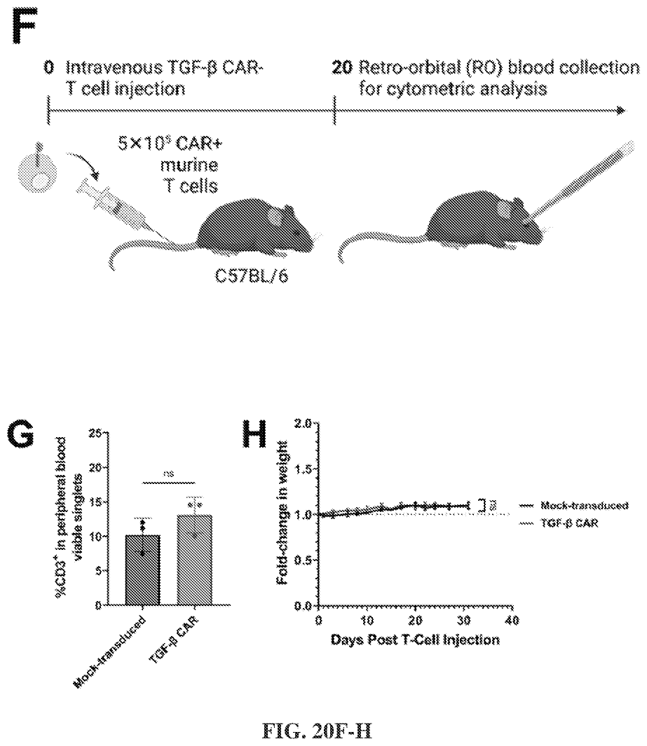

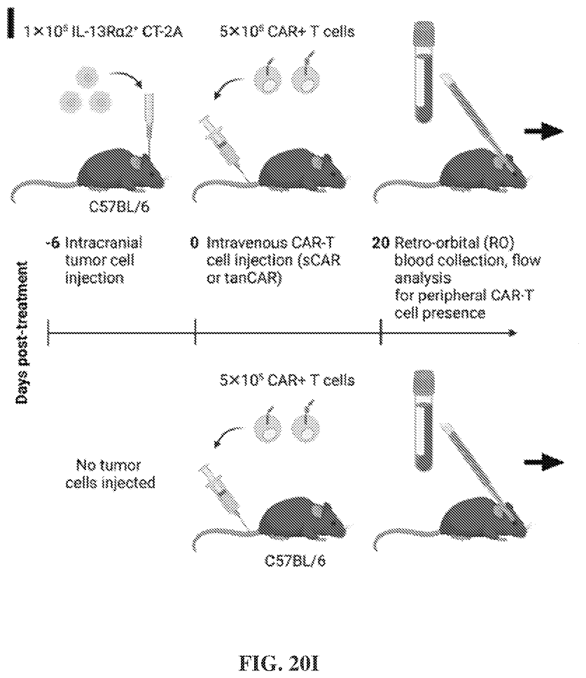

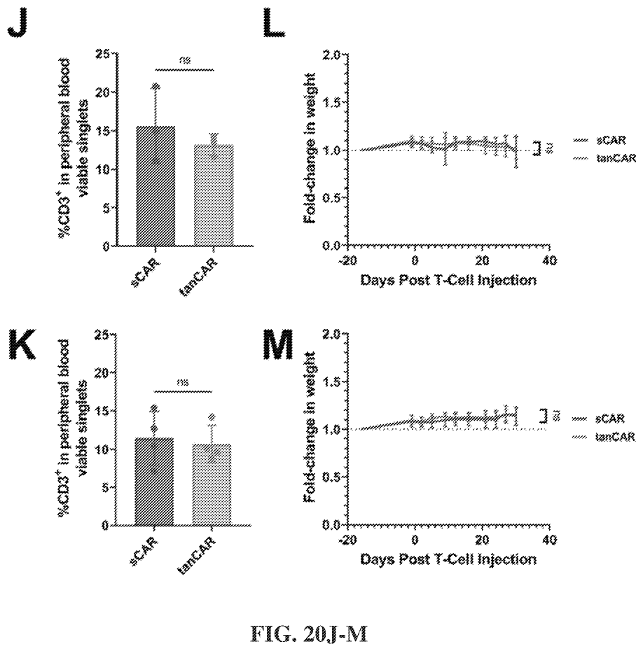

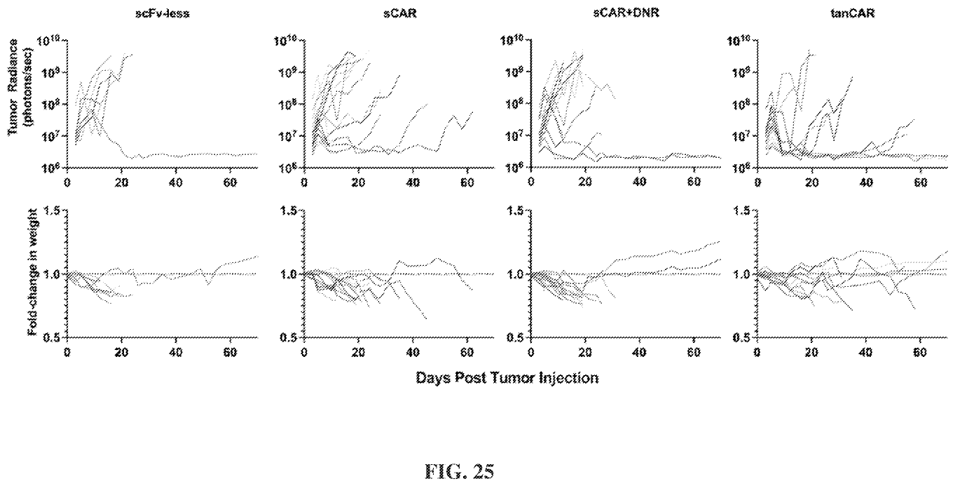

Given reports of toxicity induced by systemic inhibition of TGF-β (41,42), the inventors sought to ensure that TGF-β-responsive CAR-T cells can be well-tolerated. As shown in both NSG and C57BL/6 mouse models (FIGS. 16G, 2H, 5B), GBM-bearing animals treated with IL-13Rα2/TGF-β tanCAR-T cells survived for months unless they succumbed to tumor progression, suggesting lack of treatment-related toxicity. Indeed, after intratumoral injection in both NSG and C57BL/6 mice bearing orthotopic GBM xenografts FIG. 20A), tanCAR-T cells showed no detectable presence above background in the peripheral blood two weeks after treatment (FIGS. 20B, C). Furthermore, no evidence of toxicity was observed based on animal behavior or post-mortem dissection, and mouse weights remained stable over time (FIGS. 20D, E). Specifically, all instances of weight reduction showed temporal correlation with tumor outgrowth (FIG. 25), suggesting changes in weight were due to tumor burden rather than CAR-T cell administration. Together, these results demonstrate that locally administered tanCAR-T cells show no evidence of systemic toxicity.

CAR-T cells were intracranially injected in all experiments performed in this study, a choice that was informed by preclinical and clinical evidence indicating direct CAR-T cell infusion into the central nervous system (CNS) is safe and potentially more efficacious compared to peripheral infusion 11,14,54,77). To investigate whether TGF-β-responsive CAR-T cells would exhibit greater toxicity risk when administered systemically, an intravenous dose of single-input TGF-β CAR-T cells was given via tail-vein injection to healthy immunocompetent C57BL/6 mice (FIG. 20F). No significant expansion of circulating T cells was observed 20 days after T-cell administration (FIG. 20G), and mouse weights remained stable through one month following T-cell injection (FIG. 20H). Moreover, no evidence of gross toxicity was observed upon histological analysis of liver, spleen, and kidney samples (Table 2). To confirm the tanCAR configuration was also safe, an intravenous dose of murine tanCAR- or sCAR-T cells was given via tail-vein injection to either tumor-bearing or healthy C57BL/6 mice (FIG. 20I). Again, there was no significant systemic expansion of circulating T cells 20 days after treatment (FIGS. 20J, K), and mouse weights remained stable over time (FIGS. 20L, M). Taken together, TGF-β-targeting CAR-T cells with or without simultaneous IL-13Rα2 recognition capability show no detectable evidence of toxicity, whether administered directly into the CNS or systemically.

### C. Discussion

Immunotherapies against GBM are limited by both the highly immunosuppressive TME and tumor heterogeneity. Indeed, CAR-T cells, peptide vaccines, and DC vaccines administered as monotherapies have largely been ineffective in clinical trials, and development of multi-pronged approaches to counter tumor-mediated immune inhibition is an area of active investigation (78). Previous efforts in CAR-T cell development to address GBM antigen heterogeneity have relied on hard-coding CAR specificities against two or three target antigens present on the surface of GBM cells (79-81). However, such dual- or triple-targeting therapies may still be susceptible to antigen escape by highly heterogeneous GBM tumors and do not directly address the immunosuppressive TME. Robust efficacy against GBM will likely require the engagement of endogenous immunity to drive antigen spreading—the phenomenon whereby immune recognition of tumors is extended beyond the antigen targeted by the therapeutic agent. Others have demonstrated that IL-13Rα2 CAR-T cells can induce endogenous anti-tumor immunity in syngeneic murine glioma models (82,83), although a similar phenomenon has not yet been definitively demonstrated in human patients. Nevertheless, these results suggest the potential to augment the efficacy of IL-13Rα2-targeting CAR-T cells through further engineering focused on overcoming immunosuppression and promoting endogenous immunity.

Here, the inventors engineer bispecific IL-13Rα2/TGF-β CARs, or tanCAR for brevity, which compactly integrate tumor-targeting and immuno-modulatory moieties. To the inventors' knowledge, this is the first single-chain bispecific CAR that can simultaneously target a membrane-bound tumor-associated antigen and convert a soluble factor in the TME into an activating signal for CAR-T cells. In the in vivo studies, which used low CAR-T cell doses in the absence of irradiation or lymphodepletion, single-input IL-13Rα2 CAR (sCAR)-T cells conferred limited control over tumor outgrowth in both NSG and C57BL/6 mice, mimicking the low clinical response rates to GBM-targeting CAR-T cells. In such treatment-refractory murine GBM models, tanCAR-T cells significantly outperformed sCAR-T cells against both patient-derived GBM xenograft and syngeneic GBM tumor. By converting TGF-β from a critical immunosuppressive factor into an immunostimulant, tanCAR-T cells not only protect and activate themselves, but also modify the TME into one that is more conducive to endogenous immune response against GBM. Specifically, treatment with tanCAR-T cells reduces the presence of suppressive cell types including MDSCs and Tregs, while increasing native T-cell infiltration, activation, and cytotoxicity.

The TGF-β DNR is a previously established strategy aimed at countering the inhibitory effects of TGF-β on T cells. Both the DNR and the tanCAR can sequester TGF-β and reduce the effective concentration of TGF-β in the microenvironment, but only the tanCAR can activate engineered T cells. Interestingly, sCAR and sCAR+DNR T cells were similarly ineffective in controlling human GBM xenograft models, resulting in significantly inferior survival compared to tanCAR-T cells. And yet, sCAR+DNR T cells exhibited statistically comparable anti-tumor efficacy as tanCAR-T cells in the syngeneic CT-2A glioma model. In the context of human xenografts in immunodeficient mice, the primary factor determining anti-tumor efficacy is the interaction between adoptively transferred T cells and tumor cells. In such a system, the tanCAR's unique ability to not only block endogenous TGF-β signaling but also activate the engineered T cell may have resulted in the clear advantage over both sCAR and SCAR+DNR. Furthermore, the TGF-β production level in the human GS001 tumor (FIG. 15C) is substantially higher than that in the murine CT-2A tumor (Data not shown). Given that the CAR's response to TGF-β is dose-dependent (FIGS. 22B, C) (43), the higher TGF-β level in the human xenograft may have also contributed to the tanCAR's superior performance in this model.

The present work examines the impact of tanCAR-T cells in vivo in both patient-derived xenograft and immunocompetent mouse models, leveraging high-dimensional experimental techniques for deep characterization of tanCAR-T cells and their impact on the TME at both proteomic (CyTOF, FIGS. 17E-J) and transcriptomic (scRNA-seq. FIG. 18) levels.

In the context of syngeneic tumors in immunocompetent mice, there are numerous endogenous immune components that can interact with tumor cells and CAR-T cells, as well as produce TGF-β and/or be influenced by TGF-β sequestration. In such a system, the DNR's ability to sequester TGF-β can, in principle, positively influence the TME and enhance the DNR's performance in the immunocompetent mice relative to the NSG mouse model.

However, the DNR's impact on the TME was not clearly detected by CyTOF and scRNA-seq analyses, as sCAR and sCAR+DNR samples yielded similar profiles. In fact, by some measures—e.g., frequencies of CD8⁺ T cells (FIG. 17G), peripheral macrophages (FIG. 18C), and Tregs (FIG. 18F)—treatment with sCAR+DNR T cells may have yielded a slightly more immunosuppressive TME compared to treatment with sCAR-T cells.

The inability to detect obvious effects on the TME associated with DNR treatment may be linked to the nearly bimodal pattern in tumor control among mice treated with SCAR+DNR T cells—i.e., over 40% of mice exhibited rapid tumor progression and early death, while another 25% of mice achieved complete tumor eradication and long-term survival (FIG. 19B, C). Heterogeneous responses to immunotherapy in syngeneic mouse models of glioma, such as the mix of early progression and durable tumor clearance observed here, have previously been reported (84). Although CT-2A has been described as an immunogenically silent tumor model (63), the CT-2A cells used in this work transgenically expressed human IL-13Rα2 (Data not shown) and exhibited a certain level of immunogenicity, as evidenced by occasional clearance of established tumor even in the negative-control scFv-less CAR-T cell group (FIG. 19C). In this complex immunocompetent GBM TME, treatment with sCAR+DNR T cells may be at the cusp of meaningful therapeutic benefit, resulting in the bimodal response observed in this study.

Despite the lack of statistically significant survival difference between tanCAR and sCAR+DNR treatment groups in the CT-2A model, scRNA-seq analysis revealed increased presence of suppressive myeloid cells in mice treated with sCAR+DNR T cells compared to not only tanCAR-T cells but also sCAR-T cells. This observation is consistent with prior reports of upregulation of suppressive, myeloid-associated molecules in patients with metastatic castration-resistant prostate cancer treated with PSMA CAR-T cells co-expressing the TGF-β DNR (47). Notably, the high-dimensional analyses (FIG. 17E-J, 4) uncovered mechanisms by which tanCAR-T cells favorably impact the GMB TME—namely, via downregulation of the immunosuppressive PD-1 signaling axis in T cells and TGF-β/LGALS signaling axes in tumor-infiltrating macrophages. Taken together, the inventors show that remodeling of the TME to potentiate anti-tumor immunity can be more effectively achieved by not only inhibiting, but also converting immunosuppressive TGF-β signaling into a T-cell stimulant.

One advantage of the IL-13Rα2/TGF-β tanCAR is its cross-reactivity with both human and murine TGF-β, which enabled the use of the same CAR construct in both xenograft and syngeneic tumor models; thus, CAR protein sequence is not a confounding factor in the interpretation of the results in syngeneic models. Nevertheless, the in vivo studies have some limitations. In xenograft models using patient-derived GBM neurospheres, the inventors observed that tumor burden did not correlate with bioluminescent signal. In fact, brains recovered from a handful of mice which died without detectable tumor radiance exhibited abnormal features upon gross examination, and IHC staining revealed strong IL-13Rα2 expression, confirming tumor outgrowth to be the cause of death. Therefore, the main criterion for evaluating CAR-T cell efficacy against xenograft tumors was survival outcome, where tanCAR-T cells significantly increased survival in mice compared to sCAR-T cells alone or co-expressing the TGF-β DNR. The in vivo studies also utilized tumor cells that were highly homogeneous in IL-13Rα2 expression, which does not capture the antigen heterogeneity typical of GBM tumors. Future studies will incorporate tumor models with heterogeneous antigen expression to stringently evaluate whether antigen spreading can be induced by tanCAR-T cells, either administered alone or in combination with other forms of immunotherapy. Potential combination regimens may include radiotherapy and PD-1/PD-L1 checkpoint blockade, which have both been shown to synergize with TGF-β antagonists (85-89). Finally, the studies performed in immunocompetent mice enabled the interrogation of immunosuppressive macrophages, which have been shown to impair clinical response of GBM to immunotherapy (64,84,90,91). However, given differences in murine and human immunology, studies with either ex vivo human GBM specimens or humanized mouse models could provide useful validation of the interactions between tanCAR-T cells and immune cells in the TME.

Besides assessing anti-tumor efficacy, the inventors also sought to evaluate the safety of bispecific tanCAR-T cells. In both immunodeficient and immunocompetent murine hosts, there was no observable toxicity resulting from either intracranial or intravenous administration of TGF-β-targeting CAR-T cells. Nevertheless, approaches involving modulation of T-cell responses to TGF-β warrant caution, particularly in light of recently observed fatalities in patients treated with PSMA CAR-T cells co-expressing the TGF-β DNR (47,92). The cause of fatality in these trials is still unresolved, but no definitive evidence links TGF-β DNR expression to the toxicity observed (47). Moreover, previous clinical trials have demonstrated that virus-specific T cells expressing a TGF-β DNR are well-tolerated in patients through a four-year monitoring period (45), whereas patient death has previously been observed following PSMA CAR-T cell treatment (93,94). Taken together, evidence suggests rewiring T-cell response to TGF-β can be a safe and effective approach to increasing CAR-T cell function. Nevertheless, the true safety profile of a novel therapy can only be determined in a clinical setting, and the incorporation of suicide genes (95-98) or high-dose steroid administration, which is common clinical practice used to control cerebral edema in GBM patients (14,99), should still be contemplated as safeguards against unanticipated toxicity.

In addition to implementing measures to ensure patient safety, locoregional delivery of tanCAR-T cells in GBM patients should further minimize the risk of unanticipated adverse events. In clinical trials, IL-13Rα2-targeting CAR-T cells are well-tolerated when administered intraventricularly or into the resection cavity (11,18,19). More recently, intraventricular administration of GD2 CAR-T cells in diffuse intrinsic pontine and diffuse midline glioma patients exhibited less evidence of systemic toxicities such as cytokine release syndrome compared to intravenous CAR-T cell administration (14). Taken together with these clinical observations, the study findings provide support for the clinical translation of bispecific IL-13Rα2/TGF-β CAR-T cells as a safe novel therapy to effectively combat the immunosuppressive TME in GBM.

### D. Methods

DNA constructs: Single-chain bispecific and CARs were constructed by isothermal assembly of DNA fragments (100). The IL-13Rα2-binding domain was encoded by an IL-13 mutein as reported by Debinski et al. (52,53), while the TGF-β-binding domain was encoded by a previously described scFv sequence⁴³. The IL-13Rα2/TGF-β CAR encoded an IgG4 hinge extracellular spacer, a CD28 transmembrane domain, 4-1BB co-stimulatory domain, and CD35 signaling chain. The single-input IL-13Rα2 CAR encoded an IgG4 hinge-CH2-CH3 long extracellular spacer bearing previously described L235E and N297Q mutations (101), a CD28 transmembrane domain, 4-1BB co-stimulatory domain, and CD35 signaling domain (54). In some experiments, CARs were co-expressed with a truncated epidermal growth factor receptor (EGFRt), which served as a transduction marker (102), via a “self-cleaving” T2A peptide. N-terminal FLAG tags were also encoded in order to assess surface receptor expression levels. The TGF-β DNR, which encodes the first 199 amino acids of TGFBR2, was co-expressed with the single-input IL-13Rα2 CAR via a self-cleaving P2A peptide. Human IL-13Rα2 was cloned into a lentiviral vector backbone and co-expressed with ffLuc via a self-cleaving T2A peptide.

Cell lines: GS001 neurospheres were derived from the patient-derived PBT106 neurosphere line generated from discard tumor material from a patient with recurrent GBM (103). PBT106 neurospheres stably expressing EGFP and ffLuc were sorted by consecutive rounds of magnetism-activated cell sorting (MACS; Miltenyi Biotec) followed by fluorescence-activated cell sorting (FACS) of IL-13Rα2+ cells to obtain GS001. FACS sorting was performed on a BD FACSAria II at the UCLA Flow Cytometry Core Facility. Intraoperative samples from both recurrent and newly diagnosed GBM patients obtained at UCLA were used to establish a panel of neurosphere lines (GS270, GS121, GS181, GS304, GS013, GS054). All neurosphere lines were maintained in DMEM/F12 media with 15 mM HEPES, 1× serum-free B27 (Gibco), 5 g/mL heparin (STEMCELL Technologies), and 1× GlutaMax (Gibco). Cultures were supplemented with 20 ng/ml epidermal growth factor (PeproTech) and 20 ng/mL basic fibroblast growth factor (PeproTech) every 3-4 days. For in vitro and in vivo experiments, neurospheres were dissociated into single-cell suspensions with accutase or TrypLE. CT-2A murine glioma cells were lentivirally transduced to express human IL-13Rα2 and ffLuc, and subsequently FACS-sorted for IL-13Rα2+ cells on a BD FACSAria II at the UCLA Flow Cytometry Core Facility. CT-2A cells were maintained in DMEM+10% heat-inactivated fetal bovine serum (HI-FBS). HEK 293T cells were obtained from ATCC. Phoenix-Eco cells were a generous gift from Dr. Antoni Ribas (UCLA).

Retrovirus production: Retroviral supernatants for human T-cell transduction were produced by co-transfection of HEK 293T cells with plasmids encoding CAR constructs and pRD114/pHIT60 virus-packaging plasmids (gifts from Dr. Steven Feldman, National Cancer Institute), using linear polyethylenimine (PEI, 25 kDa; Polysciences). Supernatant was collected 48 hours after transfection, and cell debris removed using a 0.45 μm membrane filter.

Retroviral supernatants for murine T-cell transduction were produced by co-transfection of Phoenix-Eco cells with plasmids encoding CAR constructs and the pCL-Eco packaging plasmid, using linear PEI. Supernatant was collected 48 hours after transfection, and cell debris removed using a 0.45 μm membrane filter.

Primary human T-cell culture: T cells were isolated from healthy donor whole-blood obtained from the UCLA Blood and Platelet Center. CD3⁺ T cells were isolated using the RosetteSep CD3⁺ T Cell Enrichment kit (STEMCELL Technologies) following the manufacturer's protocol. In some experiments, naïve/memory T cells were isolated as previously described (104). Isolated T cells were activated with CD3/CD28 Dynabeads (Gibco) at a 1:3 bead:cell ratio, and two rounds of retroviral transduction were performed at 48 hours and 72 hours following activation. T cells were maintained in complete RPMI (RPMI-1640+10% HI-FBS) and cultures were supplemented with 50 U/mL IL-2 and 1 ng/ml IL-15 every 2-3 days. Dynabeads were removed on day 7. All downstream assays were performed between day 9 and day 15 of culture.

Murine T-cell culture: Spleens were harvested from healthy, six- to eight-week-old C57BL/6J mice. Single-cell suspensions were obtained by gentle maceration in 70-μm cell strainers placed over 50 mL Falcon tubes. CD3⁺ T cells were enriched from bulk splenocytes using the Pan T Cell Isolation kit II, mouse (Miltenyi Biotec) following the manufacturer's protocol. Isolated murine T cells were activated with anti-mouse CD3/CD28 Dynabeads (Gibco) at a 1:1 bead-to-cell ratio. One day prior to transduction, 12-well non-TC-treated plates were coated overnight with 15 μg/mL RetroNectin (Takara) diluted in PBS at 4° C. 24 hours following T-cell activation, retroviral supernatant was added to RetroNectin-coated plates and centrifuged at 2000×g for 2 hours (no brakes). Activated T cells were subsequently applied to spinoculated plates and centrifuged at 2000×g for 15 minutes (no brakes). T cells were maintained in RPMI-1640+10% HI-FBS+50 μM β-mercaptoethanol. Cell cultures were supplemented with 50 U/mL human IL-2 every 2-3 days. Dynabeads were removed on day 5 of cell culture, and cells were used for in vitro and in vivo experiments.

In vitro killing and proliferation assays: Cytotoxic killing of tumor cells was assessed using the xCELLigence Real-Time Cell Analyzer System (Agilent Technologies). 96-well E-Plates® were coated with mouse laminin (Corning) prior to the addition of target cells. Target neurosphere lines were plated on day 0 (2×10⁴ cells/well) in 100 μL of complete RPMI medium. After overnight tumor-cell adherence to the well bottom, CAR-T cells were added at effector:target (E:T) ratios of 3:1 and 1:1 in complete RPMI to a final volume of 200 μL. Equal numbers of CAR+ and total T cells were plated for each construct, adding untransduced cells as necessary to normalize for differences in transduction efficiency. Maximal cell release was obtained by adding 1% Triton X-100 to the wells. Cell index values (relative cell impedance) were collected over 72 hours and normalized to the maximal cell index value after addition of T cells. The percentage lysis was calculated as a proportion of the normalized cell index at a time point of interest versus the normalized cell index after effector cell plating.

To measure murine T-cell proliferation and cytotoxicity, CAR-T cells were labeled with CellTrace Yellow (CTY; ThermoFisher Scientific). 2.5×10⁴ IL-13Rα2+CT-2A glioma cells were seeded in each well of a 96-well flat-bottom plate, and co-incubated with labeled T cells at specified effector:target (E:T) ratios, where the number of effectors was determined by CAR-positive T cell counts. To account for differences in transduction efficiency across CAR constructs, untransduced cells were added as necessary to achieve equal numbers of total T cells. After 4 days, cells were harvested as previously described (105). T-cell counts, tumor-cell counts, and CTY dilution were assessed by flow cytometry using a MACSQuant VYB.

T-cell activation marker upregulation: 1×10⁵ human or murine T cells were seeded in 96-well flat-bottom tissue culture plates in 100 μL complete RPMI with or without recombinant human or murine TGF-β1 (PeproTech). Following overnight culture at 37° C., cells were transferred to a 96-well U-bottom plate and activation marker expression was assessed by antibody staining and flow cytometry.

Phospho-SMAD Western blotting: To reduce background SMAD phosphorylation, primary human T cells were cultured overnight in serum-free, CTS OpTmizer media. Following overnight culture, cells were incubated at 37° C. with or without 5 ng/mL recombinant TGF-β1 (Peprotech) for 1 hour, then washed in PBS prior to cell lysis. Cell lysis and phospho-SMAD immunoblotting was performed as previously described (43).

ELISA: Cell culture supernatants were collected 24 hours after seeding 2×10⁵ cells in 24-well plates. TGF-β concentrations in supernatant were determined using Human or Murine TGF-1 DuoSet ELISA kits (R&D Systems) or ELISA MAX™ Deluxe Sets (BioLegend) following the manufacturer's protocols.

In vivo studies: All in vivo experiments were approved by the UCLA Animal Research Committee (ARC). NSG and C57BL/6J mice were purchased from UCLA Department of Radiation and Oncology. For GBM xenografts, 2.5×10⁵ GS001 neurospheres were stereotactically implanted into the right forebrains (1.5 mm lateral, 0.5 mm anterior to the bregma) of six- to eight-week-old NSG mice. 5×10⁵ CAR-T cells were administered intratumorally seven days following tumor implantation. For syngeneic CT-2A glioma studies, 1×10⁵ cells were stereotactically implanted into the right forebrains of six- to eight-week-old C57BL/6J mice. 5×10⁵ CAR-T cells were administered intratumorally six days following tumor implantation. In all in vivo studies, untransduced cells were added as necessary to normalize percent CAR positivity across all CAR constructs, such that each treatment group received the same number of total T cells as well as the same number of CAR⁺ T cells. Figures shown for survival studies with the CT-2A model contain the combined data from two independent experiments; each experiment included n=12 for each of the three CAR-treated groups, and n=6 or 7 for the scFv-less control group. The physical limitation on how many brain surgeries can be performed in one sitting necessitated the need to split the study into two halves.

Tumor burden was monitored by bioluminescent imaging. Mice were injected subcutaneously with 3 mg D-luciferin and imaged on an IVIS Lumina III LT Imaging System (Perkin Elmer). Photon flux was analyzed with LivingImage Software (Perkin Elmer). All studies were blinded, and animals were euthanized at the humane endpoint.

Antibody staining for flow cytometry: EGFRt expression was measured by staining with biotinylated cetuximab (Eli Lilly; biotinylated in-house), followed by PE-conjugated streptavidin (Jackson ImmunoResearch catalog no. 016-110-084). CAR expression on the cell-surface was measured by staining with anti-DYKDDDDK (SEQ ID NO:3-FLAG) tag conjugated to APC or PE/Cy7 (clone L5, BioLegend catalog nos. 637308 or 637324, respectively).

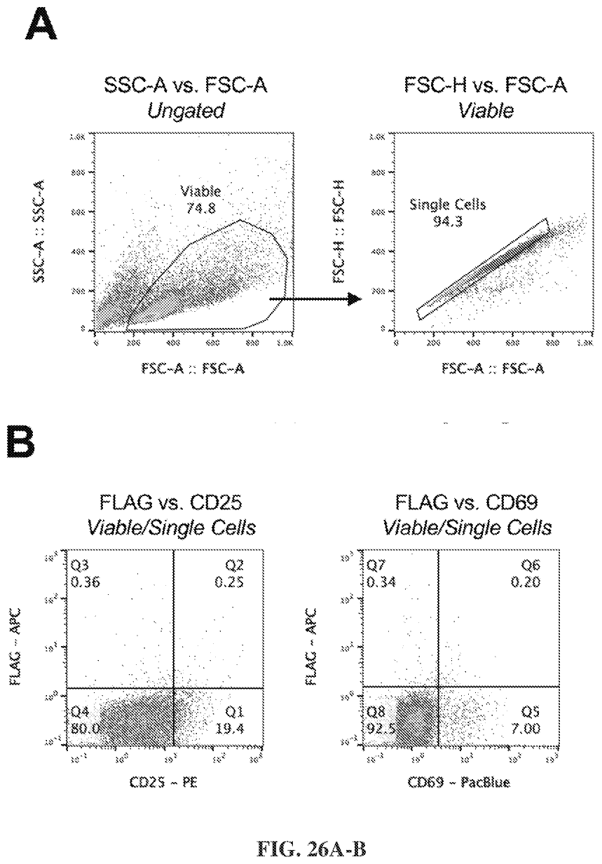

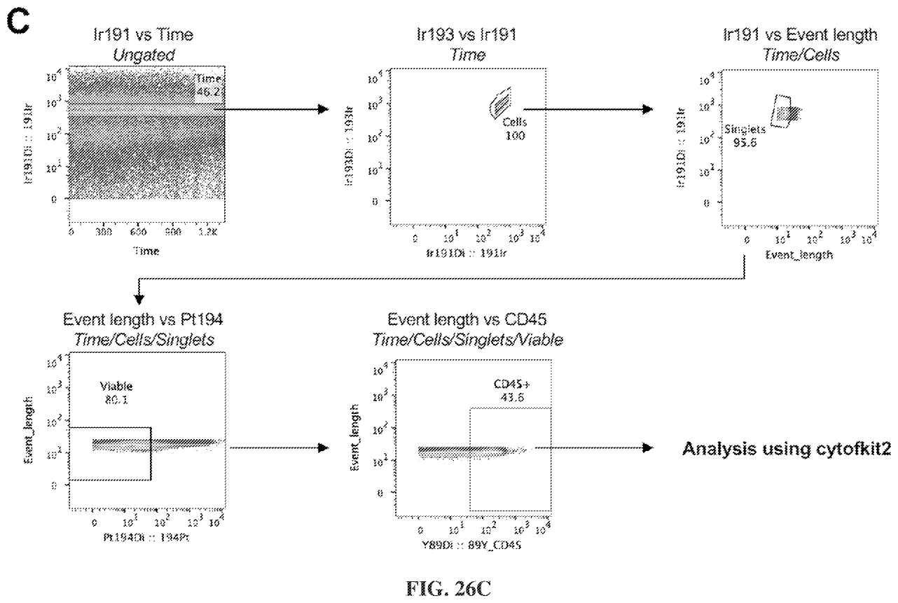

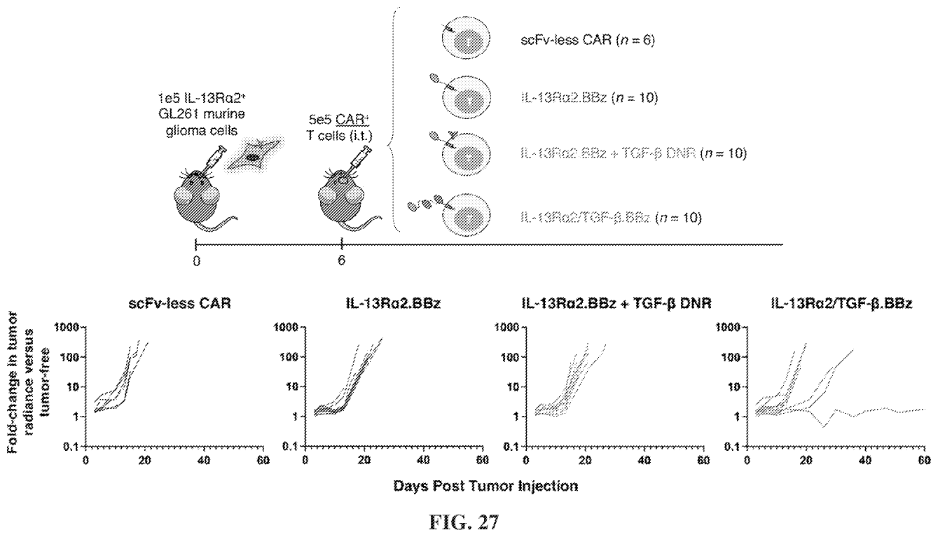

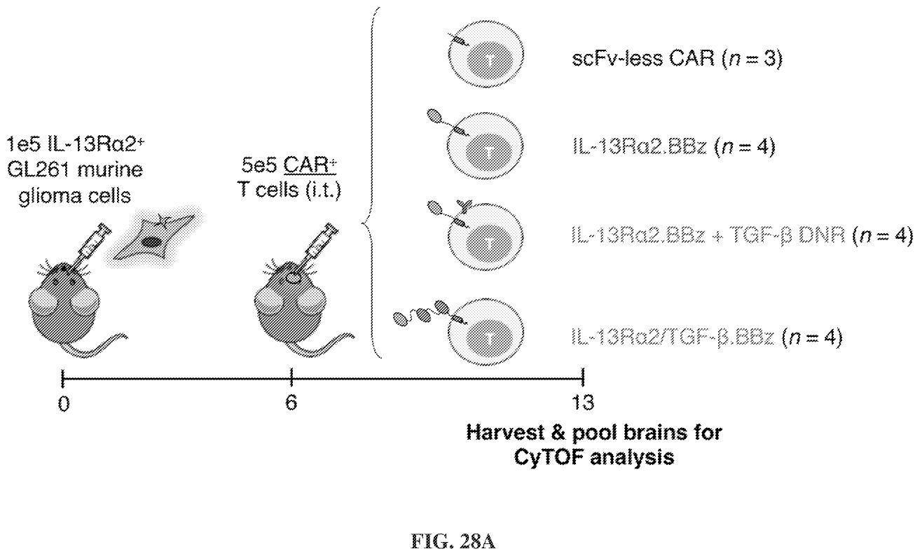

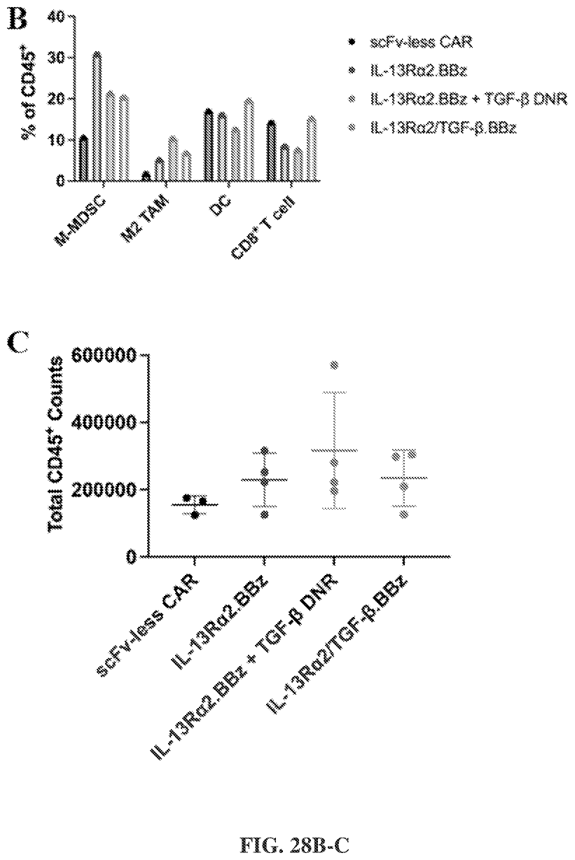

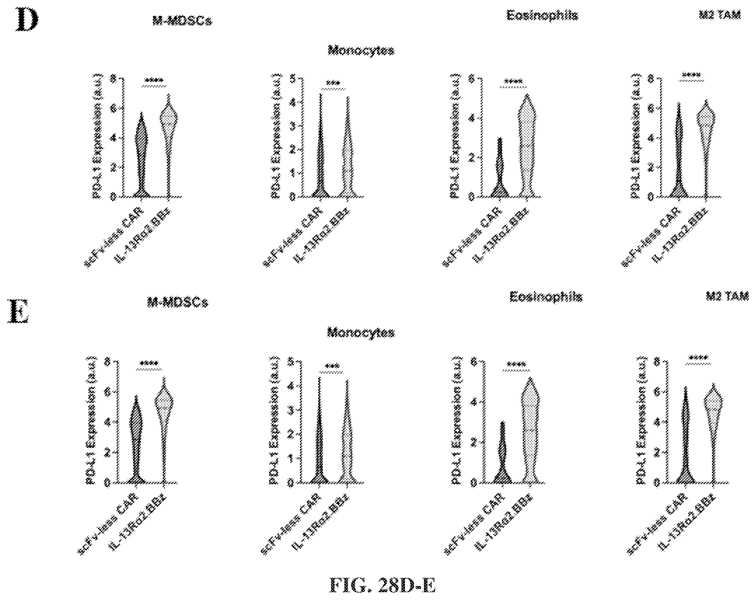

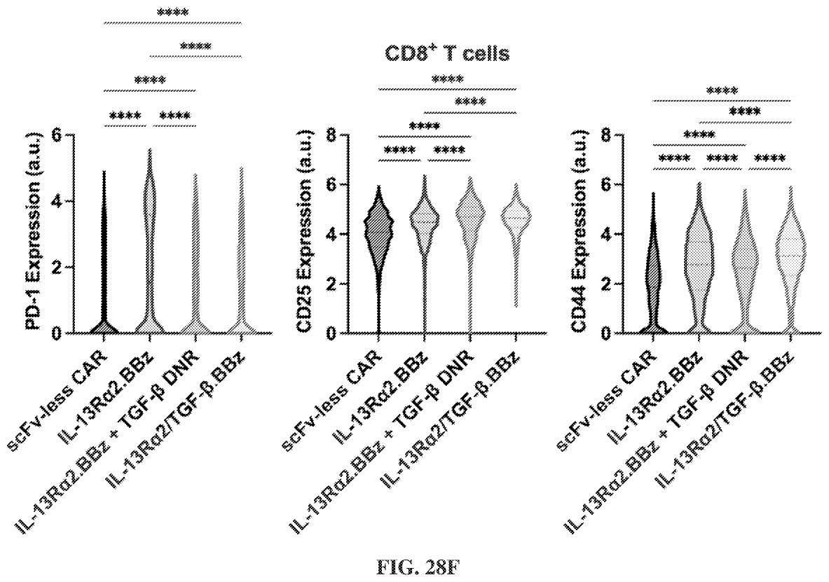

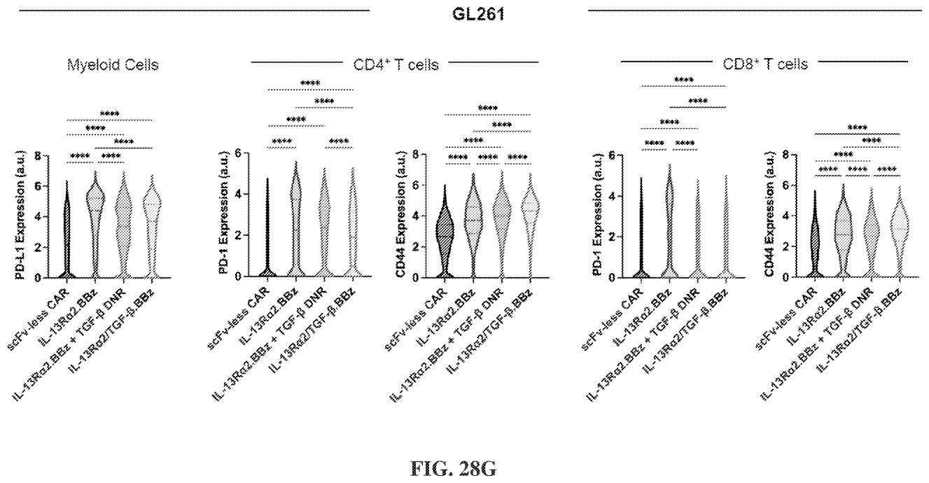

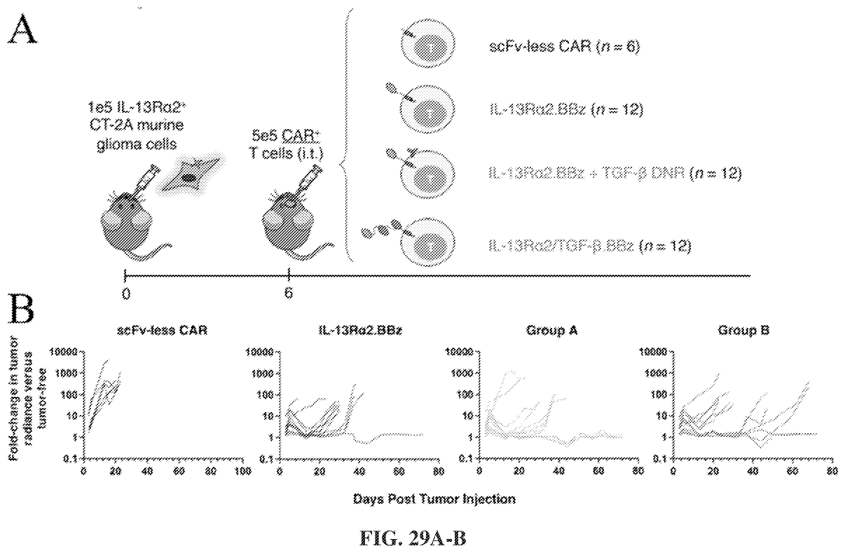

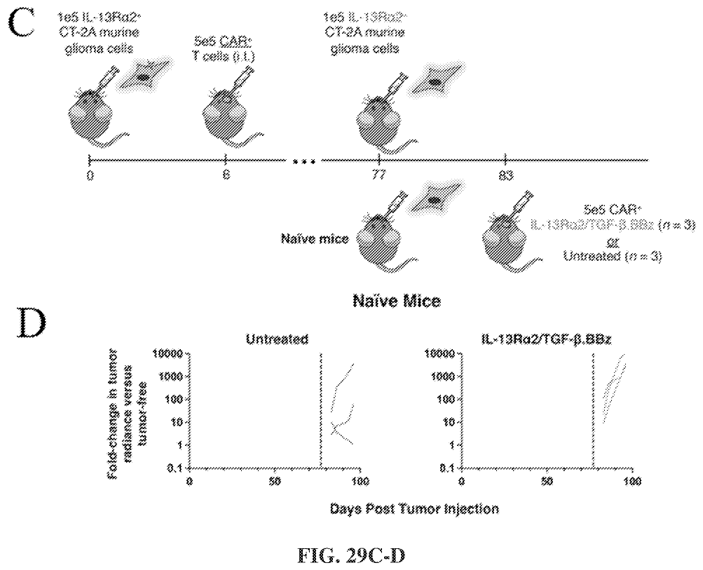

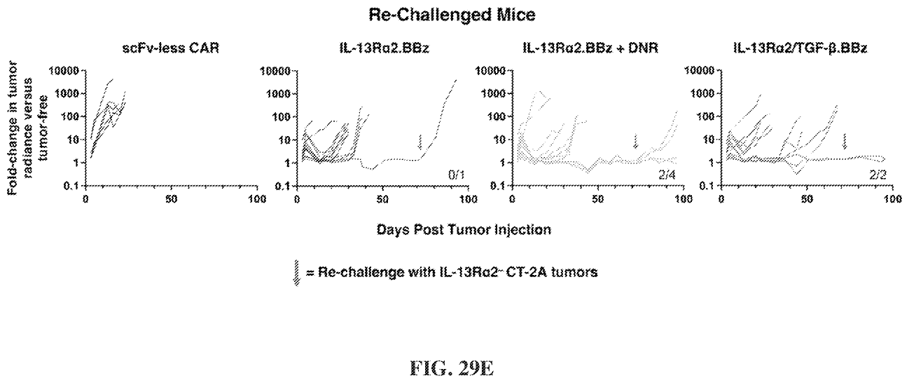

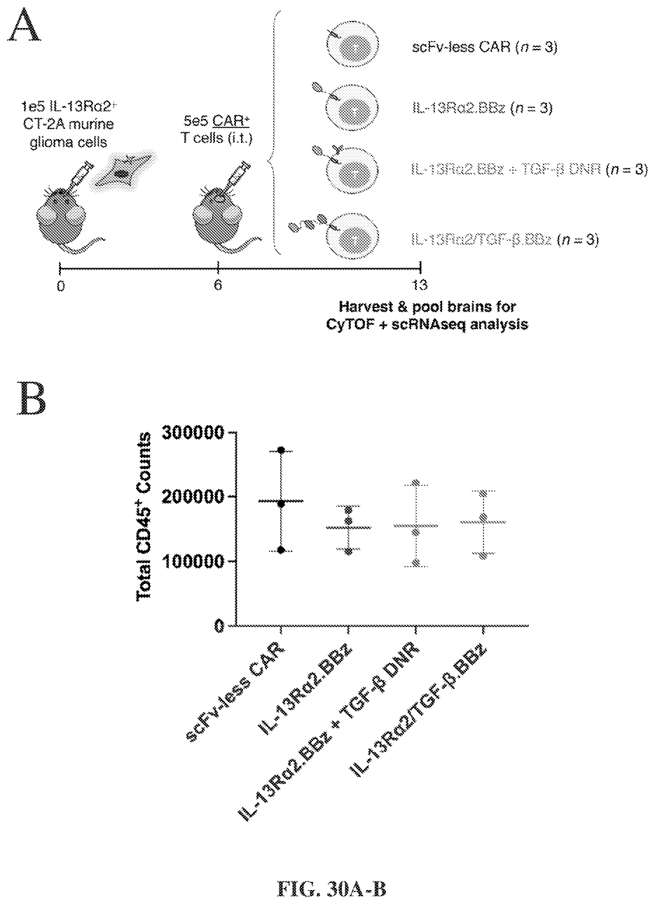

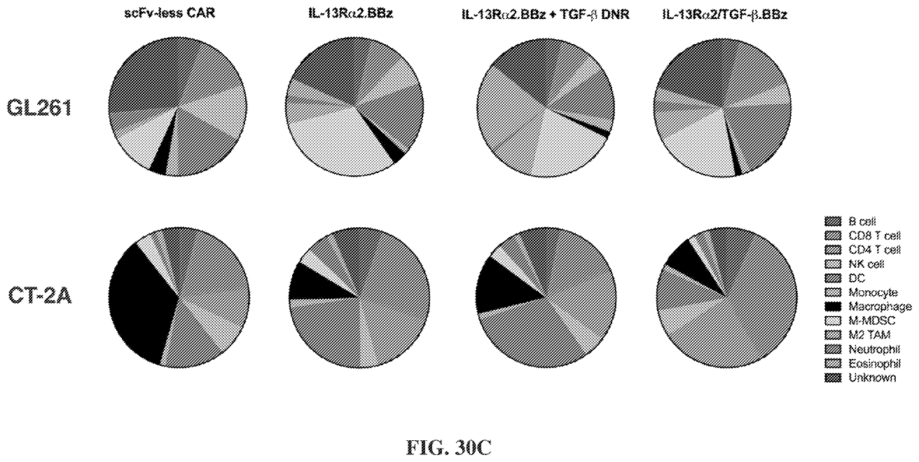

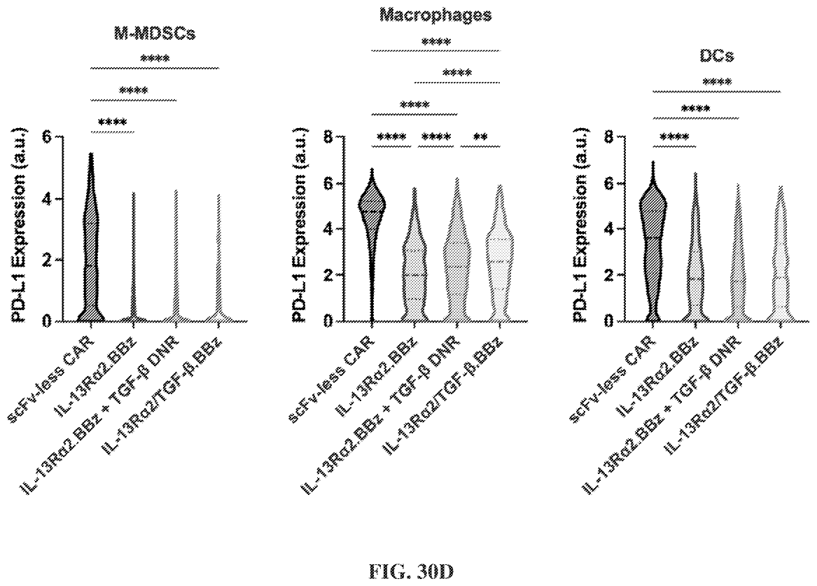

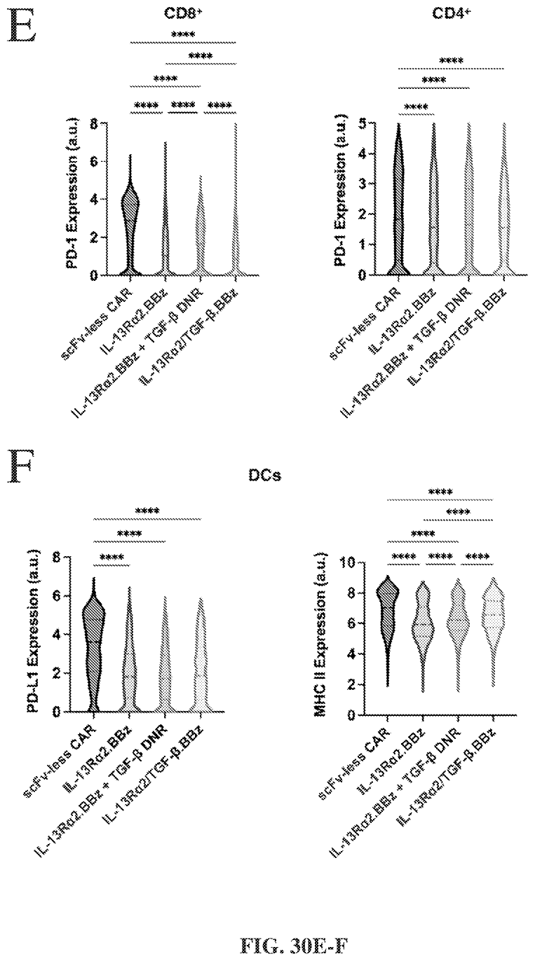

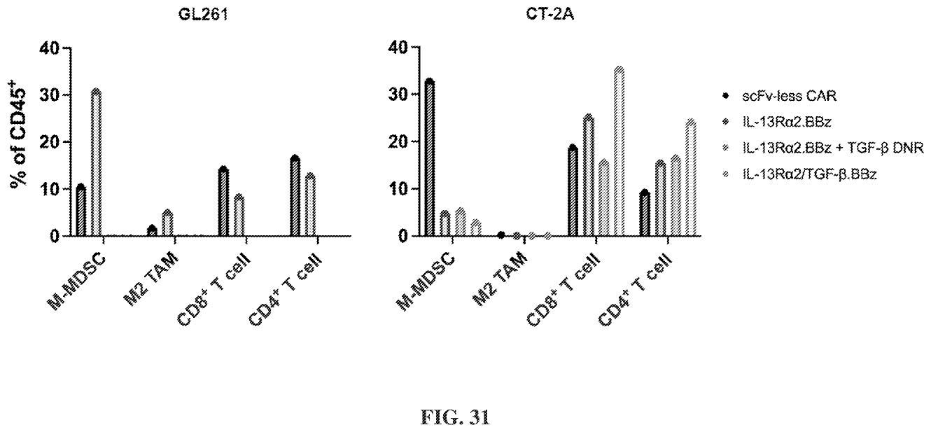

Activation marker upregulation in the presence of TGF-β was performed by staining human T cells with PE-conjugated anti-human CD25 (clone BC96, BioLegend catalog no. 302606), PacificBlue-conjugated anti-CD69 (clone FN50, BioLegend catalog no. 310920), and APC-conjugated anti-FLAG tag. Activation marker upregulation in murine T cells was assessed by staining with APC-conjugated anti-mouse CD25 (clone PC61, BioLegend catalog no. 102012), PacificBlue-conjugated anti-mouse CD69 (clone H1.2F3, BioLegend catalog no. 104524), and PE/Cy7-conjugated anti-FLAG tag. An exemplary gating path for flow cytometry analysis is shown in FIGS. 26A-B.

To monitor for expansion of adoptively transferred T cells in NSG mice, retro-orbital blood samples were treated with 1× Red Blood Cell Lysis Solution (Miltenyi Biotec), then stained with PacificBlue-conjugated anti-human CD45 (clone HI30, BioLegend catalog no. 304029) and APC-conjugated anti-human CD3 (clone UCHT1, BioLegend catalog no. 300412). Peripheral blood samples collected from C57BL/6 mice were stained with stained with PE/Cy7-conjugated anti-mouse CD3 (clone 17A2, BioLegend catalog no. 100220), PacificBlue-conjugated anti-mouse CD45 (clone S18009F. BioLegend catalog no. 157212), and APC-conjugated anti-FLAG tag following red blood cell lysis.

Flow cytometry data were acquired by a MACSQuant VYB (Miltenyi Biotec). For all experiments, cells were stained, washed, and re-suspended in PBS+2% HI-FBS. Data were analyzed and gated using FlowJo software (TreeStar).

Sample preparation for scRNAseq: Brain tumor samples were harvested from C57BL/6J mice, and single-cell suspensions obtained by gentle mechanical dissociation using the gentleMACS dissociator (Miltenyi Biotec), Multi Tissue Dissociation kit I (Miltenyi Biotec), Debris Removal Solution (Miltenyi Biotec), and Red Blood Cell Lysis Solution (Miltenyi Biotec) following the manufacturer's protocol for dissociation of inflamed neural tissue. High-viability samples were obtained using Miltenyi Dead Cell Removal kit, and cells re-suspended in PBS+0.04% BSA. Single-cell RNA sequencing libraries were prepared using a 10× Genomics Chromium Controller, and libraries were sequenced on the Illumina NovaSeq S1 platform with 50-bp paired-end reads at the UCLA Technology Center for Genomics & Bioinformatics (TCGB).

scRNAseq analysis: Before analysis, the mm10 murine reference genome was modified to include sequences for EGFP-ffLuc-huIL-13Rα2 (tumor marker) as well as the four different treatment constructs evaluated (scFv-less, sCAR, sCAR+DNR, tanCAR). Sequencing libraries were read using the Read10× function from 10× Genomics Cell Ranger 7.0.0 and aligned to the modified murine reference genome. Analysis in R v4.2.0 was conducted with the Seurat package v4.1.1 (106) and guided by Seurat tutorials and vignettes. Data sets were converted into Seurat objects and filtered based on total RNA, unique transcript count, and mitochondrial content to exclude non-viable cells and artifacts. Data for each treatment group were integrated, clustered using default parameters (resolution=0.5), and visualized via uniform manifold and approximation projection (UMAP). Differentially expressed features defining each cluster were interrogated, and curated markers for known cell populations were examined by dot plot. Clusters 1, 6, 12, 13, 19, and 25 (containing CD4⁺, CD8⁺, and γδ T cells) were then re-clustered using an identical process to uncover T cell subtypes and phenotypic markers. This re-clustered scheme contained 13 clusters, two of which were found to have dual expression of T cell and microglial markers. These clusters were discarded and the remaining 11 clusters were re-clustered. Curated and differentially expressed features (sorted by log 2 (fold change)) among clusters were subsequently visualized by dot plot and heatmap, respectively.

CyTOF: Brain tumor samples were harvested from C57BL/6J mice, and single-cell suspensions obtained by gentle mechanical dissociation using the gentleMACS dissociator and Multi Tissue Dissociation kit I (Miltenyi Biotec), Debris Removal Solution (Miltenyi Biotec), and Red Blood Cell Lysis Solution (Miltenyi Biotec) following the manufacturer's protocol for dissociation of inflamed neural tissue. Single-cell suspensions were incubated with 2.5 μM monoisotopic cisplatin-194 Pt (Fluidigm) at room temperature for 5 minutes, then washed with Maxpar Cell Staining Buffer (MCSB, Fluidigm). Cells were subsequently incubated with FcR blocking reagent, mouse (Miltenyi) at room temperature for 10 minutes, then incubated with surface marker antibody cocktail at room temperature for 30 minutes. Cells were washed with MCSB, then fixed with 4% paraformaldehyde by incubation at room temperature for 10 minutes. Cells were washed with Perm-S buffer (Fluidigm) to permeabilize, and incubated with intracellular antibody cocktail at room temperature for 1 hour. Cells were washed and then incubated overnight at 4° C. with 200 nM iridium intercalating reagent (Fluidigm) diluted in Maxpar Fix and Perm buffer (Fluidigm). Following overnight incubation, cells were washed twice with MCSB, and twice more with deionized water. Cells were re-suspended in deionized water and run on a Fluidigm Helios mass cytometer (UCLA Flow Cytometry Core Facility). Cells were stained with a previously described mouse immune cell marker panel (107). Acquired events were filtered by time and manually gated for CD45⁺ viable singlets using FlowJo (FIG. 26C). CD45⁺ viable singlets were analyzed using the cytofkit2 package in R. 30,000 events per treatment group were randomly subsampled for analysis, marker expression values were transformed using the cytofAsinh function, and unsupervised clustering was performed using the RPhenograph algorithm.

IHC: Whole brains were fixed in 10% formalin overnight at room temperature, dehydrated in decreasing concentrations of ethanol, and embedded in paraffin by UCLA Translational Pathology Core Laboratory (TPCL). Five-micron coronal sections were cut by TPCL. Heat-induced antigen retrieval was performed using 1× Universal HIER antigen retrieval reagent (Abcam), and sections were stained with goat anti-IL-13Rα2 (R&D Systems catalog no. AF146) followed by secondary staining with donkey anti-goat IgG conjugated to AlexaFluor594 (Invitrogen catalog no. A32758). Samples were coverslipped with mounting media with DAPI (Abcam). IHC-stained sections were imaged on a Zeiss LSM880 confocal microscope at 20× magnification (UCLA Broad Stem Cell Research Center).

Statistics: All statistical tests were performed using GraphPad Prism (version 9). Sigmoidal curve fits (FIG. 15D, E) with variable slope were performed in Prism, with the following constraints: Bottom=0, Top=100, and Slope >0.

Abbreviations: CAR: Chimeric antigen receptor; DC: Dendritic cell; DNR: Dominant-negative receptor; ffLuc: Firefly luciferase; GBM: Glioblastoma multiforme; GLuc: Gaussia luciferase; IDH1/2: Isocitrate dehydrogenase-1/2; IL-13Rα2: Interleukin-13 receptor subunit alpha-2; (M-)MDSC: (Monocytic-) Myeloid-derived suppressor cell; TAM: Tumor-associated macrophage; TGF-β: Transforming growth factor-beta; TME: Tumor microenvironment

TABLE 2
Histopathological findings of tissue samples from immunocompetent
mice following systemic administration of TGF-β CAR-T cells.

| | Mock | Mock | TGF-β CAR | TGF-β CAR |
|---|---|---|---|---|
| | Mouse 1 | Mouse 2 | Mouse 1 | Mouse 2 |
| Gross Findings | NGL | NGL | Mild, diffuse | NGL |
| | | | pale-tan liver | |
| Liver | Minimal, | NA | NSF | NSF |
| | multifocal | | | |
| | sinusoidal | | | |
| | congestion | | | |
| Spleen | NSF | NSF | NSF | NSF |
| Kidneys | Focal | NSF | NSF | NSF |
| | perivascular | | | |
| | lymphocytic | | | |
| | aggregate | | | |

NGL = No gross lesions
NSF = No significant findings
NA = Not applicable, information not available

### E. References

1. Stupp, R. et al. Radiotherapy plus concomitant and adjuvant temozolomide for glioblastoma. N. Engl. J. Med. 352, 987-996 (2005).
2. Stupp, R. et al. Effects of radiotherapy with concomitant and adjuvant temozolomide versus radiotherapy alone on survival in glioblastoma in a randomised phase III study: 5-year analysis of the EORTC-NCIC trial. Lancet Oncol. 10, 459-466 (2009).
3. Stupp. R. et al. Effect of Tumor-Treating Fields Plus Maintenance Temozolomide vs Maintenance Temozolomide Alone on Survival in Patients With Glioblastoma: A Randomized Clinical Trial. JAMA 318, 2306-2316 (2017).
4. Kirkpatrick, J. P., Laack, N. N., Shih, H. A. & Gondi, V. Management of GBM: a problem of local recurrence. J. Neurooncol. 134, 487-493 (2017).
5. Alexander, B. M. & Cloughesy, T. F. Adult Glioblastoma. J. Clin. Oncol. 35, 2402-2409 (2017).
6. Schuster, S. J. et al. Long-term clinical outcomes of tisagenlecleucel in patients with relapsed or refractory aggressive B-cell lymphomas (JULIET): a multicentre, open-label, single-arm, phase 2 study. Lancet Oncol. 22, 1403-1415 (2021).
7. Jacobson, C. et al. Long-Term (≥4 Year and ≥5 Year) Overall Survival (OS) By 12- and 24-Month Event-Free Survival (EFS): An Updated Analysis of ZUMA-1, the Pivotal Study of Axicabtagene Ciloleucel (Axi-Cel) in Patients (Pts) with Refractory Large B-Cell Lymphoma (LBCL). Blood 138, 1764 (2021).
8. Neelapu, S. S. et al. Axicabtagene ciloleucel as first-line therapy in high-risk large B-cell lymphoma: the phase 2 ZUMA-12 trial. Nat. Med. 28, 735-742 (2022).
9. Berdeja. J. G. et al. Ciltacabtagene autoleucel, a B-cell maturation antigen-directed chimeric antigen receptor T-cell therapy in patients with relapsed or refractory multiple myeloma (CARTITUDE-1): a phase 1b/2 open-label study. The Lancet 398, 314-324 (2021).
10. Abramson, J. S. et al. Lisocabtagene maraleucel for patients with relapsed or refractory large B-cell lymphomas (TRANSCEND NHL 001): a multicentre seamless design study. The Lancet 396, 839-852 (2020).
11. Brown, C. E. et al. Regression of Glioblastoma after Chimeric Antigen Receptor T-Cell Therapy. N. Engl. J. Med. 375, 2561-2569 (2016).
12. O'Rourke, D. M. et al. A single dose of peripherally infused EGFRvIII-directed CAR T cells mediates antigen loss and induces adaptive resistance in patients with recurrent glioblastoma. Sci. Transl. Med. 9, (2017).
13. Ahmed, N. et al. HER2-Specific Chimeric Antigen Receptor-Modified Virus-Specific T Cells for Progressive Glioblastoma: A Phase 1 Dose-Escalation Trial. JAMA Oncol. 3, 1094-1101 (2017).
14. Majzner, R. G. et al. GD2-CAR T cell therapy for H3K27M-mutated diffuse midline gliomas. Nature 603, 934-941 (2022).
15. Tang. X. et al. Administration of B7-H3 targeted chimeric antigen receptor-T cells induce regression of glioblastoma. Signal Transduct. Target. Ther. 6, 1-3 (2021).
16. Brown, C. E. et al. Glioma IL13Rα2 Is Associated with Mesenchymal Signature Gene Expression and Poor Patient Prognosis. PLOS ONE 8, e77769 (2013).
17. Bhardwaj, R., Suzuki, A., Leland, P., Joshi, B. H. & Puri, R. K. Identification of a novel role of IL-13Rα2 in human Glioblastoma multiforme: interleukin-13 mediates signal transduction through AP-1 pathway. J. Transl. Med. 16, 369 (2018).
18. Brown, C. E. et al. Bioactivity and Safety of IL13Rα2-Redirected Chimeric Antigen Receptor CD8+ T Cells in Patients with Recurrent Glioblastoma. Clin. Cancer Res. 21, 4062-4072 (2015).
19. Yaghoubi, S. S. et al. Noninvasive detection of therapeutic cytolytic T cells with 18F-FHBG PET in a patient with glioma. Nat. Clin. Pract. Oncol. 6, 53-58 (2009).
20. Iwami, K. et al. Peptide-pulsed dendritic cell vaccination targeting interleukin-13 receptor a2 chain in recurrent malignant glioma patients with HLA-A*24/A*02 allele. Cytotherapy 14, 733-742 (2012).
21. Kunwar, S. et al. Direct Intracerebral Delivery of Cintredekin Besudotox (IL13-PE38QQR) in Recurrent Malignant Glioma: A Report by the Cintredekin Besudotox Intraparenchymal Study Group. J. Clin. Oncol. 25, 837-844 (2007).
22. Hou, A. J., Chen, L. C. & Chen, Y. Y. Navigating CAR-T cells through the solid-tumour microenvironment. Nat. Rev. Drug Discov. 20, 531-550 (2021).
23. Hanahan, D. & Weinberg, R. A. Hallmarks of Cancer: The Next Generation. Cell 144, 646-674 (2011).
24. Han, J., Alvarez-Breckenridge, C. A., Wang, Q.-E. & Yu, J. TGF-β signaling and its targeting for glioma treatment. Am. J. Cancer Res. 5, 945-955 (2015).
25. Ikushima, H. et al. Autocrine TGF-β Signaling Maintains Tumorigenicity of Glioma-Initiating Cells through Sry-Related HMG-Box Factors. Cell Stem Cell 5, 504-514 (2009).
26. Peñuelas, S. et al. TGF-β Increases Glioma-Initiating Cell Self-Renewal through the Induction of LIF in Human Glioblastoma. Cancer Cell 15, 315-327 (2009).
27. Bruna, A. et al. High TGFβ-Smad Activity Confers Poor Prognosis in Glioma Patients and Promotes Cell Proliferation Depending on the Methylation of the PDGF-B Gene. Cancer Cell 11, 147-160 (2007).
28. Thomas, D. A. & Massagué, J. TGF-β directly targets cytotoxic T cell functions during tumor evasion of immune surveillance. Cancer Cell 8, 369-380 (2005).
29. Flavell, R. A., Sanjabi, S., Wrzesinski, S. H. & Licona-Limón, P. The polarization of immune cells in the tumour environment by TGF-β. Nat. Rev. Immunol. 10, 554-567 (2010).
30. Chen, M.-L. et al. Regulatory T cells suppress tumor-specific CD8 T cell cytotoxicity through TGF-β signals in vivo. Proc. Natl. Acad. Sci. 102, 419-424 (2005).
31. Wu, A. et al. Glioma cancer stem cells induce immunosuppressive macrophages/microglia. Neuro-Oncol. 12, 1113-1125 (2010).
32. Biswas, S. K. & Mantovani, A. Macrophage plasticity and interaction with lymphocyte subsets: cancer as a paradigm. Nat. Immunol. 11, 889-896 (2010).
33. Gabrilovich, D. I., Ostrand-Rosenberg, S. & Bronte, V. Coordinated regulation of myeloid cells by tumours. Nat. Rev. Immunol. 12, 253-268 (2012).
34. Rodon, J. et al. First-in-Human Dose Study of the Novel Transforming Growth Factor-β Receptor I Kinase Inhibitor LY2157299 Monohydrate in Patients with Advanced Cancer and Glioma. Clin. Cancer Res. 21, 553-560 (2015).
35. Brandes, A. A. et al. A Phase II randomized study of galunisertib monotherapy or galunisertib plus lomustine compared with lomustine monotherapy in patients with recurrent glioblastoma. Neuro-Oncol. 18, 1146-1156 (2016).
36. Wick, A. et al. Phase 1b/2a study of galunisertib, a small molecule inhibitor of transforming growth factor-beta receptor I, in combination with standard temozolomide-based radiochemotherapy in patients with newly diagnosed malignant glioma. Invest. New Drugs 38, 1570-1579 (2020).
37. Tomaszewski, W., Sanchez-Perez, L., Gajewski, T. F. & Sampson, J. H. Brain Tumor Microenvironment and Host State: Implications for Immunotherapy. Clin. Cancer Res. 25, 4202-4210 (2019).
38. Quail. D. F. & Joyce, J. A. The Microenvironmental Landscape of Brain Tumors. Cancer Cell 31, 326-341 (2017).
39. Pardridge, W. M. Blood-Brain Barrier and Delivery of Protein and Gene Therapeutics to Brain. Front. Aging Neurosci. 11, (2020).
40. Siegel, P. M. & Massagué, J. Cytostatic and apoptotic actions of TGF-β in homeostasis and cancer. Nat. Rev. Cancer 3, 807-820 (2003).
41. Anderton, M. J. et al. Induction of heart valve lesions by small-molecule ALK5 inhibitors. Toxicol. Pathol. 39, 916-924 (2011).
42. Lacouture, M. E. et al. Cutaneous keratoacanthomas/squamous cell carcinomas associated with neutralization of transforming growth factor β by the monoclonal antibody fresolimumab (GC1008). Cancer Immunol. Immunother. 64, 437-446 (2015).
43. Chang. Z. L. et al. Rewiring T-cell responses to soluble factors with chimeric antigen receptors. Nat. Chem. Biol. 14, 317-324 (2018).
44. Bollard, C. M. et al. Adapting a transforming growth factor β-related tumor protection strategy to enhance antitumor immunity. Blood 99, 3179-3187 (2002).
45. Bollard, C. M. et al. Tumor-Specific T-Cells Engineered to Overcome Tumor Immune Evasion Induce Clinical Responses in Patients With Relapsed Hodgkin Lymphoma. J. Clin. Oncol. 36, 1128-1139 (2018).
46. Kloss, C. C. et al. Dominant-Negative TGF-β Receptor Enhances PSMA-Targeted Human CAR T Cell Proliferation And Augments Prostate Cancer Eradication. Mol. Ther. 26, 1855-1866 (2018).
47. Narayan, V. et al. PSMA-targeting TGFβ-insensitive armored CAR T cells in metastatic castration-resistant prostate cancer: a phase 1 trial. Nat. Med. 28, 724-734 (2022).
48. Sukumaran, S. et al. Enhancing the potency and specificity of engineered T cells for cancer treatment. Cancer Discov. CD-17-1298 (2018) doi: 10.1158/2159-8290.CD-17-1298.
49. Roth, T. L. et al. Pooled Knockin Targeting for Genome Engineering of Cellular Immunotherapies. Cell 181, 728-744.c21 (2020).
50. Noh. K.-E. et al. TGF-β/IL-7 Chimeric Switch Receptor-Expressing CAR-T Cells Inhibit Recurrence of CD19-Positive B Cell Lymphoma. Int. J. Mol. Sci. 22, 8706 (2021).
51. Hou, A. J., Chang, Z. L., Lorenzini, M. H., Zah, E. & Chen, Y. Y. TGF-β-responsive CAR-T cells promote anti-tumor immune function. Bioeng. Transl. Med. 3, 75-86 (2018).
52. Debinski, W., Gibo, D. M., Obiri, N. I., Kealiher, A. & Puri, R. K. Novel anti-brain tumor cytotoxins specific for cancer cells. Nat. Biotechnol. 16, 449-453 (1998).
53. Debinski, W. & Thompson, J. P. Retargeting interleukin 13 for radioimmunodetection and radioimmunotherapy of human high-grade gliomas. Clin. Cancer Res. 5, 3143s-3147s (1999).
54. Brown, C. E. et al. Optimization of IL13Rα2-Targeted Chimeric Antigen Receptor T Cells for Improved Anti-tumor Efficacy against Glioblastoma. Mol. Ther. 26, 31-44 (2018).
55. Batlle, E. & Massagué, J. Transforming Growth Factor-β Signaling in Immunity and Cancer. Immunity 50, 924-940 (2019).
56. Moore-Smith, L. D., Isayeva, T., Lec, J. H., Frost, A. & Ponnazhagan, S. Silencing of TGF-β1 in tumor cells impacts MMP-9 in tumor microenvironment. Sci. Rep. 7, 8678 (2017).
57. Shi, M. et al. Latent TGF-β structure and activation. Nature 474, 343-349 (2011).
58. Krstic, J. & Santibanez, J. F. Transforming Growth Factor-Beta and Matrix Metalloproteinases: Functional Interactions in Tumor Stroma-Infiltrating Myeloid Cells. Sci. World J. 2014, e521754 (2014).
59. Vroemen, M., Weidner, N. & Blesch, A. Loss of gene expression in lentivirus- and retrovirus-transduced neural progenitor cells is correlated to migration and differentiation in the adult spinal cord. Exp. Neurol. 195, 127-139 (2005).
60. Lee, A. H. et al. Neoadjuvant PD-1 blockade induces T cell and cDC1 activation but fails to overcome the immunosuppressive tumor associated macrophages in recurrent glioblastoma. Nat. Commun. 12, 6938 (2021).
61. Li, J. et al. Tumor Cell-Intrinsic Factors Underlie Heterogeneity of Immune Cell Infiltration and Response to Immunotherapy. Immunity 49, 178-193.e7 (2018).
62. Woroniecka, K. et al. T-Cell Exhaustion Signatures Vary with Tumor Type and Are Severe in Glioblastoma. Clin. Cancer Res. 24, 4175-4186 (2018).
63. Khalsa, J. K. et al. Immune phenotyping of diverse syngeneic murine brain tumors identifies immunologically distinct types. Nat. Commun. 11, 3912 (2020).
64. Antonios, J. P. et al. Immunosuppressive tumor-infiltrating myeloid cells mediate adaptive immune resistance via a PD-1/PD-L1 mechanism in glioblastoma. Neuro-Oncol. 19, 796-807 (2017).
65. Müller, S. et al. Single-cell profiling of human gliomas reveals macrophage ontogeny as a basis for regional differences in macrophage activation in the tumor microenvironment. Genome Biol. 18, 234 (2017).
66 Chen, Z. et al. Cellular and Molecular Identity of Tumor-Associated Macrophages in Glioblastoma. Cancer Res. 77, 2266-2278 (2017).
67. Cao, W., Lee, S. H. & Lu, J. CD83 is preformed inside monocytes, macrophages and dendritic cells, but it is only stably expressed on activated dendritic cells. Biochem. J. 385, 85-93 (2004).
68. Sharanek, A. et al. Transcriptional control of brain tumor stem cells by a carbohydrate binding protein. Cell Rep. 36, (2021).
69. Hu, W.-M., Yang, Y.-Z., Zhang, T.-Z., Qin, C.-F. & Li, X.-N. LGALS3 Is a Poor Prognostic Factor in Diffusely Infiltrating Gliomas and Is Closely Correlated With CD163+ Tumor-Associated Macrophages. Front. Med. 7, (2020).
70. Yuan, F. et al. Molecular and clinical characterization of Galectin-9 in glioma through 1,027 samples. J. Cell. Physiol. 235, 4326-4334 (2020).
71. He, X., Zhang, S., Chen, J. & Li, D. Increased LGALS3 expression independently predicts shorter overall survival in patients with the proneural subtype of glioblastoma. Cancer Med. 8, 2031-2040 (2019).
72. Rabinovich, G. A. & Conejo-García, J. R. Shaping the Immune Landscape in Cancer by Galectin-Driven Regulatory Pathways. J. Mol. Biol. 428, 3266-3281 (2016).
73. Girotti, M. R., Salatino, M., Dalotto-Moreno, T. & Rabinovich, G. A. Sweetening the hallmarks of cancer: Galectins as multifunctional mediators of tumor progression. J. Exp. Med. 217, e20182041 (2019).
74. Wu, C. et al. Galectin-9-CD44 Interaction Enhances Stability and Function of Adaptive Regulatory T Cells. Immunity 41, 270-282 (2014).
75. Daroqui, C. M. et al. Regulation of galectin-1 expression by transforming growth factor β1 in metastatic mammary adenocarcinoma cells: implications for tumor-immune escape. Cancer Immunol. Immunother. 56, 491-499 (2007).
76. Chou, C. et al. Programme of self-reactive innate-like T cell-mediated cancer immunity. Nature 605, 139-145 (2022).
77. Theruvath, J. et al. Locoregionally administered B7-H3-targeted CAR T cells for treatment of atypical teratoid/rhabdoid tumors. Nat. Med. 26, 712-719 (2020).
78. Bausart, M., Préat, V. & Malfanti, A. Immunotherapy for glioblastoma: the promise of combination strategies. J. Exp. Clin. Cancer Res. 41, 35 (2022).
79. Hegde, M. et al. Tandem CAR T cells targeting HER2 and IL13Rα2 mitigate tumor antigen escape. J. Clin. Invest. 126, 3036-3052.
80. Bielamowicz, K. et al. Trivalent CAR T cells overcome interpatient antigenic variability in glioblastoma. Neuro-Oncol. 20, 506-518 (2018).
81. Choe, J. H. et al. SynNotch-CAR T cells overcome challenges of specificity, heterogeneity, and persistence in treating glioblastoma. Sci. Transl. Med. 13, eabe7378 (2021).
82. Pituch, K. C. et al. Adoptive Transfer of IL13Rα2-Specific Chimeric Antigen Receptor T Cells Creates a Pro-inflammatory Environment in Glioblastoma. Mol. Ther. 26, 986-995 (2018).
83. Alizadeh, D. et al. IFNγ Is Critical for CAR T Cell-Mediated Myeloid Activation and Induction of Endogenous Immunity. Cancer Discov. 11, 2248-2265 (2021).
84. Aslan, K. et al. Heterogeneity of response to immune checkpoint blockade in hypermutated experimental gliomas. Nat. Commun. 11, 931 (2020).
85. Tauriello, D. V. F. et al. TGFβ drives immune evasion in genetically reconstituted colon cancer metastasis. Nature 554, 538-543 (2018).
86. Mariathasan, S. et al. TGFβ attenuates tumour response to PD-L1 blockade by contributing to exclusion of T cells. Nature 554, 544-548 (2018).
87. Lan, Y. et al. Simultaneous targeting of TGF-β/PD-L1 synergizes with radiotherapy by reprogramming the tumor microenvironment to overcome immune evasion. Cancer Cell 39, 1388-1403.e10 (2021).
88. Vanpouille-Box, C. et al. TGFβ Is a Master Regulator of Radiation Therapy-Induced Antitumor Immunity. Cancer Res. 75, 2232-2242 (2015).
89. Hamon, P. et al. TGFβ receptor inhibition unleashes interferon-β production by tumor-associated macrophages and enhances radiotherapy efficacy. J. Immunother. Cancer 10, e003519 (2022).
90. Bloch, O. et al. Autologous Heat Shock Protein Peptide Vaccination for Newly Diagnosed Glioblastoma: Impact of Peripheral PD-L1 Expression on Response to Therapy. Clin. Cancer Res. 23, 3575-3584 (2017).
91. Chen, Z. & Hambardzumyan, D. Immune Microenvironment in Glioblastoma Subtypes. Front. Immunol. 9, (2018).
92. Exclusive: Carl June's Tmunity encounters a lethal roadblock as 2 patient deaths derail lead trial, raise red flag forcing a rethink of CAR-T for solid tumors. Endpoints News https://endpts.com/exclusive-carl-junes-tmunity-encounters-a-lethal-roadblock-as-2-patient-deaths-derail-lead-trial-raise-red-flag-forcing-a-rethink-of-car-t-for-solid-tumors/.
93. Poseida Therapeutics, Inc. Form 8-K. https://sec.report/Document/0001193125-20-222442/.
94. Poseida Therapeutics Announces Clinical Hold Lifted on Phase I Autologous CAR-T Study in Prostate Cancer|Poseida Therapeutics, Inc. https://investors.poseida.com/news-releases/news-release-details/poseida-therapeutics-announces-clinical-hold-lifted-phase-i/.
95. Bonini, C. et al. HSV-TK Gene Transfer into Donor Lymphocytes for Control of Allogeneic Graft-Versus-Leukemia. Science 276, 1719-1724 (1997).
96. Berger, C., Flowers, M. E., Warren, E. H. & Riddell, S. R. Analysis of transgene-specific immune responses that limit the in vivo persistence of adoptively transferred HSV-TK-modified donor T cells after allogeneic hematopoietic cell transplantation. Blood 107, 2294-2302 (2006).
97. Ciceri, F. et al. Infusion of suicide-gene-engineered donor lymphocytes after family haploidentical haemopoietic stem-cell transplantation for leukaemia (the TK007 trial): a non-randomised phase I-II study. Lancet Oncol. 10, 489-500 (2009).
98. Di Stasi, A. et al. Inducible Apoptosis as a Safety Switch for Adoptive Cell Therapy. N. Engl. J. Med. 365, 1673-1683 (2011).
99 Petrelli, F. et al. Steroids use and survival in patients with glioblastoma multiforme: a pooled analysis. J. Neurol. 268, 440-447 (2021).
100. Gibson, D. G. et al. Enzymatic assembly of DNA molecules up to several hundred kilobases. Nat. Methods 6, 343-345 (2009).
101. Jonnalagadda, M. et al. Chimeric Antigen Receptors With Mutated IgG4 Fc Spacer Avoid Fc Receptor Binding and Improve T Cell Persistence and Antitumor Efficacy. Mol. Ther. 23, 757-768 (2015).
102. Wang, X. et al. A transgene-encoded cell surface polypeptide for selection, in vivo tracking, and ablation of engineered cells. Blood 118, 1255-1263 (2011).
103. Wang, D. et al. Chlorotoxin-directed CAR T cells for specific and effective targeting of glioblastoma. Sci. Transl. Med. 12, (2020).
104. Zah, E. et al. Systematically optimized BCMA/CS1 bispecific CAR-T cells robustly control heterogeneous multiple myeloma. Nat. Commun. 11, 1-13 (2020).
105. Wang, D. et al. In vitro tumor cell rechallenge for predictive evaluation of chimeric antigen receptor T cell antitumor function. J. Vis. Exp. (2019) doi: 10.3791/59275.
106. Hao, Y. et al. Integrated analysis of multimodal single-cell data. Cell 184, 3573-3587.e29 (2021).
107. Torrejon, D. Y. et al. Overcoming Genetically Based Resistance Mechanisms to PD-1 Blockade. Cancer Discov. 10, 1140-1157 (2020).

All of the disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

The references recited in the application, to the extent that they provide exemplary procedural or other details supplementary to those set forth herein, are specifically incorporated herein by reference.

### REFERENCES

The following references and the publications referred to throughout the specification, to the extent that they provide exemplary procedural or other details supplementary to those set forth herein, are specifically incorporated herein by reference.
Chang, Z. L., Lorenzini, M. H., Zah, E., Tran, U., Chen, Y. Y. (2018). Rewiring T-cell responses to soluble factors with chimeric antigen receptors. Nature Chemical Biology, 14 (3): 317-324.
Hou, A. J., Chang, Z. L., Lorenzini, M. H., Zah, E., and Chen, Y. Y. (2018). TGF-β-responsive CAR-T cells promote anti-tumor immune function. Bioengineering and Translational Medicine, 3 (2): 75-86.
Chang, Z. L., Hou, A. J., and Chen, Y. Y. (2020). Engineering primary T cells with chimeric antigen receptors for rewired responses to soluble ligands. Nature Protocols, Epub ahead of print.
Debinski, W. and Thompson J. P. (1999). Retargeting interleukin 13 for radioimmunodetection and radioimmunotherapy of human high-grade gliomas. Clinical Cancer Research, 5: 3143s-7s.
Kahlon, K. S. et al. (2004). Specific recognition and killing of glioblastoma multiforme by interleukin 13-zetakine redirected cytolytic T cells. Cancer Research, 64:9160-6.
Brown C. E. et al. (2015). Bioactivity and safety of IL13Rα2-redirected chimeric antigen receptor CD8+ T cells in patients with recurrent glioblastoma. Clinical Cancer Research, 21:4062-72.
Brown, C. E. et al. (2016). Regression of Glioblastoma after Chimeric Antigen Receptor T-Cell Therapy. New England Journal of Medicine, 375 (26): 2561-2596.

1. A method for stimulating an immune response in a subject and/or for treating a subject with cancer comprising administering to the subject an effective amount of the composition comprising cells comprising a heterologous nucleic acid encoding for a polypeptide, wherein the polypeptide comprises:
i.) a multi-specific chimeric antigen receptor (CAR) comprising an IL13 polypeptide, a TGF-β binding region, a peptide spacer, a transmembrane domain, and a cytoplasmic region comprising a co-stimulatory region and a primary intracellular signaling domain;
ii.) a multi-specific chimeric antigen receptor comprising an IL13Rα binding region, a tumor antigen binding region, a peptide spacer, a transmembrane domain, and a cytoplasmic region comprising a co-stimulatory region and a primary intracellular signaling domain; wherein the tumor antigen binding region comprises a GD2 or EGFRvIII binding region;
iii.) a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO: 53 (LCDR3);
iv.) a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47; or
v.) a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an EGFRvIII binding region;
vi.) a multi-specific chimeric antigen receptor comprising an IL13 polypeptide with the amino acid sequence of SEQ ID NO:4 or 20, a tumor antigen binding region, a peptide spacer, a transmembrane domain, and a cytoplasmic region comprising a co-stimulatory region and a primary intracellular signaling domain; wherein the tumor antigen binding region comprises a GD2 or EGFRvIII binding region;
vii.) a multi-specific chimeric antigen receptor (CAR) comprising an IL13 polypeptide with the amino acid sequence of SEQ ID NO:4 or 20, a TGF-β binding region, a peptide spacer, a transmembrane domain, and a cytoplasmic region comprising a co-stimulatory region and a primary intracellular signaling domain;
wherein the subject has and/or the cancer is selected from malignant glioma, diffuse midline glioma, neuroblastoma, sarcoma, osteosarcoma, diffuse intrinsic pontine glioma, and melanoma.

2. The method of claim 1, wherein the polypeptide comprises a multi-specific chimeric antigen receptor (CAR) comprising an IL13 polypeptide, a TGF-β binding region, a peptide spacer, a transmembrane domain, and a cytoplasmic region comprising a co-stimulatory region and a primary intracellular signaling domain.

3. The method of claim 1, wherein the polypeptide comprises an IL13Rα binding region, a tumor antigen binding region, a peptide spacer, a transmembrane domain, and a cytoplasmic region comprising a co-stimulatory region and a primary intracellular signaling domain; wherein the tumor antigen binding region comprises a GD2 or EGFRvIII binding region.

4. The method of claim 1 or 3, wherein the tumor antigen binding region comprises a GD2 binding region.

5. The method of claim 4, wherein the GD2 binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47.

6. The method of claim 5, wherein the GD2 binding region comprises an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3).

7. The method of any one of claims 4-6, wherein the GD2 binding region comprises a VH with an amino acid sequence having at least 80% sequence identity to SEQ ID NO:46 and/or a VL with an amino acid sequence having at least 80% sequence identity to SEQ ID NO:47.

8. The method of claim 7, wherein the GD2 binding region comprises a VH with the amino acid sequence of SEQ ID NO:46 and/or a VL with the amino acid sequence of SEQ ID NO:47.

9. The method of any one of claims 4-8, wherein the GD2 binding region comprises an anti-GD2 scFv having an amino acid sequence with at least 80% sequence identity to SEQ ID NO: 26.

10. The method of claim 9, wherein the GD2 binding region comprises an anti-GD2 scFv having the amino acid sequence of SEQ ID NO:26.

11. The method of claim 1 or 3, wherein the tumor antigen binding region comprises an EGFRvIII binding region.

12. The method of claim 11, wherein the EGFRvIII binding region comprises an anti-EGFRvIII scFv having a variable heavy (VH) and variable light (VL) region, wherein the VH region comprises the HCDR1, HCDR2; and HCDR3 from the VH of SEQ ID NO:38 and the VL region comprises LCDR1, LCDR2; and LCDR3 from the VL of SEQ ID NO:39.

13. The method of claim 11 or 12, wherein the EGFRvIII binding region comprises an anti-EGFRvIII scFv having a variable heavy (VH) and variable light (VL) region, wherein the VH region comprises SEQ ID NO:40 (HCDR1), SEQ ID NO:41 (HCDR2); and SEQ ID NO:42 (HCDR3) and the VL region comprises SEQ ID NO:43 (LCDR1), SEQ ID NO:44 (LCDR2); and SEQ ID NO:45 (LCDR3).

14. The method of any one of claims 11-13, wherein the EGFRvIII binding region comprises a VH with an amino acid sequence having at least 80% sequence identity to SEQ ID NO: 38 and/or a VL with an amino acid sequence having at least 80% sequence identity to SEQ ID NO: 39.

15. The method of claim 14, wherein the EGFRVIII binding region comprises a VH with the amino acid sequence of SEQ ID NO:38 and/or a VL with the amino acid sequence of SEQ ID NO: 39.

16. The method of any one of claims 11-15, wherein the EGFRVIII binding region comprises an anti-EGFRvIII scFv having an amino acid sequence with at least 80% sequence identity to SEQ ID NO:27.

17. The method of claim 16, wherein the EGFRVIII binding region comprises an anti-EGFRvIII scFv having the amino acid sequence of SEQ ID NO:27.

18. The method of any one of claims 1-17, wherein the CAR comprises in order from amino-proximal end to carboxy-proximal end: an IL13Rα binding region, a tumor antigen binding region, a peptide spacer, a transmembrane domain, and a cytoplasmic region comprising a co-stimulatory region and a primary intracellular signaling domain.

19. The method of any one of claims 1-18, wherein the polypeptide comprises a linker between the IL13Rα binding region and the tumor antigen binding region.

20. The method of any one of claims 1-19, wherein the polypeptide comprises a tri-specific CAR comprising a TGF-β binding region.

21. The method of claim 20, wherein the CAR comprises in order from amino-proximal end to carboxy-proximal end: an IL13Rα binding region, a tumor antigen binding region, a TGF-binding region, a peptide spacer, a transmembrane domain, and a cytoplasmic region comprising a co-stimulatory region and a primary intracellular signaling domain.

22. The method of claim 20 or 21, wherein the polypeptide comprises a linker between the tumor antigen binding region or the IL13Rα binding region and the TGF-β binding region.

23. The method of any one of claims 19-22, wherein the linker comprises glycine and serine amino acids.

24. The method of claim 23, wherein the linker comprises or consists of a polypeptide with the amino acid sequence of SEQ ID NO: 10 or 28.

25. The method of any one of claims 1-24, wherein the IL13Rα binding region comprises an IL13Rα2-specific binding region.

26. The method of any one of claims 1-25, wherein the IL13Rα binding region comprises an IL13 polypeptide.

27. The method of claim 1, wherein the polypeptide comprises a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises SEQ ID NO:48 (HCDR1), SEQ ID NO:49 (HCDR2); and SEQ ID NO:50 (HCDR3) and the VL region comprises SEQ ID NO:51 (LCDR1), SEQ ID NO:52 (LCDR2); and SEQ ID NO:53 (LCDR3).

28. The method of claim 1, wherein the polypeptide comprises a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an anti-GD2 scFv having a variable heavy (VH) and variable light (VL) region, wherein the VH region comprises the HCDR1, HCDR2; and HCDR3 from the VH of SEQ ID NO:46 and the VL region comprises LCDR1, LCDR2; and LCDR3 from the VL of SEQ ID NO:47.

29. The method of claim 27 or 28, wherein the GD2 binding region comprises a VH with an amino acid sequence having at least 80% sequence identity to SEQ ID NO:46 and/or a VL with an amino acid sequence having at least 80% sequence identity to SEQ ID NO:47.

30. The method of claim 29, wherein the GD2 binding region comprises a VH with the amino acid sequence of SEQ ID NO:46 and/or a VL with the amino acid sequence of SEQ ID NO: 47.

31. The method of any one of claims 27-30, wherein the GD2 binding region comprises an anti-GD2 scFv having an amino acid sequence with at least 80% sequence identity to SEQ ID NO: 26.

32. The method of claim 31, wherein the GD2 binding region comprises an anti-GD2 scFv having the amino acid sequence of SEQ ID NO:26.

33. The method of claim 1, wherein the polypeptide comprises a multi-specific chimeric antigen receptor (CAR) comprising a tumor antigen binding region, a TGF-β binding region, a peptide spacer, a transmembrane domain, and a cytoplasmic region comprising a co-stimulatory region and a primary intracellular signaling domain; wherein the tumor antigen binding region comprises an EGFRvIII binding region.

34. The method of claim 33, wherein the EGFRvIII binding region comprises an anti-EGFRvIII scFv having a variable heavy (VH) and variable light (VL) region, wherein the VH region comprises the HCDR1, HCDR2; and HCDR3 from the VH of SEQ ID NO:38 and the VL region comprises LCDR1, LCDR2; and LCDR3 from the VL of SEQ ID NO:39.

35. The method of claim 33 or 34, wherein the EGFRvIII binding region comprises an anti-EGFRvIII scFv having a variable heavy (VH) and variable light (VL) region, wherein the VH region comprises SEQ ID NO:40 (HCDR1), SEQ ID NO:41 (HCDR2); and SEQ ID NO:42 (HCDR3) and the VL region comprises SEQ ID NO:43 (LCDR1), SEQ ID NO:44 (LCDR2); and SEQ ID NO:45 (LCDR3).

36. The method of any one of claims 33-35, wherein the EGFRVIII binding region comprises a VH with an amino acid sequence having at least 80% sequence identity to SEQ ID NO: 38 and/or a VL with an amino acid sequence having at least 80% sequence identity to SEQ ID NO: 39.

37. The method of claim 36, wherein the EGFRVIII binding region comprises a VH with the amino acid sequence of SEQ ID NO:38 and/or a VL with the amino acid sequence of SEQ ID NO: 39.

38. The method of any one of claims 33-37, wherein the EGFRvIII binding region comprises an anti-EGFRvIII scFv having an amino acid sequence with at least 80% sequence identity to SEQ ID NO:27.

39. The method of claim 38, wherein the EGFRvIII binding region comprises an anti-EGFRvIII scFv having the amino acid sequence of SEQ ID NO:27.

40. The method of any one of claims 20-39, wherein the TGF-β binding region comprises a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30.

41. The method of any one of claims 20-40, wherein the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO: 36 (LCDR3).

42. The method of any one of claims 20-41, wherein the scFv comprises a linker between the VH and VL regions.

43. The method of claim 42, wherein the linker comprises glycine and serine amino acid residues.

44. The method of claim 43, wherein the linker comprises or consists of the amino acid sequence of SEQ ID NO: 10 or 28.

45. The method of any one of claims 1-44, wherein the TGF-β binding region comprises a VH with an amino acid sequence having at least 80% sequence identity to SEQ ID NO:29 and/or a VL with an amino acid sequence having at least 80% sequence identity to SEQ ID NO: 30.

46. The method of claim 45, wherein the TGF-β binding region comprises a VH with the amino acid sequence of SEQ ID NO:29 and/or a VL with the amino acid sequence of SEQ ID NO: 30.

47. The method of any one of claims 1-46, wherein the TGF-β binding region comprises an anti-TGF-β scFv having an amino acid sequence with at least 80% sequence identity to SEQ ID NO: 11.

48. The method of claim 47, wherein the TGF-β binding region comprises an anti-TGF-scFv having the amino acid sequence of SEQ ID NO:11.

49. The method of any one of claims 1-48, wherein the IL13 polypeptide comprises an IL13 mutein.

50. The method of claim 49, wherein the IL13 mutein is further characterized as having a tyrosine substitution at a position corresponding to position 13 of SEQ ID NO:4, or position 21 of SEQ ID NO:20.

51. The method of claim 50, wherein the IL13 mutein comprises SEQ ID NO:4.

52. The method of claim 50, wherein the IL13 mutein comprises SEQ ID NO:20.

53. The method of any one of claims 1-52, wherein the polypeptide further comprises a second chimeric antigen receptor comprising at least one antigen binding region, a second peptide spacer, a second transmembrane domain, and a second cytoplasmic region comprising a second co-stimulatory region and a second primary intracellular signaling domain.

54. The method of claim 53, wherein the second CAR is a mono-specific or multi-specific CAR.

55. The method of claim 53 or 54, wherein the second CAR comprises an antigen binding region to TGF-β.

56. The method of claim 55, wherein the TGF-β binding region comprises a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30.

57. The method of claim 55 or 56, wherein the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

58. The method of claim 56 or 57, wherein the scFv comprises a linker between the VH and VL regions.

59. The method of claim 58, wherein the linker comprises glycine and serine amino acid residues.

60. The method of claim 59, wherein the linker comprises or consists of the amino acid sequence of SEQ ID NO: 10 or 28.

61. The method of any one of claims 55-60, wherein the TGF-β binding region comprises a VH with an amino acid sequence having at least 80% sequence identity to SEQ ID NO:29 and/or a VL with an amino acid sequence having at least 80% sequence identity to SEQ ID NO: 30.

62. The method of claim 61, wherein the TGF-β binding region comprises a VH with the amino acid sequence of SEQ ID NO:29 and/or a VL with the amino acid sequence of SEQ ID NO: 30.

63. The method of any one of claims 55-62, wherein the TGF-β binding region comprises an anti-TGF-β scFv having an amino acid sequence with at least 80% sequence identity to SEQ ID NO: 11.

64. The method of claim 63, wherein the TGF-β binding region comprises an anti-TGF-β scFv having the amino acid sequence of SEQ ID NO:11.

65. The method of any one of claims 53-63, wherein the first CAR and the second CAR are separated by one or more peptide cleavage site(s).

66. The method of claim 65, wherein the wherein the one or more cleavage sites comprise a 2A cleavage site.

67. The method of claim 66, wherein the 2A cleavage site comprises one or more of a P2A, F2A, E2A, or T2A cleavage site.

68. The method of claim 67, wherein the cleavage site comprise a T2A cleavage site with an amino acid sequence of SEQ ID NO:24 or with an amino acid sequence with at least 80% sequence identity to SEQ ID NO:24.

69. The method of any one of claims 1-68, wherein the peptide spacer is between the antigen binding domains and the transmembrane domain and/or the second peptide spacer is between the antigen binding domains and the second transmembrane domain of the second CAR.

70. The method of any one of claims 1-69, wherein the peptide spacer or second peptide spacer comprises an IgG4 hinge region.

71. The method of claim 70, wherein the IgG4 hinge region comprises a polypeptide having an amino acid sequence with at least 80% sequence identity to SEQ ID NO:12 or 5.

72. The method of claim 71, wherein the IgG4 hinge region comprises a polypeptide having the amino acid sequence of SEQ ID NO:12 or 5.

73. The method of any one of claims 1-72, wherein the peptide spacer or second peptide spacer comprises or further comprises an IgG4 CH2 and CH3 region.

74. The method of claim 73, wherein the IgG4 CH2 and CH3 region comprises a polypeptide having an amino acid sequence with at least 80% sequence identity to SEQ ID NO: 37.

75. The method of claim 73, wherein the IgG4 CH2 and CH3 region comprises a polypeptide having the amino acid sequence of SEQ ID NO:37.

76. The method of any one of claims 1-75, wherein the transmembrane domain or second transmembrane domain comprises the transmembrane domain from the CD28 protein.

77. The method of any one of claims 1-76, wherein the transmembrane domain or second transmembrane domain comprises a transmembrane domain having an amino acid sequence with at least 80% sequence identity to SEQ ID NO:6.

78. The method of any one of claims 1-77, wherein the transmembrane domain or second transmembrane domain comprises a transmembrane domain having the amino acid sequence of SEQ ID NO:6.

79. The method of any one of claims 1-78, wherein the co-stimulatory region or second co-stimulatory region comprises the co-stimulatory region from the 4-1BB protein or from the CD28 protein.

80. The method of any one of claims 1-79, wherein the co-stimulatory region or second co-stimulatory region comprises a co-stimulatory region having an amino acid sequence with at least 80% sequence identity to SEQ ID NO:7, 14, or 18.

81. The method of any one of claims 1-80, wherein the co-stimulatory region or second co-stimulatory region comprises a co-stimulatory region having the amino acid sequence of SEQ ID NO: 7, 14, or 18.

82. The method of any one of claims 1-81, wherein the primary intracellular signaling domain or second primary intracellular signaling domain comprises an intracellular signaling domain from the CD35 protein.

83. The method of any one of claims 1-82, wherein the primary intracellular signaling domain or second primary intracellular signaling domain comprises an intracellular signaling domain having an amino acid sequence with at least 80% sequence identity to SEQ ID NO:8 or 15.

84. The method of any one of claims 1-82, wherein the primary intracellular signaling domain or second primary intracellular signaling domain comprises an intracellular signaling domain having the amino acid sequence of SEQ ID NO:8 or 15.

85. The method of any one of claims 1-84, wherein the polypeptide further comprises one or more molecular tag(s).

86. The method of claim 85, wherein the one or more molecular tags comprise FLAG and/or HA tag.

87. The method of any one of claims 1-86, wherein the CAR and/or second CAR comprises a torsional linker between the transmembrane domain and the cytoplasmic region.

88. The method of claim 87, wherein the torsional linker comprises 1, 2, 3, 4, 5, 6, 7, 8, 9, or 10 amino acid residues.

89. The method of claim 88, wherein the amino acid residues comprise or consist of alanine residues.

90. The method of claim 89, wherein the torsional linker consists of 2 or 4 alanine residues.

91. The method of any one of claims 1-90, wherein the polypeptide comprises one of SEQ ID NOS: 1, 9, 13, 16, 17, 19, 21-23, 25, 136-145, 159, or 160 or an amino acid sequence having at least 80% sequence identity to one of SEQ ID NOS: 1, 9, 13, 16, 17, 19, 21-23, 25, 136-145, 159, or 160.

92. The method of any one of claims 1-90, wherein the polypeptide further comprises one or more signal sequence(s).

93. The method of claim 92, wherein the signal sequence(s) comprise an amino acid sequence with at least 80% sequence identity to SEQ ID NO:2.

94. The method of claim 93, wherein the signal sequence(s) comprise the amino acid sequence of SEQ ID NO:2.

95. The method of any one of claims 1-94, wherein the nucleic acid is an expression construct.

96. The method of claim 95, wherein the expression construct is a viral vector.

97. The method of claim 96, wherein the viral vector comprises a retroviral vector a vector derived from a retrovirus.

98. The method of claim 97, wherein the viral vector is a lentiviral vector or a vector derived from a lentivirus.

99. The method of any one of claims 96-98, wherein the viral vector has integrated into the cell's genome.

100. The method of any one of claims 1-99, wherein the cell further comprises a second nucleic acid encoding a second CAR.

101. The method of claim 100, wherein the second CAR is a mono-specific or multi-specific CAR.

102. The method of claim 100 or 101, wherein the second CAR comprises an antigen binding region to TGF-β.

103. The method of claim 102, wherein the TGF-β binding region comprises a scFv having a variable heavy (VH) and variable light (VL) region, wherein the VH region comprises the HCDR1, HCDR2; and HCDR3 from the VH of SEQ ID NO:29 and the VL region comprises LCDR1, LCDR2; and LCDR3 from the VL of SEQ ID NO:30.

104. The method of claim 102 or 103, wherein the TGF-β binding region comprises a scFv having a variable heavy (VH) and variable light (VL) region, wherein the VH region comprises SEQ ID NO:31 (HCDR1), SEQ ID NO:32 (HCDR2); and SEQ ID NO:33 (HCDR3) and the VL region comprises SEQ ID NO:34 (LCDR1), SEQ ID NO:35 (LCDR2); and SEQ ID NO:36 (LCDR3).

105. The method of claim 103 or 104, wherein the scFv comprises a linker between the VH and VL regions.

106. The method of claim 105, wherein the linker comprises glycine and serine amino acid residues.

107. The method of claim 106, wherein the linker comprises or consists of the amino acid sequence of SEQ ID NO:10 or 28.

108. The method of any one of claims 102-107, wherein the TGF-β binding region comprises a VH with an amino acid sequence having at least 80% sequence identity to SEQ ID NO:29 and/or a VL with an amino acid sequence having at least 80% sequence identity to SEQ ID NO: 30.

109. The method of any one of claims 102-108, wherein the TGF-binding region comprises a VH with the amino acid sequence of SEQ ID NO:29 and/or a VL with the amino acid sequence of SEQ ID NO:30.

110. The method of any one of claims 102-109, wherein the TGF-β binding region comprises an anti-TGF-β scFv having an amino acid sequence with at least 80% sequence identity to SEQ ID NO: 11.

111. The method of claim 110, wherein the TGF-β binding region comprises an anti-TGF-β scFv having the amino acid sequence of SEQ ID NO:11.

112. The method of any one of claims 100-111, wherein the second CAR comprises an antigen binding region to EGFRvIII.

113. The method of any one of claims 100-112, wherein the second CAR comprises an antigen binding region to GD2.

114. The method of any one of claims 1-113, wherein the cell is ex vivo.

115. The method of any one of claims 1-114, wherein the cell expresses the polypeptide encoded on the nucleic acid.

116. The method of any of claims 1-115, wherein the cell is a T cell, a natural killer (NK) cell, a natural killer T cell (NKT), an invariant natural killer T cell (INKT), stem cell, lymphoid progenitor cell, peripheral blood mononuclear cell (PBMC), bone marrow cell, fetal liver cell, embryonic stem cell, hematopoietic stem or progenitor cell (HSPC), cord blood cell, or induced pluripotent stem cell (iPS cell).

117. The method of claim 116, wherein the cell is a T cell or an NK cell.

118. The method of claim 117, wherein the T cell comprises a naïve memory T cell.

119. The method of claim 118, wherein the naïve memory T cell comprises a CD4+ or CD8+ T cell.

120. The method of any one of claims 1-119, wherein the cell is further defined as a population of cells.

121. The method of claim 120, wherein the population comprises 103-108 cells.

122. The method of any one of claims 1-121, wherein the method further comprises administering an additional therapy to the subject.

123. The method of claim 122, wherein the additional therapy comprises an immunotherapy.

124. The method of any one of claims 1-123, wherein the composition is administered intraventricularly, intracerebroventricularly, intratumorally, intravenously, or into a tumor resection cavity.

125. The method of any one of claims 1-124, wherein stimulating an immune response comprises increasing expression and/or secretion of immune stimulating cytokines and/or molecules.

126. The method of claim 125, wherein the immune stimulating cytokines and/or molecules are one or more of TNF-α, IFN-β, IFN-γ, IL-1, IL-2, IL-4, IL-6, IL-8, IL-10, IL-12, IL-18 and granulocyte-macrophage colony stimulating factor.

127. The method of any one of claims 1-126, wherein stimulating an immune response comprises increasing proliferation of immune cells.

128. The method of claim 127, wherein the immune cells are T cells.

129. The method of any one of claims 1-128, wherein the cell is in vivo in a subject in need of immune stimulation.

130. The method of claim 129, wherein the subject is one that produces endogenous TGF-β.

131. The method of any one of claims 1-130, wherein the cancer comprises diffuse midline glioma.

132. The method of any one of claims 1-131 wherein the wherein the subject is a human subject.

133. The method of any one of claims 1-132, wherein the method further comprises administering TGF-β to the subject.


SEQUENCE LISTING (161 sequences)

SEQ ID NO: 1 - protein, 560 aa, synthetic construct
  REGION 1..560: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 20 aa, synthetic construct
  REGION 1..20: note = Synthetic polypeptide
METDTLLLWVLLLWVPGSTG

SEQ ID NO: 3 - protein, 8 aa, synthetic construct
  REGION 1..8: note = Synthetic polypeptide
DYKDDDDK

SEQ ID NO: 4 - protein, 114 aa, synthetic construct
  REGION 1..114: note = Synthetic polypeptide
SPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 5 - protein, 229 aa, synthetic construct
  REGION 1..229: note = Synthetic polypeptide
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 28 aa, synthetic construct
  REGION 1..28: note = Synthetic polypeptide
MFWVLVVVGGVLACYSLLVTVAFIIFWV

SEQ ID NO: 7 - protein, 42 aa, synthetic construct
  REGION 1..42: note = Synthetic polypeptide
KRGRKKLLYIFKQPFMRPVQTTQEEDGCSCRFPEEEEGGCEL

SEQ ID NO: 8 - protein, 112 aa, synthetic construct
  REGION 1..112: note = Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 606 aa, synthetic construct
  REGION 1..606: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 20 aa, synthetic construct
  REGION 1..20: note = Synthetic polypeptide
GGGGSGGGGSGGGGSGGGGS

SEQ ID NO: 11 - protein, 243 aa, synthetic construct
  REGION 1..243: note = Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 12 - protein, 12 aa, synthetic construct
  REGION 1..12: note = Synthetic polypeptide
ESKYGPPCPPCP

SEQ ID NO: 13 - protein, 560 aa, synthetic construct
  REGION 1..560: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 14 - protein, 42 aa, synthetic construct
  REGION 1..42: note = Synthetic polypeptide
RRGRRRLLYIFRQPFMRPVQTTQEEDGCSCRFPEEEEGGCEL

SEQ ID NO: 15 - protein, 112 aa, synthetic construct
  REGION 1..112: note = Synthetic polypeptide
RVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 16 - protein, 606 aa, synthetic construct
  REGION 1..606: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 17 - protein, 562 aa, synthetic construct
  REGION 1..562: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 41 aa, synthetic construct
  REGION 1..41: note = Synthetic polypeptide
RSKRSRGGHSDYMNMTPRRPGPTRKHYQPYAPPRDFAAYRS

SEQ ID NO: 19 - protein, 568 aa, synthetic construct
  REGION 1..568: note = Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 122 aa, synthetic construct
  REGION 1..122: note = Synthetic polypeptide
LTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 21 - protein, 614 aa, synthetic construct
  REGION 1..614: note = Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 570 aa, synthetic construct
  REGION 1..570: note = Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 616 aa, synthetic construct
  REGION 1..616: note = Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 19 aa, synthetic construct
  REGION 1..19: note = Synthetic polypeptide
EGRGSLLTCGDVEENPGPR

SEQ ID NO: 25 - protein, 608 aa, synthetic construct
  REGION 1..608: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 246 aa, synthetic construct
  REGION 1..246: note = Synthetic polypeptide
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGSTSGSGKPGSGEGSTKGDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRA

SEQ ID NO: 27 - protein, 244 aa, synthetic construct
  REGION 1..244: note = Synthetic polypeptide
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSTSGSGKPGSGEGSTKGDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 28 - protein, 15 aa, synthetic construct
  REGION 1..15: note = Synthetic polypeptide
GGGGSGGGGSGGGGS

SEQ ID NO: 29 - protein, 120 aa, synthetic construct
  REGION 1..120: note = Synthetic polypeptide
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSS

SEQ ID NO: 30 - protein, 108 aa, synthetic construct
  REGION 1..108: note = Synthetic polypeptide
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 31 - protein, 5 aa, synthetic construct
  REGION 1..5: note = Synthetic polypeptide
SNVIS

SEQ ID NO: 32 - protein, 17 aa, synthetic construct
  REGION 1..17: note = Synthetic polypeptide
GVIPIVDIANYAQRFKG

SEQ ID NO: 33 - protein, 11 aa, synthetic construct
  REGION 1..11: note = Synthetic polypeptide
PRAFVLDAMDY

SEQ ID NO: 34 - protein, 12 aa, synthetic construct
  REGION 1..12: note = Synthetic polypeptide
RASQSLGSSYLA

SEQ ID NO: 35 - protein, 7 aa, synthetic construct
  REGION 1..7: note = Synthetic polypeptide
GASSRAP

SEQ ID NO: 36 - protein, 9 aa, synthetic construct
  REGION 1..9: note = Synthetic polypeptide
QQYADSPIT

SEQ ID NO: 37 - protein, 217 aa, synthetic construct
  REGION 1..217: note = Synthetic polypeptide
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 114 aa, synthetic construct
  REGION 1..114: note = Synthetic polypeptide
EIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSS

SEQ ID NO: 39 - protein, 112 aa, synthetic construct
  REGION 1..112: note = Synthetic polypeptide
DVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIK

SEQ ID NO: 40 - protein, 5 aa, synthetic construct
  REGION 1..5: note = Synthetic polypeptide
DYYIH

SEQ ID NO: 41 - protein, 17 aa, synthetic construct
  REGION 1..17: note = Synthetic polypeptide
RIDPENDETKYGPIFQG

SEQ ID NO: 42 - protein, 5 aa, synthetic construct
  REGION 1..5: note = Synthetic polypeptide
RGGVY

SEQ ID NO: 43 - protein, 16 aa, synthetic construct
  REGION 1..16: note = Synthetic polypeptide
KSSQSLLDSDGKTYLN

SEQ ID NO: 44 - protein, 7 aa, synthetic construct
  REGION 1..7: note = Synthetic polypeptide
LVSKLDS

SEQ ID NO: 45 - protein, 9 aa, synthetic construct
  REGION 1..9: note = Synthetic polypeptide
WQGTHFPGT

SEQ ID NO: 46 - protein, 113 aa, synthetic construct [sequence omitted]

SEQ ID NO: 47 - protein, 115 aa, synthetic construct
  REGION 1..115: note = Synthetic polypeptide
DVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRA

SEQ ID NO: 48 - protein, 5 aa, synthetic construct
  REGION 1..5: note = Synthetic polypeptide
GYNMN

SEQ ID NO: 49 - protein, 17 aa, synthetic construct
  REGION 1..17: note = Synthetic polypeptide
AIDPYYGGTSYNQKFKG

SEQ ID NO: 50 - protein, 4 aa, synthetic construct
  REGION 1..4: note = Synthetic polypeptide
GMEY

SEQ ID NO: 51 - protein, 16 aa, synthetic construct
  REGION 1..16: note = Synthetic polypeptide
RSSQSLVHRNGNTYLH

SEQ ID NO: 52 - protein, 7 aa, synthetic construct
  REGION 1..7: note = Synthetic polypeptide
KVSNRFS

SEQ ID NO: 53 - protein, 10 aa, synthetic construct
  REGION 1..10: note = Synthetic polypeptide
SQSTHVPPLT

SEQ ID NO: 54 - protein, 124 aa, synthetic construct
  REGION 1..124: note = Synthetic polypeptide
EVQLVESGGGLVQPGGSLRLSCAASGYAFTNYLIEWVRQAPGKGLEWVGVINPGSGGSNYNEKFKGRATISADNSKNTLYLQMNSLRAEDTAVYYCARSGGFYFDYWGQGTLVTVSSASTKGPS

SEQ ID NO: 55 - protein, 116 aa, synthetic construct
  REGION 1..116: note = Synthetic polypeptide
DIQMTQSPSSLSASVGDRVTITCRASQSVLYSSNQKNYLAWYQQKPGKAPKLLIYWASTRESGVPSRFSGSGSGTDFTLTISSLQPEDFATYYCHQYLSSDTFGQGTKVEIKRTVA

SEQ ID NO: 56 - protein, 10 aa, synthetic construct
  REGION 1..10: note = Synthetic polypeptide
GYAFTNYLIE

SEQ ID NO: 57 - protein, 17 aa, synthetic construct
  REGION 1..17: note = Synthetic polypeptide
VINPGSGGSNYNEKFKG

SEQ ID NO: 58 - protein, 8 aa, synthetic construct
  REGION 1..8: note = Synthetic polypeptide
SGGFYFDY

SEQ ID NO: 59 - protein, 17 aa, synthetic construct
  REGION 1..17: note = Synthetic polypeptide
RASQSVLYSSNQKNYLA

SEQ ID NO: 60 - protein, 7 aa, synthetic construct
  REGION 1..7: note = Synthetic polypeptide
WASTRES

SEQ ID NO: 61 - protein, 8 aa, synthetic construct
  REGION 1..8: note = Synthetic polypeptide
HQYLSSDT

SEQ ID NO: 62 - protein, 123 aa, synthetic construct
  REGION 1..123: note = Synthetic polypeptide
EVQLVESGGGVVQPGRSLRLSCAASGFTFSSYGMHWVRQAPGKELEWVAVISYDGSIKYYADSVKGRFTISRDNSKNTLYLQMNSLRAEDTAVYYCARTGEYSGYDTDPQYSWGQGTTVTVSS

SEQ ID NO: 63 - protein, 107 aa, synthetic construct
  REGION 1..107: note = Synthetic polypeptide
EIVLTQSPSSLSASVGDRVTITCRSSQGIGDDLGWYQQKPGKAPILLIYGTSTLQSGVPSRFSGSGSGTDFTLTINSLQPEDFATYYCLQDSNYPLTFGGGTRLEIK

SEQ ID NO: 64 - protein, 5 aa, synthetic construct
  REGION 1..5: note = Synthetic polypeptide
SYGMH

SEQ ID NO: 65 - protein, 17 aa, synthetic construct
  REGION 1..17: note = Synthetic polypeptide
VISYDGSIKYYADSVKG

SEQ ID NO: 66 - protein, 14 aa, synthetic construct
  REGION 1..14: note = Synthetic polypeptide
TGEYSGYDTDPQYS

SEQ ID NO: 67 - protein, 11 aa, synthetic construct
  REGION 1..11: note = Synthetic polypeptide
RSSQGIGDDLG

SEQ ID NO: 68 - protein, 7 aa, synthetic construct
  REGION 1..7: note = Synthetic polypeptide
GTSTLQS

SEQ ID NO: 69 - protein, 9 aa, synthetic construct
  REGION 1..9: note = Synthetic polypeptide
LQDSNYPLT

SEQ ID NO: 70 - protein, 5 aa, synthetic construct
  REGION 1..5: note = Synthetic polypeptide
DKTHT

SEQ ID NO: 71 - protein, 4 aa, synthetic construct
  REGION 1..4: note = Synthetic polypeptide
CPPC

SEQ ID NO: 72 - protein, 15 aa, synthetic construct
  REGION 1..15: note = Synthetic polypeptide
CPEPKSCDTPPPCPR

SEQ ID NO: 73 - protein, 12 aa, synthetic construct
  REGION 1..12: note = Synthetic polypeptide
ELKTPLGDTTHT

SEQ ID NO: 74 - protein, 10 aa, synthetic construct
  REGION 1..10: note = Synthetic polypeptide
KSCDKTHTCP

SEQ ID NO: 75 - protein, 7 aa, synthetic construct
  REGION 1..7: note = Synthetic polypeptide
KCCVDCP

SEQ ID NO: 76 - protein, 7 aa, synthetic construct
  REGION 1..7: note = Synthetic polypeptide
KYGPPCP

SEQ ID NO: 77 - protein, 15 aa, synthetic construct
  REGION 1..15: note = Synthetic polypeptide
EPKSCDKTHTCPPCP

SEQ ID NO: 78 - protein, 17 aa, synthetic construct
  REGION 1..17: note = Synthetic polypeptide
ELKTPLGDTTHTCPRCP

SEQ ID NO: 79 - protein, 12 aa, synthetic construct
  REGION 1..12: note = Synthetic polypeptide
SPNMVPHAHHAQ

SEQ ID NO: 80 - protein, 12 aa, synthetic construct
  REGION 1..12: note = Synthetic polypeptide
ESKYGPPCPPCP

SEQ ID NO: 81 - protein, 15 aa, synthetic construct
  REGION 1..15: note = Synthetic polypeptide
EPKSCDKTYTCPPCP

SEQ ID NO: 82 - protein, 45 aa, synthetic construct
  REGION 1..45: note = Synthetic polypeptide
TTTPAPRPPTPAPTIASQPLSLRPEACRPAAGGAVHTRGLDFACD

SEQ ID NO: 83 - protein, 110 aa, synthetic construct
  REGION 1..110: note = Synthetic polypeptide
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 84 - protein, 107 aa, synthetic construct
  REGION 1..107: note = Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 27 aa, synthetic construct
  REGION 1..27: note = Synthetic polypeptide
FWVLVVVGGVLACYSLLVTVAFIIFWV

SEQ ID NO: 86 - protein, 23 aa, synthetic construct
  REGION 1..23: note = Synthetic polypeptide
LGLLVAGVLVLLVSLGVAIHLCC

SEQ ID NO: 87 - protein, 25 aa, synthetic construct
  REGION 1..25: note = Synthetic polypeptide
ALIVLGGVAGLLLFIGLGIFFCVRC

SEQ ID NO: 88 - protein, 23 aa, synthetic construct
  REGION 1..23: note = Synthetic polypeptide
LCYLLDGILFIYGVILTALFLRV

SEQ ID NO: 89 - protein, 26 aa, synthetic construct
  REGION 1..26: note = Synthetic polypeptide
WVLVVVGGVLACYSLLVTVAFIIFWV

SEQ ID NO: 90 - protein, 26 aa, synthetic construct
  REGION 1..26: note = Synthetic polypeptide
VAAILGLGLVLGLLGPLAILLALYLL

SEQ ID NO: 91 - protein, 24 aa, synthetic construct
  REGION 1..24: note = Synthetic polypeptide
ALPAALAVISFLLGLGLGVACVLA

SEQ ID NO: 92 - protein, 113 aa, synthetic construct
  REGION 1..113: note = Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 93 - protein, 112 aa, synthetic construct
  REGION 1..112: note = Synthetic polypeptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 94 - protein, 102 aa, synthetic construct
  REGION 1..102: note = Synthetic polypeptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 95 - protein, 101 aa, synthetic construct
  REGION 1..101: note = Synthetic polypeptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 96 - protein, 86 aa, synthetic construct
  REGION 1..86: note = Synthetic polypeptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 97 - protein, 21 aa, synthetic construct
  REGION 1..21: note = Synthetic polypeptide
DGVYTGLSTRNQETYETLKHE

SEQ ID NO: 98 - protein, 171 aa, synthetic construct
  REGION 1..171: note = Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 99 - protein, 127 aa, synthetic construct
  REGION 1..127: note = Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 100 - protein, 21 aa, synthetic construct
  REGION 1..21: note = Synthetic polypeptide
DQVYQPLRDRDDAQYSHLGGN

SEQ ID NO: 101 - protein, 207 aa, synthetic construct
  REGION 1..207: note = Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 102 - protein, 21 aa, synthetic construct
  REGION 1..21: note = Synthetic polypeptide
NPDYEPIRKGQRDLYSGLNQR

SEQ ID NO: 103 - protein, 182 aa, synthetic construct
  REGION 1..182: note = Synthetic polypeptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 104 - protein, 21 aa, synthetic construct
  REGION 1..21: note = Synthetic polypeptide
DQLYQPLKDREDDQYSHLQGN

SEQ ID NO: 105 - protein, 163 aa, synthetic construct
  REGION 1..163: note = Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 164 aa, synthetic construct
  REGION 1..164: note = Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 107 - protein, 21 aa, synthetic construct
  REGION 1..21: note = Synthetic polypeptide
NQLYNELNLGRREEYDVLDKR

SEQ ID NO: 108 - protein, 22 aa, synthetic construct
  REGION 1..22: note = Synthetic polypeptide
EGLYNELQKDKMAEAYSEIGMK

SEQ ID NO: 109 - protein, 21 aa, synthetic construct
  REGION 1..21: note = Synthetic polypeptide
DGLYQGLSTATKDTYDALHMQ

SEQ ID NO: 110 - protein, 226 aa, synthetic construct
  REGION 1..226: note = Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 111 - protein, 188 aa, synthetic construct
  REGION 1..188: note = Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 112 - protein, 21 aa, synthetic construct
  REGION 1..21: note = Synthetic polypeptide
ENLYEGLNLDDCSMYEDISRG

SEQ ID NO: 113 - protein, 68 aa, synthetic construct
  REGION 1..68: note = Synthetic polypeptide
FWVLVVVGGVLACYSLLVTVAFIIFWVRSKRSRLLHSDYMNMTPRRPGPTRKHYQPYAPPRDFAAYRS

SEQ ID NO: 114 - protein, 44 aa, synthetic construct
  REGION 1..44: note = Synthetic polypeptide
FWVRSKRSRLLHSDYMNMTPRRPGPTRKHYQPYAPPRDFAAYRS

SEQ ID NO: 115 - protein, 35 aa, synthetic construct
  REGION 1..35: note = Synthetic polypeptide
TKKKYSSSVHDPNGEYMFMRAVNTAKKSRLTDVTL

SEQ ID NO: 116 - protein, 37 aa, synthetic construct
  REGION 1..37: note = Synthetic polypeptide
RRDQRLPPDAHKPPGGGSFRTPIQEEQADAHSTLAKI

SEQ ID NO: 117 - protein, 114 aa, synthetic construct
  REGION 1..114: note = Synthetic polypeptide
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 118 - protein, 49 aa, synthetic construct
  REGION 1..49: note = Synthetic polypeptide
HQRRKYRSNKGESPVEPAEPCRYSCPREEEGSTIPIQEDYRKPEPACSP

SEQ ID NO: 119 - protein, 187 aa, synthetic construct
  REGION 1..187: note = Synthetic polypeptide
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 120 - protein, 54 aa, synthetic construct
  REGION 1..54: note = Synthetic polypeptide
HIWQLRSQCMWPRETQLLLEVPPSTEDARSCQFPEEERGERSAEEKGRLGDLWV

SEQ ID NO: 121 - protein, 60 aa, synthetic construct
  REGION 1..60: note = Synthetic polypeptide
CVKRRKPRGDVVKVIVSVQRKRQEAEGEATVIEALQAPPDVTTVAVEETIPSFTGRSPNH

SEQ ID NO: 122 - protein, 9 aa, synthetic construct
  REGION 1..9: note = Synthetic polypeptide
YPYDVPDYA

SEQ ID NO: 123 - protein, 10 aa, synthetic construct
  REGION 1..10: note = Synthetic polypeptide
EQKLISEEDL

SEQ ID NO: 124 - protein, 5 aa, synthetic construct
  REGION 1..5: note = Synthetic polypeptide
GSSSG

SEQ ID NO: 125 - protein, 4 aa, synthetic construct
  REGION 1..4: note = Synthetic polypeptide
GGSG

SEQ ID NO: 126 - protein, 5 aa, synthetic construct
  REGION 1..5: note = Synthetic polypeptide
GGSGG

SEQ ID NO: 127 - protein, 5 aa, synthetic construct
  REGION 1..5: note = Synthetic polypeptide
GSGSG

SEQ ID NO: 128 - protein, 5 aa, synthetic construct
  REGION 1..5: note = Synthetic polypeptide
GSGGG

SEQ ID NO: 129 - protein, 5 aa, synthetic construct
  REGION 1..5: note = Synthetic polypeptide
GGGSG

SEQ ID NO: 130 - protein, 5 aa, synthetic construct
  REGION 1..5: note = Synthetic polypeptide
EAAAK

SEQ ID NO: 131 - protein, 792 aa, synthetic construct
  REGION 1..792: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGRGLLRGLWPLHIVLWTRIASTIPPYPYDVPDYAHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 132 - protein, 568 aa, synthetic construct
  REGION 1..568: note = Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 133 - protein, 614 aa, synthetic construct
  REGION 1..614: note = Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 134 - protein, 800 aa, synthetic construct
  REGION 1..800: note = Synthetic polypeptide
METDTLLLWVLLLWVPGSTGAGGSDYKDDDDKGGSLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGRGLLRGLWPLHIVLWTRIASTIPPYPYDVPDYAHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 135 - protein, 794 aa, synthetic construct
  REGION 1..794: note = Synthetic polypeptide
METDTLLLWVLLLWVPGSTGAGGSDYKDDDDKGGSSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGRGLLRGLWPLHIVLWTRIASTIPPYPYDVPDYAHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 136 - protein, 545 aa, synthetic construct
  REGION 1..545: note = Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 591 aa, synthetic construct
  REGION 1..591: note = Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 545 aa, synthetic construct
  REGION 1..545: note = Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 139 - protein, 591 aa, synthetic construct
  REGION 1..591: note = Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRRGRRRLLYIFRQPFMRPVQTTQEEDGCSCRFPEEEEGGCELRVRFSRSADAPAYQQGQNQLYNELNLGRREEYDVLDRRRGRDPEMGGRPRRRNPQEGLYNELQRDRMAEAYSEIGMRGERRRGRGHDGLYQGLSTATRDTYDALHMQALPPR

SEQ ID NO: 140 - protein, 547 aa, synthetic construct
  REGION 1..547: note = Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 593 aa, synthetic construct
  REGION 1..593: note = Synthetic polypeptide
METDTLLLWVLLLWVPGSTGSPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 553 aa, synthetic construct
  REGION 1..553: note = Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 599 aa, synthetic construct
  REGION 1..599: note = Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 555 aa, synthetic construct
  REGION 1..555: note = Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 601 aa, synthetic construct
  REGION 1..601: note = Synthetic polypeptide
METDTLLLWVLLLWVPGSTGLTCLGGFASPGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 558 aa, synthetic construct
  REGION 1..558: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 112 aa, synthetic construct
  REGION 1..112: note = Synthetic polypeptide
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 148 - protein, 599 aa, synthetic construct
  REGION 1..599: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 604 aa, synthetic construct
  REGION 1..604: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 605 aa, synthetic construct
  REGION 1..605: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSTSGSGKPGSGEGSTKGDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 822 aa, synthetic construct
  REGION 1..822: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSTSGSGKPGSGEGSTKGDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 607 aa, synthetic construct
  REGION 1..607: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGSTSGSGKPGSGEGSTKGDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 824 aa, synthetic construct
  REGION 1..824: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGSTSGSGKPGSGEGSTKGDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRAESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 1055 aa, synthetic construct
  REGION 1..1055: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMETDTLLLWVLLLWVPGSTGTSYPYDVPDYAGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 1102 aa, synthetic construct
  REGION 1..1102: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSTSGSGKPGSGEGSTKGDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMETDTLLLWVLLLWVPGSTGTSYPYDVPDYAGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 1321 aa, synthetic construct
  REGION 1..1321: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGSTSGSGKPGSGEGSTKGDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRAESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMETDTLLLWVLLLWVPGSTGTSYPYDVPDYAGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 870 aa, synthetic construct
  REGION 1..870: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGSTSGSGKPGSGEGSTKGDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRAGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 868 aa, synthetic construct
  REGION 1..868: note = Synthetic polypeptide
METDTLLLWVLLLWVPGSTGAGGSDYKDDDDKGGSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGGGGSGGGGSGGGGSGGGGSEIQLVQSGAEVKKPGESLRISCKGSGFNIEDYYIHWVRQMPGKGLEWMGRIDPENDETKYGPIFQGHVTISADTSINTVYLQWSSLKASDTAMYYCAFRGGVYWGQGTTVTVSSGSTSGSGKPGSGEGSTKGDVVMTQSPDSLAVSLGERATINCKSSQSLLDSDGKTYLNWLQQKPGQPPKRLISLVSKLDSGVPDRFSGSGSGTDFTLTISSLQAEDVAVYYCWQGTHFPGTFGGGTKVEIKGGGGSGGGGSGGGGSGGGGSQVQLVQSGAEVKKPGSSVKVSCKASGYTFSSNVISWVRQAPGQGLEWMGGVIPIVDIANYAQRFKGRVTITADESTSTTYMELSSLRSEDTAVYYCALPRAFVLDAMDYWGQGTLVTVSSGGGGSGGGGSGGGGSETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 5 aa, synthetic construct
  REGION 1..5: note = Synthetic polypeptide
GSGGS

SEQ ID NO: 160 - protein, 4 aa, synthetic construct
  REGION 1..4: note = Synthetic polypeptide
GGGS

SEQ ID NO: 161 - protein, 5 aa, synthetic construct
  REGION 1..5: note = Synthetic polypeptide
GGGGS